(19) United States
(12) Patent Application Publication
(10) Pub. No.: US 2015/0031861 A1
(43) Pub. Date: Jan. 29, 2015

(54) Chemical Modification of Antibodies

(71) Applicant: UCL Business PLC, London (GB)

(72) Inventors: Mark Smith, London (GB); James Baker, London (GB); Felix Schumacher, London (GB); Stephen Caddick, London (GB); Vijay Chudasama, London (GB); Antoine Maruani, London (GB)

(21) Appl. No.: 14/383,260

(22) PCT Filed: Mar. 8, 2013
(86) PCT No.: PCT/GB2013/050581

§ 371 Date: Sep. 5, 2014

Related U.S. Application Data
(60) Provisional application No. 61/608,709, filed on Mar. 9, 2012.

(51) Int. Cl. A61K 47/48; A61K 31/704; C07K 16/18

(52) U.S. Cl. CPC A61K 47/48384; C07K 16/18; A61K 31/704; C07K 2317/50
USPC 530/387.3; 530/391.7; 530/391.3

(57) ABSTRACT

The present invention relates to antibodies and antibody fragments, one or more of whose native inter-chain disulfide bridges have been replaced with a specific bridging moiety. The bridging moiety can be selectively targeted to inter-chain disulfide bonds within the antibody or antibody fragment, enabling the construction of more homogenously modified products such as antibody-drug conjugates.

The invention relates to chemical modification of antibodies and antibody fragments. In particular, the invention relates to methods for achieving selective modification of antibodies and antibody fragments across one or more their native inter-chain disulfide bridges, as well as to related and product obtainable via such selective methods.

### BACKGROUND

Monoclonal antibodies (mAbs) represent the fastest growing class of therapeutics and have the potential to provide effective treatments across a range of clinical areas, including oncology, infectious diseases, inflammatory diseases and cardiovascular medicine. The global market for antibodies is currently estimated at around $50 billion.

The chemical modification of antibodies is a key technological challenge in the area, as it allows the attachment of “cargo” (or “functional”) moieties that enable optimisation of the in vivo properties of the antibody (e.g. improved pharmacokinetics) or confer upon it new functions and activities (e.g. the attachment of a drug or an imaging agent).

Currently, however, the state-of-the-art in the chemical modification of antibodies is far from ideal. It relies upon the following methods:
a) the unselective conjugation to native lysine residues, which affords heterogeneous mixtures and frequently loss of activity;
b) mutagenesis to incorporate single cysteines as sites for attachment, which is synthetically inconvenient and can lead to problematic protein expression and disulfide exchange and aggregation; or
c) reduction of native disulfide bonds, to afford two cysteines residues for conjugation, which can lead to reduced stability of the antibody due to loss of the key bridging motif, and again heterogeneous mixtures of products formed.

Benefits of achieving a greater degree of homogeneity in antibody modification in affording antibody-drug-conjugates (“ADCs”)—a key, and rapidly growing, part of the global antibody market—would include improved therapeutic index and pharmacokinetics. New methods for selective modification of antibodies to afford more homogeneous conjugates are thus currently being keenly sought.

Consequently, there is a need in the art for new methods to selectively modify antibodies and for provision of chemically modified antibodies that have a greater degree of homogeneity than is generally achieved using prior art methods.

This patent application describes antibodies and antibody fragments, one or more of whose native inter-chain disulfide bridges have been replaced with a specific, synthetic bridging moiety. The bridging moiety can be selectively targeted to inter-chain, rather than intra-chain, disulfide bonds, and moreover to specific inter-chain disulfide bonds, enabling the construction of more homogeneous chemically modified antibodies (for example, more homogeneous bioconjugates such as ADCs when the bridging moiety also carries one or more cargo moieties).

### SUMMARY

The present inventors have identified that a specific class of maleimide and 3,6-dioxopyridazine compounds can be used to selectively target, and replace, inter-chain disulfide bridges in antibodies and antibody fragments when reacted therewith under suitable reaction conditions. The chemical modification occurs preferentially at inter-chain disulfide bridges rather than intra-chain disulfide bridges and can also be controlled so as to occur at selected inter-chain disulfide bridges in preference to other inter-chain disulfide bridges present in the antibody or antibody fragment.

Chemically modified antibodies and antibody fragments incorporating these inter-chain bridging moieties are thus less heterogeneous than in prior art methods. Furthermore, there is generally no need to effect mutagenesis synthetic steps to introduce artificial residues that can then serve as the basis for chemical modification. Still further, the inter-chain bridging moieties described herein ensure that the structural integrity, and functionality, of the native antibody or antibody fragment is retained since they mimic the structure of the native inter-chain disulfide bridges that they have replaced.

Consequently, the present inventors have obtained selectively modified antibodies and antibody fragments that carry characteristic inter-chain bridging moieties. The bridging moieties may themselves further carry one or more cargo moieties, thus leading to the provision of conjugates whose antibody (or antibody fragment) component has been selectively functionalised. In the case of 3,6-dioxopyridazine modification, it is particularly facile to incorporate multiple cargo moieties, for example both a drug or imaging agent and a half-life extending agent, on a single inter-chain bridging moiety scaffold. Related synthetic methods, products and uses are also provided, as described in more detail herein.

Thus, the present invention provides a chemically modified antibody AB that:
(i) is capable of specific binding to an antigen AG;
(ii) comprises four chains, two of which are heavy chains and two of which are light chains; and
(iii) comprises at least one inter-chain bridging moiety of the formula (IA) or at least one inter-chain bridging moiety of the formula (IB)

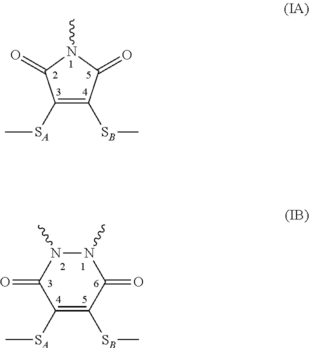

wherein S_{A} and S_{B} are sulfur atoms that are attached to different chains of said chemically modified antibody.

Also provided is a process for selectively producing a chemically modified antibody of the present invention, which process comprises:
reducing at least one inter-chain disulfide bridge of an antibody in the presence of a reducing agent; and
reacting said antibody with at least one inter-chain bridging reagent of the formula (HA) or at least one inter-chain bridging moiety of the formula (IIB)

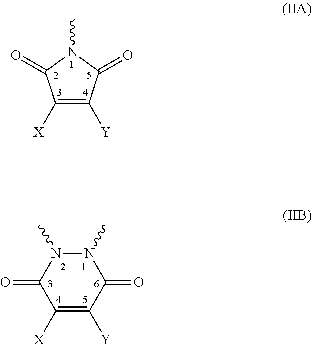

wherein X and Y each independently represent an electrophilic leaving group; thereby introducing the desired number of inter-chain bridging moieties of the formula (IA) or (IB) at the desired locations of said antibody and producing said chemically modified antibody.

The present invention further provides a chemically modified antibody AB that:
(i) is capable of specific binding to an antigen AG;
(ii) comprises four chains, two of which are heavy chains and two of which are light chains; and
(iii) comprises at least one inter-chain bridging moiety of the formula (III)

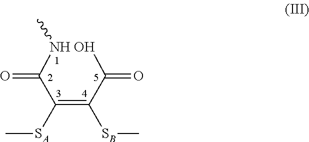

wherein S_{A} and S_{B} are sulfur atoms that are attached to different chains of said chemically modified antibody.

The present invention also provides a chemically modified antibody fragment AB_{F} that:
(i) is capable of specific binding to an antigen AG;
(ii) comprises at least two chains; and
(iii) comprises at least one inter-chain bridging moiety of the formula (IA_{F}) or at least one inter-chain bridging moiety of the formula (IB_{F})

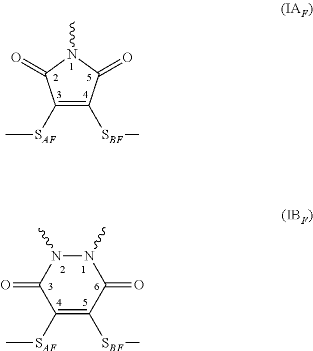

wherein S_{AF} and S_{BF} are sulfur atoms that are attached to different chains of said chemically modified antibody fragment.

Still further, the present invention provides a process for producing a chemically modified antibody fragment of the present invention, which process comprises:
reducing at least one inter-chain disulfide bridge of an antibody fragment in the presence of a reducing agent; and
reacting said antibody fragment with at least one inter-chain bridging reagent comprising a moiety of the formula (IIA) or at least one inter-chain bridging reagent comprising a moiety of the formula (IIB)

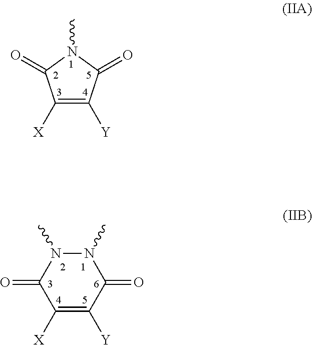

wherein X and Y each independently represent an electrophilic leaving group;
thereby introducing the desired number of inter-chain bridging moieties of the formula (IA_{F}) or (IB_{F}) at the desired locations of said antibody fragment and producing said chemically modified antibody fragment.

The present invention further provides a chemically modified antibody fragment AB_{F} that:
(i) is capable of specific binding to an antigen AG;
(ii) comprises at least two chains; and
(iii) comprises at least one inter-chain bridging moiety of the formula (III_{F})

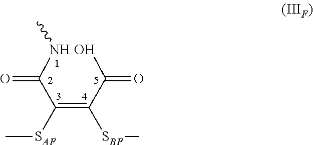

wherein S_{AF} and S_{BF} are sulfur atoms that are attached to different chains of said chemically modified antibody fragment.

In addition, the present invention provides a composition comprising one or more chemically modified antibodies of the present invention and which are each capable of binding to the antigen AG, wherein a specific chemically modified antibody of said one or more chemically modified antibodies is:
(i) present in a greater amount by weight than any other of the said one or more chemically modified antibodies; and
(ii) present in an amount of at least 30% by weight of the total amount of said one or more chemically modified antibodies.

Still further, the present invention provides use of an inter-chain bridging reagent of the formula (IIA) or (IIB)

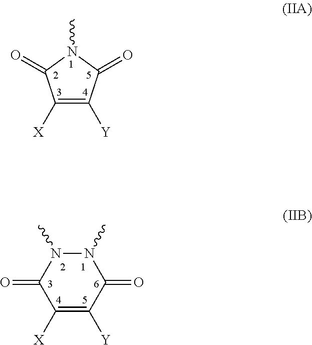

wherein X and Y each independently represent an electrophilic leaving group,
for effecting selective chemical modification of an antibody via the selective replacement of one or more of the inter-chain disulfide bonds in said antibody by inter-chain bridging moieties of the formula (IA) or (IB)

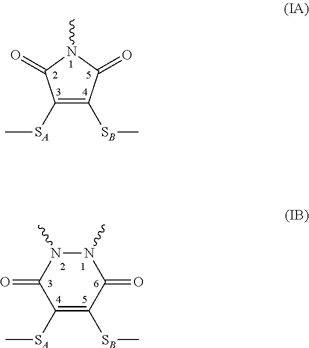

wherein S_{A} and S_{B} are sulfur atoms that are attached to different chains of the resulting chemically modified antibody.

Further preferred features and embodiments are described in the accompanying description and the appended claims.

### BRIEF DESCRIPTION OF THE FIGURES

FIG. 1 depicts LCMS spectra obtained when reducing anti-CEA as in Example 2.3: A corresponds to unmodified anti-CEA; B corresponds to reduction with TCEP; C corresponds to reduction with 2-mercaptoethanol; and D corresponds to reduction with DTT.

FIG. 2 depicts the results of adding DTT to anti-CEA and incubating the mixture over time, as monitored by LCMS, as described in Example 2.4: filled circles correspond to 10 equivalents of DTT under low-salt conditions; filled triangles correspond to 10 equivalents of DTT under high-salt conditions; open circles correspond to 20 equivalents of DTT under low-salt conditions; and open triangles correspond to 20 equivalents of DTT under high-salt conditions.

FIG. 3 depicts LCMS spectra obtained when bridging anti-CEA according to Example 2.5: A corresponds to unmodified anti-CEA; B corresponds to the sample after reaction for 5 minutes.

FIG. 4 depicts results of bridging anti-CEA according to Example 2.6 using various amounts of reducing agent and bridging reagent: A shows the performance of various sample mixtures; B is an LCMS spectrum of unmodified anti-CEA; and C is an LCMS spectrum obtained when bridging with 15 equivalents of both reducing agent and bridging reagent.

FIG. 5 depicts the results of bridging anti-CEA, as monitored by LCMS, according to Example 2.7.

FIG. 6 depicts the results of modification and functionalisation of anti-CEA according to Example 2.8: A is an LCMS spectrum of unmodified anti-CEA; B is an LCMS spectrum of biotinylated anti-CEA; C is an LCMS spectrum of anti-CEA-fluorescein; D is an LCMS spectrum of alkylated anti-CEA; E is a UV trace of unmodified anti-CEA; F is a UV trace of PEGylated anti-CEA; G is an SDS-PAGE analysis of PEGylated anti-CEA; and H is a MALDI-TOF analysis of PEGylated anti-CEA (the left peak is de-PEGylated protein generated by the laser impact).

FIG. 7 depicts the results of in situ functionalisation of anti-CEA, as monitored by LCMS, according to Example 2.9.

FIG. 8 depicts the results of in situ functionalisation of anti-CEA, as monitored by LCMS, according to Example 2.10: closed circles are results obtained using 2 equivalents of bridging reagent and open squares are results obtained using 5 equivalents of bridging reagent.

FIG. 9 shows the results of in situ bridging of anti-CEA in a two-step protocol with 2 equivalents of bridging reagents and variable amounts of reducing agent as monitored by LCMS, according to Example 2.11. Also shown are results obtained where a total of 20 equivalents of reducing agent were used when 1.5 equivalents or 1.2 equivalents of bridging reagent were used (white column and black column, respectively).

FIG. 10 depicts the fluorescence of anti-CEA-fluorescein monitored by UV/Vis spectroscopy according to Example 2.12: dotted line is unmodified anti-CEA; filled line is 5 μg/ml anti-CEA-fluorescein and hashed line is 25 μg/ml anti-CEA-fluorescein.

FIG. 11 depicts SDS-PAGE analysis of the synthesis of anti-CEA-HRP conjugate according to Example 2.13: (1) Biotinylated anti-CEA; (2) Unmodified anti-CEA; (3) Mix of unmodified anti-CEA and the HRP/STREP conjugate; (4) HRP/STREP conjugate; (5) 15 μl; (6) 12 μl; (7) 10 μl; (8) 8 μl; (9) 6 μl; (10) 4 μl; (11) 2 μl; and (12) 1 μl.

FIG. 12 depicts the results of one step ELISA with an anti-CEA-HRP conjugate according to Example 2.14: A shows an SDS-PAGE analysis of the purified conjugate in which 1 is unmodified anti-CEA, 2 is biotinylated anti-CEA, 3 is HRP/STREP conjugate, 4 is a mix of anti-CEA with HRP/STREP conjugate and 5 is purified anti-CEA-HRP conjugate; B shows an activity test of the anti-CEA-HRP conjugate; C shows the results of one-step ELISA against increasing amounts of antigen and D shows the results of one-step ELISA with decreasing amounts of the anti-CEA-HRP conjugate.

FIG. 13 depicts the results of two-step ELISA with anti-CEA-HRP according to Example 2.15: open circles are anti-CEA-biotin with the primary and secondary antibody mix; open triangles are results with a 1:460 dilution of the HRP/STREP conjugate; and filled circles are results with a 1:4600 dilution of the HRP/STREP conjugate.

FIG. 14 depicts ELISA studies of functionally bridged anti-CEAs as described in Example 2.16: in the left-hand graph open circles are anti-CEA, open triangles are processed anti-CEA, filled circles are sequentially bridged anti-CEA and filled triangles are in situ bridged anti-CEA; in the right-hand graph open circles are processed anti-CEA, open triangles are anti-CEA-biotin, filled circles are anti-CEA-fluorescein and filled triangles are anti-CEA-PEG5000.

FIG. 15 depicts ELISA results on functionally bridged anti-CEA as described in Example 2.17: open circles are “old” bridge anti-CEA, open triangles are “fresh” bridged anti-CEA, closed circles are “old” anti-CEA-PEG5000 and closed triangles are “fresh” anti-CEA-PEG5000.

FIG. 16 depicts the results of fluorescence-based cell ELISA as described in Example 2.18, where black columns relate to CAPAN-1 cells and grey columns relate to control A375 cells.

FIG. 17 depicts LCMS results of a stability test of bridged anti-CEA against various reducing agents as described in Example 2.20: filled circles relate to 2-mercaptoethanol, open squares to dithiothreitol and filled triangles to glutathione.

FIG. 18 depicts the results of tests on the plasma stability of bridged anti-CEA as described in Example 2.21: A shows SDS-PAGE after short incubation in human plasma, where 1+2+3 are loading control with unmodified anti-CEA (1, 3, 5 μg, respectively), 4 shows nickel beads purification background, 5 shows results at 1 h, 6 at 4 h and 7 at 24 h; B shows SDS-PAGE after long incubation in human plasma, where 1+2+3 are loading control with unmodified anti-CEA (1, 3, 5 μg, respectively), 4 shows results at 3 d, 5 at 5 d, 6 at 7 d, 7 at 7 d with unmodified anti-CEA and 8 at 7 d with alkylated anti-CEA; C shows SDS-PAGE of nickel beads performance control, where 1 is unmodified anti-CEA, 2 is bridged anti-CEA, 3 is alkylated anti-CEA, 4 is a mix purified from PBS and 5 is a mix purified from human plasma; D shows MS after 1 h in human plasma; E shows MS after 3 d in human plasma; F shows MS after 7 d in human plasma; G shows MS of unmodified anti-CEA after 7 d in human plasma; and H shows MS of alkylated anti-CEA after 7 d in human plasma.

FIG. 19 depicts the results of ELISA measurement of the activity of anti-CEA analogues following incubation in human plasma as described in Example 2.22: open circles are processed sscFv, open triangles are bridged sscFv, filled circles are alkylated sscFv and filled squares are PEG-sscFv.

FIG. 20 depicts the results of reduction of Rituximab according to Example 3.2: A is an SDS-PAGE analysis showing reduction with TCEP where 1 is unmodified antibody, 2 is antibody+DMF, 3 is 5 equiv., 4 is 10 equiv., 5 is 20 equiv., 6 is 40 equiv., 7 is 60 equiv., 8 is 80 equiv. and 9 is 100 equiv; B shows an MS of intact antibody; and C shows an MS of reduced antibody.

FIG. 21 shows an SDS-PAGE analysis of the in situ antibody bridging described in Example 3.3: 1) unmodified antibody. 2) antibody+DMF. 3) 3 equiv. 4) 5 equiv. 5) 10 equiv. 6) 20 equiv. 7) 5 equiv. 8) 20 equiv. 9) 40 equiv. and 10) 80 equiv.

FIG. 22 shows an SDS-PAGE analysis of in situ PEGylation of antibody as described in Example 3.4: 1) unmodified antibody. 2) antibody+DMF. 3) 3 equiv. 4) 5 equiv. 5) 10 equiv. 6) 20 equiv. 7) 5 equiv. 8) 20 equiv. 9) 40 equiv. and 10) 80 equiv.

FIG. 23 depicts the results of PEGylation of Rituximab as described in Example 3.5: A shows SDS-PAGE analysis of in situ PEGylation with various reducing agents, as follows: 1) unmodified antibody; 2) antibody+80 equiv PEG; 3) 10 equiv TCEP/20 equiv PEG; 4) 10 equiv TCEP; 5) 40 equiv TCEP/80 equiv PEG; 6) 40 equiv TCEP; 7) 10 equiv Se/20 equiv PEG; 8) 10 equiv Se; 9) 40 equiv Se/80 equiv PEG; and 10) 40 equiv Se; B shows an MS of unmodified antibody; C shows an MS of sample 3; D shows an MS of sample 5; E shows an MS of sample 7; and F shows an MS of sample 9.

FIG. 24 depicts an SDS-PAGE analysis of sequential bridging of Rituximab as described in Example 3.6: 1) unmodified antibody. 2) antibody+80 equiv+DMF. 3) antibody+TCEP. 4) 5 equiv. 5) 10 equiv. 6) 20 equiv. 7) 30 equiv. 8) 40 equiv. 9) 60 equiv. and 10) 80 equiv.

FIG. 25 depicts an SDS-PAGE analysis of stepwise in situ PEGylation of Rituximab as described in Example 3.6: 1) unmodified antibody. 2) antibody+80 equiv. 3) antibody+TCEP. 4) 5 equiv. 5) 10 equiv. 6) 20 equiv. 7) 30 equiv. 8) 40 equiv. 9) 60 equiv. 10) 80 equiv. 11) antibody+25 equiv. 12) 5 equiv. 13) 10 equiv. 14) 20 equiv. and 15) 25 equiv.

FIG. 26 depicts an SDS-PAGE analysis of an “alternative” reduction of Rituximab as described in Example 3.7: 1) unmodified antibody. 2) 5 equiv DTT. 3) 10 equiv DTT. 4) 20 equiv DTT. 5) 50 equiv DTT. 6) 5 equiv bME. 7) 10 equiv bME. 8) 20 equiv bME. And 9) 50 equiv bME.

FIG. 27 depicts an SDS-PAGE analysis of an “alternative” PEGylation of Rituximab as described in Example 3.8: 1) unmodified antibody. 2) 15 equiv. 3) 20 equiv. 4) 25 equiv. 5) 30 equiv. and 6) antibody+30 equiv.

FIG. 28 depicts an SDS-PAGE analysis of mixed reduction of Rituximab as described in Example 3.9: 1) unmodified antibody. 2) antibody+TCEP. 3) 10 equiv. 4) 20 equiv. and 5) 50 equiv.

FIG. 29 depicts an SDS-PAGE analysis of mixed PEGylation of Rituximab as described in Example 3.10: 1) unmodified antibody. 2) antibody+10 equiv. 3) antibody+TCEP+DTT. 4) 3 equiv. 5) 5 equiv. 6) 10 equiv. 7) antibody+30 equiv. 8) 15 equiv. 9) 20 equiv. 10) 25 equiv. and 11) 30 equiv.

FIG. 30 depicts the results of comparison between the “in situ” vs. “sequential” conditions for PEGylation of Rituximab as described in Example 3.11: A shows an SDS-PAGE analysis where 1 is unmodified antibody, 2 is antibody+DMF+60 equiv PEG, 3 is 40 equiv Se, 4 is 40 equiv Se+10 equiv PEG, 5 is 30 equiv Se, 6 is 30 equiv Se+60 equiv PEG, 7 is 20 equiv Se, 8 is 20 equiv Se+40 equiv PEG, 9 is antibody+25 equiv PEG, 10 is 5 equiv TCEP/10 equiv DTT, 11 is 5 equiv TCEP/10 equiv DTT/20 equiv PEG, 12 is 20 equiv DTT, 13 is 20 equiv DTT/25 equiv PEG, 14 is 10 equiv TCEP and 15 is 10 equiv TCEP/20 equiv PEG; B shows an MS of product lane 4; C shows an MS of product lane 6; D shows an MS of product lane 8; E shows an MS of product lane 11; F shows an MS of product lane 13; and G shows an MS of product lane 15.

FIG. 31 depicts an SDS-PAGE analysis of the in situ fluorescent labelling of Rituximab described in Example 3.12: 1) unmodified antibody. 2) antibody+DMF+60 equiv dithiophenolmaleimide. 3) 20 equiv DTT. 4) fluorescein-labelled antibody. 5) 30 equiv Se. and 6) bridged antibody.

FIG. 32 depicts the site-selective PEGylation results described in Example 3.14: A shows SDS-PAGE of digests as follows: 1) unmodified antibody, 2+6) digest of unmodified antibody, 3+7) digest of in situ PEGylated antibody—Yield of Fab=25.0%, 4+8) digest of sequentially PEGylated antibody (TCEP)—Yield of Fab=14.3%, 5+9) digest of sequentially PEGylated antibody (DTT)—Yield of Fab=7.9%; B shows an MS of the Fc region of unmodified antibody; C shows an MS of the Fc region of in situ PEGylated antibody; D shows an MS of the Fc region of sequentially PEGylated antibody (TCEP); E shows an MS of the Fc region of sequentially PEGylated antibody (DTT); F shows an MS of the Fab region of unmodified antibody; G shows an MS of the Fab region of in situ PEGylated antibody; H shows an MS of the Fab region of sequentially PEGylated antibody (TCEP); and I shows an MS of the Fab region of sequentially PEGylated antibody (DTT).

FIG. 33 shows the results of step-wise PEGylation of Rituximab as described in Example 3.15: A shows SDS-PAGE of the reaction wherein 1 is unmodified antibody, 2 is unmodified antibody+10 equiv., 3 is reduced antibody, 4 is 5 equiv., 5 is 8 equiv. and 6 is 10 equiv.; B is an MS of sample lane 4 (LMW species are PEGylated HHL fragments); and C is an MS of sample lane 6.

FIG. 34 depicts the results of a re-oxidation study of Rituximab as described in Example 3.16 (numbers in brackets indicate estimated amount of disulfide bonds present under the assumption that both hinge-region cysteines are formed): 1) reduced antibody. 2) 5 min (4%). 3) 20 min (3%). 4) 40 min (3%). 5) 60 min (3%). 6) 2 h (2%). 7) 4 h (2%). 8) 20 h (1%). 9) 30 h (1%). 10) 40 h (1%).

FIG. 35 depicts the results of step-wise modification of Rituximab according to Example 3.17: A shows SDS-PAGE of reaction (bands on top of the gel (bottom of the wells) indicate aggregation): 1) reduced antibody. 2) reduced antibody+20% v/v DMF. 3) 4 equiv PEG. 4) 8 equiv PEG. 5) 12 equiv PEG. 6) 16 equiv PEG. 7) 4 equiv diTPMM. 8) 8 equiv diTPMM. 9) 12 equiv diTPMM. 10) 16 equiv diTPMM; B is an MS of sample lane 6 (LMW species are PEGylated HHL fragments); and C is an MS of sample lane 10 (LMW species are potentially bridged HHL fragments).

FIG. 36 depicts flow-cytometric analysis of the activity of functionalised Rituximab, as described in Example 3.18: A shows cell viability and staining efficiency where sample ID is as follows: 1) Isotype control. 2) Unmodified/untreated antibody. 3) Processed antibody. 4) In situ PEGylated antibody (40 equiv benzeneselenol+10 equiv PEG). 5) In situ PEGylated antibody (30 equiv benzene-selenol+60 equiv PEG). 6) In situ PEGylated antibody (20 equiv benzeneselenol+40 equiv PEG). 7) Sequentially PEGylated antibody (TCEP+DTT). 8) Sequentially PEGylated antibody (TCEP). 9) Sequentially PEGylated antibody (DTT). 10) Sequentially functionalised antibody (fluorescein-labelled). 11) In situ functionalised antibody (bridged). 12) In situ functionalised antibody (n.a.); B shows relative staining efficiency where sample ID is as in A; C-G shows histograms where sample ID is as in A, filled dark grey=negative control, filled light grey=positive control and in which: C shows influence of antibody treatment (black=unmodified antibody, grey=processed antibody); D shows dilution series (black=10 μg/ml, grey=5 μg/ml, light grey=1 μg/ml); E shows in situ PEGylation (black=4, grey=5, light grey=6); F shows sequential PEGylation (black=7, grey=8, light grey=9); and G shows functionalisation (black=10, grey=11, light grey=12).

FIG. 37 depicts the samples for the stability test of variously modified Rituximab as described in Example 3.19: M) Molecular weight marker; lanes from top are 250, 150, 100, 100, 80, 60, 40, 30, 25, 20 and 15 kDa. AB) Unmodified antibody. 1) With dibromomaleimide sequential bridged antibody. 2) With N-phenyldibromomaleimide bridged and hydrolysed antibody. 3) Partial reduced and alkylated antibody.

FIG. 38 depicts the thermostability assay with rituximab analogues of Example 3.19. Melting temperatures shown are the calculated average. (A) In situ PEGylated antibody. Numbers in brackets are equiv used of benzeneselenol: N-PEG5000-dithiophenolmaleimide. (B) Sequential PEGylated antibody. (C) Controls and in situ bridged antibody. (D) Samples with various cysteine modifications.

FIG. 39 depicts PEGylation of rituximab fragments as described in Example 3.20. M) Molecular weight marker; lanes from top are 250, 150, 100, 100, 80, 60, 40, 30, 25, 20 and 15 kDa. 1+7) Reduction control with 40 equiv benzeneselenol. 2+8) In situ PEGylation with a 40:10 ratio of benzeneselenol:N-PEG5000-dtihiophenolmaleimide. 3+9) Reduction control with 10 equiv TCEP (1 h). 4+10) Sequential PEGylation with 20 equiv of PEGylation reagent after reduction with 10 equiv TCEP (1 h). 5+11) Reduction control with 20 equiv DTT (4 h). 6+12) Sequential PEGylation with 25 equiv of N-PEG5000-dithiophenolmaleimide after reduction with 20 equiv DTT (4 h).

FIG. 40 depicts the sequential PEGylation of a mix of rituximab Fab and Fc fragments as described in Example 3.21. Samples were treated with TCEP for 1 h, followed by addition of 20 equiv N-PEG5000-dithiophenolmaleimide. M) Molecular weight marker; lanes from top are 250, 150, 100, 100, 80, 60, 40, 30, 25, 20 and 15 kDa. 1) Fab fragment treated with 10 equiv TCEP and PEGylation reagent. 2) Fc fragment treated with 10 equiv TCEP and PEGylation reagent. 3) 2:1 mix of Fab and Fc treated with 2 equiv, 4) 4 equiv, 5) 6 equiv, 6) 8 equiv, 7) 10 equiv and 8) 15 equiv TCEP before addition of the PEGylation reagent.

FIG. 41 depicts the in situ PEGylation of a mix of rituximab Fab and Fc fragments as described in Example 3.21. Samples were treated with following ratios of benzeneselenol: N-PEG5000-dithiophenolmaleimide. M) Molecular weight marker; lanes from top are 250, 150, 100, 100, 80, 60, 40, 30, 25, 20 and 15 kDa. 1) Fab fragment treated with 30:5. 2) Fc fragment treated with 30:5. 3) 2:1 mix of Fab and Fc treated with 30:2, 4) 60:2, 5) 30:5, 6) 60:5, 7) 30:10 and 8) 60:10.

FIG. 42 depicts the reduction study of Trastuzumab with TCEP under optimised conditions, as described in Example 4.2. M) Molecular weight marker; lanes from top are 250, 150, 100, 100, 80, 60, 40, 30, 25, 20 and 15 kDa. AB) Unmodified antibody. 1) 1 equiv, 2) 2 equiv, 3) 3 equiv, 4) 4 equiv, 5) 5 equiv, 6) 6 equiv and 7) 7 equiv of TCEP.

FIG. 43 depicts in situ bridging and following functionalization with doxorubicin of Trastuzumab as described in Example 4.4. M) Molecular weight marker; lanes from top are 250, 150, 100, 100, 80, 60, 40, 30, 25, 20 and 15 kDa. AB) Unmodified antibody. 1) Sample A (DAR 1.1). 2) Sample B (DAR 2.0). 3) Sample C (DAR 3.1). 4) Sample D (DAR 4.0). The gel was overloaded to visualize the fragmentation pattern.

FIG. 44 depicts treatment of Trastuzumab-DOX with TCEP according to Example 4.6. M) Molecular weight marker; lanes from top are 250, 150, 100, 100, 80, 60, 40, 30, 25, 20 and 15 kDa. 1) Untreated material. 2) 3 equiv TCEP. 3) 5 equiv TCEP. 4) 7 equiv TCEP.

FIG. 45 depicts digest of in situ bridged and functionalised Trastuzumab as described in Example 4.7. M) Molecular weight marker; lanes from top are 250, 150, 100, 100, 80, 60, 40, 30, 25, 20 and 15 kDa. AB) Unmodified antibody. 1) Bridged antibody. 2) Functionalised antibody. 3) Pepsin digest of the functionalised antibody (generating Fab₂) fragments. 4) Papain digest of the Fab₂ fragments of the functionalised antibody (generating Fab fragments). 5) Pepsin digest of the unmodified antibody (generating Fab₂) fragments. 6) Papain digest of the Fab₂ fragments of the modified antibody (generating Fab fragments).

FIG. 46 depicts the stepwise protocol for the modification of Trastuzumab as described in Example 4.8.1. M) Molecular weight marker; lanes from top are 250, 150, 100, 100, 80, 60, 40, 30, 25, 20 and 15 kDa. AB) Unmodified antibody. R sample of reduced Trastuzumab prior to aliquoting and addition of bridging reagent. 1-4) reactions with different bridging reagents at 5 eq.; 1) DTL-1-DOX; 2) DTL-2-DOX; 3) DTL-3-DOX; 4) no bridging reagent added, only DMF was added.

FIG. 47 depicts the sequential protocol for the modification of Trastuzumab as described in Example 4.8.2.1. M) Molecular weight marker; lanes from top are 250, 150, 100, 100, 80, 60, 40, 30, 25, 20 and 15 kDa. AB) Unmodified antibody. R sample of reduced Trastuzumab prior to aliquoting and addition of bridging reagent. 1-5) reactions with different bridging reagents at 5 eq.; 1) DTL-1-DOX; 2) DTL-2-DOX; 4) no bridging reagent added, only DMF was added, reaction at 4° C. 5) no bridging reagent added, only DMF was added.

FIG. 48 depicts the sequential protocol for the modification of Herceptin with DTL-3-DOX as described in Example 4.8.2.2. M) Molecular weight marker; lanes from top are 250, 150, 100, 100, 80, 60, 40, 30, 25, 20 and 15 kDa. AB) Unmodified antibody. R) sample of reduced Herceptin prior to addition of bridging reagent. 1) reaction with DTL-3-DOX (20 eq.) at 25° C., shaking at 400 rpm with added DMF to correct to 10% (v/v) in DMF in the buffer system.

FIG. 49 depicts the in situ protocol for the modification of Trastuzumab as described in Example 4.8.3. M) Molecular weight marker; lanes from top are 250, 150, 100, 100, 80, 60, 40, 30, 25, 20 and 15 kDa. AB) Un-modified antibody. R sample of reduced Trastuzumab without bridging reagent nor DMF. 1-7) reactions with different bridging reagents at 5 eq.; 1) DTL-1-DOX; 2) DTL-2-DOX; 3) DTL-3-DOX; 7) no bridging reagent added, only DMF was added. All reactions were incubated at 37° C., shaking at 400 rpm.

FIG. 50 depicts the stepwise protocol for the modification of Trastuzumab Fab as described in Example 4.8.4. M) Molecular weight marker; lanes from top are 250, 150, 100, 100, 80, 60, 40, 30, 25, 20 and 15 kDa. Fab) Unmodified Fab. R sample of reduced Fab prior to aliquoting and addition of bridging reagent. 1-3) reactions with different bridging reagents at 5 eq. 1) DTL-1-DOX; 2) DTL-2-DOX; 3) DTL-3-DOX; 4) no bridging reagent added, only DMF was added. All reactions were incubated at 25° C., shaking at 400 rpm.

FIG. 51 depicts typical ES-LCMS spectra obtained according to Example 4.8.4, showing Trastuzumab Fab ADC present in sample after conjugation for stepwise protocol with A) DTL-1-DOX with DAR of 1.16, B) DTL-2-DOX with DAR of 0.51, C) DTL-3-DOX with DAR of 0.63.

FIG. 52 depicts the sequential protocol for the modification of Trastuzumab Fab as described in Example 4.8.5. M) Molecular weight marker; lanes from top are 250, 150, 100, 100, 80, 60, 40, 30, 25, 20 and 15 kDa. Fab) Unmodified Fab. R sample of reduced Fab prior to aliquoting and addition of bridging reagent. 1-5) reactions with different bridging re-agents at 5 eq.; 1) DTL-1-DOX; 2) DTL-2-DOX; 3) DTL-3-DOX; 4) no bridging reagent added, only DMF was added; 5) unreduced Fab treated with DTL-1-DOX under same conditions as in 1). All reactions were incubated at 25° C., shaking at 400 rpm.

FIG. 53 depicts typical ES-LCMS spectra obtained according to Example 4.8.5, showing Trastuzumab Fab ADC present in sample after conjugation for sequential protocol with A) DTL-1-DOX with DAR of 1.21, B) DTL-2-DOX with DAR of 0.64, C) DTL-3-DOX with DAR of 0.94.

FIG. 54 depicts an in situ protocol for the modification of Trastuzumab Fab as described in Example 4.8.6. M) Molecular weight marker; lanes from top are 250, 150, 100, 100, 80, 60, 40, 30, 25, 20 and 15 kDa. Fab) Unmodified Fab. 1-4) reactions with different bridging reagents at 5 eq.; 1) DTL-1-DOX; 2) DTL-2-DOX; 3) DTL-3-DOX; 4) no bridging reagent added, only DMF was added. All reactions were incubated at 37° C., shaking at 400 rpm. The gel was overloaded to visualize the fragmentation pattern. Samples were not boiled prior to SDS-PAGE gel analysis.

FIG. 55 depicts typical ES-LCMS spectra obtained according to Example 4.8.6, showing Trastuzumab Fab ADC present in sample after conjugation for in situ protocol with A) DTL-1-DOX with DAR of 1.43, B) DTL-2-DOX with DAR of 0.74, C) DTL-3-DOX with DAR of 1.12.

FIG. 56 depicts binding affinity by ELISA assay for Trastuzumab ADC conjugated with DTL-1-DOX, DTL-2-DOX and DTL-3-DOX via stepwise protocol, as described in Example 4.9.

FIG. 57 depicts binding affinity by ELISA assay for Trastuzumab ADC conjugated with DTL-1-DOX, DTL-2-DOX and DTL-3-DOX via sequential protocol, as described in Example 4.9.

FIG. 58 depicts binding affinity by ELISA assay for Trastuzumab ADC conjugated with DTL-1-DOX, DTL-2-DOX and DTL-3-DOX via in situ protocol, as described in Example 4.9.

FIG. 59 depicts an analysis of ADCs Using Capillary Gel Electrophoresis, as described in detail in Example 4.5.

FIG. 60 depicts binding affinity by ELISA assay for Trastuzumab Fab ADC conjugated with DTL-1-DOX, DTL-2-DOX and DTL-3-DOX via stepwise protocol, as described in Example 4.9

FIG. 61 depicts binding affinity by ELISA assay for Trastuzumab Fab ADC conjugated with DTL-1-DOX, DTL-2-DOX and DTL-3-DOX via sequential protocol, as described in Example 4.9.

FIG. 62 depicts binding affinity by ELISA assay for Trastuzumab Fab ADC conjugated with DTL-1-DOX, DTL-2-DOX and DTL-3-DOX via in situ protocol, as described in Example 4.9.

FIG. 63 depicts modification of Trastuzumab, as described in Example 5.5.2. M) Molecular weight marker; lanes from top are 250, 150, 100, 100, 80, 60, 40, 30, 25, 20 and 15 kDa. AB) Unmodified antibody. 1) In situ, 6 eq of DiSH-Diet; 2) Stepwise, 6 eq DiBr-Diet 3) Stepwise, 6 eq DiSH-Diet; 4) In situ, 50 eq of DiSH-Diet; 5) Stepwise, 50 eq DiBr-Diet; 6) Stepwise, 50 eq DiSH-Diet. All reactions were incubated at 37° C.

FIG. 64 depicts binding affinity by ELISA assay for pyridazine-modified Trastuzumab-Fab conjugated with Astra-PEG, as described in Example 5.5.2.

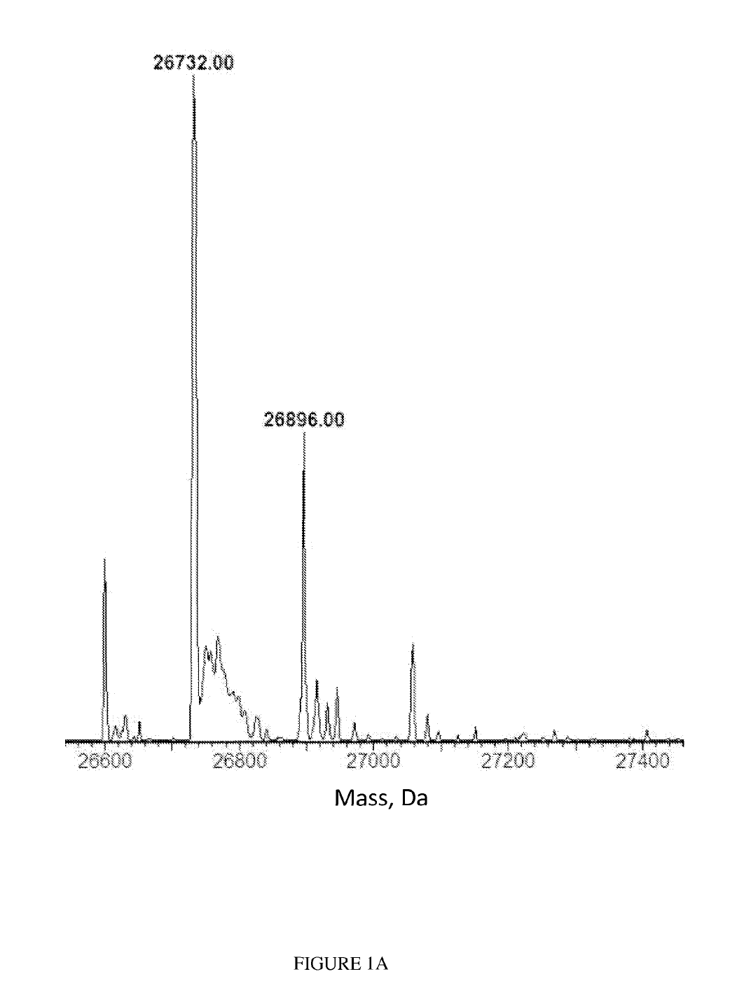

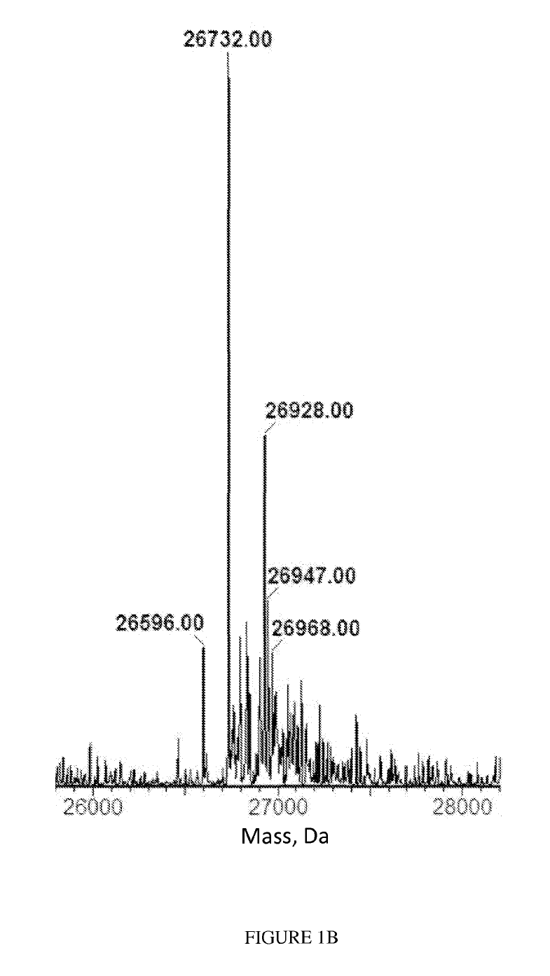

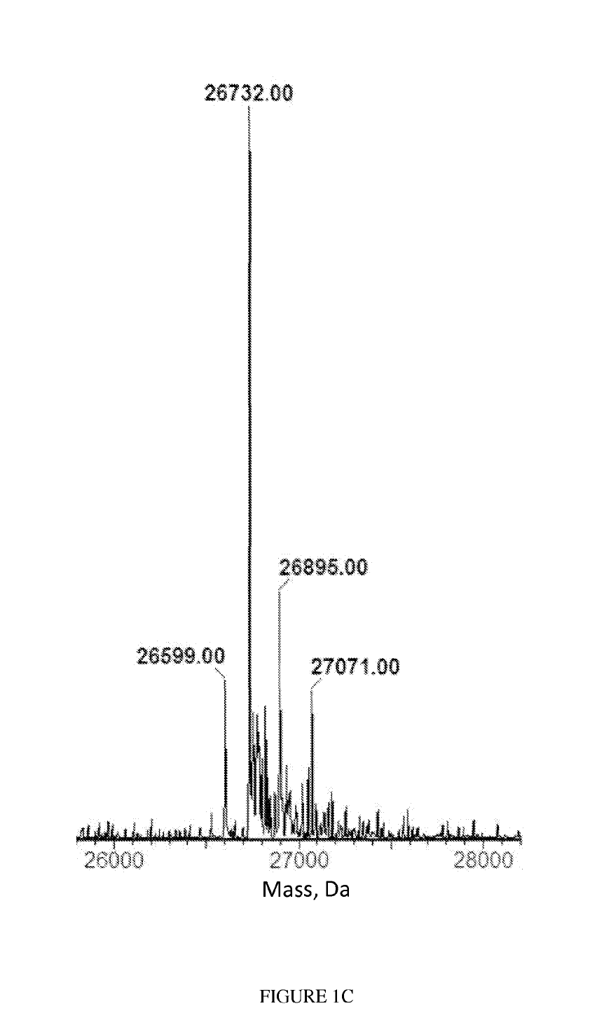

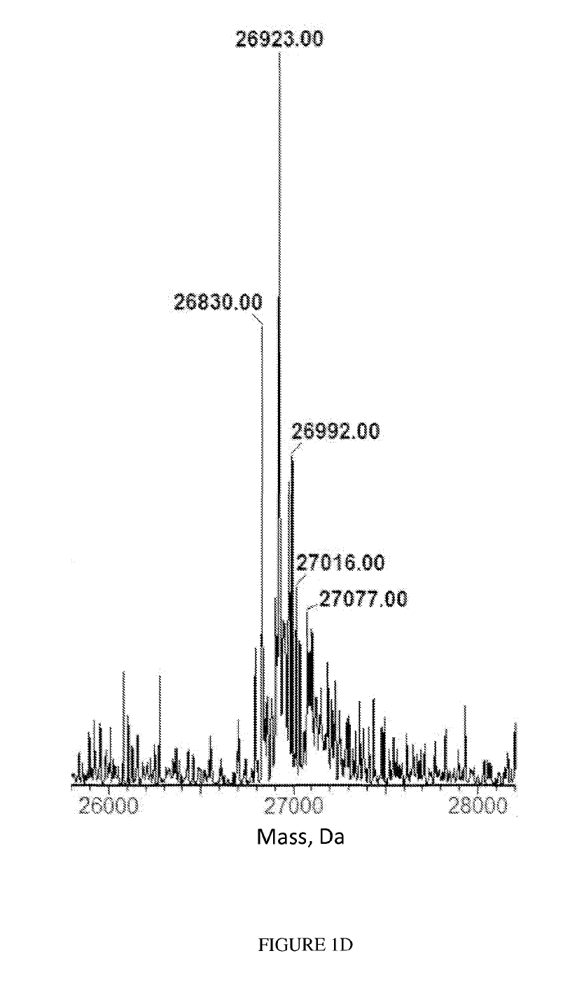

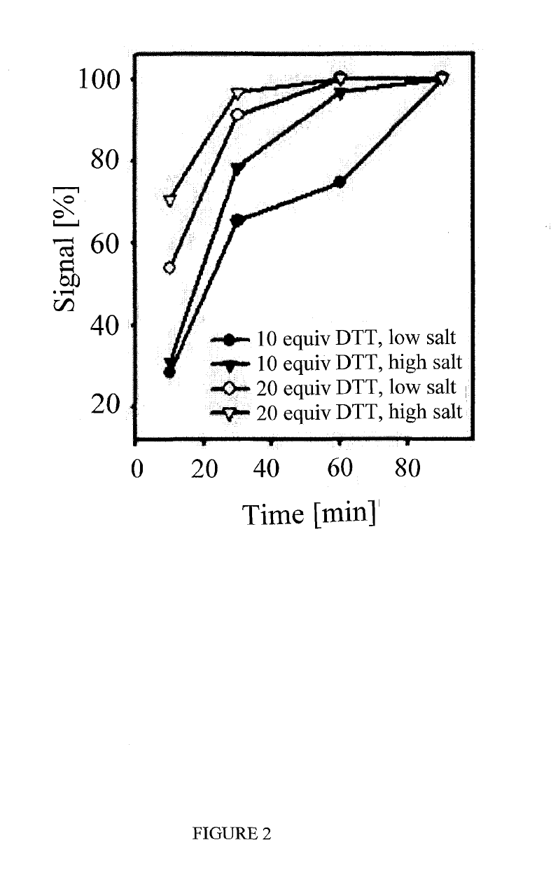

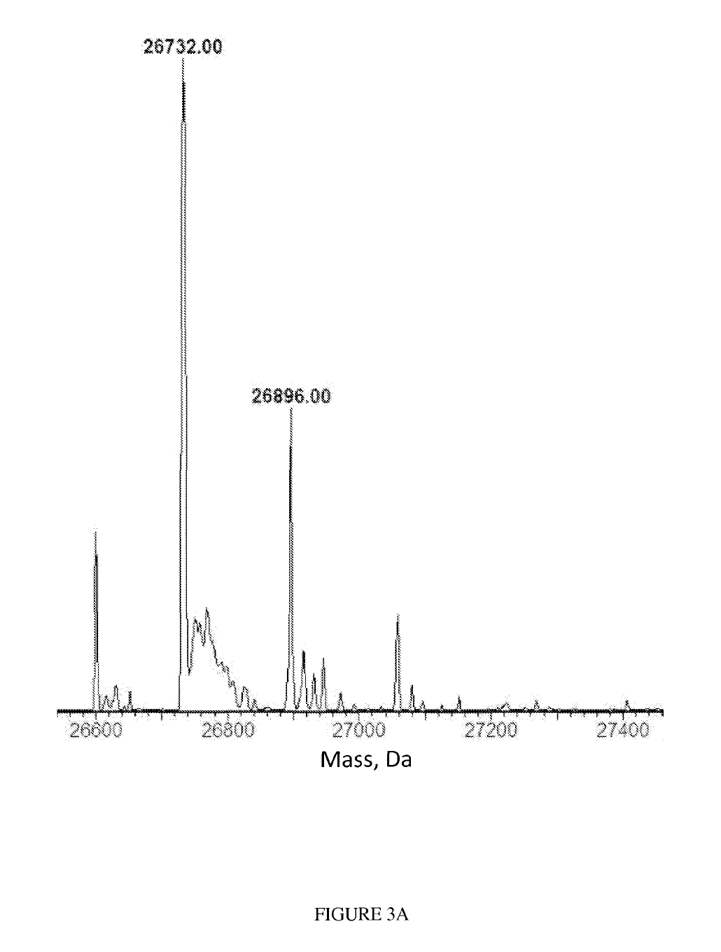

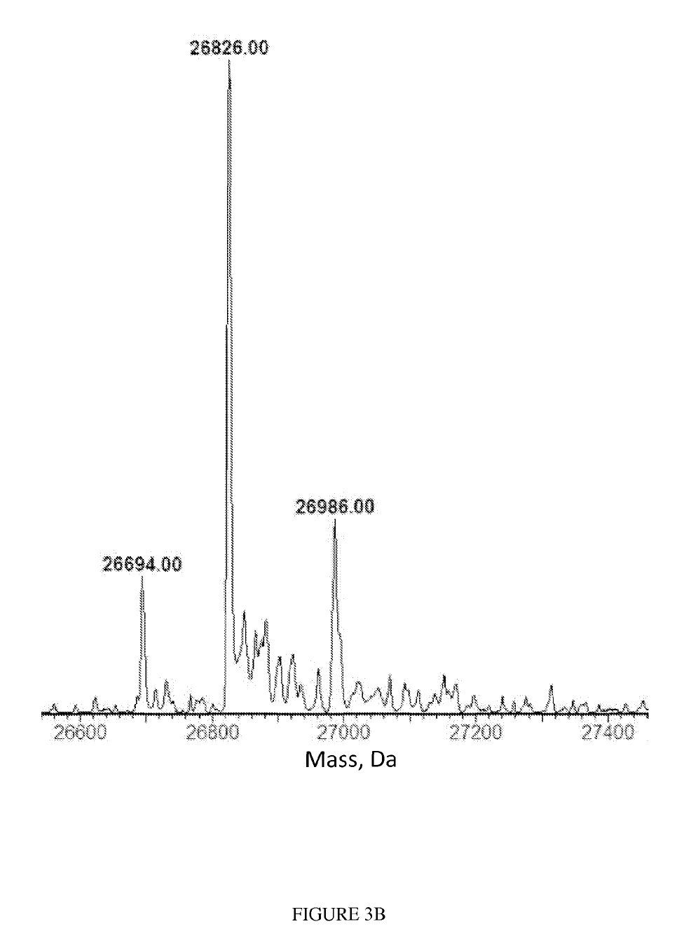

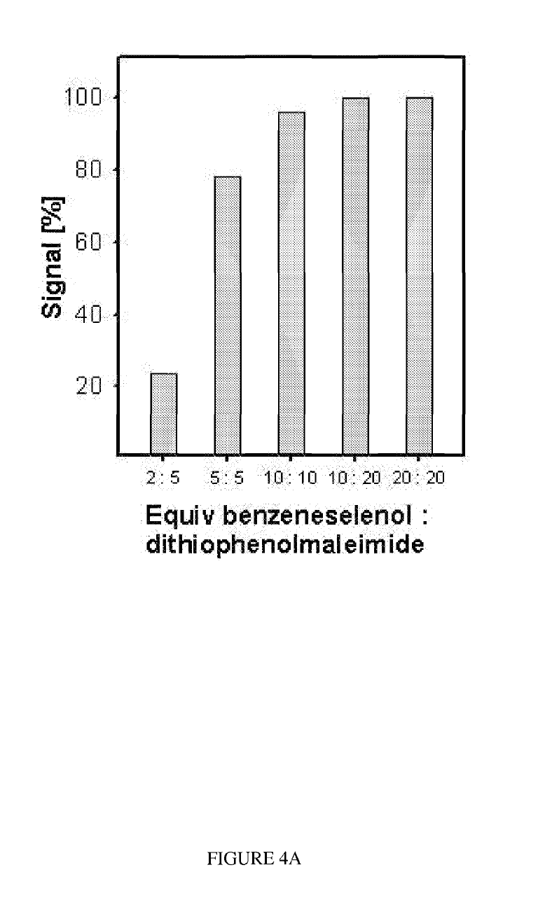

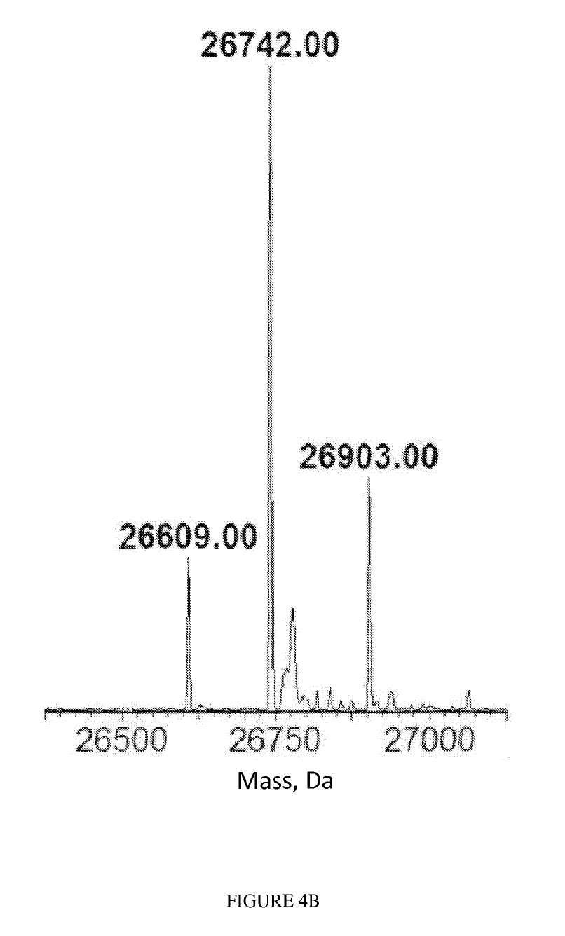

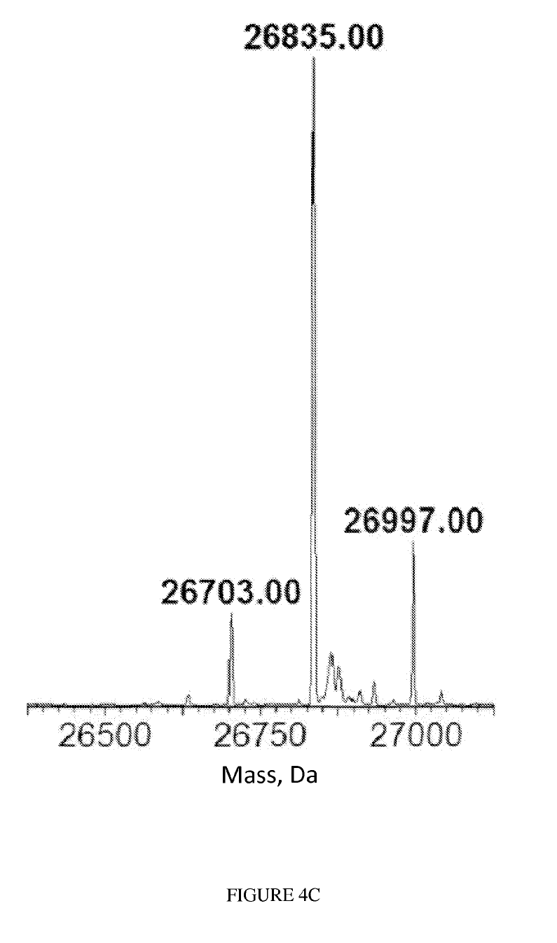

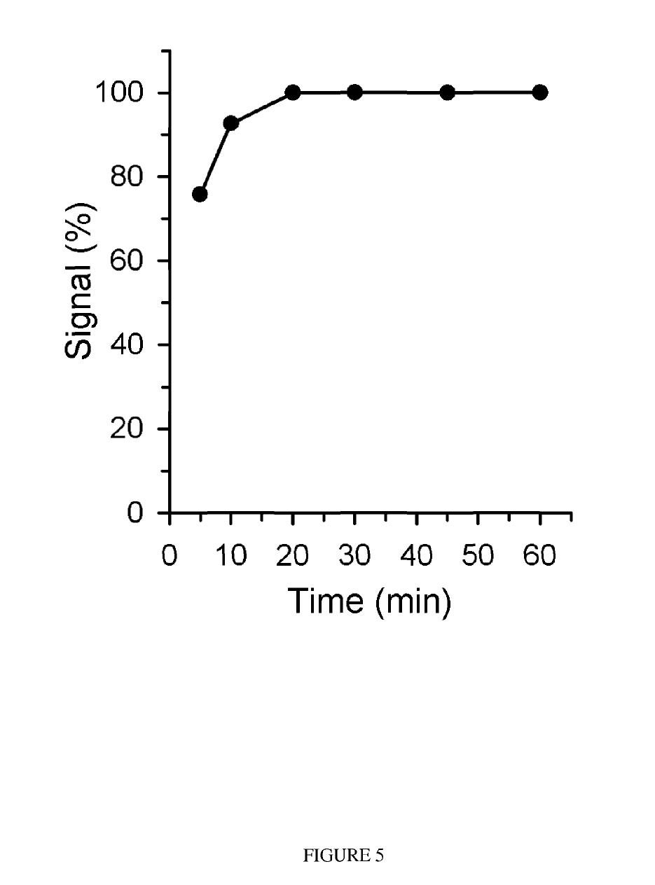

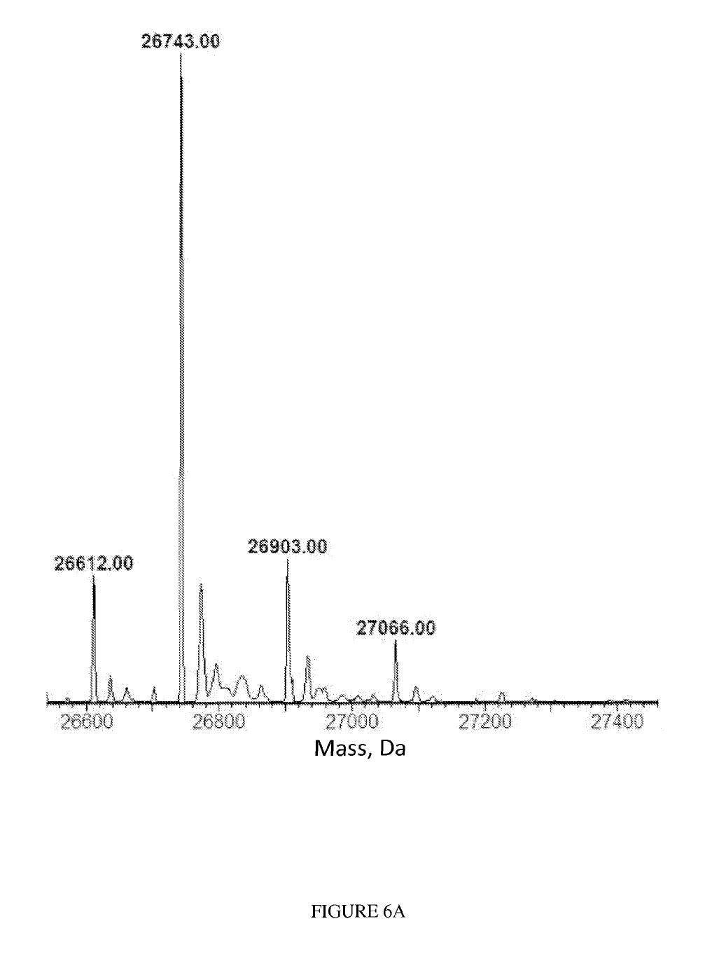

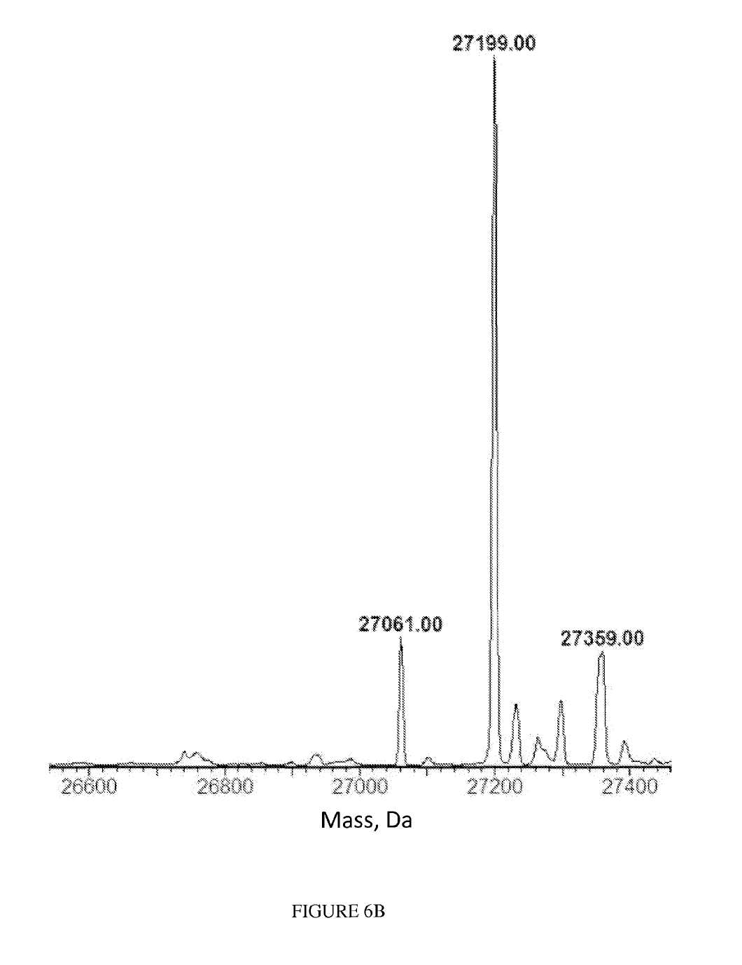

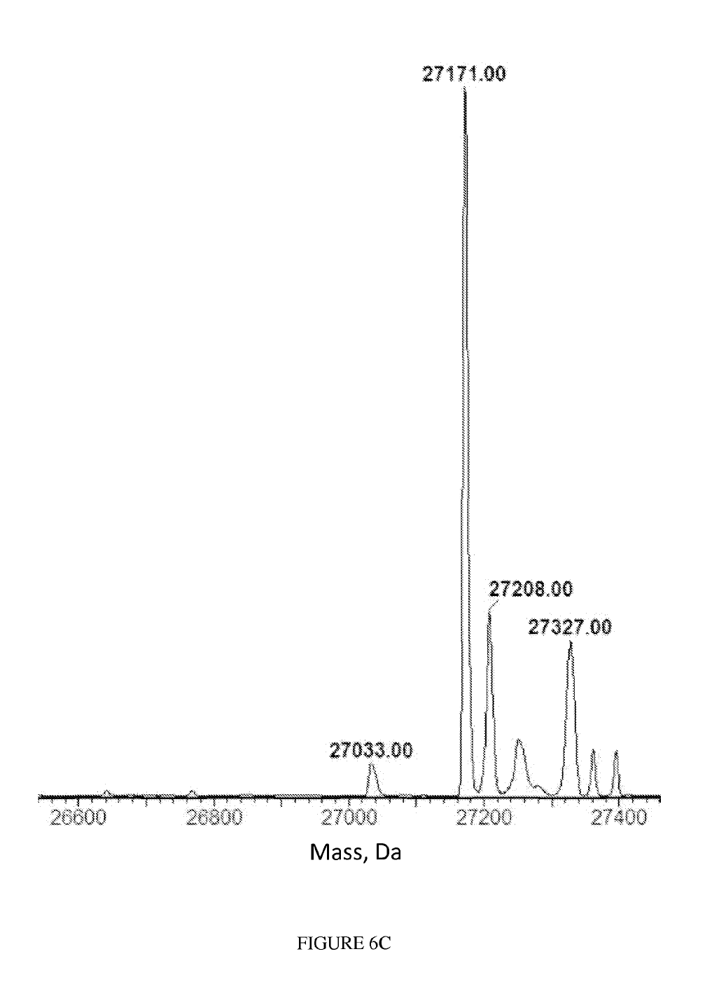

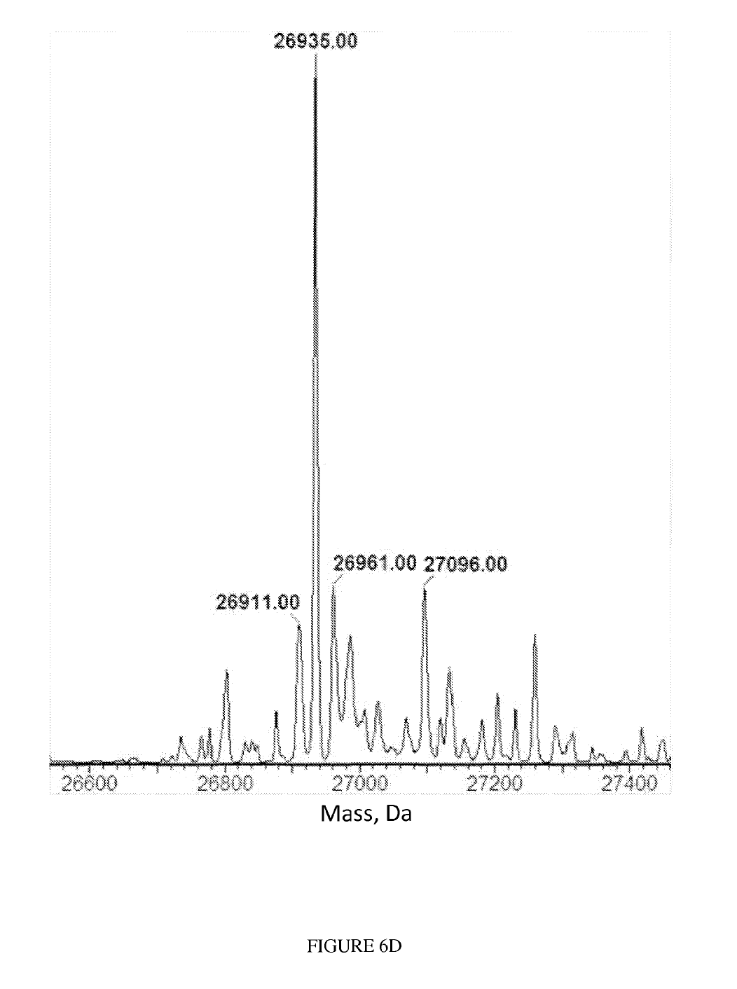

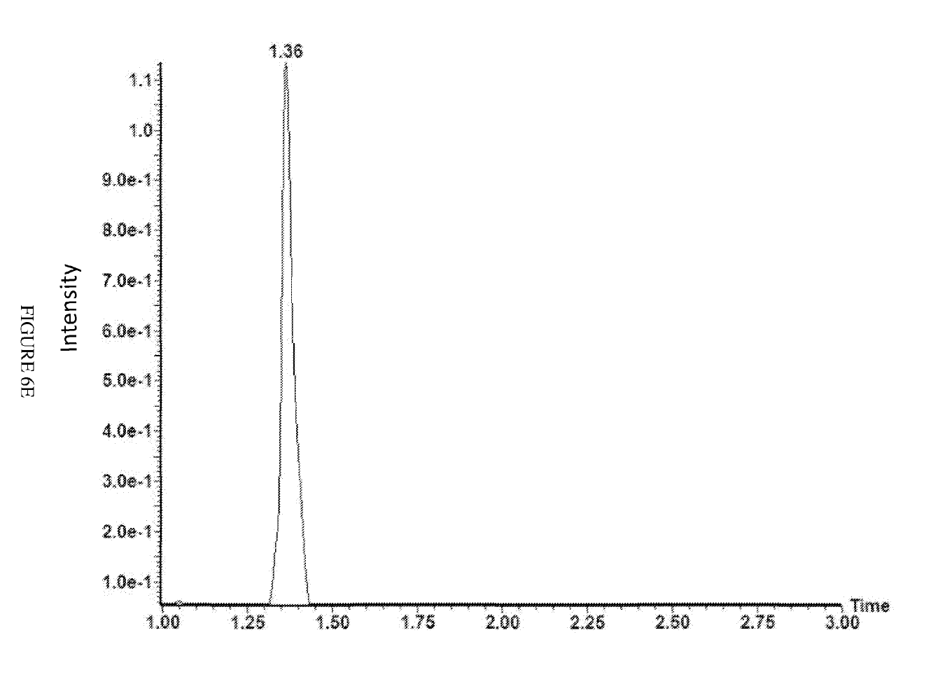

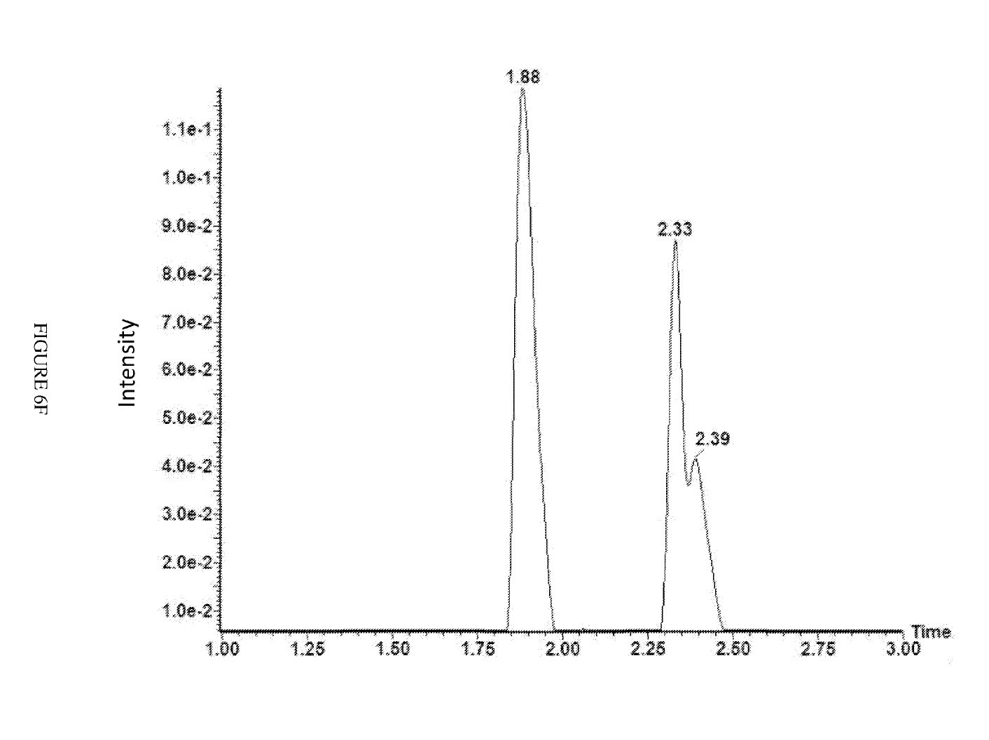

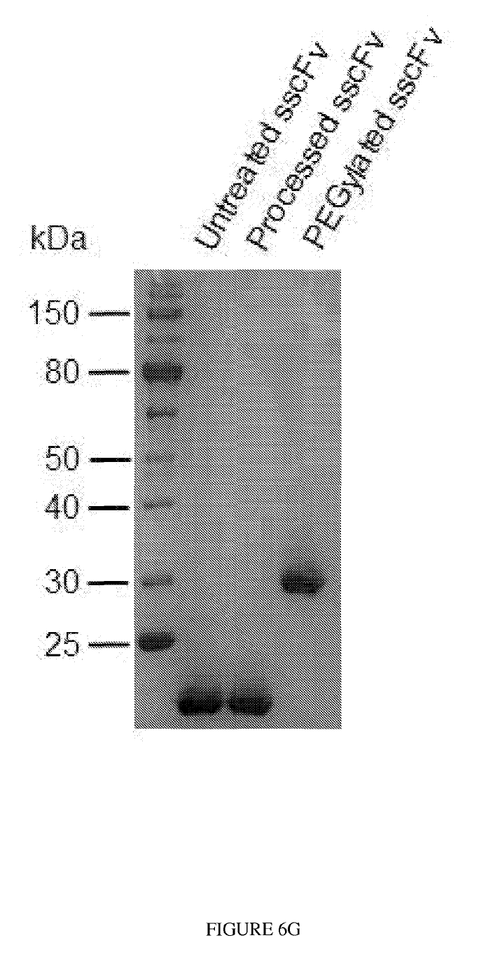

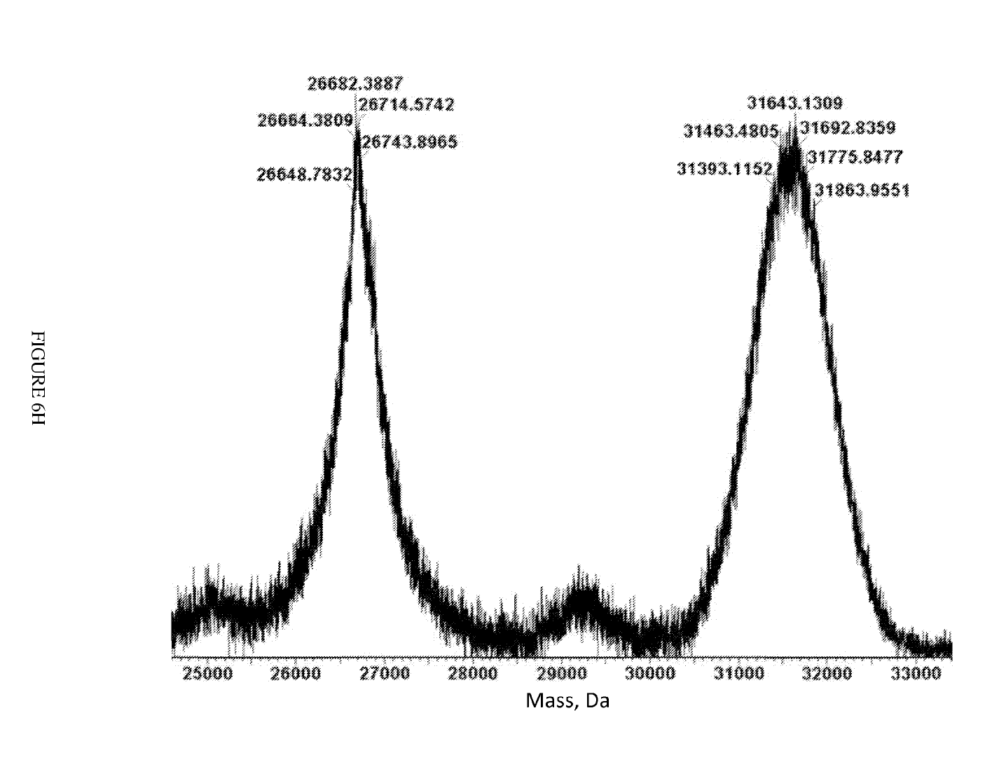

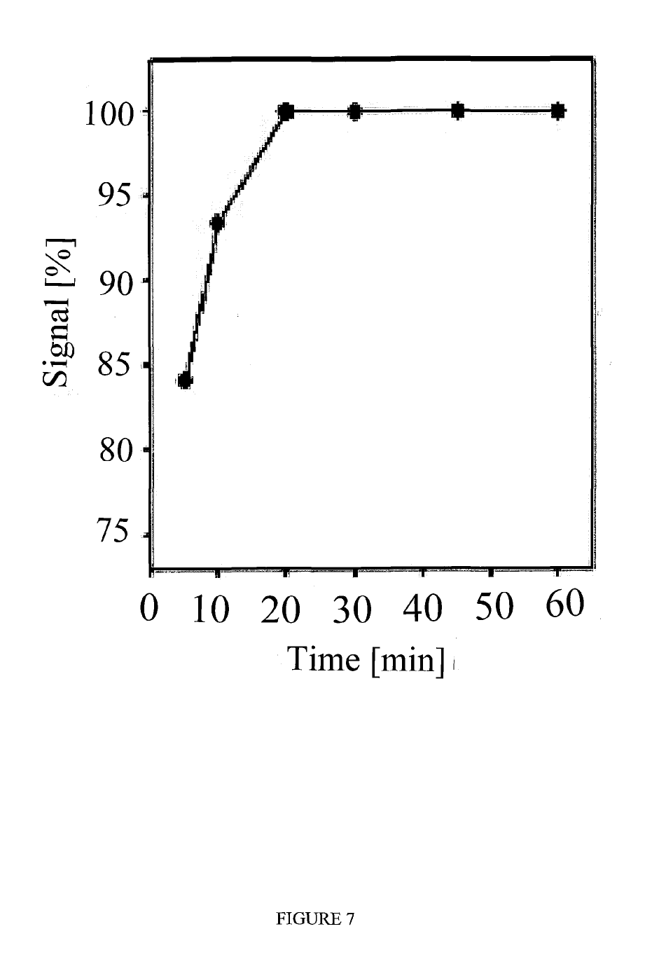

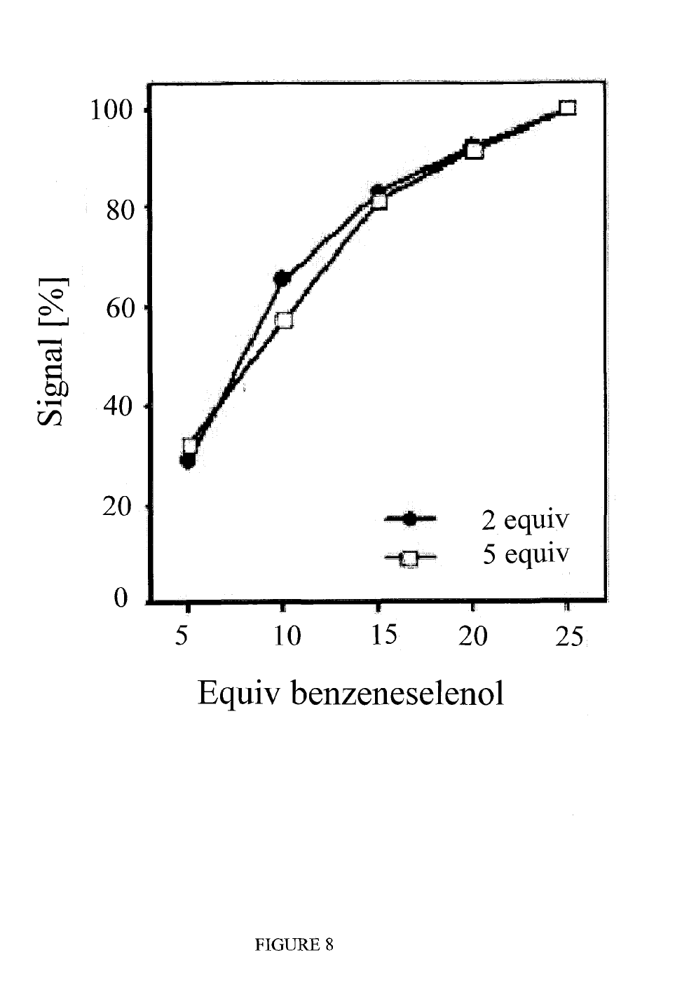

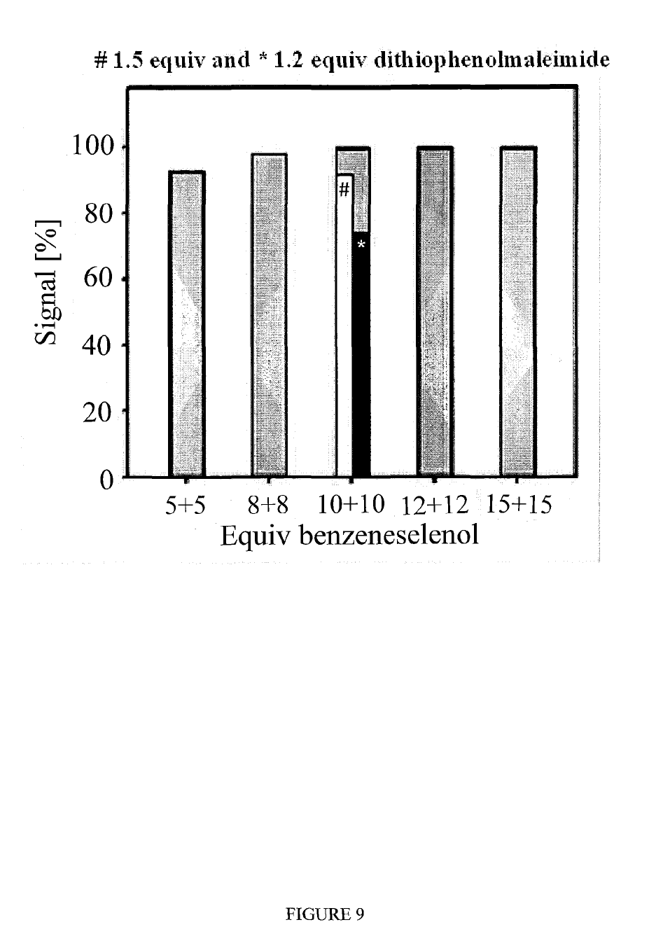

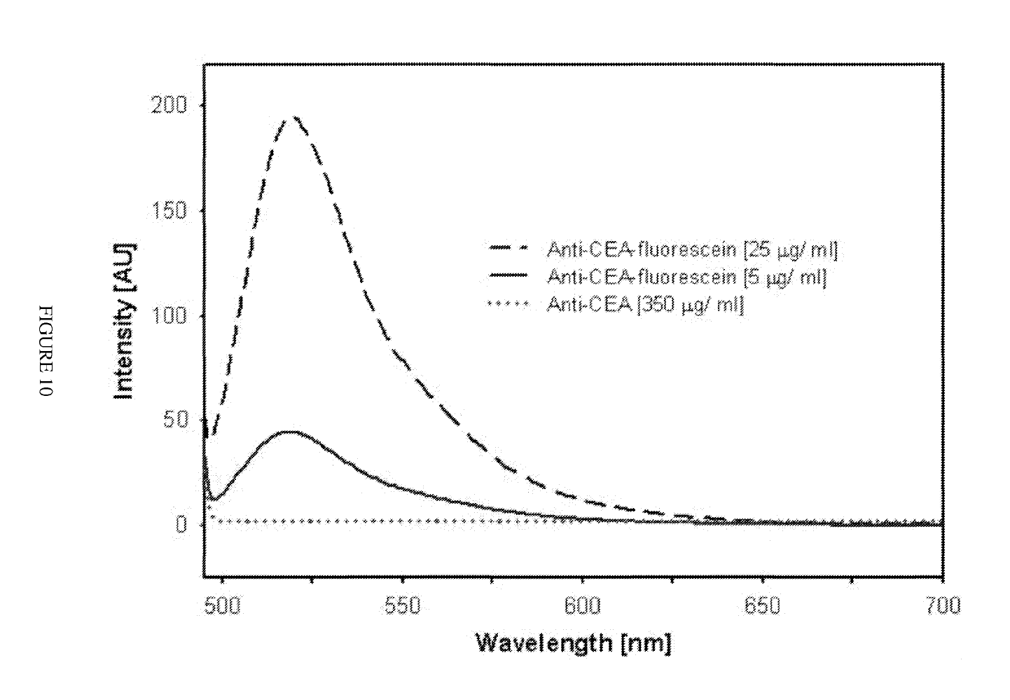

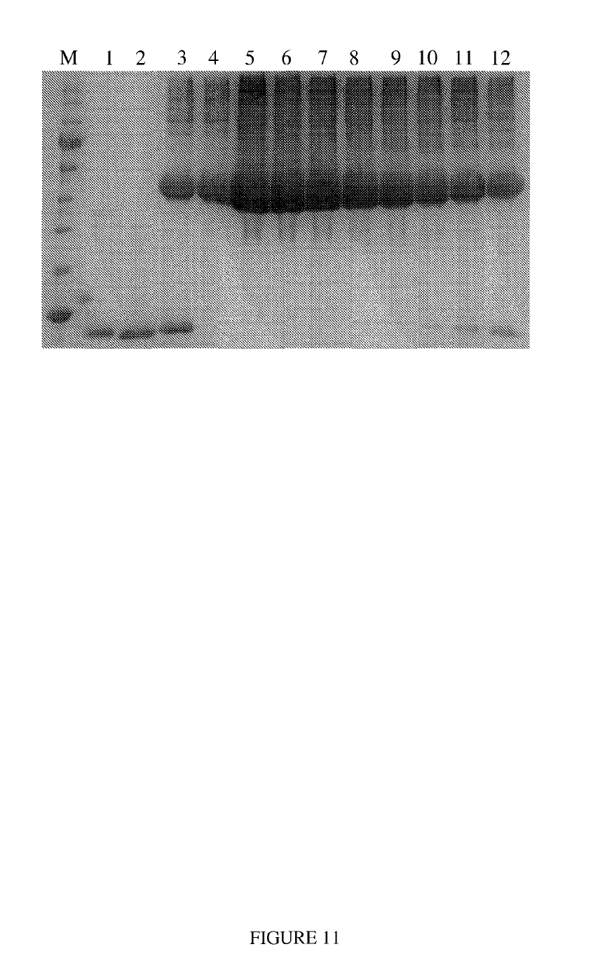

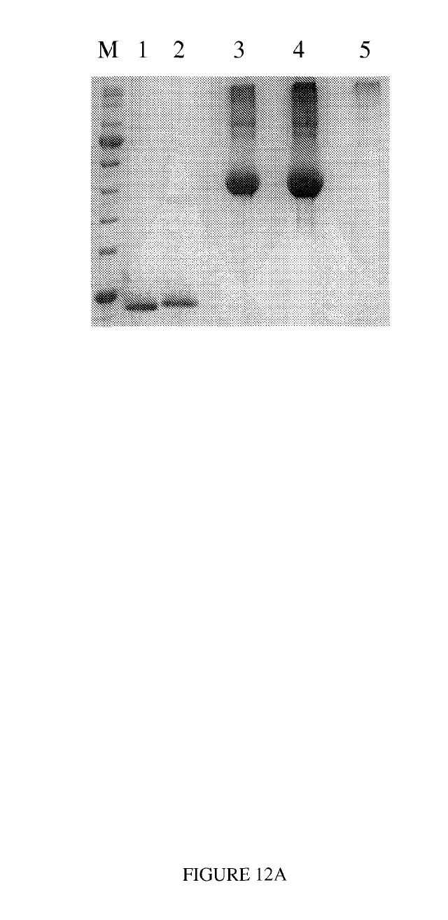

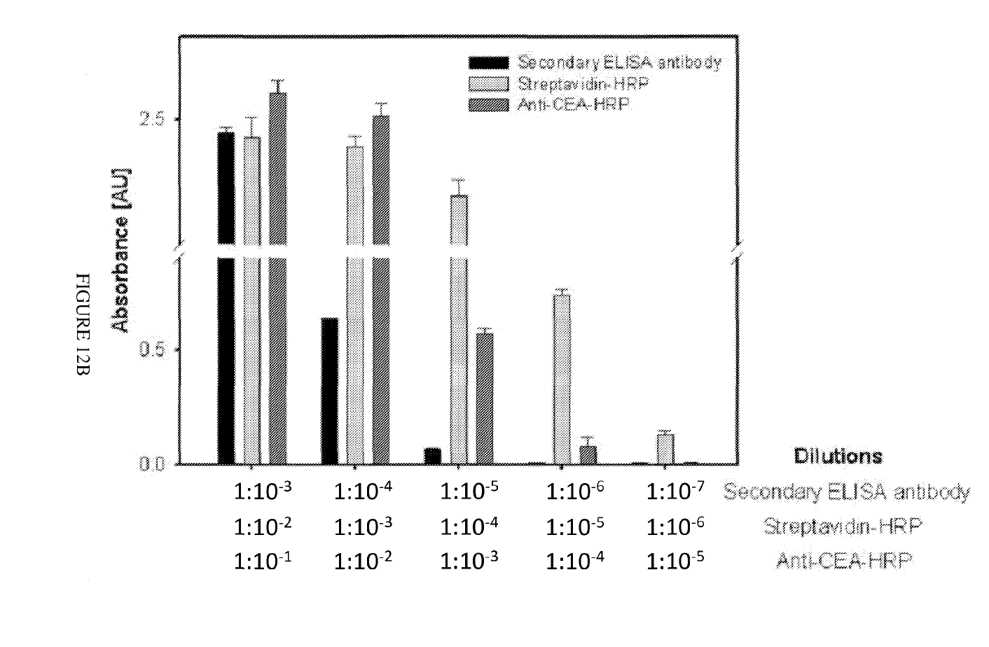

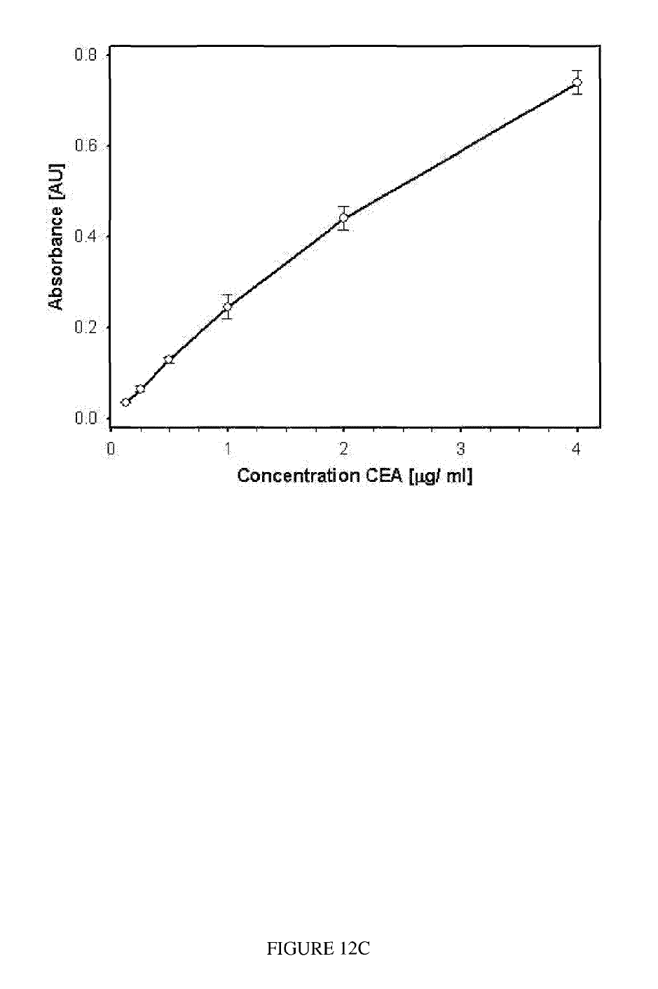

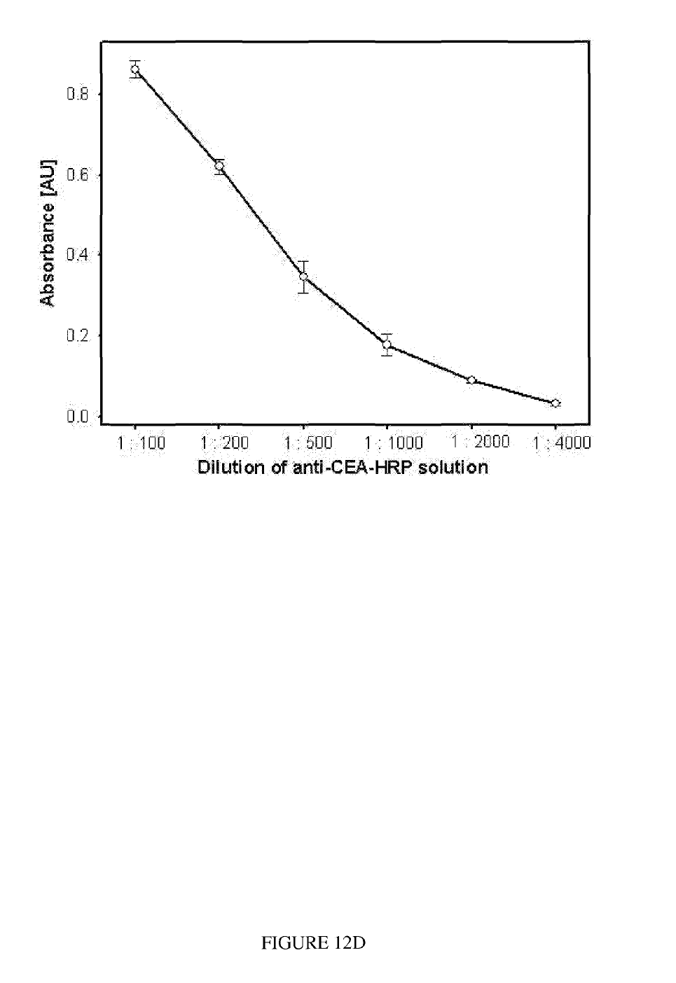

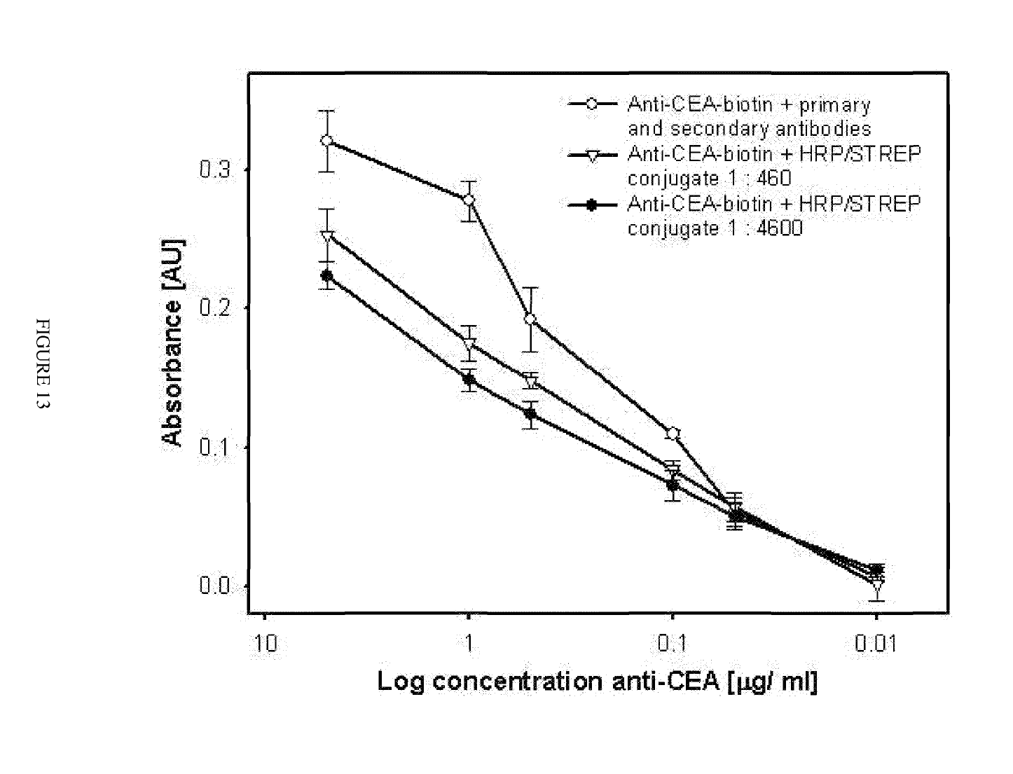

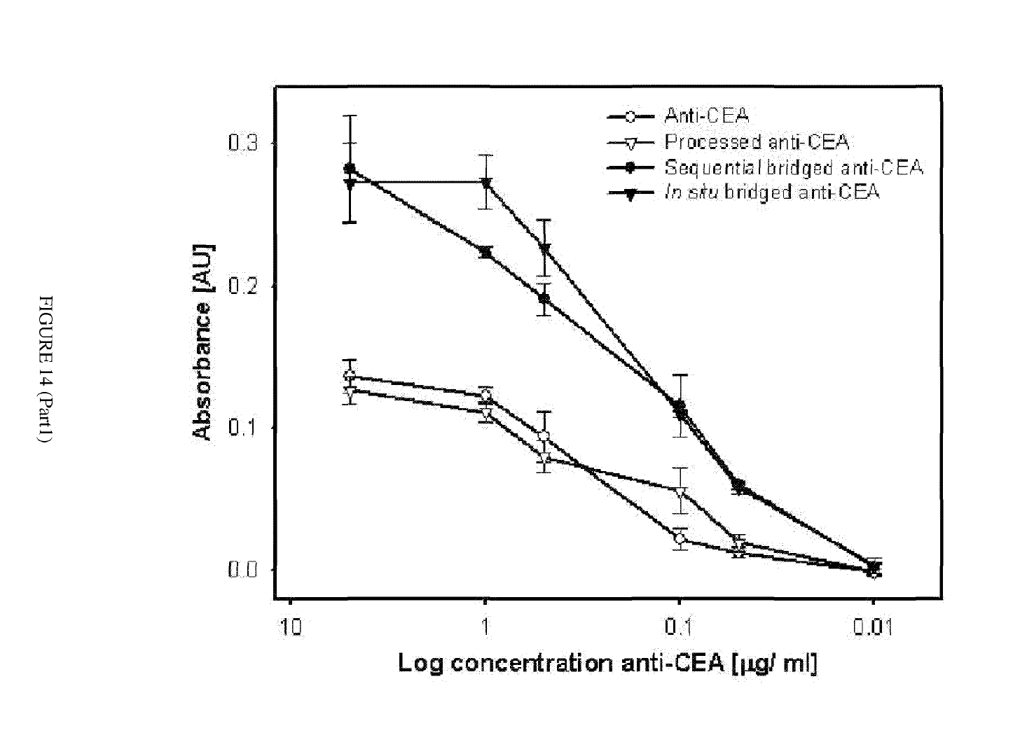

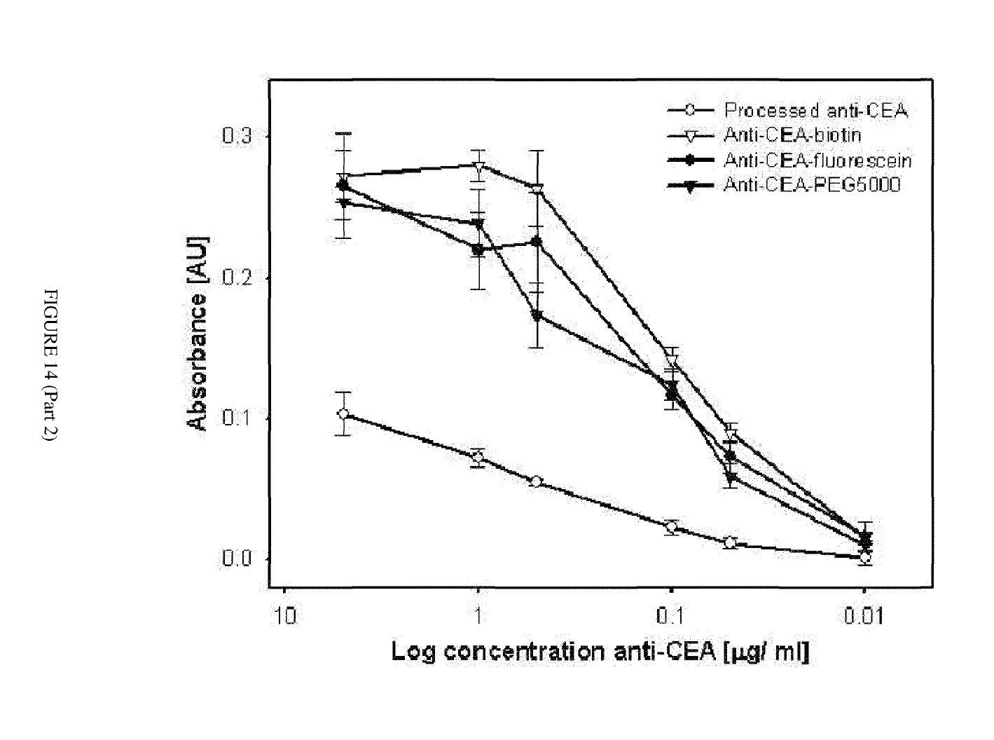

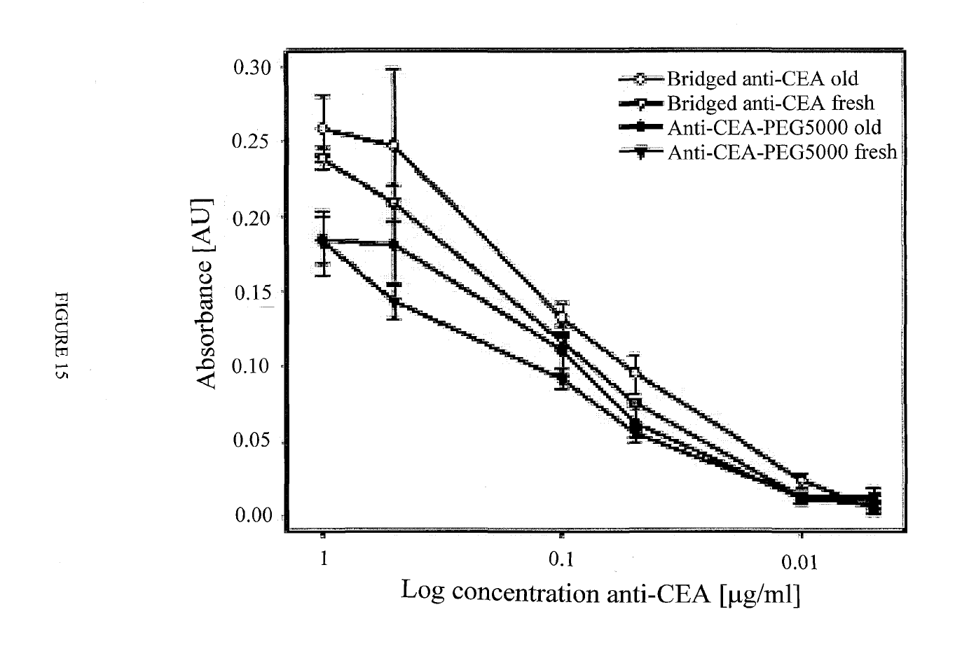

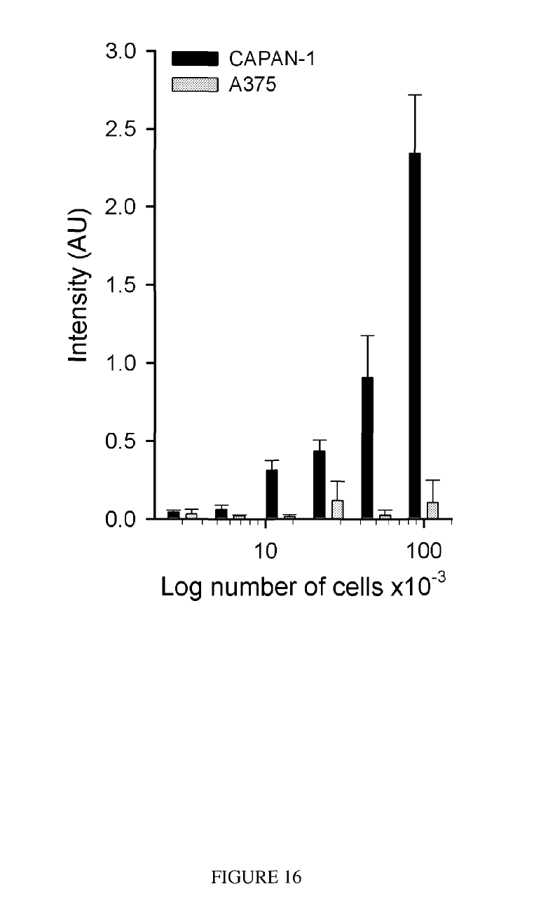

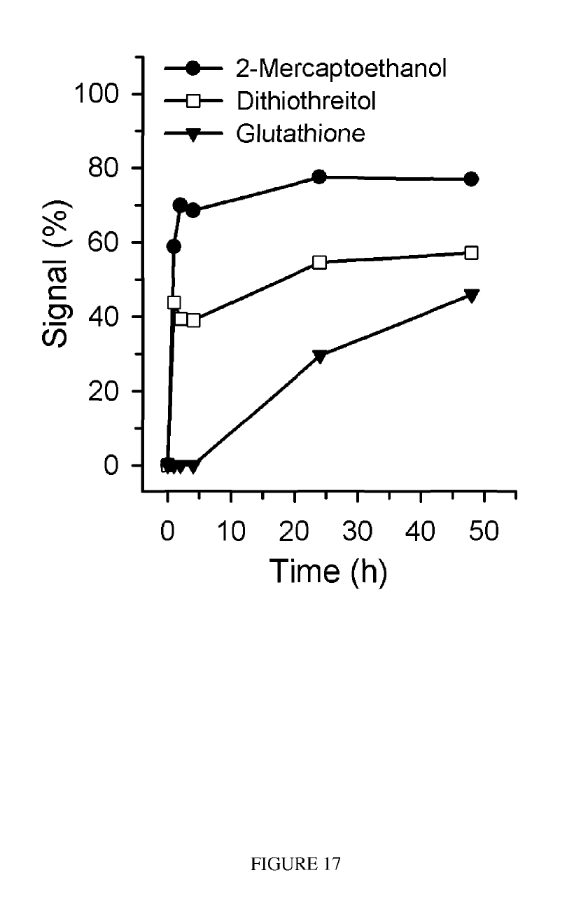

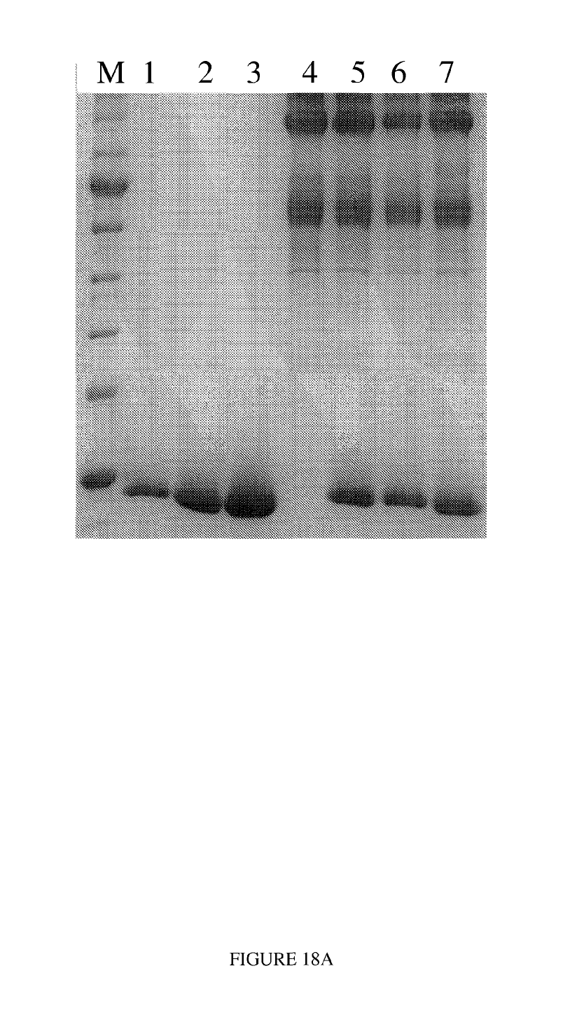

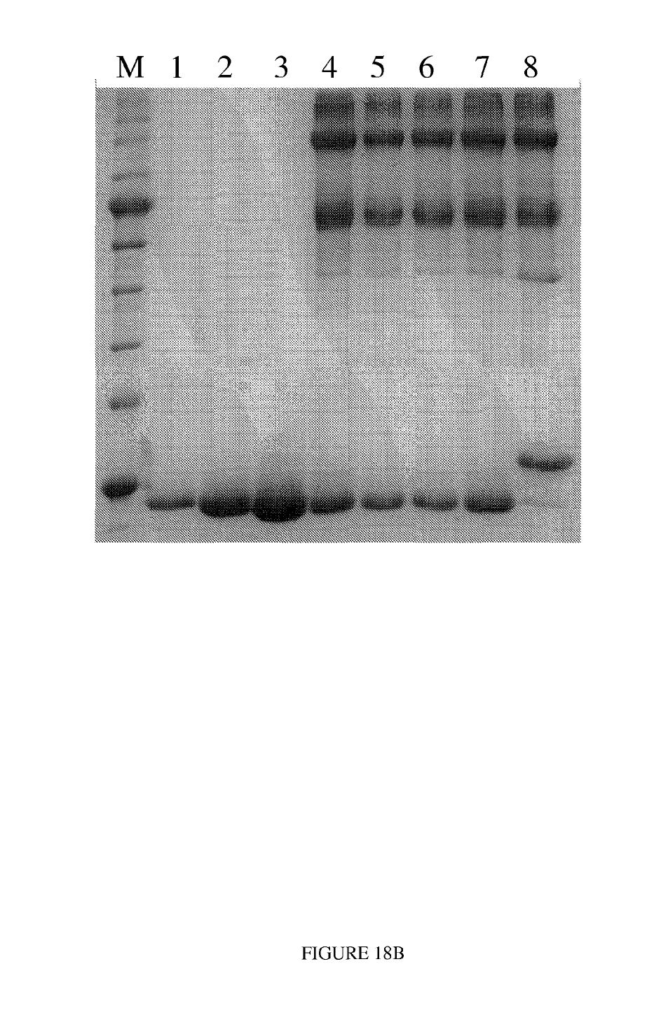

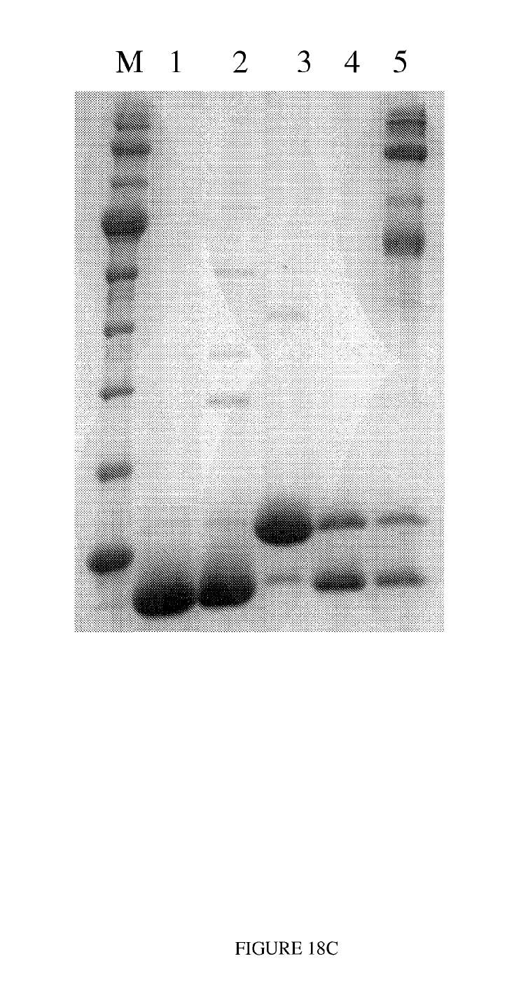

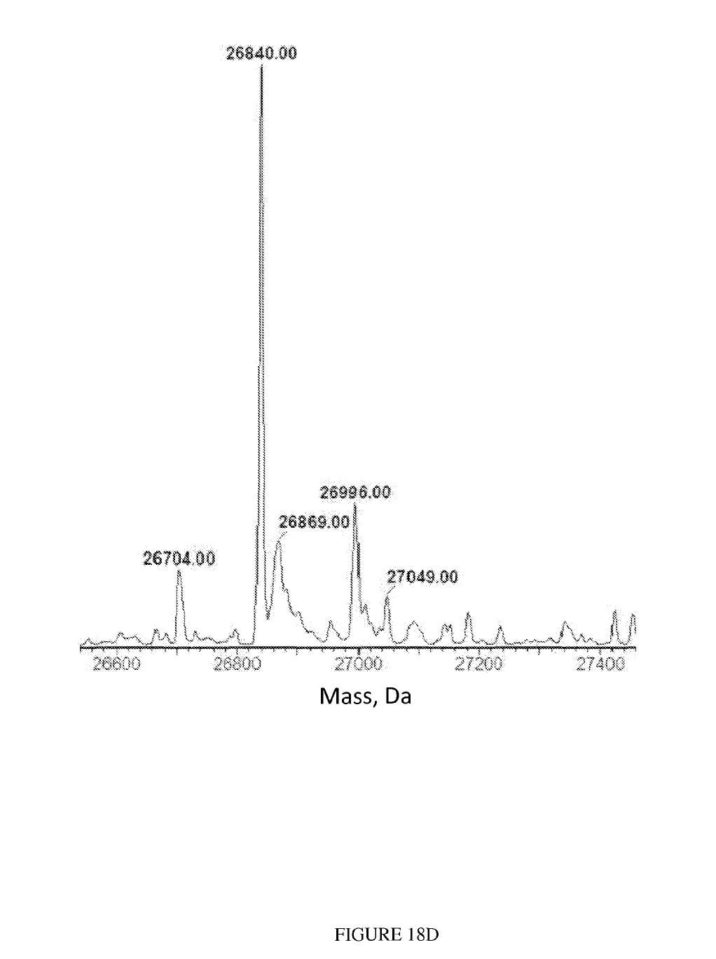

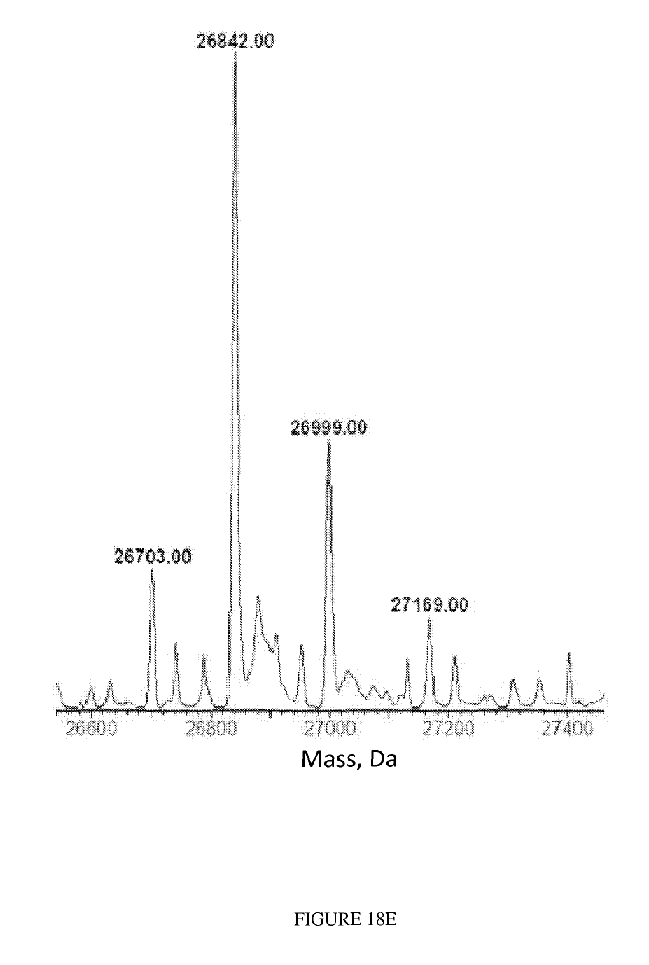

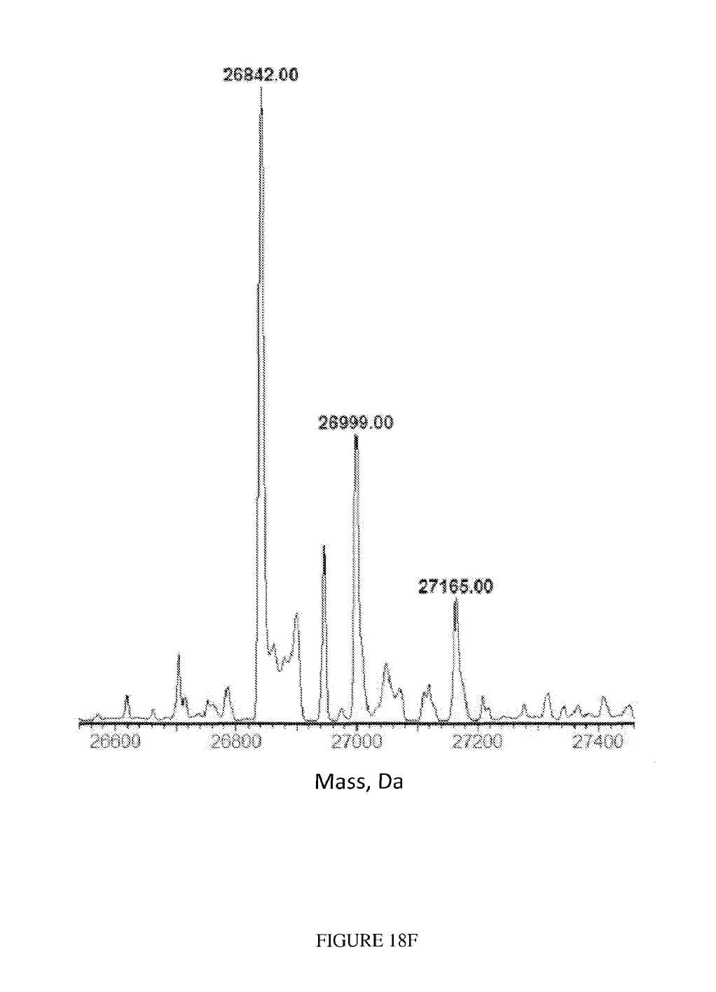

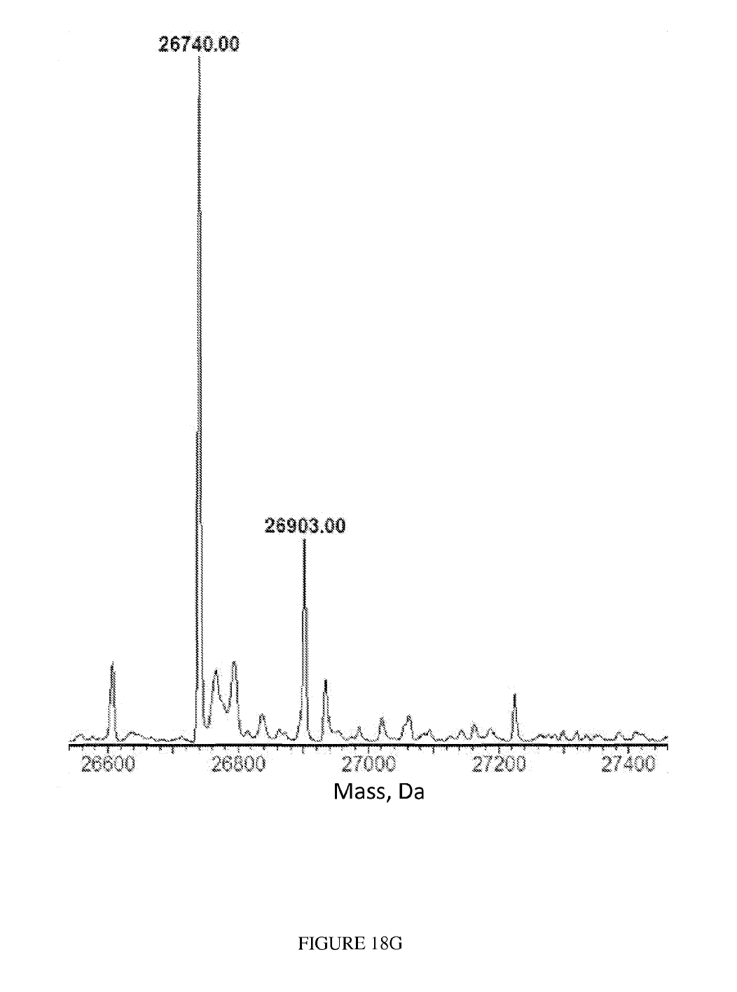

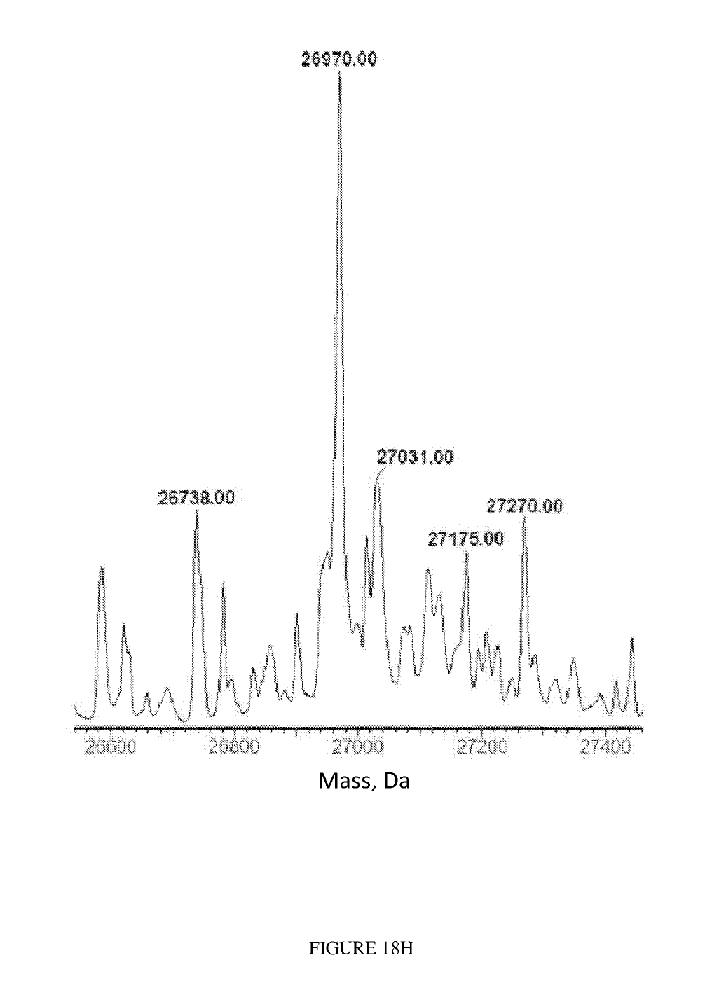

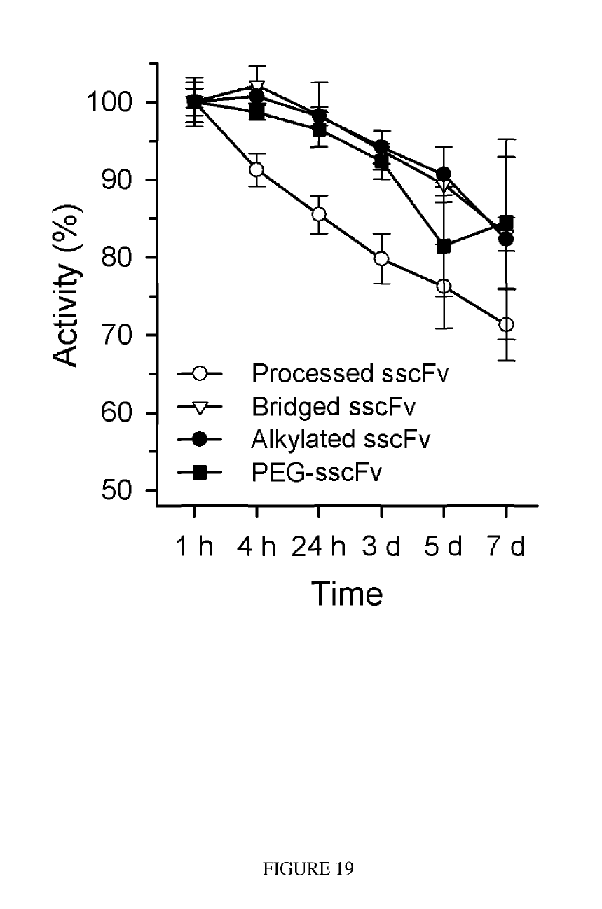

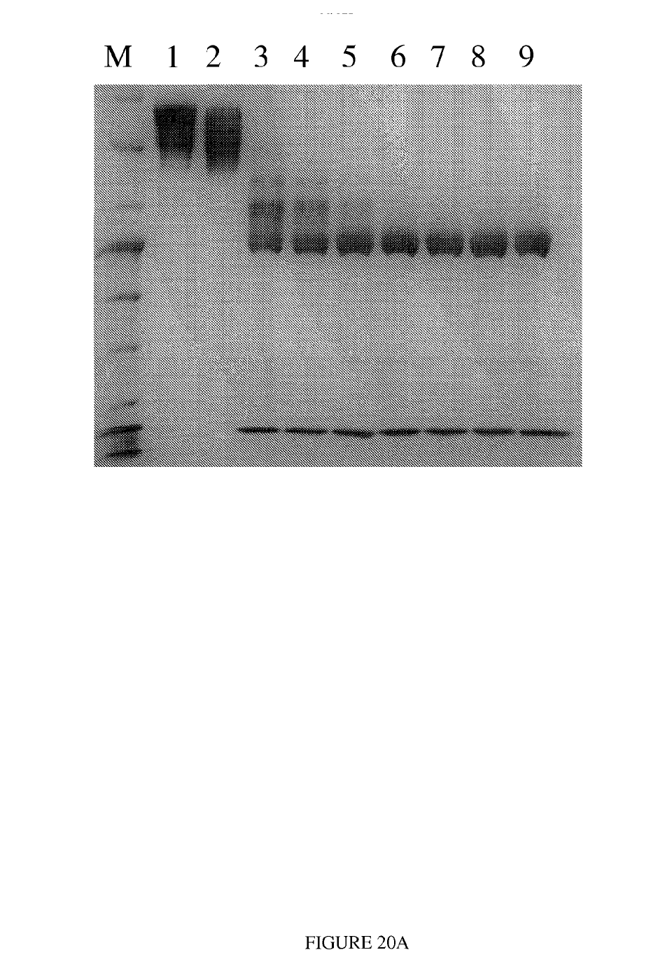

### DETAILED DESCRIPTION

As used herein, an “antibody” includes monoclonal antibodies, polyclonal antibodies, monospecific antibodies and multispecific antibodies (e.g., bispecific antibodies). An “antibody fragment” is a fragment of such an antibody that exhibits the desired biological activity, e.g. the activity or substantially the activity of its corresponding “intact” antibody (for example, which retains the capability of specific binding the antigen to which the “intact” antibody is capable of specifically binding).

Antibodies (and antibody fragments) as used herein include fusion proteins of antibodies (and antibody fragments) where a protein is fused via a covalent bond to the antibody (or antibody fragment). Also included are chemical analogues and derivatives of antibodies and antibody fragments, provided that the antibody or antibody fragment maintains its ability to bind specifically to its target antigen. Thus, for example, chemical modifications are possible (e.g., glycosylation, acetylation, PEGylation and other modifications without limitation) provided specific binding ability is retained. It is emphasised that such possible “chemical modifications” are in addition to the specific chemical modifications via the bridging moieties as described in detail herein.

An antibody comprises a variable region, which is capable of specific binding to a target antigen, and a constant region. An antibody as defined herein can be of any type or class (e.g., IgG, IgE, IgM, IgD, and IgA) or subclass (e.g., IgG1, IgG2, IgG3, IgG4, IgA1 and IgA2). The antibody can be derived from any suitable species. In some embodiments, the antibody is of human or murine origin. An antibody can be, for example, human, humanized or chimeric.

As used herein a “monoclonal antibody” is an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site.

“Monoclonal antibodies” as defined herein may be chimeric antibodies in which a portion of the heavy and/or light chain is identical to or homologous with the corresponding sequence of antibodies derived from a particular species or belonging to a particular antibody class or subclass, while the remainder of the chain(s) is identical to or homologous with the corresponding sequences of antibodies derived from another species or belonging to another antibody class or subclass, as well as fragments of such antibodies, so long as they exhibit the desired biological activity.

An “intact antibody” is one that comprises an antigen-binding variable region as well as a light chain constant domain (CL) and heavy chain constant domains, CHI, CH2, CR3 and CH4, as appropriate for the antibody class. The constant domains may be native sequence constant domains such as human native sequence constant domains or amino acid sequence variants thereof.

An intact antibody may have one or more “effector functions”, which refers to those biological activities attributable to the Fc region (e.g., a native sequence Fc region or amino acid sequence variant Fc region) of an antibody. Examples of antibody effector functions include complement dependent cytotoxicity, antibody-dependent cell-mediated cytotoxicity (ADCC) and antibody-dependent cell-mediated phagocytosis.

An “antibody fragment” comprises a portion of an intact antibody, preferably comprising the antigen-binding or variable region thereof. Examples of antibody fragments include Fab, Fab′, F(ab′)₂, and Fv fragments, diabodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragments of any of the above which immunospecifically bind to a target antigen (e.g., a cancer cell antigen).

“Humanized” forms of non-human (e.g., rodent) antibodies are chimeric antibodies that contain minimal sequence derived from non-human immunoglobulin.

The term “capable of specific binding to an antigen AG” refers to binding of the antibody (or antibody fragment) to a particular, predetermined target antigen, AG. Typically, the antibody (or antibody fragment) binds with an affinity of at least about 1×10⁷ M⁻¹, and/or binds to the predetermined target antigen with an affinity that is at least two-fold greater than its affinity for binding to a non-specific control substance (e.g., BSA, casein) other than the predetermined target antigen or a closely-related target antigen. For the avoidance of doubt, references herein to compositions of matter that comprise a plurality of chemically modified antibodies or antibody fragments of the present invention typically refer to a plurality of chemically modified antibodies or antibody fragments that are each capable of specific binding to the same antigen, AG (e.g., a composition that comprises a plurality of chemically modified antibodies that are each derived from the same native antibody or antibody fragment, but which differ in respect of the number or location of chemical modifications).

As used herein, a “chain” of an antibody or antibody fragment takes its normal meaning in the art, i.e. it refers to an “antibody chain”, namely an entity comprising a polypeptide sequence that forms or comprises one of the constituent parts of a (native) antibody. For the avoidance of doubt, it is emphasised that scFv antibody fragments, for example, comprise two such chains (i.e., the variable region of the heavy chain of an antibody and the variable region of the light chain of an antibody; in an scFv antibody fragment, the said chains are connected via a peptide linker, but are regarded herein nonetheless to comprise discrete chains).

A chain may be a heavy chain or a light chain. Light chains may be either κ (“kappa”) light chains or λ (“lambda”) light chains.

An inter-chain disulfide bond is a disulfide bond (—S—S—) that connects together discrete chains in an antibody or antibody fragment. Inter-chain disulfide bonds can be contrasted with intra-chain disulfide bonds, which connect together discrete sections of a single chain. The terms “inter-chain disulfide bond” is used interchangeably herein with the term “inter-chain disulfide bridge”. It will be understood that an inter-chain disulfide bond “bridges” discrete chains in an antibody or antibody fragment.

As is well known in the art, different classes and subclasses of antibodies contain different numbers of inter-chain disulfide bonds. For example, in an IgG1 antibody, there are four inter-chain disulfide bonds: one linking the first light chain to the first heavy chain, one linking the second light chain to the second heavy chain, and two linking the first heavy chain to the second heavy chain.

Thus, references herein to an “inter-chain bridging moiety” in a chemically modified antibody or antibody fragment typically mean that the moiety as defined in that context is present in place of (i.e., instead of) an inter-chain disulfide bond that would otherwise exist in the corresponding, unmodified (i.e., native) antibody or antibody fragment. Typically, therefore, for each inter-chain bridging moiety present in a chemically modified antibody or antibody fragment, there is one fewer inter-chain disulfide bond than would be present in the corresponding, unmodified (i.e., native) antibody or antibody fragment. For example, for a chemically modified IgG1 antibody having two inter-chain bridging moieties, there would typically be a total of (only) two inter-chain disulfide bridges remaining Note that references herein to an antibody or antibody fragment that “has” (or “having”) a given number of inter-chain bridging moieties typically means that the antibody or antibody fragment has specifically that number of such inter-chain bridging moieties (rather than potentially having more, not explicitly specified, such inter-chain bridging moieties).

As used here, the term “native” refers to a substance (e.g., an antibody, antibody fragment, cargo moiety) in its ambient form prior to incorporation into a chemically modified antibody or antibody fragment of the present invention. For example, references to a “native” antibody typically refer to the antibody as it exists in the absence of the chemical modifications effected according to the present invention so as to introduce one or more inter-chain bridging moieties as defined herein. References to a “native” antibody fragment typically refer to the antibody fragment as it exists in the absence of the chemical modifications effected according to the present invention so as to introduce one or more inter-chain bridging moieties as defined herein. Similarly, references to a “native” cargo moiety refer to the cargo moiety prior to its incorporation into a chemically modified antibody or antibody fragment of the present invention.

As used herein, a “cargo moiety” constitutes any moiety that may be attached to an antibody or antibody fragment in order to modify the characteristics of the said antibody or antibody fragment in a manner desired in view of the intended application of the particular antibody or antibody fragment. One of ordinary skill in the art would be familiar with the concept of chemical modification of antibodies and antibody fragments and could therefore select suitable cargo moieties to adapt the chemically modified antibody or antibody fragment for its intended practical purpose.

Exemplary cargo moieties include the following: a detectable moiety (for example, an imaging agent), an enzymatically active moiety, an affinity tag, a hapten, an immunogenic carrier, an antigen, a ligand, a biologically active moiety, a liposome, a polymeric moiety, a half-life-extending agent, an amino acid, a peptide, a protein, a cell, a carbohydrate, a DNA, an RNA and a solid substrate.

As will be readily understood by those of skill in the art, a cargo moiety comprised within a compound (e.g., within a chemically modified antibody or antibody fragment) is obtainable by attaching a corresponding native “cargo substance” (e.g., a cargo molecule) thereto. When a cargo substance attaches to a secondary compound, it is necessary for a bond somewhere in that cargo substance to be broken so that a new bond can form to the secondary compound. Examples of such processes include the loss of a leaving group from the cargo substance when it becomes a cargo moiety bound to the secondary molecule, the loss of a proton when the cargo substance reacts via a hydrogen-atom containing nucleophilic group such as an —OH or —SH group, or the conversion of a double bond in the cargo substance to a single bond when the cargo substance reacts with the secondary compound via an electrophilic or nucleophilic additional reaction. Those skilled in the art would thus understand that a cargo moiety that is, for example, a “drug” means a moiety that is formed by incorporation of the native drug into a secondary molecule, with concomitant loss of a internal bond compared to the corresponding, native drug compound (for example, loss of a proton from an —OH, —SH or —NH₂ moiety when such a moiety forms the bond to the secondary molecule).

A cargo moiety may be a moiety that has a discrete biological significance in its native form (i.e., when it is not part of a chemically modified antibody or antibody fragment). Preferably any cargo moiety used in the present invention has a molecular weight of at least 200 Daltons, more preferably at least 500 Daltons, most preferably at least 1000 Daltons. A cargo moiety as described herein may be a biomolecule moiety.

As used herein, the term “detectable moiety” means a moiety that is capable of generating detectable signals in a test sample. Clearly, the detectable moiety can be understood to be a moiety which is derived from a corresponding “detectable compound” and which retains its ability to generate a detectable signal when it is linked to an antibody or antibody fragment in the manner described herein. Detectable moieties are also commonly known in the art as “tags”, “probes” and “labels”. Examples of detectable moieties include chromogenic moieties, fluorescent moieties, radioactive moieties and electrochemically active moieties. In the present invention, preferred detectable moieties are chromogenic moieties and fluorescent moieties. Fluorescent moieties are most preferred.

A chromogenic moiety is a moiety which is coloured, which becomes coloured when it is incorporated into a chemically modified antibody or antibody fragment of the present invention, or which becomes coloured when it is incorporated into a chemically modified antibody or antibody fragment of the present invention and the chemically modified antibody or antibody fragment subsequently interacts with a secondary target species (for example, where the chemically modified antibody or antibody fragment specifically binds to its corresponding antigen AG).

Typically, the term “chromogenic moiety” refers to a group of associated atoms which can exist in at least two states of energy, a ground state of relatively low energy and an excited state to which it may be raised by the absorption of light energy from a specified region of the radiation spectrum. Often, the group of associated atoms contains delocalised electrons. Chromogenic moieties suitable for use in the present invention include conjugated moieties containing Π systems and metal complexes. Examples include porphyrins, polyenes, polyenes and polyaryls. Preferred chromogenic moieties are

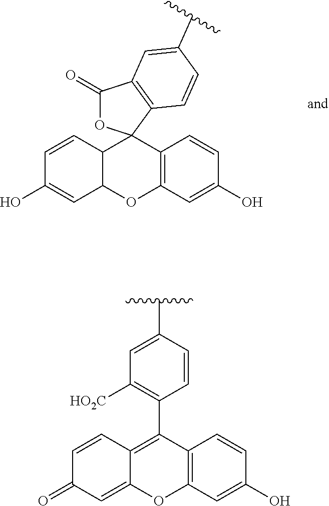 [SMILES: *c1ccc2c(c1)C(=O)OC21c2ccc(O)cc2OC2C=C(O)C=CC21 ; *c1ccc(-c2c3ccc(=O)cc-3oc3cc(O)ccc23)c(C(=O)O)c1]

A fluorescent moiety is a moiety that comprises a fluorophore, which is a fluorescent chemical moiety. Examples of fluorescent compounds that are commonly incorporated as fluorescent moieties into secondary molecules such as the chemically modified antibodies and antibody fragments of the present invention include:
the Alexa Fluor® dye family available from Invitrogen;
cyanine and merocyanine;
the BODIPY (boron-dipyrromethene) dye family, available from Invitrogen;
the ATTO dye family manufactured by ATTO-TEC GmbH;
fluorescein and its derivatives;
rhodamine and its derivatives;
naphthalene derivatives such as its dansyl and prodan derivatives;
pyridyloxazole, nitrobenzoxadiazole and benzoxadiazole derivatives;
coumarin and its derivatives;
pyrene derivatives; and
Oregon green, eosin, Texas red, Cascade blue and Nile red, available from Invitrogen.

Preferred fluorescent moieties for use in the present invention include fluorescein, rhodamine, coumarin, sulforhodamine 101 acid chloride (Texas Red) and dansyl. Fluorescein and dansyl are especially preferred.

A radioactive moiety is a moiety that comprises a radionuclide. Examples of radionuclides include iodine-131, iodine-125, bismuth-212, yttrium-90, yttrium-88, technetium-99m, copper-67, rhenium-188, rhenium-186, gallium-66, gallium-67, indium-111, indium-114m, indium-114, boron-10, tritium (hydrogen-3), carbon-14, sulfur-35, fluorine-18 and carbon-11. Fluorine-18 and carbon-11, for example, are frequently used in positron emission tomography.

In one embodiment, the radioactive moiety may consist of the radionuclide alone. In another embodiment, the radionuclide may be incorporated into a larger radioactive moiety, for example by direct covalent bonding to a linker group (such as a linker containing a thiol group) or by forming a co-ordination complex with a chelating agent. Suitable chelating agents known in the art include DTPA (diethylenetriamine-pentaacetic anhydride), NOTA (1,4,7-triazacyclononane-N,N′,N″-triacetic acid), DOTA (1,4,7,10-tetraazacyclododecane-N,N′,N″,N′″-tetraacetic acid), TETA (1,4,8,11-tetraazacyclotetra-decane-N,N′,N″,N′″-tetraacetic acid), DTTA (N¹-(p-isothiocyanatobenzyl)-diethylene-triamine-N¹,N²,N³-tetraacetic acid) and DFA (N′-[5-[[5-[[5-acetylhydroxyamino)pentyl]amino]-1,4-dioxobutyl]hydroxyamino]pentyl]-N-(5-aminopentyl)-N-hydroxybutanediamide).

An electrochemically active moiety is a moiety that comprises a group that is capable of generating an electrochemical signal in an electrochemical method such as an amperometric or voltammetric method. Typically, an electrochemically active moiety is capable of existing in at least two distinct redox states.

A person of skill in the art would of course easily be able to select a detectable compound that would be suitable for use in accordance with the present invention from the vast array of detectable compounds that are routinely available. The methodology of the present invention can thus be used to produce a chemically modified antibody or antibody fragment comprising a detectable moiety, which can then be used in any routine biochemical technique that involves detection of such species.

One particularly useful class of detectable moiety is an imaging agent. Imaging agents (which as defined herein include contrast agents) are widely used in medicine, for example in diagnosis and for monitoring the efficacy of ongoing therapeutic interventions. A large number of imaging agents have been used in vivo in human and animal subjects. For example, a detailed list of many hundreds of such imaging agents is available from the Molecular Imaging and Contrast Agent Database (accessible online at Molecular Imaging and Contrast Agent Database (MICAD) [Internet]. Bethesda (Md.): National Center for Biotechnology Information (US); 2004-2013. Available from: http://wvvw.nchi.nlm.nih.gov/books/NBK5330/).

A person of skill in the art would thus readily be able to select an imaging agent that would be suitable for use in accordance with the present invention from the vast array of imaging agents that are routinely available, and then to incorporate the selected imaging agent as a cargo moiety within a product of the present invention. The methodology of the present invention can thus be used to produce a chemically modified antibody or antibody fragment comprising an imaging agent, which can then be used in any routine technique that involves the use of that imaging agent.

Examples of particularly preferred imaging agents include an imaging agent selected from the group consisting of radionuclide probes (including Technetium-99m, Indium-111, Iodine-123, Iodine-124, Iodine-125, Gallium-67, Gallium-68, Lutetium-177, Fluorine-18 (18F), Zirconium-89, Copper-64, Techetium-94m and Bromine-76), fluorescent optical probes (including a compound from the Alexa Fluor dye family, the cyanine dye family, the BODIPY (boron-dipyrromethene) dye family, the ATTO dye family; fluorescein and its derivatives; rhodamine and its derivatives; naphthalene derivatives, for example its dansyl and prodan derivatives; pyridyloxazole, nitrobenzoxadiazole and benzoxadiazole derivatives; coumarin and its derivatives; pyrene derivatives; and Oregon green, eosin, Texas red, Cascade blue and Nile red).

As used herein, the term “enzymatically active moiety” means an enzyme, a substrate for an enzyme or a cofactor for an enzyme. Preferably, the enzymatically active moiety is an enzyme.

As used herein, the term “affinity tag” means a chemical moiety that is capable of interacting with an “affinity partner”, which is a second chemical moiety, when both the affinity tag and the affinity partner are present in a single sample. Typically, the affinity tag is capable of forming a specific binding interaction with the affinity partner. A specific binding interaction is a binding interaction that is stronger than any binding interaction that may occur between the affinity partner and any other chemical substance present in a sample.

One affinity tag/affinity partner pair that is particularly widely used in biochemistry is the biotin/(strept)avidin pair. Avidin and streptavidin are proteins which can be used as affinity partners for binding with high affinity and specificity to an affinity tag derived from biotin (5-[(3aS,4S,6aR)-2-oxohexahydro-1H-thieno[3,4-d]imidazol-4-yl]pentanoic acid). Other affinity tag/affinity partner pairs commonly used in the art include amylase/maltose binding protein, glutathione/glutathione-S-transferase and metal (for example, nickel or cobalt)/poly(His). As one of skill in the art would appreciate, either member of the pair could function as the “affinity tag”, with the other member of the pair functioning as the “affinity partner”. The terms “affinity tag” and “affinity partner” are thus interchangeable.

A person of skill in the art would be aware of the routine use of affinity tag/affinity partner interactions in biochemistry and in particular in the context of bioconjugate technology. A person of skill in the art would thus have no difficulty in selected an affinity tag for use in accordance with the present invention. The methodology of the present invention can therefore be used to produce chemically modified antibodies and antibody fragments adapted for use in routine biochemical techniques that make use of affinity tag/affinity partner interactions.

Preferred affinity tags according to the present invention are biotin, amylase, glutathione and poly(His). A particularly preferred affinity tag is biotin.

As used herein, the term “hapten” means a moiety that comprises an epitope, which is not capable of stimulating an in vivo immune response in its native form, but which is capable of stimulating an in vivo immune response when linked to an immunogenic carrier molecule. Typically, a hapten is a non-proteinaceous moiety of relatively low molecular weight (for example, a molecular weight of less than 1000). An epitope is the part of a molecule or moiety that is recognized by the immune system and stimulates an immune response.

As used herein, the term “immunogenic carrier” means an antigen that is able to facilitate an immune response when administered in vivo and which is capable of being coupled to a hapten. Examples of immunogenic carriers include proteins, liposomes, synthetic or natural polymeric moieties (such as dextran, agarose, polylysine and polyglutamic acid moieties) and synthetically designed organic moieties. Commonly used protein immunogenic carriers have included keyhole limpet hemocyanin, bovine serum albumin, aminoethylated or cationised bovine serum albumin, thyroglobulin, ovalbumin and various toxoid proteins such as tetanus toxoid and diphtheria toxoid. Well known synthetically designed organic molecule carriers include the multiple antigentic peptide (MAP).

As used herein, the term “antigen” means a substance that is capable of instigating an immune response when administered in vivo and which is capable of binding to an antibody produced during said immune response.

As used herein, the term “ligand” means a moiety that is able to interact with a biomolecule (for example, a protein) in such a way as to modify the functional properties of the biomolecule. Typically, the ligand is a moiety that binds to a site on a target protein. The interaction between the ligand and the biomolecule is typically non-covalent. For example, the interaction may be through ionic bonding, hydrogen bonding or van der Waals' interactions. However, it is also possible for some ligands to form covalent bonds to biomolecules. Typically, a ligand is capable of altering the chemical conformation of the biomolecule when it interacts with it.

Examples of ligands capable of interacting with a protein include substrates (which are acted upon by the enzyme upon binding, for example by taking part in a chemical reaction catalysed by the enzyme), inhibitors (which inhibit protein activity on binding), activators (which increase protein activity on binding) and neurotransmitters.

As used herein, the term “biologically active moiety” means a moiety that is capable of inducing a biochemical response when administered in vivo.

The biologically active moiety can be a drug (otherwise referred to herein as a “drug moiety”). Drugs include cytotoxic agents such as doxorubicin, methotrexate and derivatives thereof, cytotoxin precursors which are capable of metabolising in vivo to produce a cytotoxic agent, anti-neoplastic agents, anti-hypertensives, cardioprotective agents, anti-arrhythmics, ACE inhibitors, anti-inflammatories, diuretics, muscle relaxants, local anaesthetics, hormones, cholesterol lowering drugs, anti-coagulants, anti-depressants, tranquilizers, neuroleptics, analgesics such as a narcotic or anti-pyretic analgesics, anti-virals, anti-bacterials, anti-fungals, bacteriostats, CNS active agents, anti-convulsants, anxiolytics, antacids, narcotics, antibiotics, respiratory agents, anti-histamines, immunosuppressants, immunoactivating agents, nutritional additives, anti-tussives, diagnostic agents, emetics and anti-emetics, carbohydrates, glycosoaminoglycans, glycoproteins and polysaccharides, lipids, for example phosphatidyl-ethanolamine, phosphtidylserine and derivatives thereof, sphingosine, steroids, vitamins, antibiotics, including lantibiotics, bacteristatic and bactericidal agents, antifungal, anthelminthic and other agents effective against infective agents including unicellular pathogens, small effector molecules such as noradrenalin, alpha adrenergic receptor ligands, dopamine receptor ligands, histamine receptor ligands, GABA/benzodiazepine receptor ligands, serotonin receptor ligands, leukotrienes and triodothyronine, and derivatives thereof.

The biologically active moiety can also be a moiety derived from a compound which is capable of readily crossing biological membranes and which, when forming a conjugate molecule with a secondary functional moiety, is capable of enhancing the ability of the secondary functional moiety to cross the biological membrane. For example, the biologically active moiety may be a “protein transduction domain” (PTD) or a small molecule carrier (“SMC” or “molecular tug”) such as those described in WO 2009/027679, the content of which is hereby incorporated by reference in its entirety.

In a preferred embodiment of the present invention, the biologically active moiety is a drug, for example one of the specific classes of drug further defined herein.

As used herein, the term “liposome” means a structure composed of phospholipid bilayers which have amphiphilic properties. Liposomes suitable for use in accordance with the present invention include unilamellar vesicles and multilamellar vesicles.

As used herein, the term “polymeric moiety” means a single polymeric chain (branched or unbranched), which is derived from a corresponding single polymeric molecule. Polymeric moieties may be natural polymers or synthetic polymers. Typically, though, the polymeric molecules are not polynucleotides.

As is well known in the biochemical field, creation of conjugates comprising a polymeric moiety is useful in many in vivo and in vitro applications. For example, various properties of a macromolecule such as a protein (including antibodies and antibody fragments) can be modified by attaching a polymeric moiety thereto, including solubility properties, surface characteristics and stability in solution or on freezing.

A person of skill in the art would therefore recognise that the methodology of the present invention can be used to prepare a chemically modified antibody or antibody fragment comprising a polymeric moiety. A person of skill in the art would easily be able to select suitable polymeric moieties for use in accordance with the present invention, on the basis of those polymeric moieties used routinely in the art.

The nature of the polymeric moiety will therefore depend upon the intended use of the chemically modified antibody or antibody fragment. Exemplary polymeric moieties for use in accordance with the present invention include polysaccharides, polyethers, polyamino acids (such as polylysine), polyvinyl alcohols, polyvinylpyrrolidinones, poly(meth)acrylic acid and derivatives thereof, polyurethanes and polyphosphazenes. Typically such polymers contain at least ten monomeric units. Thus, for example, a polysaccharide typically comprises at least ten monosaccharide units.

Two particularly preferred polymeric molecules are dextran and polyethylene glycol (“PEG”), as well as derivatives of these molecules (such as monomethoxypolyethylene glycol, “mPEG”). Preferably, the PEG or derivative thereof has a molecular weight of less than 20,000. Preferably, the dextran or derivative thereof has a molecular weight of 10,000 to 500,000.

The above polymers may, in particular, be useful for extending the half-life of the chemically modified antibodies and antibody fragments of the present invention in vivo (i.e., increasing their stability under physiological, e.g. cellular, conditions). A particular type of cargo moiety is thus a “half-life-extending agent”, namely a cargo moiety that is capable of increasing the half-life (for example under (e.g., human) physiological conditions) of the chemically modified antibody or antibody fragment compared with the half-life of an otherwise corresponding chemically modified antibody or antibody fragment that lacks this cargo moiety. The half-life-extending agent may be a polymeric moiety such as those described above or it may be an non-polymeric moiety. Typically the half-life-extending agent is a relatively high molecular weight substance, e.g. it may have a molecular weight of at least 500 Daltons, preferably at least 1000 Daltons, for example at least 2000 Daltons.

Exemplary half-life extending agents include a half-life extending agent selected from the group consisting of polyalkylene glycols, polyvinylpyrrolidones, polyacrylates, polymethacrylates, polyoxazolines, polyvinylalcohols, polyacrylamides, polymethacrylamides, HPMA copolymers, polyesters, polyacetals, poly(ortho ester)s, polycarbonates, poly(imino carbonate)s, polyamides, copolymers of divinylether-maleic anhydride and styrene-maleic anhydride, polysaccharides and polyglutamic acids.

As used herein, the term “amino acid” means a moiety containing both an amine functional group and a carboxyl functional group. However, preferably the amino acid is an α-amino acid. Preferably, the amino acid is a proteinogenic amino acid, i.e. an amino acid selected from alanine, arginine, asparagine, aspartic acid, cysteine, glutamic acid, glutamine, glycine, histidine, isoleucine, leucine, lysine, methionine, proline, phenylalanine, pyrrolysine, selenocysteine, serine, threonine, tryptophan, tyrosine and valine. However, the amino acid can also be a non-proteinogenic amino acid. Examples of non-proteinogenic amino acids include lanthionine, 2-aminoisobutyric acid, dehydroalanine, gamma-aminobutyric acid, ornithine, citrulline, canavanine and mimosine. A particularly preferred amino acid according to the present invention is cysteine.

As used herein, the terms “peptide” and “protein” mean a polymeric moiety made up of amino acid residues. As a person of skill in the art will be aware, the term “peptide” is typically used in the art to denote a polymer of relatively short length and the term “protein” is typically used in the art to denote a polymer of relatively long length. As used herein, the convention is that a peptide comprises up to 50 amino acid residues whereas a protein comprises more than 50 amino acids. However, it will be appreciated that this distinction is not critical since the cargo moieties identified in the present application can typically represent either a peptide or a protein.

As used herein, the term “polypeptide” is used interchangeable with “protein”. Furthermore, proteins include antibodies, antibody fragments and enzymes.

As used herein, a peptide or a protein can comprise any natural or non-natural amino acids. For example, a peptide or a protein may contain only α-amino acid residues, for example corresponding to natural α-amino acids. Alternatively the peptide or protein may additionally comprise one or more chemical modifications. For example, the chemical modification may correspond to a post-translation modification, which is a modification that occurs to a protein in vivo following its translation, such as an acylation (for example, an acetylation), an alkylation (for example, a methylation), an amidation, a biotinylation, a formylation, glycosylation, a glycation, a hydroxylation, an iodination, an oxidation, a sulfation or a phosphorylation. A person of skill in the art would of course recognise that such post-translationally modified peptides or proteins still constitute a “peptide” or a “protein” within the meaning of the present invention. For example, it is well established in the art that a glycoprotein (a protein that carries one or more oligosaccharide side chains) is a type of protein.

As used herein, the term “cell” means a single cell of a living organism.

As used herein, the term “carbohydrate” includes monosaccharides and oligosaccharides. Typically an oligosaccharide contains from two to nine monosaccharide units. Thus, as used herein, a polysaccharide is classified as a “polymeric moiety” rather than as a carbohydrate. However, a person of skill in the art will appreciate that this distinction is not important, since the cargo moieties used in accordance with the invention can typically constitute either of a “carbohydrate” and a “polysaccharide”.

As used herein, the term “DNA” means a deoxyribonucleic acid made up of one or more nucleotides. The DNA may be single stranded or double stranded. Preferably, the DNA comprises more than one nucleotide.

As used herein, the term “RNA” means a ribonucleic acid comprising one or more nucleotides. Preferably, the RNA comprises more than one nucleotide.

As used herein, the term “solid substrate” means an object which is a solid under standard conditions (temperature of about 20° C. and pressure of about 100 kPa) and which is capable of interacting with the inter-chain bridging moieties described herein, to form a conjugate comprising both the solid substrate and an antibody or antibody fragment. The solid substrates used in the present invention may be microscopic or macroscopic in dimension, but typically have at least one dimension that is greater than or equal to 0.001 μm, preferably 0.1 μm and most preferably 1 μm. The solid substrates used in the present invention can have any shape, including substrates having at least one substantially flat surface (for example, “slide”-, “membrane”- or “chip”-shaped substrates) and substrates having a curved surface (for example, bead-shaped substrates and tube-shaped substrates).

Those of skill in the art will be familiar with the variety of materials, shapes and sizes of solid substrates that are used routinely in the art. Typically, the solid substrates used in the present invention are solid substrates that are suitable for immobilising biomolecules (e.g., antibodies and antibody fragments) or other molecules of biological interest and thus they include any solid substrate that is known in the art to be suitable for such purposes. Commercial suppliers of such materials include Pierce, Invitrogen and Sigma Aldrich.

Solid substrates suitable for use in the present invention include nanotubes, metallic substrates, metal oxide substrates, glass substrates, silicon substrates, silica substrates, mica substrates and polymeric substrates. Preferred metallic substrates include gold, silver, copper, platinum, iron and/or nickel substrates, with gold substrates being particularly preferred.

Polymeric substrates include natural polymers and synthetic polymers. Clearly, a “polymeric substrate” is a substrate comprising a plurality of polymer molecules. Preferred polymeric substrates include polystyrene substrates, polypropylene substrates, polycarbonate substrates, cyclo-olefin polymer substrates, cross-linked polyethylene glycol substrates, polysaccharide substrates, such as agarose substrates, and acrylamide-based resin substrates, such as polyacrylamide substrates and polyacrylamine/azlactone copolymeric substrates. Preferred substrates include gold substrates, glass substrates, silicon substrates, silica substrates and polymeric substrates, particularly those polymeric substrates specified herein. Particularly preferred substrates are glass substrates, silicon substrates, silica substrates, polystyrene substrates, cross-linked polyethylene glycol substrates, polysaccharide substrates (for example, agarose substrates) and acrylamide-based resin substrates. In another preferred embodiment, the solid substrate is a nanotube, particularly a carbon nanotube.

As used herein, the term “nanotube” means a tube-shaped structure, the width of which tube is of the order of nanometres (typically up to a maximum of ten nanometres). Nanotubes can be carbon nanotubes or inorganic nanotubes. Carbon nanotubes can be single-walled nanotubes (SWNTs) or multi-walled nanotubes (MWNTs). Inorganic nanotubes are nanotubes made of elements other than carbon, such as silicon, copper, bismuth, metal oxides (for example, titanium dioxide, vanadium dioxide and manganese dioxide), sulfides (for example, tungsten disulphide and molybdenum disulphide), nitrides (for example, boron nitride and gallium nitride) and selenides (for example, tungsten selenide and molybdenum selenide). Preferably, the nanotube is a carbon nanotube.

As used herein, “conjugate” means a molecule which comprises an antibody or antibody fragment and at least one cargo moiety. The antibody or antibody fragment and the at least one cargo moiety are covalently linked to one another via an inter-chain bridging moiety attached to the antibody or antibody fragment, as described herein.

As used herein, the terms “group” and “moiety” are used interchangeably.

As used herein, a “reactive group” means a functional group on a first molecule that is capable of taking part in a chemical reaction with a functional group on a second molecule such that a covalent bond forms between the first molecule and the second molecule. Reactive groups include leaving groups, nucleophilic groups, and other reactive groups as described herein.

As used herein, the term “electrophilic leaving group” means a substituent attached to a saturated or unsaturated carbon atom that can be replaced by a nucleophile following a nucleophilic attack at that carbon atom. Those of skill in the art are routinely able to select electrophilic leaving groups that would be suitable for locating on a particular compound and for reacting with a particular nucleophile.

As used herein, the term “nucleophile” means a functional group or compound which is capable of forming a chemical bond by donating an electron pair.

As used herein, the terms “linker group”, “linker moiety”, “linking group”, or “linking moiety” (herein referred to for convenience as a “linker moiety” but noting that the terms are fully interchangeable) all mean a moiety that is capable of linking one chemical moiety to another. The nature of the linker moieties used in accordance with the present invention is not important, provided of course that the resulting chemically modified antibodies and antibody fragments are capable of fulfilling their intended purpose. A person of skill in the art would recognise that linker moieties are routinely used in the construction of conjugate molecules and could easily select appropriate linker moieties for use in conjunction with particular embodiments of the present invention.

Typically, a linker moiety for use in the present invention is an organic group. Typically, such a linker moiety has a molecular weight of 50 to 1000, preferably 100 to 500. Examples of linker moieties appropriate for use in accordance with the present invention are common general knowledge in the art and described in standard reference text books such as “Bioconjugate Techniques” (Greg T. Hermanson, Academic Press Inc., 1996), the content of which is herein incorporated by reference in its entirety.

As used herein, the term “alkyl” includes both saturated straight chain and branched alkyl groups. Preferably, an alkyl group is a C₁₋₂₀ alkyl group, more preferably a C₁₋₁₅, more preferably still a C₁₋₁₂ alkyl group, more preferably still, a C₁₋₆ alkyl group, and most preferably a C₁₋₄ alkyl group. Particularly preferred alkyl groups include, for example, methyl, ethyl, propyl, isopropyl, butyl, isobutyl, tert-butyl, pentyl and hexyl. The term “alkylene” should be construed accordingly.

As used herein, the term “alkenyl” refers to a group containing one or more carbon-carbon double bonds, which may be branched or unbranched. Preferably the alkenyl group is a C₂₋₂₀ alkenyl group, more preferably a C₂₋₁₅ alkenyl group, more preferably still a C₂₋₁₂ alkenyl group, or preferably a C₂₋₆ alkenyl group, and most preferably a C₂₋₄ alkenyl group. The term “alkenylene” should be construed accordingly.

As used herein, the term “alkynyl” refers to a carbon chain containing one or more triple bonds, which may be branched or unbranched. Preferably the alkynyl group is a C₂₋₂₀ alkynyl group, more preferably a C₂₋₁₅ alkynyl group, more preferably still a C₂₋₁₂ alkynyl group, or preferably a C₂₋₆ alkynyl group and most preferably a C₂₋₄ alkynyl group. The term “alkynylene” should be construed accordingly.

Unless otherwise specified, an alkyl, alkenyl or alkynyl group is typically unsubstituted. However, where such a group is indicated to be unsubstituted or substituted, one or more hydrogen atoms are optionally replaced by halogen atoms or —NH₂ or sulfonic acid groups. Preferably, a substituted alkyl, alkenyl or alkynyl group has from 1 to 10 substituents, more preferably 1 to 5 substituents, more preferably still 1, 2 or 3 substituents and most preferably 1 or 2 substituents, for example 1 substituent. Preferably a substituted alkyl, alkenyl or alkynyl group carries not more than 2 sulfonic acid substituents. Halogen atoms are preferred substituents. Preferably, though, an alkyl, alkenyl or alkynyl group is unsubstituted.

In the moiety that is an alkyl, alkenyl or alkynyl group or an alkylene, alkenylene or alkynylene group, in which (a) 0, 1 or 2 carbon atoms may be replaced by groups selected from C₆₋₁₀ arylene, 5- to 10-membered heteroarylene, C₃₋₇ carbocyclylene and 5- to 10-membered heterocyclylene groups, and (b) 0 to 6 —CH₂— groups may be replaced by groups selected from —O—, —S—, —S—S—, —C(O)—, —C(O)—O—, —O—C(O)—, —NH—, —N(C₁₋₆ alkyl)-, —NH—C(O)—, —C(O)—NH—, —O—C(O)—NH—, and —NH—C(O)—O— groups, a total of 0 to 6 of said carbon atoms and —CH₂— groups are preferably replaced, more preferably a total of 0 or 4 and more preferably still a total of 0, 1 or 2. Most preferably, none of the carbon atoms or —CH₂— groups is replaced.

Preferred groups for replacing a —CH₂— group are O—, —S—, —C(O)—, —C(O)—O—, —O—C(O)—, —NH—, —NH—C(O)— and —C(O)—NH— groups. Preferred groups for replacing a carbon atom are phenylene, 5- to 6-membered heteroarylene, C₅₋₆ carbocyclylene and 5- to 6-membered heterocyclylene groups. As used herein, the reference to “0, 1 or 2 carbon atoms” means any terminal or non-terminal carbon atom in the alkyl, alkenyl or alkynyl chain, including any hydrogen atoms attached to that carbon atom. As used herein, the reference to “0 to 6 —CH₂— groups” means 0, 1, 2, 3, 4, 5 or 6 —CH₂— groups and each said —CH₂— group refers to a group which does not correspond to a terminal carbon atom in the alkyl, alkenyl or alkynyl chain or to a terminal carbon atom, where the residual hydrogen atom is retained (e.g., where a —CH₃ is replaced by an —O—, the result is an —OH group).

As used herein, a C₆₋₁₀ aryl group is a monocyclic or polycyclic 6- to 10-membered aromatic hydrocarbon ring system having from 6 to 10 carbon atoms. Phenyl is preferred. The term “arylene” should be construed accordingly.

As used herein, a 5- to 10-membered heteroaryl group is a monocyclic or polycyclic 5- to 10-membered aromatic ring system, such as a 5- or 6-membered ring, containing at least one heteroatom, for example 1, 2, 3 or 4 heteroatoms, selected from O, S and N. When the ring contains 4 heteroatoms these are preferably all nitrogen atoms. The term “heteroarylene” should be construed accordingly.

Examples of monocyclic heteroaryl groups include thienyl, furyl, pyrrolyl, imidazolyl, thiazolyl, isothiazolyl, pyrazolyl, oxazolyl, isoxazolyl, triazolyl, thiadiazolyl, oxadiazolyl, pyridinyl, pyridazinyl, pyrimidinyl, pyrazinyl, triazinyl and tetrazolyl groups.

Examples of polycyclic heteroaryl groups include benzothienyl, benzofuryl, benzimidazolyl, benzothiazolyl, benzisothiazolyl, benzoxazolyl, benzisoxazolyl, benztriazolyl, indolyl, isoindolyl and indazolyl groups. Preferred polycyclic groups include indolyl, isoindolyl, benzimidazolyl, indazolyl, benzofuryl, benzothienyl, benzoxazolyl, benzisoxazolyl, benzothiazolyl and benzisothiazolyl groups, more preferably benzimidazolyl, benzoxazolyl and benzothiazolyl, most preferably benzothiazolyl. However, monocyclic heteroaryl groups are preferred.

Preferably the heteroaryl group is a 5- to 6-membered heteroaryl group. Particularly preferred heteroaryl groups are thienyl, pyrrolyl, imidazolyl, thiazolyl, isothiazolyl, pyrazolyl, oxazolyl, isoxazolyl, triazolyl, pyridinyl, pyridazinyl, pyrimidinyl and pyrazinyl groups. More preferred groups are thienyl, pyridinyl, pyridazinyl, pyrimidinyl, pyrazinyl, pyrrolyl and triazinyl, most preferably pyridinyl.

As used herein, a 5- to 10-membered heterocyclyl group is a non-aromatic, saturated or unsaturated, monocyclic or polycyclic C₅₋₁₀ carbocyclic ring system in which one or more, for example 1, 2, 3 or 4, of the carbon atoms are replaced with a moiety selected from N, O, S, S(O) and S(O)₂. Preferably, the 5- to 10-membered heterocyclyl group is a 5- to 6-membered ring. The term “heterocyclyene” should be construed accordingly.

Examples of heterocyclyl groups include azetidinyl, oxetanyl, thietanyl, pyrrolidinyl, imidazolidinyl, oxazolidinyl, isoxazolidinyl, thiazolidinyl, isothiazolidinyl, tetrahydrofuranyl, tetrahydrothienyl, tetrahydropyranyl, tetrahydrothiopyranyl, dithiolanyl, dioxolanyl, pyrazolidinyl, piperidinyl, piperazinyl, hexahydropyrimidinyl, methylenedioxyphenyl, ethylenedioxyphenyl, thiomorpholinyl, S-oxo-thiomorpholinyl, S,S-dioxo-thiomorpholinyl, morpholinyl, 1,3-dioxolanyl, 1,4-dioxolanyl, trioxolanyl, trithianyl, imidazolinyl, pyranyl, pyrazolinyl, thioxolanyl, thioxothiazolidinyl, 1H-pyrazol-5-(4H)-onyl, 1,3,4-thiadiazol-2(3H)-thionyl, oxopyrrolidinyl, oxothiazolidinyl, oxopyrazolidinyl, succinimido and maleimido groups and moieties. Preferred heterocyclyl groups are pyrrolidinyl, imidazolidinyl, oxazolidinyl, isoxazolidinyl, thiazolidinyl, isothiazolidinyl, tetrahydrofuranyl, tetrahydrothienyl, tetrahydropyranyl, tetrahydrothiopyranyl, dithiolanyl, dioxolanyl, pyrazolidinyl, piperidinyl, piperazinyl, hexahydropyrimidinyl, thiomorpholinyl and morpholinyl groups and moieties. More preferred heterocyclyl groups are tetrahydropyranyl, tetrahydrothiopyranyl, thiomorpholinyl, tetrahydrofuranyl, tetrahydrothienyl, piperidinyl, morpholinyl and pyrrolidinyl groups.

For the avoidance of doubt, although the above definitions of heteroaryl and heterocyclyl groups refer to an “N” moiety which can be present in the ring, as will be evident to a skilled chemist the N atom will be protonated (or will carry a substituent as defined below) if it is attached to each of the adjacent ring atoms via a single bond.

As used herein, a C₃₋₇ carbocyclyl group is a non-aromatic saturated or unsaturated hydrocarbon ring having from 3 to 7 carbon atoms. Preferably it is a saturated or mono-unsaturated hydrocarbon ring (i.e. a cycloalkyl moiety or a cycloalkenyl moiety) having from 3 to 7 carbon atoms, more preferably having from 5 to 6 carbon atoms. Examples include cyclopropyl, cyclobutyl, cyclopentyl and cyclohexyl and their mono-unsaturated variants. Particularly preferred carbocyclic groups are cyclopentyl and cyclohexyl. The term “carbocyclylene” should be construed accordingly.

Where specified, 0, 1 or 2 carbon atoms in a carbocyclyl or heterocyclyl group may be replaced by —C(O)— groups. As used herein, the “carbon atoms” being replaced are understood to include the hydrogen atoms to which they are attached. When 1 or 2 carbon atoms are replaced, preferably two such carbon atoms are replaced. Preferred such carbocyclyl groups include a benzoquinone group and preferred such heterocyclyl groups include succinimido and maleimido groups.

Unless otherwise specified, an aryl, heteroaryl, carbocyclyl or heterocyclyl group is typically unsubstituted. However, where such a group is indicated to be unsubstituted or substituted, one or more hydrogen atoms are optionally replaced by halogen atoms or nitro, carboxyl, cyano, acyl, acylamino, carboxamide, sulfonamide, trifluoromethyl, phosphate, C₁₋₆ alkyl, C₆₋₁₀ aryl, 5- to 10-membered heteroaryl, C₃₋₇ carbocyclyl, 5- to 10-membered heterocyclyl, —ORₓ, —SRₓ, —N(Rₓ)(R_{y}) and —SO₂—Rₓ groups, wherein Rₓ and R_{y} are independently selected from hydrogen atoms and C₁₋₆ alkyl and C₆₋₁₀ aryl groups.

Preferably, a substituted aryl, heteroaryl, carbocyclyl or heterocyclyl group has from 1 to 4 substituents, more preferably 1 to 2 substituents and most preferably 1 substituent. Preferably a substituted aryl, heteroaryl, carbocyclyl or heterocyclyl group carries not more than 2 nitro substituents and not more than 2 sulfonic acid substituents. Preferred substituents include C₁₋₆ alkyl, —(C₁₋₆ alkyl), carboxamide and acyl. Preferably, though, an aryl, heteroaryl, carbocyclyl or heterocyclyl group is unsubstituted.

As used herein, halogen atoms are typically F, Cl, Br or I atoms, preferably Br or Cl atoms, more preferably Br atoms.

As used herein, a C₁₋₆ alkoxy group is a C₁₋₆ alkyl (e.g. a C₁₋₄ alkyl) group which is attached to an oxygen atom.

As used herein, a C₁₋₆ alkylthiol group is a C₁₋₆ alkyl (e.g. a C₁₋₄ alkyl) group which is attached to a sulfur atom.

As used herein, a 5- to 10-membered heterocyclylthiol is a 5- to 10-membered (e.g., a 5- to 6-membered) heterocyclyl group which is attached to a sulfur atom.

As used herein, a C₆₋₁₀ arylthiol is a C₆₋₁₀ aryl (e.g., a phenyl) group which is attached to a sulfur atom.

As used herein, a C₃₋₇ carbocyclylthiol is a C₃₋₇ carbocyclyl (e.g., a C₅₋₆ carbocyclyl) group which is attached to a sulfur atom.

### Number and Location of Chemical Modifications of the Antibody AB

In the inter-chain bridging moiety of formula (IA) or (IB) of the chemically modified antibody AB of the present invention,

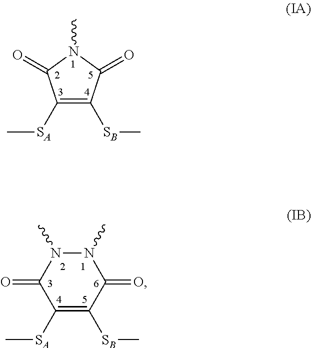

S_{A} and S_{B} are sulfur atoms that are attached to different chains of said chemically modified antibody. As explained elsewhere herein, in the chemically modified antibody AB of the present invention, each said at least one inter-chain bridging moiety typically replaces one inter-chain disulfide bond that is present in the corresponding, unmodified antibody. Furthermore, the sulfur atoms S_{A} and S_{B} correspond to the sulfur atoms of the said inter-chain disulfide present in the corresponding, unmodified antibody. It can therefore be seen that the inter-chain disulfide bridge has been replaced by an inter-chain bridging moiety that comprises the bridging unit —S_{A}—C═C—S_{B}—. The present inventors have found that this bridging unit helps to retain, and sometimes even to enhance, the structural integrity and specific binding ability, of the antibody.

The chemically modified antibody AB of the present invention is preferably an IgG1 antibody. Thus, typically each said inter-chain bridging moiety of formula (IA) or (IB) replaces one of the four inter-chain disulfide bonds present in the corresponding, unmodified IgG1 antibody.

By suitably adjusting the reaction conditions used to generate the chemically modified antibody AB from its corresponding antibody, the present inventors have found that a chemically modified antibody carrying a specific number of inter-chain bridging moieties in specific locations (i.e., bridging particular chains) can be obtained. Accordingly, the chemically modified antibody AB of the present invention may be an IgG1 antibody which:
(i) has one inter-chain bridging moiety of the formula (IA) or (IB) and whose chains are otherwise bridged by disulfide bridges —S—S— (i.e., which retains three inter-chain disulfide bonds);
(ii) has two inter-chain bridging moieties of the formula (IA) or (IB) and whose chains are otherwise bridged by disulfide bridges —S—S— (i.e., which retains two inter-chain disulfide bonds);
(iii) has three inter-chain bridging moieties of the formula (IA) or (IB) and whose chains are otherwise bridged by disulfide bridges —S—S— (i.e., which retains one inter-chain disulfide bond); or
(iv) has four inter-chain bridging moieties of the formula (IA) or (IB) (i.e., which retains no inter-chain disulfide bonds).

In (i), the said inter-chain bridging moiety of the formula (IA) or (IB) may bridge the two heavy chains, or alternatively may bridge a light chain to a heavy chain.

In (ii), each of the two inter-chain bridging moieties of the formula (IA) or (IB) may bridge one of the two heavy chains to one of the two light chains (i.e., the inter-chain bridging moieties may be confined to the Fab region of the antibody). Alternatively, each of the two inter-chain bridging moieties of the formula (IA) or (IB) may bridge the two heavy chains (i.e., the inter-chain bridging moieties may be confined to the Fc region of the antibody). Still further, one of the inter-chain bridging moieties of the formula (IA) or (IB) may bridge the two heavy chains and the other of the inter-chain bridging moieties of the formula (IA) or (IB) may bridge a light chain to a heavy chain.

In (iii), the chemically modified antibody may retain one inter-chain disulfide bond between the two heavy chains (i.e., in the Fc region), or alternatively it may retain one inter-chain disulfide bond between a heavy chain and a light chain (i.e., in the Fab region).

For the avoidance of doubt, in (ii), (iii), (iv), typically all of the inter-chain bridging moieties that are present on the chemically modified antibody AB are either: (A) inter-chain bridging moieties of the formula (IA); or (B) inter-chain bridging moieties of the formula (IB). As will be evident to one of skill in the art, typically a chemically modified antibody is produced using a reagent that introduces either moieties of the formula (IA) or moieties of the formula (IB), rather than a mixture of both. Nonetheless, it will be appreciated that construction of chemically modified antibodies comprising both moieties of formula (IA) and moieties of formula (IB), i.e. by using multiple reagents.

The present invention also provides compositions that comprise one or more chemically modified antibodies of the present invention.

One exemplary composition of the present invention contains a specific chemically modified antibody AB of the present invention that is capable of specific binding to a particular antigen AG, and which comprises substantially no other such chemically modified antibodies AB of the present invention that are capable of specific binding to the antigen AG. By “substantially no” is meant less than 10% by weight, for example less than 5% or less than 1% by weight. In other words, the said composition may comprise a chemically modified antibody containing a specific number of inter-chain bridging moieties, in specific locations, with substantially no chemically modified antibodies based on the same corresponding antibody (and which therefore can specifically bind to the same antigen AG) but with a different number and/or location of inter-chain bridging moieties. In this composition the said specific chemically modified antibody AB of the present invention is preferably as defined in (i), (ii), (iii) or (iv) above. The said composition may of course comprise other components, including other antibodies or chemically modified antibodies (such as antibodies or chemically modified antibodies that are capable of specific binding to an antigen other than the antigen AG).

This exemplary composition can thus be regarded as a substantially homogeneous chemically modified antibody composition. By “substantially homogeneous” is meant that substantially no chemically modified antibodies AB of the present invention capable of specific binding to the antigen AG other than the said specific chemically modified antibody is present in the composition.

More generally, exemplary compositions of the present invention may comprise a plurality of chemically modified antibodies of the present invention (plurality here meaning more than one chemically modified antibody that is capable of binding to a particular antigen AG, i.e. which is based on a particular native antibody), but nonetheless contain a specific chemically modified antibody of the present invention in a greater than statistical amount. Such compositions may be, but are not necessarily, substantially homogeneous as defined above. However, they nonetheless reflect the selectivity of the synthetic methods of the present invention in that they lead to an “over-population” of chemically modified antibodies of the present invention that have a specific number, and location, of inter-chain bridging moieties.

Thus, an exemplary composition of the present invention comprises one or more chemically modified antibodies AB of the present invention and which are capable of specific binding to a particular antigen AG. Furthermore, a specific chemically modified antibody of said one or more chemically modified antibodies is present in an amount of at least 30% by weight of the total amount of said one or more chemically modified antibodies. Typically in such a composition the said specific chemically modified antibody is present in a greater amount, by weight, than any other of the one or more chemically modified antibodies.

By “specific chemically modified antibody” is meant a chemically modified antibody having a specific number of (specific) inter-chain bridging moieties in specific locations. In particular, the said specific chemically modified antibody is preferably as defined in (i), (ii), (iii) or (iv) above, i.e. it preferably is an IgG1 antibody comprising one, two, three or four inter-chain bridging moieties.

Preferably, the amount of said specific chemically modified antibody is at least 40% by weight, more preferably at least 50% by weight and most preferably at least 60% by weight, of the total amount of the said chemically modified antibodies. It will be appreciated that in a “substantially homogeneous” composition as defined above, the amount of said specific chemically modified antibody is at least 90% by weight of the total amount of the said chemically modified antibodies. That constitutes a particularly preferred embodiment of the present invention.

Again, for the avoidance of doubt it is emphasised that the composition may comprise other components in any relative quantities. For example, difference antibodies or chemically modified antibodies that are capable of specific binding to different antigens from AG may be present in arbitrary quantities.

### Structure of the Inter-Chain Bridging Moiety of Formula (IA) or (IB)

In the inter-chain bridging moiety of formula (IA) or (IB),

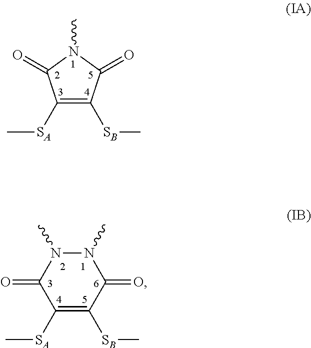

the symbol [symbol] means a point of attachment to another group. The identity of the group is not critical to the present invention, which is based on the finding that the specific maleimide and 3,6-dioxopyridazine bridging reagents can be used to selectively functionalise antibodies and antibody fragments. Exemplary such groups are nonetheless discussed in further detail herein.

Preferably, in the chemically modified antibody of the present invention, each said at least one inter-chain bridging moiety of the formula (IA) is the same or different and is a moiety of the formula (IA′):

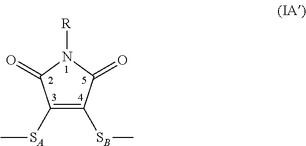

wherein:
R is (i) a hydrogen atom, (ii) a cargo moiety or (iii) a linker moiety, said linker moiety optionally being linked to a cargo moiety; and
S_{A} and S_{B} are sulfur atoms that are attached to different chains of said chemically modified antibody.

Usually, each said at least one inter-chain bridging moiety of the formula (IA) is the same. Chemically modified antibodies in which each said at least one inter-chain bridging moiety of the formula (IA) is the same are easier to synthesise. However, it is also possible for the inter-chain bridging moieties of the formula (IA) to be different. This can be achieved, for example, by using a plurality of different reagents during synthesis of the chemically modified antibody from its corresponding antibody.

It will be understood that an inter-chain bridging moiety of the formula (IA′) may constitute either (a) a chemically reactive moiety that is suitable for effecting further functionalisation of the chemically modified antibody, or (b) a moiety that carries a cargo moiety and which thus renders the chemically modified antibody a bioconjugate construct. Specifically, where R is a hydrogen atom or a linker moiety not linked to a cargo moiety, then the inter-chain bridging moiety of the formula (IA′) constitutes a moiety (a). Further, where R is a cargo moiety or a linker moiety linked to at least one cargo moiety, then the inter-chain bridging moiety of the formula (IA′) constitutes a moiety (b).

The terms “cargo moiety” and “linker moiety” as used in the context of the inter-chain bridging moiety of the formula (IA′) are as defined herein. One of ordinary skill in the art would readily appreciate that both the cargo moiety and the linker moiety can be routinely selected according to the intended function of the chemically modified antibody.

In a preferred embodiment, the chemically modified antibody of the present invention comprises at least one cargo moiety, for example at least one (such as one) cargo moiety attached to each inter-chain bridging moiety of the formula (IA). In a particularly preferred embodiment, each inter-chain bridging moiety of the formula (IA) is an inter-chain bridging moiety of the formula (IA′) that comprises at least one (e.g., one) cargo moiety. In this embodiment, the chemically modified antibody constitutes a conjugate, since it contains both the antibody and at least one cargo moiety.

In an alternative preferred embodiment, the chemically modified antibody of the present invention comprises no cargo moieties. For example, in this chemically modified antibody, each inter-chain bridging moiety of the formula (IA) may be an inter-chain bridging moiety of the formula (IA′) that comprises no cargo moieties (i.e., where R is a hydrogen atom or a linker moiety that is not linked to a cargo moiety). In this embodiment, the chemically modified antibody is not a conjugate, but it is susceptible to further chemical functionalisation in order to introduce cargo moieties of interest for a given application.

In one currently particularly preferred embodiment, if present the, or each (preferably each), cargo moiety in the chemically modified antibody comprising the inter-chain bridging moiety of formula (IA) is a drug moiety. It will be appreciated that in this embodiment the chemically modified antibody is an “antibody-drug conjugate”, or “ADC”. ADCs combine the power of antibody selectivity with the therapeutic activity of small drugs and are currently of significant research and clinical interest in the field of cancer therapy.

Thus, particularly preferred drug moieties are cytotoxic agents. Preferred cytotoxic agents include anthracyclines, auristatins, maytansinoids, calicheamicins, taxanes, benzodiazepines and duocarmycins. Other preferred drug moieties include radionuclide drugs and photosensitisers.

The skilled person would be aware that in the context of a chemically modified antibody carrying a cytotoxic agent, an exemplary application lies in the field of cancer therapy, in which the antibody specifically targets cancer cells in vivo, and therefore leads to selective delivery of cytotoxic agent thereto.

Typically where an ADC is intended to target a cell such as a cancer cell the antibody will be selected so that its antigen AG is an antigen over-expressed by that cell with respect to expression on non-cancer cells, e.g. an antigen that is over-expressed on the surface of a particular type of cancer cell, or an antigen AG that is otherwise associated with cancer cells. This enables the ADC to be targeted specifically to the cells on which the therapeutic effect (e.g., a cytotoxic effect achieved via a cytotoxic agent) is desired. Consequently in a preferred embodiment, the chemically modified antibody comprises at least one cytotoxic agent and the antigen AG is an antigen that is over-expressed by, or otherwise associated with, cancer cells, such as the exemplary such antigens described herein.

Numerous ADCs have already been developed wherein an antibody fragment is conjugated to a drug moiety via a known linker. Chemically modified antibodies of the present invention include compounds that comprise any of these previously known “pairs” of antibody and drug moiety, but modified to be conjugated in a selective manner via the inter-chain bridging moieties of the present invention.

Antibodies immunospecific for a cancer cell antigen can be obtained commercially or produced by any method known to one of skill in the art such as, e.g., recombinant expression techniques. The nucleotide sequence encoding antibodies immunospecific for a cancer cell antigen can be obtained, e.g., from the GenBank database or a database like it, the literature publications, or by routine cloning and sequencing.

Non-limiting exemplary antibodies for use in the present invention include antibodies that are capable of specific binding to the following antigens (exemplary, but non-limiting, corresponding disease states being listed in parentheses): CA125 (ovarian), CA15-3 (carcinomas), CA19-9 (carcinomas), CA 242 (colorectal), L6 (carcinomas), CD2 (Hodgkin's Disease or non-Hodgkin's lymphoma), CD3, CD4, CD5, CD6, CD11, CD25, CD26, CD37, CD44, CD64, CD74, CD205, CD227, CD79, CD105, CD138, CD20 (non-Hodgkin's lymphoma), CD52 (leukemia), CD33 (leukemia), CD22 (lymphoma), CD38 (multiple myeloma), CD40 (lymphoma), CD19 (non-Hodgkin's lymphoma), CD30 (CD30+ malignancies), CD70, CD56 (small-cell lung cancer, ovarian cancer, multiple myeloma, solid tumors), Lewis Y (carcinomas), Lewis X (carcinomas), human chorionic gonadotropin (carcinoma), alpha fetoprotein (carcinomas), placental alkaline phosphatase (carcinomas), prostate specific antigen (prostate), prostate specific membrane antigen (prostate), prostatic acid phosphatase (prostate), epidermal growth factor (carcinomas), MAGE-1 (carcinomas), MAGE-2 (carcinomas), MAGE-3 (carcinomas), MAGE-4 (carcinomas), anti-transferrin receptor (carcinomas), p97 (melanoma), MUC1 (breast cancer), CEA (colorectal), gp100 (melanoma), MARTI (melanoma), IL-2 receptor (T-cell leukemia and lymphomas), mucin (carcinomas), P21 (carcinomas), MPG (melanoma), Neu oncogene product (carcinomas), BCMA, Glypican-3, Liv-1 or Lewis Y (epithelial tumors), HER2 (breast cancer), GPNMB (breast cancer), CanAg (solid tumors), DS-6 (breast cancer, ovarian cancer, solid tumors), HLA-Dr10 (non-Hodgkin's lymphoma), VEGF (lung and colorectal cancers), MY9, B4, EpCAM, EphA receptors, EphB receptors, EGFR, EGFRvIII, HER2, HERS, BCMA, PSMA, mesothelin, cripto, alpha(v)beta3, alpha(v)beta5, alpha(v) beta6 integrin, C242, EDB, TMEFF2, FAP, TAG-72, GD2, CAIX and 5T4.

Currently particularly preferred antibodies include those capable of specific binding to the following antigens: MY9, B4, EpCAM, CD2, CD3, CD4, CD5, CD6, CD11, CD19, CD20, CD22, CD25, CD26, CD30, CD33, CD37, CD38, CD40, CD44, CD56, CD64, CD70, CD74, CD79, CD105, CD138, CD205, CD227, EphA receptors, EphB receptors, EGFR, EGFRvIII, HER2, HER3, BCMA, PSMA, Lewis Y, mesothelin, cripto, alpha(v)beta3, alpha(v)beta5, alpha(v) beta6 integrin, C242, CA125, GPNMB, ED-B, TMEFF2, FAP, TAG-72, GD2, CAIX and 5T4.

Examples of antibodies known for use in the treatment of cancer include RITUXAN® (rituximab; Genentech) which is a chimeric anti-CD20 monoclonal antibody for the treatment of patients with non-Hodgkin's lymphoma; OVAREX which is a murine antibody for the treatment of ovarian cancer; PANOREX (Glaxo Wellcome, NC) which is a murine IgG₂ₐ antibody for the treatment of colorectal cancer; Cetuximab ERBITUX (Imclone Systems Inc., NY) which is an anti-EGFR IgG chimeric antibody for the treatment of epidermal growth factor positive cancers, such as head and neck cancer; Vitaxin (Medlmmune, Inc., MD) which is a humanized antibody for the treatment of sarcoma; CAMPATH I/H (Leukosite, MA) which is a humanized IgG₁ antibody for the treatment of chronic lymphocytic leukemia (CLL); SMART MI95 (Protein Design Labs, Inc., CA) and SGN-33 (Seattle Genetics, Inc., WA) which is a humanized anti-CD33 IgG antibody for the treatment of acute myeloid leukemia (AML); LYMPHOCIDE (Immunomedics, Inc., NJ) which is a humanized anti-CD22 IgG antibody for the treatment of non-Hodgkin's lymphoma; SMART ID10 (Protein Design Labs, Inc., CA) which is a humanized anti-HLA-DR antibody for the treatment of non-Hodgkin's lymphoma; ONCOLYM (Techniclone, Inc., CA) which is a radiolabeled murine anti-HLA-Dr10 antibody for the treatment of non-Hodgkin's lymphoma; ALLOMUNE (BioTransplant, CA) which is a humanized anti-CD2 mAb for the treatment of Hodgkin's Disease or non-Hodgkin's lymphoma; AVASTIN (Genentech, Inc., CA) which is an anti-VEGF humanized antibody for the treatment of lung and colorectal cancers; Epratuzamab (Immunomedics, Inc., NJ and Amgen, Calif.) which is an anti-CD22 antibody for the treatment of non-Hodgkin's lymphoma; CEACIDE (Immunoniedics, NJ) which is a humanized anti-CEA antibody for the treatment of colorectal cancer; and Herceptin (TRASTUZUMAB), which is an anti-HER2/neu receptor monoclonal antibody for the treatment of breast cancer.

Preferably when R is a linker moiety, the said linker moiety is capable of undergoing chemical fragmentation by enzymatic catalysis, acidic catalysis, basic catalysis, oxidative catalysis and reductive catalysis. The use of linker moieties that are susceptible to chemical fragmentation is well established in bioconjugate technology, particularly for example in ADC technology. As would be understood by those skilled in the art, use of chemically fragmentable linker moieties is advantageous in applications where the intention is for a conjugate to have a limited lifetime, following which fragmentation occurs to release one or more cargo moieties.

A particularly well-established field in which linker moieties capable of undergoing chemical fragmentation are used is that of ADC technology. Here, an antibody is used to target a cargo moiety (typically a drug moiety) to a region of interest in vivo (e.g., to target cells that are targeted via binding of the antibody to an antigen expressed on the cell surface). The chemical fragmentation of the linker then releases the cargo moiety once the conjugate has reached the region of interest. For the avoidance of doubt, all types of linker moieties typically used in such techniques can readily be used in the present invention. One representative review of suitable linker moieties for linking together antibodies to cargo moieties, as in ADCs, and which linker moieties can be used in the present invention is provided by Ducry and Stump in Bioconjugate Chem. 2010 21 5-13, the content of which is herein incorporated by reference in its entirety.

In the embodiment where the linker moiety is capable of undergoing chemical fragmentation by enzymatic catalysis, acidic catalysis, basic catalysis, oxidative catalysis and reductive catalysis, the chemical structure of the linker moiety is selected with a view to rendering it susceptible to the desired chemical fragmentation mechanism. The skilled person would be well aware of suitable chemical motifs for achieving the desired mechanisms of chemical fragmentation.

For example, where chemical fragmentation via acidic catalysis is desired, the linker moiety must contain an acid labile motif within its overall structure (exemplary such acid labile motifs being carbamate and hydrazone motifs). One specific example of such an acid labile motif is:

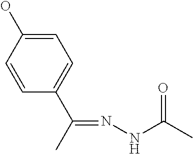

Similarly, where reductive catalysis is desired, the linker moiety must contain a motif that is susceptible to reductive cleavage (e.g., a disulfide bond).

An example of a linker moiety capable of undergoing chemical fragmentation by enzymatic catalysis is a linker comprising a protease-cleavable peptide motif. One specific example of such a protease-cleavable peptide motif is:

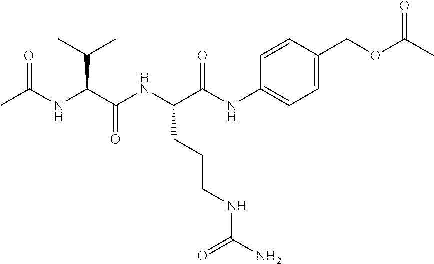 [SMILES: CC(=O)N[C@H](C(=O)N[C@@H](CCCNC(N)=O)C(=O)Nc1ccc(COC(C)=O)cc1)C(C)C]

This motif is used, for example, in the commercially available ADC product, brentuximab vedotin (a CD30-directed antibody-drug conjugate for use in treating certain cancers).

When R is a linker moiety, one exemplary structure for the said linker moiety is a moiety of the formula -L(CM)ₘ(Z)ₙ₋ₘ, wherein:
L represents a linking moiety;
each CM is the same or different and represents a cargo moiety;
each Z is the same or different and represents a reactive group attached to L and which is capable of reacting with a cargo moiety such that said cargo moiety becomes linked to L;
n is 1, 2 or 3; and
m is an integer of from zero to n.

For the avoidance of doubt, in the formula L(CM)ₘ(Z)ₙ₋ₘ the linking moiety L carries m cargo moieties CM and n-m reactive groups Z. Each said cargo moiety CM and reactive group Z may be attached at any location on the linking moiety L.

When R is a linker moiety of the formula -L(CM)ₘ(Z)ₙ₋ₘ, L is preferably a moiety which is a C₁₋₂₀ alkylene group, a C₂₋₂₀ alkenylene group or a C₂₋₂₀ alkynylene group, which is unsubstituted or substituted by one or more substituents selected from halogen atoms and —NH₂ and sulfonic acid groups, and in which (a) 0, 1 or 2 carbon atoms are replaced by groups selected from C₆₋₁₀ arylene, 5- to 10-membered heteroarylene, C₃₋₇ carbocyclylene and 5- to 10-membered heterocyclylene groups, and (b) 0 to 6 —CH₂— groups are replaced by groups selected —O—, —S—, —S—S—, —C(O)—, —C(O)—O—, —O—C(O)—, —NH—, —N(C₁₋₆ alkyl)-, —NH—C(O)—, —C(O)—NH—, —O—C(O)—NH—, and —NH—C(O)—O— groups, wherein:
(i) said arylene, heteroarylene, carbocyclylene and heterocyclylene groups are unsubstituted or substituted by one or more substituents selected from halogen atoms and nitro, carboxyl, cyano, acyl, acylamino, carboxamide, sulfonamide, trifluoromethyl, phosphate, C₁₋₆ alkyl, C₆₋₁₀ aryl, 5- to 10-membered heteroaryl, C₃₋₇ carbocyclyl, 5- to 10-membered heterocyclyl, —ORₓ, —SRₓ, —N(Rₓ)(R_{y}) and —SO₂—Rₓ groups, wherein Rₓ and R_{y} are independently selected from hydrogen atoms and C₁₋₆ alkyl and C₆₋₁₀ aryl groups; and
(ii) 0, 1 or 2 carbon atoms in said carbocyclylene and heterocyclylene groups are replaced by —C(O)— groups.

For the avoidance of doubt, it is emphasised that while this definition of L refers to a C₁₋₂₀ alkylene group, a C₂₋₂₀ alkenylene group or a C₂₋₂₀ alkynylene group (i.e., to a divalent moiety which links a group CM or Z to the chemically modified antibody), in embodiments where n is greater than 1, it is to be understood that each additional CM and/or Z replaces a hydrogen atom on the corresponding divalent linking moiety L. Thus, for example, where n is 2, then L is a trivalent moiety (attaching the bridging moiety to two CMs, two Zs or one CM and one Z) and when n is 3, then L is a tetravalent moiety (attaching the bridging moiety to any three CMs and/or Zs).

Preferably any arylene, heteroarylene, carbocyclylene and heterocyclylene groups are substituted by at most two substituents and more preferably they are unsubstituted. Preferred substituents include C₁₋₆ alkyl, —O(C₁₋₆ alkyl), carboxamide and acyl.

In one aspect, L represents a moiety which is an unsubstituted C₁₋₁₂ alkylene group, and in which (a) 0 or 1 carbon atoms are replaced by a phenylene group, and (b) 0, 1 or 2 —CH₂— groups are replaced by groups selected —O—, —S—, —S—S—, —C(O)—, —C(O)—O—, —O—C(O)—, —NH—, —N(C₁₋₆ alkyl)-, —NH—C(O)—, —C(O)—NH—, —O—C(O)—NH—, and —NH—C(O)—O— groups, wherein said phenylene group is unsubstituted or substituted by one or more substituents selected from halogen atoms and nitro, carboxyl, cyano, acyl, acylamino, carboxamide, sulfonamide, trifluoromethyl, phosphate, C₁₋₆ alkyl, C₆₋₁₀ aryl, 5- to 10-membered heteroaryl, C₃₋₇ carbocyclyl, 5- to 10-membered heterocyclyl, —ORₓ, —SRₓ, —N(Rₓ)(R_{y}) and —SO₂—Rₓ groups, wherein Rₓ and R_{y} are independently selected from hydrogen atoms and C₁₋₆ alkyl and C₆₋₁₀ aryl groups.

For example, L may be a moiety which is an unsubstituted C₁₋₄ alkylene group, in which 0 or 1 carbon atom is replaced by an unsubstituted phenylene group and 0 or 1 —CH₂— group is replaced by groups selected —S—S—, —O—C(O)—NH—, and —NH—C(O)—O— groups.

Z represents a reactive group attached to a group of formula L which is capable of reacting with a cargo moiety such that the cargo moiety becomes linked to the group of formula L. As those of skill in the art would understand, the nature of the reactive group itself is not important. A very wide range of reactive groups are now routinely used in the art to connect cargo moieties to linkers in bionjugates. Such reactive groups may be capable, for example, of attaching an amine compound, a thiol compound, a carboxyl compound, a hydroxyl compound, a carbonyl compound or a compound containing a reactive hydrogen, to a linker. Those of skill in the art would of course immediately recognise that any such reactive group would be suitable for use in accordance with the present invention. Those of skill in the art would be able to select an appropriate reactive group from common general knowledge, with reference to standard text books such as “Bioconjugate Techniques” (Greg T. Hermanson, Academic Press Inc., 1996), the content of which is herein incorporated by reference in its entirety.

Z is preferably:
(a) a group of formula -LG, —C(O)-LG, —C(S)-LG or —C(NH)-LG wherein LG is an electrophilic leaving group;
(b) a nucleophile Nu′ selected from —OH, —SH, —NH₂, —NH(C₁₋₆ alkyl) and —C(O)NHNH₂ groups;
(c) a cyclic moiety Cyc, which is capable of a ring-opening electrophilic reaction with a nucleophile;
(d) a group of formula —S(O₂)(Hal), wherein Hal is a halogen atom;
(e) a group of formula —N═C═O or —N═C═S;
(f) a group of formula —S—S(IG′) wherein IG′ represents a group of formula IG as defined herein;
(g) a group AH, which is a C₆₋₁₀ aryl group that is substituted by one or more halogen atoms;
(h) a photoreactive group capable of being activated by exposure to ultraviolet light;
(i) a group of formula —C(O)H or —C(O)(C₁₋₆ alkyl);
(j) a maleimido group;
(k) a group of formula —C(O)CHCH₂;
(l) a group of formula —C(O)C(N₂)H or -PhN₂⁺, where Ph represents a phenyl group;
(m) an epoxide group;
(n) an azide group —N₃; and
(o) an alkyne group —C≡CH.

Most preferably, Z is selected from:
(a) groups of formula -LG, —C(O)-LG and —C(S)-LG, wherein LG is selected from halogen atoms and —O(C₁₋₆ alkyl), —SH, —S(C₁₋₆ alkyl), triflate, tosylate, mesylate, N-hydroxysuccinimidyl and N-hydroxysulfosuccinimidyl groups;
(b) groups of formula —OH, —SH and —NH₂;
(c) a group of formula

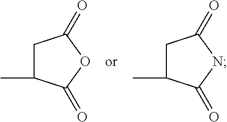

and
(d) a maleimido group.

As used herein, a “maleimido group” may be an unsubstituted maleimido group (that is typically attached to L via its nitrogen atom) or alternatively it may be a substituted maleimido group (again typically attached to L via it nitrogen atom), the said substituents being electrophilic leaving groups (e.g., groups X and Y as defined herein) located at one or both of the double-bonded ring carbon atoms (i.e., the carbon atoms at the β-positions from the nitrogen atom).

LG is preferably selected from halogen atoms and —O(IG′), —SH, —S(IG′), —NH₂, NH(IG′), —N(IG′)(IG″), —N₃, triflate, tosylate, mesylate, N-hydroxysuccinimidyl, N-hydroxysulfosuccinimidyl, imidazolyl and azide groups, wherein IG′ and IG″ are the same or different and each represents a group of formula IG.

Nu′ is preferably selected from —OH, —SH and —NH₂ groups.

Cyc is preferably selected from the groups

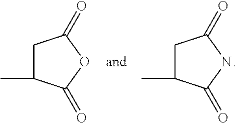

Hal is preferably a chlorine atom.

AH is preferably a phenyl group that is substituted by at least one fluorine atom.

The photoreactive group is preferably selected from:
(a) a C₆₋₁₀ aryl group which is substituted by at least one group of formula —N₃ and which is optionally further substituted by one or more halogen atoms;
(b) a benzophenone group;
(c) a group of formula —C(O)C(N₂)CF₃; and
(d) a group of formula -PhC(N₂)CF₃, wherein Ph represents a phenyl group.
   n is preferably 1 or 2, and most preferably 1.

The group IG as used herein is a chemically inert group. Typically, IG represents a moiety which is a C₁₋₂₀ alkyl group, a C₂₋₂₀ alkenyl group or a C₂₋₂₀ alkynyl group, which is unsubstituted or substituted by one or more substituents selected from halogen atoms and sulfonic acid groups, and in which (a) 0, 1 or 2 carbon atoms are replaced by groups selected from C₆₋₁₀ arylene, 5- to 10-membered heteroarylene, C₃₋₇ carbocyclylene and 5- to 10-membered heterocyclylene groups, and (b) 0, 1 or 2 —CH₂— groups are replaced by groups selected from —O—, —S—, —S—S—, —C(O)— and —N(C₁₋₆ alkyl)- groups, wherein:
(i) said arylene, heteroarylene, carbocyclylene and heterocyclylene groups are unsubstituted or substituted by one or more substituents selected from halogen atoms and C₁₋₆ alkyl, C₁₋₆ alkoxy, C₁₋₆ alkylthiol, —N(C₁₋₆ alkyl)(C₁₋₆ alkyl), nitro and sulfonic acid groups; and
(ii) 0, 1 or 2 carbon atoms in said carbocyclylene and heterocyclylene groups are replaced by —C(O)— groups

IG preferably represents a moiety which is an unsubstituted C₁₋₆ alkyl group, C₂₋₆ alkenyl group or C₂₋₆ alkynyl group, in which (a) 0 or 1 carbon atom is replaced by a group selected from phenylene, 5- to 6-membered heteroarylene, C₅₋₆ carbocyclylene and 5- to 6-membered heterocyclylene groups, wherein said phenylene, heteroarylene, carbocyclylene and heterocyclylene groups are unsubstituted or substituted by one or two substituents selected from halogen atoms and C₁₋₄ alkyl and C₁₋₄ alkoxy groups, and (b) 0, 1 or 2 —CH₂— groups are replaced by groups selected from —O—, —S— and —C(O)— groups.

More preferably, IG represents a moiety which is an unsubstituted C₁₋₆ alkyl group, in which (a) 0 or 1 carbon atom is replaced by a group selected from unsubstituted phenylene, 5- to 6-membered heteroarylene, C₅₋₆ carbocyclylene and 5- to 6-membered heterocyclylene groups.

Most preferably, IG represents an unsubstituted C₁₋₆ alkyl group.

Preferably n is 1 or 2 and most preferably n is 1. In one preferred embodiment, n and m are both equal to one (i.e., the linker moiety carries a single cargo moiety and has no reactive groups Z, thus meaning that the chemically modified antibody constitutes a conjugate). In another preferred embodiment, n is 1 and m is 0 (i.e., the linker moiety carries no cargo moiety, but carries a reactive group Z that renders the chemically modified antibody suitable for functionalisation with a cargo moiety).

In another preferred aspect of the invention, the reactive group Z is chosen such that its subsequent functionalisation to introduce a cargo moiety proceeds according to the well-known (and widely reported in the scientific literature) “Click” chemistry. “Click” chemistry encompasses a group of powerful linking reactions that are simple to perform, have high yields, require no or minimal purification, and are versatile in joining diverse structures without the prerequisite of protection steps. One representative literature article describing the Click reactions that can be utilised in the present invention, and whose content is herein incorporated by reference in its entirety, is “C. D. Hein, X.-M. Liu and D. Wang, Pharm Res 2008 25(10) 2216-2230).

“Click” reactions occur for example via the Huisgen 1,3-dipolar cycloaddition of alkynes to azides. Thus, particularly preferred reactive groups Z also include an azide group —N₃ and an alkyne group —C≡CH. As would be readily understood by those skilled in the art, such reactive groups are ideally suited for carrying out click reactions. In an especially preferred embodiment, the chemically modified antibody comprises two reactive groups Z, one of which is azide group —N₃ and the other of which is an alkyne group —C≡CH. This readily enables dual functionalisation of the chemically modified antibody using two orthogonal click reactions to introduce any two desired cargo moieties.

Preferably, in the chemically modified antibody of the present invention, each said at least one inter-chain bridging moiety of the formula (IB) is the same or different and is a moiety of the formula (IB′):

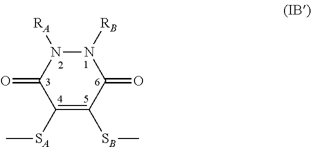

wherein:
R_{A} and R_{B} are, independently of one another, (i) a chemically inert group, (ii) a cargo moiety or (iii) a linker moiety, said linker moiety optionally being linked to at least one cargo moiety; and
S_{A} and S_{B} are sulfur atoms that are attached to different chains of said chemically modified antibody.

Usually, each said at least one inter-chain bridging moiety of the formula (IB) is the same. Chemically modified antibodies in which each said at least one inter-chain bridging moiety of the formula (IB) is the same are easier to synthesise. However, it is also possible for the inter-chain bridging moieties of the formula (IB) to be different. This can be achieved, for example, by using a plurality of different reagents during synthesis of the chemically modified antibody from its corresponding antibody.

The “chemically inert group” R_{A} and/or R_{B} is typically not hydrogen. Further, “chemically inert group” means a group that does not react (i.e., is not susceptible to reaction) under the reaction conditions in which the chemically modified antibody of the invention is produced. For example, the chemically inert group is not itself susceptible to reaction (including being susceptible to decomposition) when the corresponding inter-chain bridging reagent is reacted with the antibody to effect the desired disulfide briding. Further, the chemically inert group is typcially also not itself susceptible to reaction when reaction(s) is/are effected on a linker moiety comprised on a group R_{A} or R_{B} that is not the chemically inert group.

Typically at most one of the groups R_{A} and R_{B} is a chemically inert group. Preferably neither R_{A} nor R_{B} is a chemically inert group, i.e. R_{A} and R_{B} are, independently of one another, either (ii) a cargo moiety or (iii) a linker moiety, said linker moiety optionally being linked to at least one cargo moiety. When R_{A} and/or R_{B} is a chemically inert group, the chemically inert group is preferably a group IG as defined herein.

It will be understood that an inter-chain bridging moiety of the formula (IB′) may constitute either (a) a chemically reactive moiety that is suitable for effecting further functionalisation of the chemically modified antibody, or (b) a moiety that carries a cargo moiety and which thus renders the chemically modified antibody a bioconjugate construct. Specifically, where R_{A} and R_{B} are chemically inert groups or linker moieties not linked to a cargo moiety (typically at most one of R_{A} and R_{B} being a chemically inert group), then the inter-chain bridging moiety of the formula (IB′) constitutes a moiety (a). Further, where at least one of R_{A} and R_{B} is a cargo moiety or a linker moiety linked to at least one cargo moiety, then the inter-chain bridging moiety of the formula (IB′) constitutes a moiety (b).

The terms “cargo moiety” and “linker moiety” as used in the context of the inter-chain bridging moiety of the formula (IB′) are as defined elsewhere herein (e.g., with reference to group R). One of ordinary skill in the art would readily appreciate that both the cargo moiety and the linker moiety can be routinely selected according to the intended function of the chemically modified antibody.

In a preferred embodiment, the chemically modified antibody of the present invention comprises at least one cargo moiety, for example at least one cargo moiety (e.g. one or two, preferably two cargo moieties) attached to each inter-chain bridging moiety of the formula (IB). In a particularly preferred embodiment, each inter-chain bridging moiety of the formula (IB) is an inter-chain bridging moiety of the formula (IB′) that comprises at least one cargo moiety (e.g., two cargo moieties). In this embodiment, the chemically modified antibody constitutes a conjugate, since it contains both the antibody and at least one cargo moiety.

In one currently particularly preferred embodiment, at least one (e.g., one) cargo moiety in the chemically modified antibody comprising the inter-chain bridging moiety of formula (IB) is a drug moiety. It will be appreciated that in this embodiment the chemically modified antibody is an “antibody-drug conjugate”, or “ADC”. Preferred drug moieties include those already described elsewhere herein (e.g., the cytotoxic agents described herein).

In a particularly preferred embodiment, the inter-chain bridging moiety of the formula (IB′) comprises at least two (e.g., two) cargo moieties. For example, the inter-chain bridging moiety of the formula (IB′) may comprise both a drug moiety and an imaging agent. In this embodiment, preferably the formula R_{A} comprises said drug moiety and R_{B} comprises said imaging agent.

In an alternative embodiment, the chemically modified antibody of the present invention comprises no cargo moieties. For example, in this chemically modified antibody, each inter-chain bridging moiety of the formula (IB) may be an inter-chain bridging moiety of the formula (IB′) that comprises no cargo moieties. In this embodiment, the chemically modified antibody is not a conjugate, but it is susceptible to further chemical functionalisation in order to introduce cargo moieties of interest for a given application.

### Synthetic Methods

The present inventors have found that selective chemical modification of antibodies can be achieved by suitably adjusting the reaction conditions under which an inter-chain bridging reagent is reacted with an antibody.

By “selective” chemical modification (as in a process for “selectively” producing a chemically modified antibody) is meant effecting chemical modification of the antibody in such a way as to introduce the desired number of inter-chain bridging moieties in the desired locations. The desired number of inter-chain bridging moieties corresponds to the number of inter-chain disulfide bridges present in the antibody that are to be replaced by inter-chain bridging moieties. The desired locations corresponds to the locations of the said inter-chain disulfide bridges that are to be replaced (e.g., bridging the two heavy chains, or bridging heavy chains to light chains).

“Selective” chemical modification can be contrasted with “non-selective” chemical modification, in which the number and location of inter-chain bridging moieties introduced onto an antibody is uncontrolled and which therefore results in a heterogeneous mixture of products comprising antibodies having different numbers and/or locations of inter-chain bridging moieties.

It should be emphasised that “selective” chemical modification does not imply that pure chemically modified antibody containing only the desired number of inter-chain bridging moieties in the desired locations is obtained. A synthetic process is “selective” provided that it leads to an over-population of chemically modified antibodies of the present invention that have the desired specific number, and location, of inter-chain bridging moieties. In other words, a “selective” chemical modification constitutes a process which provides an exemplary composition of the present invention as herein defined, e.g. a composition which comprises one or more chemically modified antibodies AB of the present invention and which are capable of specific binding to a particular antigen AG, and wherein a specific chemically modified antibody of said one or more chemically modified antibodies is present in an amount of at least 30% by weight of the total amount of said one or more chemically modified antibodies (for example, at least 40% by weight, more preferably at least 50% by weight and most preferably at least 60% by weight such as at least 90% by weight, of the total amount of the said chemically modified antibodies).

In general, the process of the present invention is a process for selectively producing a chemically modified antibody and comprises both reducing at least one inter-chain disulfide bridge of an antibody in the presence of a reducing agent and reacting said antibody with at least one inter-chain bridging reagent of the formula (IIA) or (IIB)

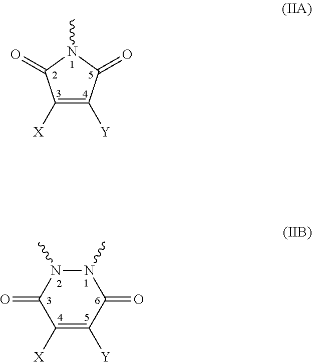

wherein X and Y each independently represent an electrophilic leaving group.

Preferably X and Y each independently represent a halogen atom or a group —SR₁, —OR₁, —NR₁R₂, —SeR₁, —SO₂R₁, —SO₂OR₁, —SO₂NR₁R₂, —SOR₁, —CN, —C(H)(COOR₁)(COOR₂) or —P(O)OR₁R₂R₃, wherein R₁, R₂ and R₃ are independently selected from hydrogen atoms and C₁₋₆ alkyl, 5- to 10-membered heterocyclyl, C₆₋₁₀ aryl and C₃₋₇ carbocyclyl groups.

More preferably, X and Y each independently represent a halogen atom or a C₁₋₆ alkylthiol, 5- to 10-membered heterocyclylthiol, C₆₋₁₀ arylthiol or C₃₋₇ carbocyclylthiol group.

Most preferably X and Y each independently represent a halogen atom, for example X and Y are each chlorine or bromine atoms.

It will be understood that the reference to “reducing at least one inter-chain disulfide bridge of an antibody” means reducing each of the inter-chain disulfide bridges that it is desired to replace with inter-chain bridging moieties. For example, if the desired product comprises two inter-chain bridging moieties, then the process comprises reducing two inter-chain disulfide bridges.

Currently preferred reducing agents include 2-mercaptoethanol, tris(2-carboxyethyl)phosphine, dithiothreitol and benzeneselenol. However, other reducing agents capable of reducing disulfide bonds may also be used, such as other phosphine, selenol, or thiol reagents.

In some embodiments the steps of reducing the at least one inter-chain disulfide bridge of an antibody in the presence of a reducing agent and of reacting said antibody with at least one inter-chain bridging reagent of the formula (IIA) or (IIB) are carried out in a single synthetic step. By a “single synthetic step” the reducing agent and the inter-chain bridging reagent of the formula (IIA) or (IIB) are added to the reaction mixture without isolation of any intermediate product formed by reducing the at least one inter-chain disulfide bridge of an antibody in the presence of a reducing agent.

When the steps of reducing the at least one inter-chain disulfide bridge of an antibody in the presence of a reducing agent and of reacting said antibody with at least one inter-chain bridging reagent of the formula (IIA) or (IIB) are carried out in a single synthetic step, the reducing agent and the inter-chain bridging reagent of the formula (IIA) or (IIB) may be added to the reaction mixture simultaneously. Alternatively, the reducing agent may be added first, with the inter-chain bridging reagent of the formula (IIA) or (IIB) being added subsequently (for example, after 0.5 to 5 hours).

In another embodiment, the steps of reducing the at least one inter-chain disulfide bridge of an antibody in the presence of a reducing agent and of reacting said antibody with at least one inter-chain bridging reagent of the formula (IIA) or (IIB) are carried out in separate synthetic steps. By “separate synthetic steps” is meant that in a first step the reducing agent is added to effect reduction of at least one inter-chain disulfide bridge of an antibody, following which excess reducing agent is removed, and thereafter in a second step the intermediate product is reacted with at least one inter-chain bridging reagent. Preferably immediately prior to the second step the intermediate product is incubated for a period of from 1 to 48 hours (such as 12 to 36 hours, for example about 24 hours); the inventors have found that such an “equilibration” period may assist in biasing the final product distribution towards production of particular desired numbers of inter-chain bridging moieties.

The relative proportions of reducing agent and inter-chain bridging reagent of the formula (IIA) or (IIB) can also be adjusted in order to increase the yield of the desired chemically modified antibody. Typical ratios of reducing agent to inter-chain bridging reagent of the formula (IIA) or (IIB) (by mole) are from 1:5 to 5:1 (for example, from 1:3 to 3:1, such as from 1:2 to 2:1).

Similarly the number of molar equivalents of reducing agent and inter-chain bridging reagent of the formula (IIA) or (IIB) with respect to the antibody can be adjusted in order to increase the yield of the desired chemically modified antibody. Typical molar equivalents of reducing agent with respect to the antibody are 2 to 100, for example 5 to 50. Typical molar equivalents of inter-chain bridging reagent of the formula (IIA) or (IIB) with respect to the antibody are 2 to 100, for example 5 to 50.

Furthermore, it is possible to carry out the process of the invention with the use of more than one reducing agent. For example, when the steps of reducing the at least one inter-chain disulfide bridge of an antibody in the presence of a reducing agent and of reacting said antibody with at least one inter-chain bridging reagent of the formula (IIA) or (IIB) are carried out in a single synthetic step, multiple reducing agents may be added simultaneously with the inter-chain bridging reagent of the formula (IIA) or (IIB), or multiple reducing agents may be added step-wise, followed by addition of the inter-chain bridging reagent of the formula (IIA) or (IIB). Similarly, when the steps of reducing the at least one inter-chain disulfide bridge of an antibody in the presence of a reducing agent and of reacting said antibody with at least one inter-chain bridging reagent of the formula (IIA) or (IIB) are carried out in separate synthetic steps, multiple reducing agents may be added simultaneously or different reducing agents may be added stepwise, prior to the step of removing excess reducing agent.

The working Examples provided herein further demonstrate the capacity of the synthetic methods of the present invention to produce chemically modified antibodies of the present invention having the desired number, and location, or inter-chain bridging moieties.

It will be appreciated that the inter-chain bridging reagent of the formula (IIA) or (IIB)

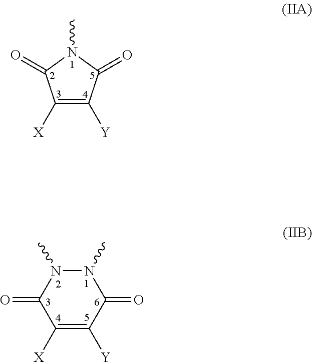

is closely related in structure to the (corresponding) inter-chain bridging moiety of moiety of the formula (IA) or (IB) that is present in the chemically modified antibodies of the present invention. It is believed that an antibody having a reduced inter-chain disulfide bridge, and therefore comprising two free thiol groups, is able to react with the inter-chain bridging reagent by attack of the respective thiol groups at the 3- and 4-positions of the inter-chain bridging reagent, with concomitant loss of the electrophilic leaving groups X and Y. This enables the antibody to “re-bridge” via the inter-chain bridging moiety of formula (IA) or (IB) as a replacement for the corresponding inter-chain disulfide bridge present in the original antibody.

Preferably the inter-chain bridging reagent of the formula (IIA) carries a group R (as defined herein) attached to the nitrogen atom at the 1-position (i.e., as in the bridging moiety of the formula (I′)). In other words, the inter-chain bridging reagent of the formula (IIA) preferably has the formula (IIA′):

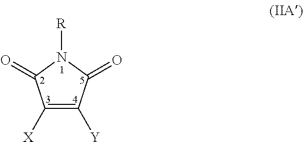

Preferably the inter-chain bridging reagent of the formula (IIB) carries the groups R_{A} and R_{B} (as defined herein) attached to the nitrogen atom at the 2-position and 1-position, respectively (i.e., as in the bridging moiety of the formula (IB′)). In other words, the inter-chain bridging reagent of the formula (IIB) preferably has the formula (IIB′):

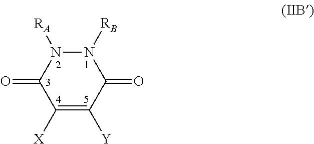

The inventors have found that the bridging reaction between the inter-chain disulfide bond in the antibody and the X-=-Y moiety within the bridging reagent of the formula (IIB) proceeds much more effectively when the nitrogen atoms at positions 2 and 1 are not attached merely to hydrogen atoms (e.g., when they are instead attached to the groups R_{A} and R_{B}, as in the formula (IIB′)). It is believed that this may be due to the pseudoaromatic character, and thus relative unresponsiveness to nucleophilic attack, of the pyridazinedione ring when it is either un- or mono-functionalised at the 1- and 2-positions. This contrasts with the reactivity behaviour of the maleimide-based bridging reagent, where the presence of a non-hydrogenic group attacged to the N-atom at the 1-position is not a prerequisite for achieving good bridging reactivity.

In the production processes of the present invention, the homogeneity (i.e., purity) of the target product can if desired be further increased by carrying out a further step, namely subsequently purifying said chemically modified antibody (or antibody fragment, where the process relates to production of chemically modified antibody fragments). Preferably the step of subsequently purifying said chemically modified antibody (or antibody fragment) comprises effecting chromatographic purification of the chemically modified antibody (or antibody fragment), for example effecting size-exclusion chromatography, immunoaffinity chromatography, ion-exchange chromatography or hydrophobic interaction chromatography. This optional purification step typically increases the relative amount of the said chemically modified antibody (or antibody fragment) with respect to any other chemically modified antibodies (or antibody fragments) that may be present in the original product mixture.

The present invention also provides the use of an inter-chain bridging reagent of the formula (IIA) or (IIB) for effecting selective chemical modification of an antibody via the selective replacement of one or more of the inter-chain disulfide bonds in said antibody by inter-chain bridging moieties of the formula (IA) or (IB).

By “selective replacement” is meant replacement of a desired number of inter-chain disulfide bonds present at desired locations on the antibody. The said inter-chain disulfide bond or bonds is or are replaced by inter-chain bridging moieties of the formula (IA) or (IB). The use may comprise carrying out the process of the present invention for producing a chemically modified antibody.

### Ring-Opening of Inter-Chain Bridging Moiety of Formula (IA)

The present invention further provides a chemically modified antibody that comprises at least one inter-chain bridging moiety of the formula (III)

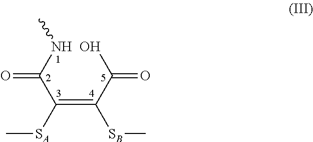

It will be appreciated that the inter-chain bridging moiety of formula (III) has a closely related chemical structure to the inter-chain bridging moiety of formula (IA). Specifically, it is a hydrolysis product of the inter-chain bridging moiety of formula (IA).

Thus, a chemically modified antibody that comprises at least one inter-chain bridging moiety of the formula (III) can be readily produced by effecting hydrolysis, and thus ring-opening, of a chemically modified antibody that comprises at least one inter-chain bridging moiety of the formula (IA). The said hydrolysis can be readily effected using known techniques for hydrolysis of maleimide compounds into maleaimic acid compounds (see for example Machida et al., Chem. Pharm. Bull. 1977 24 2739 and Ryan et al. Chem. Commun 2011 47 5452). One suitable method is to subject the corresponding chemically modified antibody comprising at least one inter-chain bridging moiety of the formula (IA) to mildly basic aqueous conditions (e.g., a pH of 7.1 or higher, for example 7.2 to 10), at a temperature of from 0 to 50° C. (e.g., from 20 to 40° C.). Any base or basic buffer solution could be used. LiOH is one suitable example. A PBS buffer solution at a pH of 7.4 is also effective.

For the avoidance of doubt, it is emphasised that preferred aspects as taught herein of the chemically modified antibody that comprises at least one inter-chain bridging moiety of the formula (IA) apply identically as preferred aspects of the chemically modified antibody that comprises at least one inter-chain bridging moiety of the formula (III). In other words, preferred numbers and locations of bridging moieties on the antibody, preferred antibodies, and preferred additional cargo moieties and linker moieties as explained in relation to the chemically modified antibody that comprises at least one inter-chain bridging moiety of the formula (IA) apply identically as preferred aspects of the chemically modified antibody that comprises at least one inter-chain bridging moiety of the formula (III).

It will, in addition, be appreciated that nitrogen at the 1-position of the bridging moiety of the formula (III) corresponds to the nitrogen at the 1-position of the bridging moiety of the formula (IA). Consequently, the group R that may be attached to the 1-position of the bridging moiety of the formula (IA) may identically be attached to the 1-position of the bridging moiety of the formula (III), with preferred embodiments of that group R as described herein being directly applicable in the context of the bridging moiety of the formula (III). In other words, a preferred bridging moiety of the formula (III) has the formula (III′):

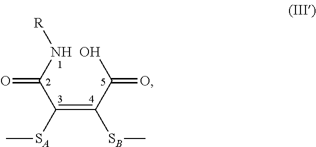

where R is as herein defined.

One advantage of effecting ring-opening in order to obtain chemically modified antibodies comprising at least one inter-chain bridging moiety of the formula (III) is that the inter-chain bridging moiety of formula (III) is less readily cleavable from the antibody than is an inter-chain bridging moiety of formula (IA).

### Application of Principles to Antibody Fragments

The principles of the present invention can also be readily applied to achieve selective chemical modification of antibody fragments.

In one aspect, the present invention thus relates to a chemically modified antibody fragment AB_{F}. The inter-chain bridging moiety of the formula (IA_{F}) or (IB_{F}) is identical to the inter-chain bridging moiety of the formula (IA) or (IB), except that its sulfur atoms S_{AF} and S_{BF} are attached to different chains of a chemically modified antibody fragment (as opposed to different chains of a chemically modified (full) antibody). Consequently, all preferred structural characteristics of the inter-chain bridging moiety of the formula (IA) or (IB), such as the identity of the group R that may be attached to the nitrogen at the 1-position in the formula (IA), and the groups R_{A} and R_{B} that are attached to the nitrogens at the 2- and 1-positions in the formula (IB), are also preferred structural characteristics of the inter-chain bridging moiety of the formula (IA_{F}) or (IB_{F}). In particular, a preferred inter-chain bridging moiety of the formula (IA_{F}) has the formula (IA_{F}′):

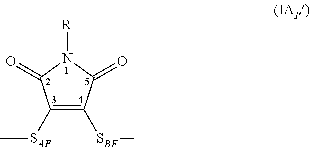

where R is as herein defined.

Further, a preferred inter-chain bridging moiety of the formula (IB_{F}) has the formula (IB_{F}′):

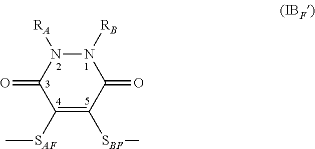

where R_{A} and R_{B} are as herein defined.

The chemically modified antibody fragment AB_{F} may be an scFv antibody fragment in which the heavy chain is bridged to the light chain via said at least one inter-chain bridging moiety of the formula (IA_{F}) or (IB_{F}).

Alternatively, the chemically modified antibody fragment AB_{F} may be a FAB antibody fragment in which the heavy chain is bridged to the light chain via said at least one inter-chain bridging moiety of the formula (IA_{F}) or (IB_{F}).

One important advantage of providing a chemically modified antibody fragment AB_{F} that comprises at least one inter-chain bridging moiety of the formula (IB_{F}) is that it provides a particularly facile means of simultaneously (a) bridging the sulfur atoms S_{AF} and S_{BF} that are attached to different chains of said chemically modified antibody fragment and (b) functionalising the said antibody fragment with at least two (e.g. two) cargo moieties. Specifically, said inter-chain bridging moiety of the formula (IB_{F}) may be linked to a first cargo moiety via the nitrogen atom at the 1-position and to a second cargo moiety via the nitrogen atom at the 2-position of the bridging moiety of the formula (IB_{F}).

In a particularly preferred embodiment, said first cargo moiety is a drug or an imaging agent and said second cargo moiety is a half-life-extending agent (these cargo moieties, and preferred embodiments thereof, being as defined elsewhere herein). More specifically, in the formula (IB_{F}′) R_{A} comprises said half-life-extending agent and R_{B} comprises said drug or imaging agent. Such a chemically modified antibody fragment, which can be regarded as an ADC owing to the presence of the drug/imaging agent component, is potentially of particularly high commercial value. That is because antibody fragments (e.g., scFV or Fab fragments) can be expressed in very high yields in bacterial hosts (rather than having to be expressed in mammalian cells, as with full antibodies). However, one ongoing issue with the use of antibody fragments in therapeutic and diagnostic applications is their tendency to be rapidly cleared in the bloodstream. Thus, in this particularly preferred chemically modified antibody fragment of the invention, one can access the advantages of facile production of the underlying fragment in a bacterial host, while mitigating the in vivo clearance problems of the underlying fragment via the presence of the half-life-extending agent.

The chemically modified antibody fragments of the present invention may be produced using the same synthetic methods as applied for producing chemically modified antibodies, but adapted to replace the antibody reagent with an appropriate antibody fragment reagent. Again, preferred aspects of the processes for producing a chemically modified antibody are also preferred aspects of the processes for producing a chemically modified antibody fragment. The present inventors have found that the synthetic methods of the present invention enable selective replacement of target inter-chain disulfide bridges with respect both to intra-chain disulfide bridges in the antibody fragment and any other (non-target) inter-chain disulfide bridges that may be present.

Typically, where a chemically modified scFv antibody fragment is to be produced, the scFv antibody fragment reagent is one that comprises a disulfide bond between the heavy chain and the light chain of the antibody fragment (e.g., an artificially introduced disulfide bond).

Similarly, the at least one inter-chain bridging moiety of the formula (IA_{F}) can be ring-opened to yield at least one inter-chain bridging moiety of the formula (III_{F}). Methods for effecting ring-opening of maleimides are as discussed elsewhere herein. A preferred inter-chain bridging moiety of the formula (III_{F}) has the formula (III_{F}′):

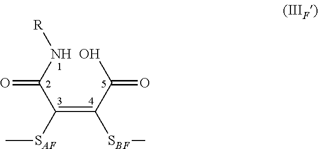

wherein R is as herein defined.

### Applications

As will be clear to those of skill in the art, the methodology and chemically modified antibodies and antibody fragments of the present invention are broadly applicable to all practical applications that rely on chemical modification of antibodies and antibody fragments. Typically, conventional processes and methods involving functionalised antibodies can straightforwardly be modified by incorporating the inter-chain bridging moieties utilised in the present invention. Advantageously, the chemically modified antibodies and antibody fragments incorporating these inter-chain bridging moieties are less heterogeneous than in prior art methods. Furthermore, there is generally no need to effect mutagenesis synthetic steps to introduce artificial residues that can then serve as the basis for chemical modification. Still further, the inter-chain bridging moieties described herein ensure that the structural integrity, and functionality, of the native antibody or antibody fragment is retained.

Examples of routine processes include processes for detecting an antigen AG, biotechnological purification processes and assay processes for identifying whether a substance interacts with such a compound. Such processes include ELISA (“enzyme-linked immunosorbent assay”) processes, LAB (“labelled avidin-biotin”) assay processes, BRAB (“bridged avidin-biotin”) assay processes, ABC (“avidin-biotin complex”) assay processes, and FRET (“Forster resonance energy transfer”) assays.

However, one particularly preferred application for the products of the present invention is in the therapy and diagnostics. As explained elsewhere herein, antibodies, and antibody fragments, have the ability to bind specifically to a target antigen AG. That ability can be exploited to direct a cargo moiety of diagnostic or therapeutic utility to a desired location in vivo, specifically by conjugating the said cargo moiety to an antibody or antibody fragment that binds specifically to a target antigen of interest (e.g., a target antigen that is expressed on the surface of cells of interest, such as cancer cells). In one particularly preferred embodiment, the chemically modified antibody or antibody fragment is capable of specific binding to an antigen of clinical significance (e.g., an antigen expressed on a cancer cell) and the said chemically modified antibody or antibody fragment further carries at least one cargo moiety that is a detectable moiety or a drug (e.g., a cytotoxic drug).

The present invention thus also provides a pharmaceutical composition comprising: (i) a chemically modified antibody (or antibody fragment) of the present invention, which comprises at least one cargo moiety that is a drug or a diagnostic agent (preferably a drug which more preferably is a cytotoxic agent); and (ii) a pharmaceutically acceptable diluent or carrier. Preferably the said component (i) is an ADC, i.e. an antibody-drug conjugate (wherein an “ADC” as defined herein may comprise either an antibody or an antibody fragment).

In one specific aspect, the present invention provides a method of ameliorating or reducing the incidence of cancer in a subject, which method comprises the administration to the said subject of an effective amount of a chemically modified antibody (or antibody fragment) of the present invention, which comprises at least one cargo moiety that is a cytotoxic agent and wherein the chemically modified antibody (or antibody fragment) is capable of specific binding to an antigen AG that is associated with cancer (e.g., an antigen that is expressed on the surface of cancer cells and/or that is capable of specific binding to one of the specific antigens described elsewhere herein).

The present invention also provides a chemically modified antibody (or antibody fragment) of the present invention, which comprises at least one cargo moiety that is a drug or a diagnostic agent (preferably a drug which more preferably is a cytotoxic agent), for use in a method of treatment of the human or animal body by therapy or for use in a diagnostic method practised on the human or animal body.

Still further, the present invention provides a chemically modified antibody (or antibody fragment) of the present invention, which comprises at least one cargo moiety that is a cytotoxic agent and wherein the chemically modified antibody (or antibody fragment) is capable of specific binding to an antigen AG that is associated with cancer (e.g., an antigen that is expressed on the surface of cancer cells and/or that is capable of specific binding to one of the specific antigens described elsewhere herein), for use in a method of treatment of cancer.

The pharmaceutical composition of the present invention is suitable for veterinary or human administration.

The present pharmaceutical compositions can be in any form that allows for the composition to be administered to a patient. The composition may for example be in the form of a solid or liquid. Typical routes of administration include, without limitation, parenteral, ocular and intra-tumor. Parenteral administration includes subcutaneous injections, intravenous, intramuscular or intrasternal injection or infusion techniques. In one aspect, the compositions are administered parenterally. In a specific embodiment, the compositions are administered intravenously.

Compositions can take the form of one or more dosage units, where for example, a tablet can be a single dosage unit, and a container of a compound of the present invention in liquid form can hold a plurality of dosage units.

Materials used in preparing the pharmaceutical compositions are preferably non-toxic in the amounts used. It will be evident to those of ordinary skill in the art that the optimal dosage of the active ingredient(s) in the pharmaceutical composition will depend on a variety of factors. Relevant factors include, without limitation, the type of animal (e.g., human), the particular form of the compound of the present invention, the manner of administration, and the composition employed.

The pharmaceutically acceptable diluent or carrier can be solid or particulate, so that the compositions are, for example, in tablet or powder form. The carrier(s) can be liquid. In addition, the carrier(s) can be particulate.

The pharmaceutical composition can be in the form of a liquid, e.g., a solution, emulsion or suspension. In a composition for administration by injection, one or more of a surfactant, preservative, wetting agent, dispersing agent, suspending agent, buffer, stabilizer and isotonic agent can also be included.

Liquid pharmaceutical compositions, whether they are solutions, suspensions or other like form, can also include one or more of the following; sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates, phosphates or amino acids and agents for the adjustment of tonicity such as sodium chloride or dextrose. A parenteral composition can be enclosed in ampoule, a disposable syringe or a multiple-dose vial made of glass, plastic or other material. Physiological saline is an exemplary adjuvant. An injectable composition is preferably sterile.

The amount of chemically modified antibody or antibody fragment that is effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

The compositions comprise an effective amount of a chemically modified antibody or antibody fragment such that a suitable dosage will be obtained. Typically, this amount is at least about 0.01% of compound chemically modified antibody or antibody fragment by weight of the composition. In an exemplary embodiment, pharmaceutical compositions are prepared so that a parenteral dosage unit contains from about 0.01% to about 2% by weight of the chemically modified antibody or antibody fragment.

For intravenous administration, the composition can comprise from about 0.01 to about 100 mg of chemically modified antibody or antibody fragment per kg of the patient's body weight. In one aspect, the composition can include from about 1 to about 100 mg of chemically modified antibody or antibody fragment per kg of the patient's body weight. In another aspect, the amount administered will be in the range from about 0.1 to about 25 mg/kg of body weight of the chemically modified antibody or antibody fragment.

Generally, the dosage of chemically modified antibody or antibody fragment administered to a patient is typically about 0.01 mg/kg to about 20 mg/kg of the patient's body weight. In one aspect, the dosage administered to a patient is between about 0.01 mg/kg to about 10 mg/kg of the patient's body weight. In another aspect, the dosage administered to a patient is between about 0.1 mg/kg and about 10 mg/kg of the patient's body weight. In yet another aspect, the dosage administered to a patient is between about 0.1 mg/kg and about 5 mg/kg of the patient's body weight. In yet another aspect the dosage administered is between about 0.1 mg/kg to about 3 mg/kg of the patient's body weight. In yet another aspect, the dosage administered is between about 1 mg/kg to about 3 mg/kg of the patient's body weight.

The chemically modified antibody or antibody fragment can be administered by any convenient route, for example by infusion or bolus injection. Administration can be systemic or local. Various delivery systems are known, e.g., encapsulation in liposomes, microparticles, microcapsules, capsules, etc., and can be used to administer a chemically modified antibody or antibody fragment. In certain embodiments, more than one chemically modified antibody or antibody fragment is administered to a patient.

In specific embodiments, it can be desirable to administer one or more chemically modified antibody or antibody fragment locally to the area in need of treatment. This can be achieved, for example, and not by way of limitation, by local infusion during surgery; topical application, e.g., in conjunction with a wound dressing after surgery; by injection; by means of a catheter; or by means of an implant, the implant being of a porous, non-porous, or gelatinous material, including membranes, such as sialastic membranes, or fibers. In one embodiment, administration can be by direct injection at the site (or former site) of a cancer, tumor or neoplastic or pre-neoplastic tissue, in another embodiment, administration can be by direct injection at the site (or former site) of a manifestation of an autoimmune disease.

The chemically modified antibody or antibody fragment can be delivered in a controlled release system, such as but not limited to, a pump or various polymeric materials can be used. Also, a controlled-release system can be placed in proximity of the target of the chemically modified antibody or antibody fragment, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer (Science 249:1527-1533 (1990)) can be used.

The term “carrier or diluent” refers to a diluent, adjuvant or excipient, with which a chemically modified antibody or antibody fragment is administered. Such pharmaceutical carriers can be liquids, such as water and oils, including those of petroleum, animal, vegetable or synthetic origin. The carriers can be saline, and the like.

In addition, auxiliary, stabilizing and other agents can be used. Preferably, when administered to a patient, the chemically modified antibody or antibody fragment and pharmaceutically acceptable carriers are sterile. Water is an exemplary carrier when the chemically modified antibody or antibody fragment is administered intravenously. Saline solutions and aqueous dextrose and glycerol solutions can also be employed as liquid carriers, particularly for injectable solutions. The present compositions, if desired, can also contain minor amounts of wetting or emulsifying agents, or pH buffering agents.

The present compositions can take the form of solutions, pellets, powders, sustained-release formulations, or any other form suitable for use. Other examples of suitable pharmaceutical carriers are described in “Remington's Pharmaceutical Sciences” by E. W. Martin.

The chemically modified antibody or antibody fragment may be formulated in accordance with routine procedures as a pharmaceutical composition adapted for intravenous administration to animals, particularly human beings. Typically, the carriers or vehicles for intravenous administration are sterile isotonic aqueous buffer solutions. Where necessary, the compositions can also include a solubilizing agent. Compositions for intravenous administration can optionally comprise a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where a chemically modified antibody or antibody fragment is to be administered by infusion, it can be dispensed, for example, with an infusion bottle containing sterile pharmaceutical grade water or saline. Where the chemically modified antibody or antibody fragment is administered by injection, an ampoule of sterile water for injection or saline can be provided so that the ingredients can be mixed prior to administration.

The composition can include various materials that modify the physical form of a solid or liquid dosage unit. For example, the composition can include materials that form a coating shell around the active ingredients. The materials that form the coating shell are typically inert, and can be selected from, for example, sugar, shellac, and other enteric coating agents. Alternatively, the active ingredients can be encased in a gelatin capsule.

Whether in solid or liquid form, the present compositions can include a pharmacological agent used in the treatment of cancer.

The chemically modified antibody or antibody fragment is particularly useful for treating cancer (i.e., when the identity of the antibody/antibody fragment and cargo moiety or moieties are suitably selected, for example as described elsewhere herein). Specifically, the chemically modified antibody or antibody fragment is useful for inhibiting the multiplication of a tumor cell or cancer cell, causing apoptosis in a tumor or cancer cell, or for treating cancer in a patient. The chemically modified antibody or antibody fragment can be used accordingly in a variety of settings for the treatment of animal cancers.

The chemically modified antibody or antibody fragment can be used to deliver a therapeutically active agent to a tumor cell or cancer cell. Examples of types of cancers that can be treated with a chemically modified antibody or antibody fragment include, but are not limited to:
Solid tumors, including but not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophogeal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma and retinoblastoma,
blood-borne cancers, including but not limited to acute lymphoblastic leukemia “ALL”, acute lymphoblastic B-cell leukemia, acute lymphoblastic T-cell leukemia, acute myeloblastic leukemia “AML”, acute promyelocyte leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, hairy cell leukaemia and multiple myelomal
acute and chronic leukemias such as lymphoblastic, myelogenous, lymphocytic and myelocytic leukemias; and
lymphomas such as Hodgkin's disease, non-Hodgkin's Lymphoma, Multiple myeloma, Waldenstrom's macroglobulinemia, Heavy chain disease and Polycythemia vera.

Further examples of cancers susceptible to treatment according to the present invention are those herein disclosed in parentheses in conjunction with specific antibodies or antibody fragments as herein disclosed.

The following Examples, which do not limit the scope of the invention, further illustrate the principles of the present invention.

### Examples

### 1. General

### 1.1 Methods

LCMS was performed on protein samples using a Waters Acquity UPLC connected to Waters Acquity Single Quad Detector [column, Acquity UPLC BEH C18 1.7 μm 2.1×50 mm; wavelength, 254 nm; mobile phase, 95:5 water (0.1% formic acid):MeCN (0.1% formic acid), gradient over 4 min to 5:95 water (0.1% formic acid):MeCN (0.1% formic acid); flow rate, 0.6 mL/min; MS mode, ES+/−; scan range, m/z=95-2000; scan time, 0.25 s]. Data was obtained in continuum mode. Sample volume was 30 μl and injection volumes were 3-9 μl with partial loop fill. The electron spray source of the MS was operated with a capillary voltage of 3.5 kV and a cone voltage of 20-200 V. Nitrogen was used as the nebulizer and desolvation gas at a total flow of 600 L/h. Total mass spectra were reconstructed from the ion series using the MaxEnt 1 algorithm pre-installed on MassLynx software.

MALDI-TOF analysis was performed on a MALDI micro MX (Micromass). Data was obtained with a source voltage of 12 kV and a reflectron voltage (if applicable) of 5 kV at a laser wavelength of 337 nm Samples were recorded as outlined below. Buffer salts were removed prior to analysis by dialysis for 24 h at 4° C. against deionised water with Slide-A-Lyzer MINI dialysis units (Thermo Scientific, 2 or 10 kDa MWCO). All proteins were spotted onto a MALDI plate after 1:1 mixture with the matrix (10 mg/ml in 1:1 H₂O:MeCN). Trifluoroacetic acid (TFA, 10 mg/ml) was pre-spotted, if necessary.

Relative quantification of MS data was carried out by normalisation of all identifiable peptide or protein signals (starting material, product, side and degradation products) to 100% according to their unmodified signal strength (relative ion count).

Absorbance measurements were carried out on a Carry Bio 100 (Varian) UV/Vis spectrophotometer equipped with a temperature-controlled 12× sample holder in quartz cuvettes (1 cm path length, volume 75 μl) at 25° C. Samples were baseline corrected and slits set to 5 nm Protein solutions were scanned from 450-250 nm and concentration calculated using either the published or calculated (based on the amino acid sequence via the ProtParam tool of the ExPASy data base; http://expasy.org/sprot/) molar extinction coefficients with Lambert Beers law. The concentration of solutions containing full antibodies were determined with a NanoDrop device (Thermo Scientific) in quadruplicates with the IgG setting and corrected for the absorbance of the buffer.

Fluorescence data was obtained on a Carry Eclipse (Varian) machine equipped with a temperature-controlled 4× sample holder in quartz cuvettes at 25° C. Blank buffer was used as zero fluorescence; slits were set to 5 nm and scan speed was average. Absorbance scans were used to determine ideal excitation wavelengths and sample concentrations diluted to obtain a maximal fluorescence signal below 1000 AU.

Non-reducing glycine-SDS-PAGE was performed following standard lab procedures. Proteins from 20 kDa to 80 kDa were separated on 16% gels; proteins above 80 kDa were separated on 12% gels. In both cases a 4% stacking gel was used and a broad-range MW marker (10 kDa-250 kDa, BioLabs) was co-run to estimate protein weights. All gels were stained following a modified literature protocol (Candiano et al., 2004), where 0.12% of the Coomassie G-250 and the Coomassie R-250 dyes were added to the staining solution instead of only the G-250 dye.

All buffer solutions were prepared with double-deionised water and filter-sterilised. Ultrapure DMF was purchased from Sigma-Aldrich and kept under dry conditions. Opened bottles of benzeneselenol were kept under argon and replaced when the solution had turned orange.

The term ‘processed’ antibody fragment or full antibody generally refers to sample of unmodified material that has been exposed to all other experimental conditions other than reducing agent e.g. purification steps.

### 1.2 MALDI Protocols

Suitable protocols to visualise individual proteins and conjugates by MALDI-TOF MS were developed.

TABLE 3.1
MALDI-TOF MS protocols.

| Sample | Matrix | Mode | Pre-spotting | Dilution | Laser | Pulse | Detector | Suppression |
|---|---|---|---|---|---|---|---|---|
| anti-CEA | CHCA | lin− | — | — | 500 | 2000 | 2750 | 8000 |
| PEG-anti-CEA | CHCA | lin− | TFA | — | 500 | 3000 | 2750 | 8000 |
| Rituximab | SA | lin− | TFA | — | 500 | 3000 | 2750 | 8000 |
| PEG-Rituximab | SA | lin− | — | — | 500 | 3000 | 2750 | 8000 |
| Rituximab Fab | SA | lin+ | — | — | 500 | 2000 | 2750 | 8000 |

CHCA = α-cyano-4-hydroxycinnamic acid. SA = sinapinic acid. Ref+ = reflectron positive, ref− = reflectron negative, lin− = linear negative, lin+ = linear positive.

### 1.3 Compound Stock Solutions

Stock solutions of chemical compounds and reducing reagents were of 100× concentration (relative to the target antibody or fragment) when 1-10 equiv were added to the proteins and of 400× or 1000× concentration if more than 10 equiv were added. Solutions of benzeneselenol were prepared immediately before the experiment and not reused. Stock solutions were stored no longer than 24 h (at 4° C.). All stocks were prepared in dry DMF with the following exceptions, which were prepared in buffer only: N-PEG5000-dibromomaleimide, N-PEG5000-dithiophenolmaleimide, 2-mercaptoethanol, TCEP and DTT.

### 2. Modification of an Anti-CEA scFv Fragment

### 2.1 Material

Anti-CEA is single chain antibody fragment directed against the most N-terminal (extracellular) Ig domain of human CEA which it binds with low nM affinity. The original scFv is a mouse antibody isolated from a phage display and is produced in large quantities in bacteria (E. coli). The construct used in this work (internal name shMFELL2Cys) is a humanised version (28 amino acid substitutions) comprising the variable domain of a heavy and a light chain respectively which are connected by a peptide linker and has a MW of 26.7 kDa (246 amino acids). A His₆-tag has been added to the C-terminus to facilitate purification and an artificial disulfide bond was introduced opposite to the antigen binding site (G44C and A239C) to stabilise the protein. A crystal structure of the parental antibody is available (PDB code: 1QOK). The material supplied by Dr Berend Tolner (UCL Cancer Institute) was to 90% pure as estimated from SDS-PAGE analysis.

### 2.2 Preparation of Anti-CEA Solutions

Anti-CEA was supplied in PBS (pH 7.4) in varying concentrations and stored in aliquots at −20° C. The antibody fragment was diluted in PBS (pH 7.4) and DMF (final amount 10% v/v, if not stated otherwise) to yield a concentration of 70.0 μM (1.87 mg/ml) prior to experimentation. An extinction coefficient of £ₘ=48,735 M⁻¹ cm was used to calculate protein concentrations.

### 2.3 Reduction Study of Anti-CEA

To anti-CEA were added 50 equiv of TCEP, 2-mercaptoethanol or DTT for 2, 4 or 6 h. The reactions were maintained at ambient temperature and after the incubation time 100 equiv of monobromomaleimide were added for 20 min to cap free cysteine generated during reduction. All samples were analysed by LCMS. DTT was shown to be an ideal reducing agent for this system.

2.4 Optimisation of Anti-CEA Reduction with DTT

To anti-CEA were added 10 or 20 equiv of DTT and the reaction was incubated for 10, 30, 60 or 90 min at ambient temperature. A 2× excess of dibromomaleimide over DTT was added for 20 mM and the samples analysed by LCMS. The same experiment was carried out under high-salt conditions for which the antibody fragment had been diluted in a PBS buffer containing an increased concentration of NaCl, so that the final salt concentration was 500 mM (instead of 137 mM).

### 2.5 Bridging of Anti-CEA by Adding Reducing Agent and Maleimide Sequentially (a Sequential Protocol)

Anti-CEA was treated with 20 equiv of DTT at ambient temperature for 1 h. Then 30 equiv of dibromomaleimide were added and samples withdrawn after 5, 10 and 15 min and analysed by LCMS. Quantitative disulfide bridging was observed.

### 2.6 Bridging of Anti-CEA by Adding Reducing Agent and Maleimide Concomitantly (an In Situ Protocol)

To anti-CEA were added various amounts of dithiophenolmaleimide and various amounts of benzeneselenol to yield the following combinations (bridging agent: reducing agent): 5:2, 5:5, 10:10, 15:15, 20:10 and 20:20. The reactions were kept at ambient temperature for 1 h and analysed by LCMS. Quantitative functional disulfide bridging achieved.

### 2.7 Time Course for the In Situ Bridging of Anti-CEA

To anti-CEA were added 15 equiv of dithiophenolmaleimide and 15 equiv of benzeneselenol. Aliquots were withdrawn after 5, 10, 20, 30, 45 and 60 min and subjected to LCMS.

### 2.8 Sequential Modification and Functionalisation of Anti-CEA

Anti-CEA was reduced with 20 equiv of DTT for 1 h at ambient temperature. Then 30 equiv of N-fluorescein-dibromomaleimide, N-biotin-dibromomaleimide or N-PEG5000-dibromomaleimide or alternatively 50 equiv of maleimide were added and the reactions analysed by LCMS after 10 min. In the case of anti-CEA PEGylation conversion was indicated by complete loss of the UV signal of the unmodified antibody compared to a non-reacted control. The identity of the product was confirmed by MALDI-TOF MS and SDS-PAGE. Quantitative and selective functional disulfide bridging was achieved with a variety of functionalities.

### 2.9 In Situ Functionalisation of Anti-CEA

To anti-CEA were added 15 equiv of N-PEG5000-dithiophenolmaleimide and 15 equiv of benzeneselenol. The reaction was maintained for 60 min at ambient temperature and aliquots withdrawn after 5, 10, 20, 30, 45 and 60 min for analysis by LCMS. The conversion of anti-CEA PEGylation was monitored as described for the sequential protocol.

### 2.10 Optimisation of the In Situ Protocol

To anti-CEA were added 2 or 5 equiv of dithiophenolmaleimide and various amounts of benzeneselenol. The reaction was maintained at ambient temperature for 20 min and analysed by LCMS.

### 2.11 Optimisation of the In Situ Bridging as a Two-Step Protocol

To anti-CEA were added 2 equiv of dithiophenolmaleimide. A variable amount of benzeneselenol was added for 15 min at ambient temperature followed by an identical amount of benzeneselenol for additional 15 min. The samples were analysed by LCMS. The best combination of reducing agent was also tested from 1.2 and 1.5 equiv of dithiophenolmaleimide.

### 2.12 Fluorescence of Anti-CEA-Fluorescein

Anti-CEA-fluorescein was synthesised via the sequential protocol and the excess of N-fluorescein-dibromomaleimide was removed by purification on PD MiniTrap G-25 desalting columns (GE Healthcare) following manufacturers' instructions. The concentration of the protein solution was determined by UV/Vis spectroscopy, the anti-CEA analogue diluted to 25 or 5 μg/ml and the fluorescence recorded at an emission wavelength of 518 nm (excitation 488 nm) alongside unmodified anti-CEA (350 μg/ml).

### 2.13 Synthesis of a Anti-CEA-HRP Conjugate

Anti-CEA-biotin was synthesised via the sequential protocol and the excess of N-biotin-dibromomaleimide was removed by purification on PD G-25 desalting columns. The concentration of the protein solution was determined by UV/Vis and adjusted to 20 μM. 15 μl of the antibody solution were mixed with increasing amounts of a HRP-Streptavidin conjugate (Invitrogen, 1.25 mg/ml), the sample volume adjusted to 30 μl and incubated for 1 h at ambient temperature. Samples were analysed by SDS-PAGE.

2.14 ‘One Step’ ELISA with Anti-CEA-HRP Conjugates

Anti-CEA-biotin was synthesised via the sequential protocol and the excess of N-biotin-dibromomaleimide was removed by purification on PD G-25 desalting columns. The concentration of the protein solution was determined by UV/Vis spectroscopy. The biotinylated antibody was incubated with a 3× excess (in mass) of a HRP/STREP conjugate for 1 h at ambient temperature and the anti-CEA-HRP conjugate purified with nickel magnetic beads (Millipore) following manufacturer's instructions. The product was analysed by SDS-PAGE and quantified by its OD₂₈₀. 10 μl of serial dilutions of the anti-CEA-HRP conjugate (1:10¹ to 1:10⁵) in PBS were mixed with 90 μl ELISA substrate solution in a 96-well plate and absorbance read after reaction stop at 490 nm. For comparison serial dilutions of the HRP/STREP conjugate (1:10² to 1:10⁶) and of the secondary antibody for the used ELISA (1:10⁴ to 1:10⁸) were tested alongside. A 1:500 dilution of an OD₂₈₀=0.4 solution of the HRP-anti-CEA conjugate was found to give a good signal comparable to the ELISA mixture used.

A 96-well plate was coated with various amounts of full length CEA (0.125 mg/ml to 4 mg/ml in PBS), blocked and washed as described and incubated with 100 μl per well of a 1:500 dilution of a OD₂₈₀=0.4 solution of the anti-CEA-HRP conjugate for 1 h at ambient temperature. Plate read-out was performed as described.

Alternatively a standard ELISA was performed with dilutions of a OD₂₈₀=0.4 solution of the anti-CEA-HRP conjugate in place of the usual antibody solutions.

2.15 ‘Two-Step’ ELISA with Anti-CEA-HRP on Plate Formation

An ELISA plate was prepared as described and treated with the usual dilutions of biotinylated anti-CEA. One sample was reacted with the described mix of primary and secondary antibody. Another sample was treated with a 1:460 dilution of the HRP/STREP conjugate (in PBS, 1% (w/v) Marvel, 20× estimated mass excess over the antibody) and a third one with a 1:4600 dilution of the HRP/STREP conjugate (in PBS, 1% (w/v) Marvel, 2× estimated mass excess over the antibody). Incubation times were staggered so that they did not exceed 1 h at ambient temperature for any of the samples. Visualisation and read-out were performed as described.

### 2.16 Functionally Bridged Anti-CEAs Retain Binding to CEA

All ELISA samples of anti-CEA and its analogues were purified on PD G-25 desalting columns after modification and concentrations were determined by UV/Vis spectroscopy.

ELISA plates were coated with full length human CEA diluted to a final concentration of 1 μg/ml in PBS for 1 h at ambient temperature, washed and blocked over night at 4° C. with a 5% (w/v) solution of Marvel milk powder (Premier Foods) in PBS. The plate was washed and anti-CEA and its analogues were added after dilution to the indicated concentrations (typically 5.0, 1.0, 0.5, 0.1, 0.05 and 0.01 μg/ml) in PBS. The assay was incubated at ambient temperature for 1 h, washed and the primary antibody (anti-tetra-His mouse IgG1, Quiagen, 1:1000 in 1% (w/v) Marvel solution) added. After 1 h at ambient temperature the ELISA plate was washed and the secondary antibody (ECL anti-mouse sheep IgG1 HRP linked, GE Healthcare, 1:1000 in 1% (w/v) Marvel solution) added for 1 h at ambient temperature. The plate was washed and freshly prepared substrate solution (one tablet of o-phenylenediamine in 25 ml 50 μM phosphate citrate buffer, Sigma-Aldrich) was added to each well. When a strong orange colour had developed the reaction was stopped by addition of 4 M HCl and the plate read at a wavelength of 490 nm Controls were included in every ELISA where PBS had been added to some of the wells instead of CEA or instead of the antibody fragment.

Each sample was tested in triplicates, and errors are shown as the standard deviation of the average.

2.17 Stability study of Functionally Bridged Anti-CEAs

Bridged anti-CEA and anti-CEA-PEG5000 were prepared via the in situ protocol, purified on PD G-25 desalting columns and stored at 4° C. for 4 d. After this time both compounds were prepared again, purified as described, the concentration determined by UV/Vis spectroscopy and binding activity tested alongside the stored compounds via ELISA. Functionally bridged anti-CEAs were stable under these conditions.

### 2.18 Fluorescence-Based Cell ELISA

Anti-CEA-fluoresceine was synthesised via the stepwise protocol and the excess of N-fluorescein-dibromomaleimide was removed by purification on PD G-25 desalting columns. The concentration of the protein solution was determined by UV/Vis spectroscopy.

Log-phase cultures of CAPAN-1 (CEA expressing cells, cultured in DMEM, 20% FCS, 1% glutamate, 1% streptomycin) and A375 (negative control, cultured in DMEM, 10% FCS, 1% glutamate, 1% streptomycin) cell lines were detached non-enzymatic, counted and diluted (3×10³ to 1×10⁵ per well) in a 96-well plate. Cells (in their respective media) were allowed to attach for 24 h in the incubator (at 37° C. in humid atmosphere, 5% CO₂ atmosphere), were gently washed twice with PBS and treated with 500 ng of the fluorescent antibody (5 μg/ml in PBS) for 1 h at ambient temperature. All samples were gently washed twice with PBS, wells filled with PBS and the fluorescence read at 518 nm (excitation 488 nm, exposure time 100 ms, slits 12 nm). Cells treated with non-fluorescent anti-CEA, untreated cells and PBS only were used to determine the background. Fluoroscein-labelled anti-CEA is selective for CEA expressing cells.

### 2.19 Kd Determination for Functionalised Anti-CEAs Using Biacore Assay

Bridged anti-CEA and anti-CEA-PEG5000 were prepared via the in situ protocol, purified on PD G-25 desalting columns and the concentrations were determined by UV/Vis spectroscopy.

The binding activity was then tested alongside unmodified (processed) anti-CEA via surface plasmon resonance on a Biacore T100. In brief a SA chip (coated with streptavidin) was loaded with 566 AU of biotynilated NA1 and serial dilutions of the anti-CEA fragment and its analogues were injected (400, 200, 100, 50, 25, 12.5 and 0 nM). The contact time was 120 s at a flow rate of 20 μl/min followed by dissociation time of 600 s. The chip was regenerated with a 10 mM glycine solution for 60 s at a flow rate of 30 μl/min. All sample runs were performed at 25° C. and binding parameters were calculated using the provided software package (Biacore T100 Evaluation Software V 2.0.3).

Kd:

| | |
|---|---|
| unmodified anti-CEA: | 20.8 ± 2.9 nM |
| bridged anti-CEA: | 6.4 ± 0.3 nM |
| PEGylated anti-CEA: | 8.7 ± 0.3 nM |

### 2.20 Stability of the Maleimide Bridge Against Reducing Agents

Dibromomaleimide-bridged anti-CEA was prepared via the in situ protocol, purified on PD G-25 desalting columns and the concentrations were determined by UV/Vis spectroscopy.

The modified antibody fragment was treated with 100 equiv of 2-mercaptoethanol, DTT or GSH for 48 h at ambient temperature. Aliquots were withdrawn at different time points and analysed by LCMS. After 48 h, all samples were reacted with 200 equiv. maleimide and again subjected to LCMS.

### 2.21 Stability of the Maleimide Bridge in Human Plasma

Dibromomaleimide-bridged anti-CEA was prepared via the in situ protocol, purified on a PD G-25 desalting column and the concentration determined by UV/Vis spectroscopy.

70 μg of the bridged anti-CEA were added to 500 μl of human plasma (Sigma-Aldrich) and incubated at 37° C. for 1 h, 4 h, 24 h, 3 d, 5 d and 7 d. The antibody fragment was purified from plasma using PureProteome Nickel Magnetic Beads (Millipore) according to manufacturers' instructions with a few exceptions: the beads were washed 4 times in wash buffer containing no imidazole and the protein eluted twice in 500 mM imidazole for 5 mM Imidazole was removed and the eluate concentrated by repeated washes in PBS in ultrafiltration spin columns. The protein solution was analysed by LCMS.

As a control anti-CEA alkylated with maleimide was prepared via the sequential protocol as described for bridged anti-CEA and 25 μg of this material were mixed with 25 μg of unmodified and 25 μg of bridged anti-CEA. The mixture was added to 500 μl of PBS or human plasma, incubated for 1 h at 37° C. and purified with nickel magnetic beads as outlined above. The purified mixtures were analysed by SDS-PAGE. Alternatively alkylated and unmodified anti-CEA were incubated in human plasma at 37° C. for 7 d and isolated and analysed as described. Dibromomaleimide-bridged anti-CEA was essentially stable in human plasma at 37° C. for 7 d.

2.22 Activity of Anti-CEA Analogues after Incubation in Human Plasma

Bridged anti-CEA and anti-CEA-PEG5000 were synthesised via the in situ protocol and alkylated anti-CEA was synthesised via the sequential protocol. All analogues were purified on PD G-25 desalting columns and the concentration determined by UV/Vis spectroscopy.

37.5 μg of the antibody analogues or the unmodified antibody were added to 500 μl of human plasma and incubated at 37° C. 12 μl were withdrawn from each sample after 1 h, 4 h, 24 h, 3 d, 5 d and 7 d, diluted in 788 μl PBS (to yield an assumed concentration of 1.1 μg/ml), flash frozen in liquid nitrogen and stored at −20° C. After all samples had been collected an ELISA assay was performed as described. As a control a dilution of 12 μl of human plasma in PBS was co-run.

### 3. Modification of a Chimeric IgG1 Full Length Antibody: Rituximab

3.1 Material and preparation

Rituximab is a chimeric IgG1 full length antibody directed against CD20. The antibody was obtained in its clinical formulation (9 mg/ml NaCl, 7.35 mg/ml Na citrate dehydrate, 0.7 mg/ml polysorbate 80) at a concentration of 10 mg/ml. This solution was dissolved in PBS and the buffer exchanged completely into PBS via ultracentrifugation (MWCO 50 kDa, Sartorius). The concentration after the exchange was determined by NanoDrop to be 3.44 mg/ml (22.9 μM) and the protein solution was stored in flash frozen aliquots at −20° C. Prior to experimentation DMF was added to a final concentration of 20% (v/v) if not stated otherwise.

### 3.2 Reduction of Rituximab

The antibody was treated various amounts of TCEP for 1 h at ambient temperature and the samples analysed on SDS-PAGE. Intact and reduced samples were dialysed and visualised by MALDI-TOF as described.

3.3 In Situ Bridging Study with Rituximab

To the antibody were added various amounts of dithiophenolmaleimide followed by 10 or 40 equiv of TCEP. The samples were incubated at ambient temperature for 1 h and analysed by SDS-PAGE. Successful bridging or rituximab was estimated by inspection of bands expected for full antibody, heavy chain and light chain.

### 3.4 Preliminary In Situ PEGylation Study of Rituximab

To the antibody were added various amounts of N-PEG5000-dithiophenolmaleimide followed by 10 or 40 equiv of TCEP. The samples were incubated at ambient temperature for 1 h and analysed by SDS-PAGE. Successful bridging or rituximab was estimated by inspection of bands expected for full antibody, heavy chain and light chain.

3.5 Detailed PEGylation Study with Rituximab

To the antibody were added various amounts of N-PEG5000-dithiophenolmaleimide followed by various amounts of either TCEP or benzeneselenol. The reactions were incubated at ambient temperature for 1 h and analysed by SDS-PAGE. PEGylated samples were purified with Protein A magnetic beads following the manufacturers' instructions with a few exceptions: The binding reaction was incubated for 1 h at ambient temperature and all elutions were incubated for 5 min at ambient temperature. The purified samples were prepared and analysed by MALDI-TOF as described.

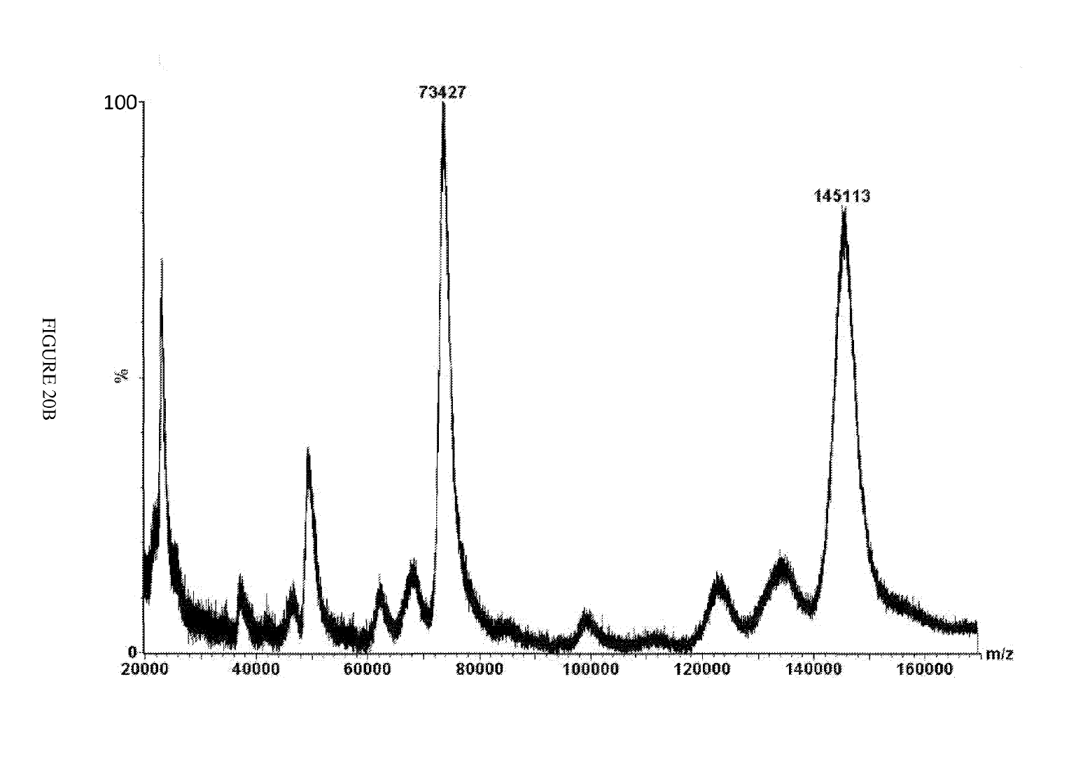

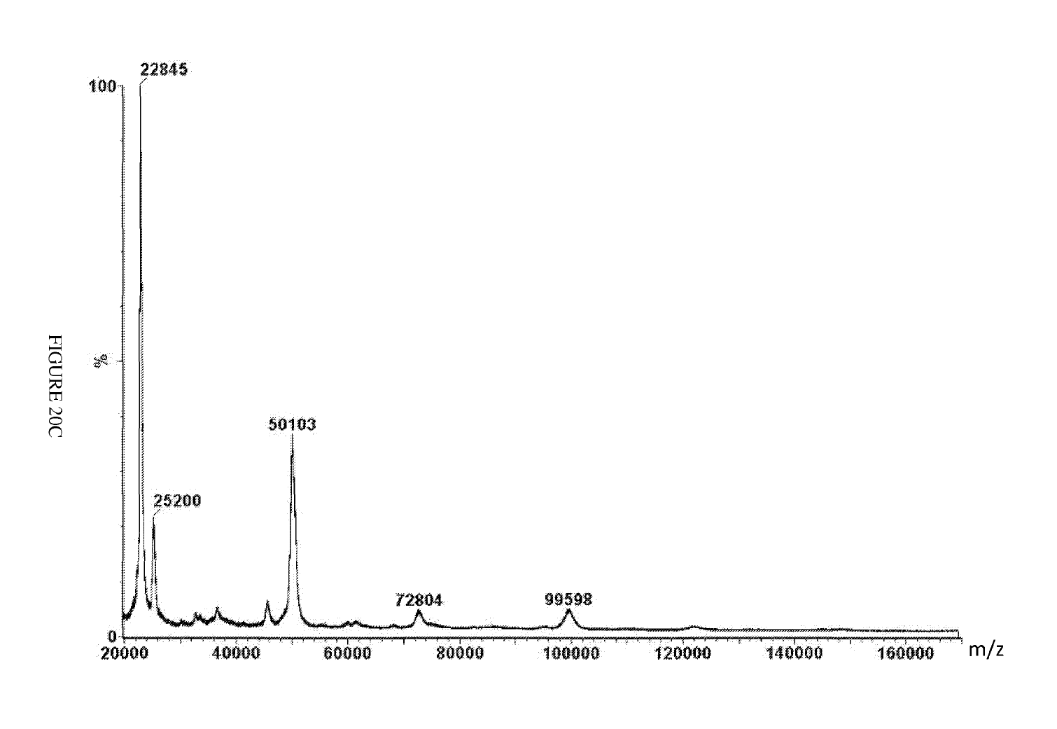

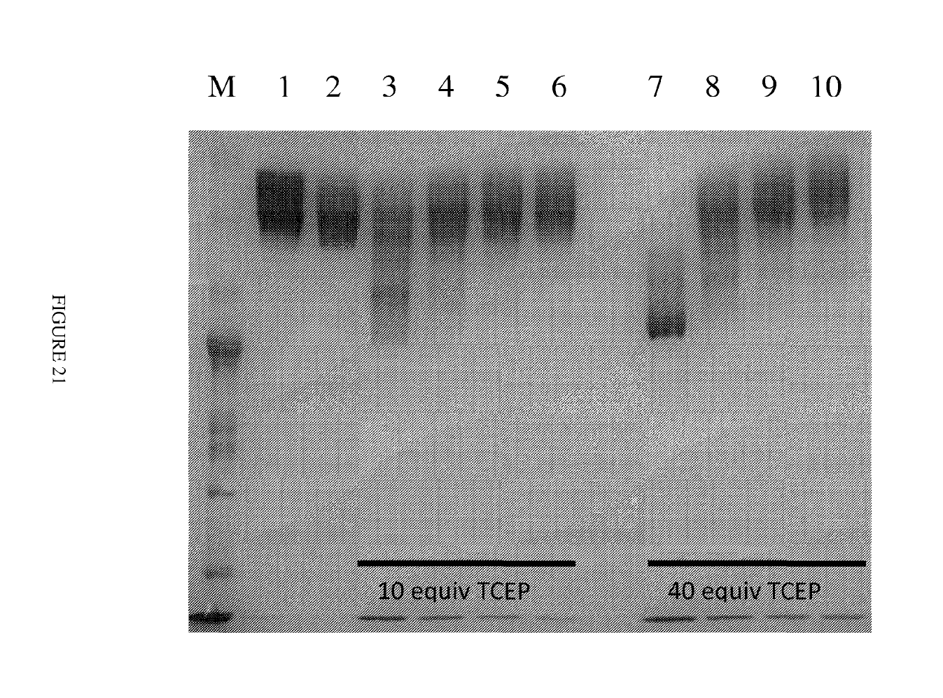

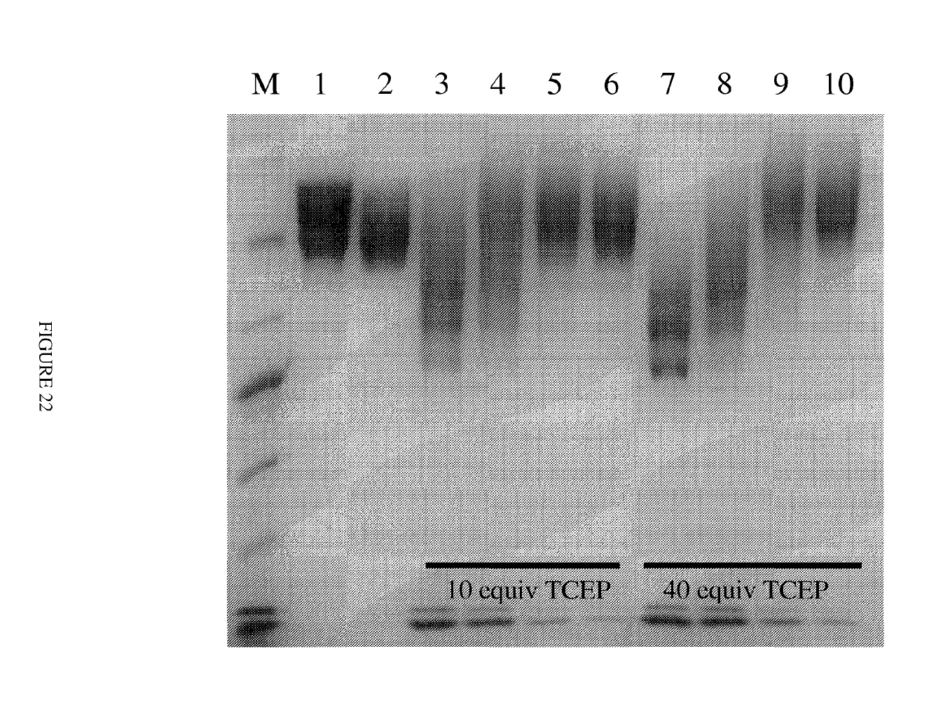

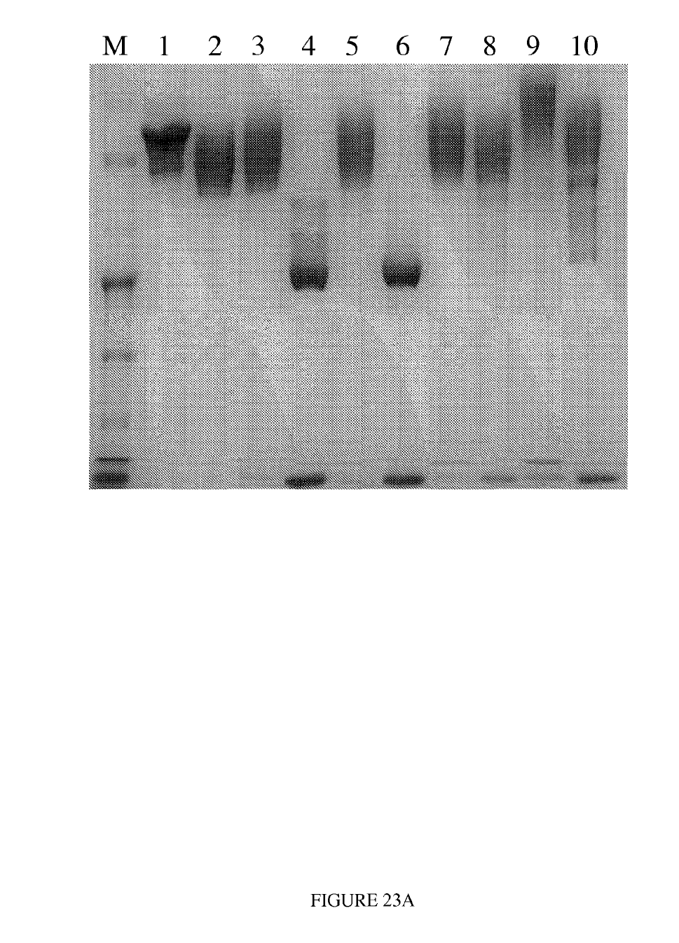

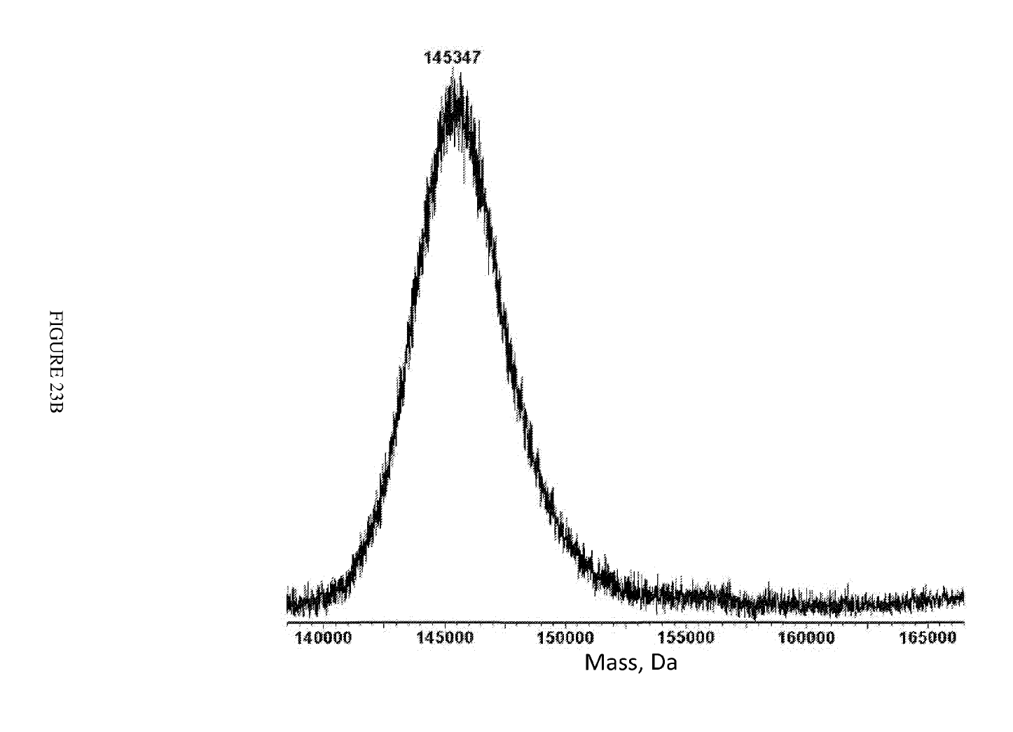

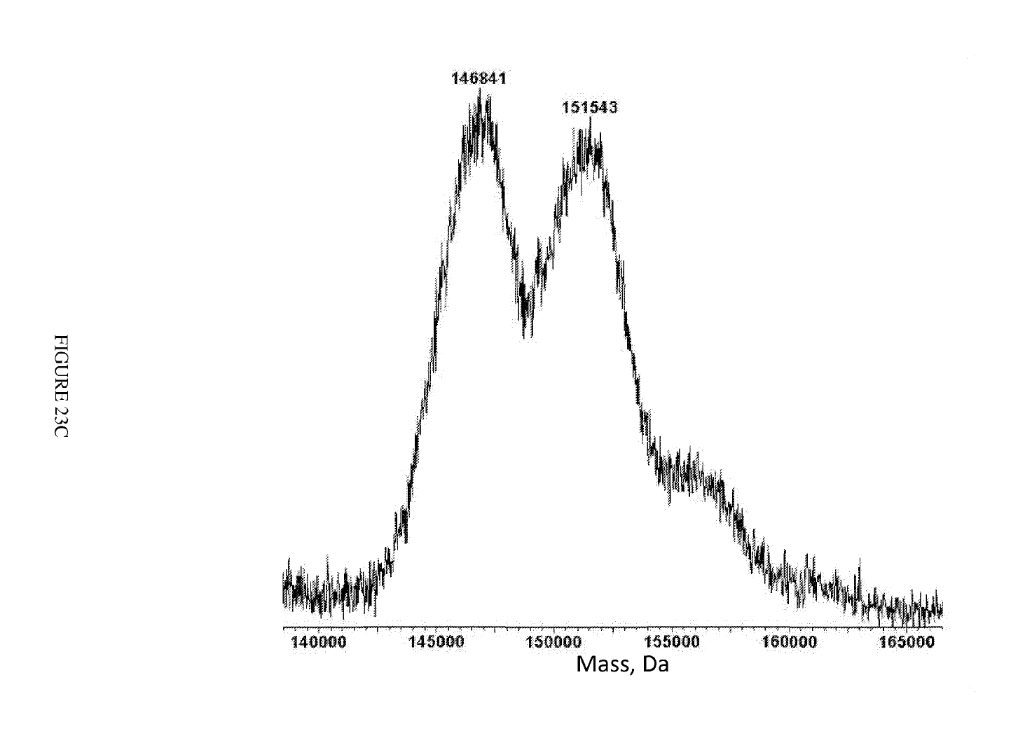

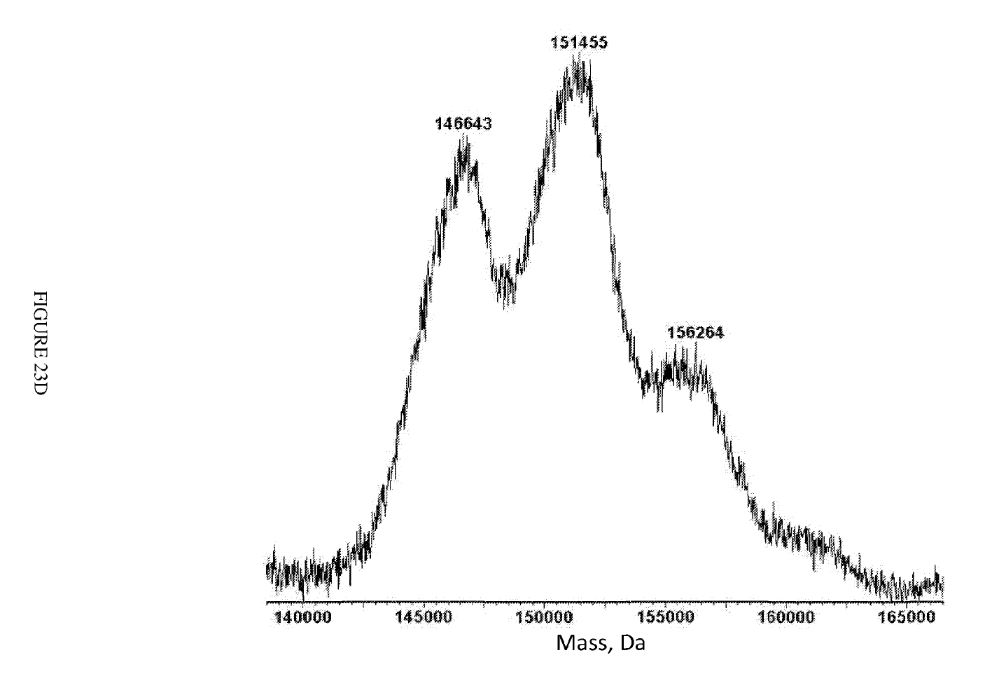

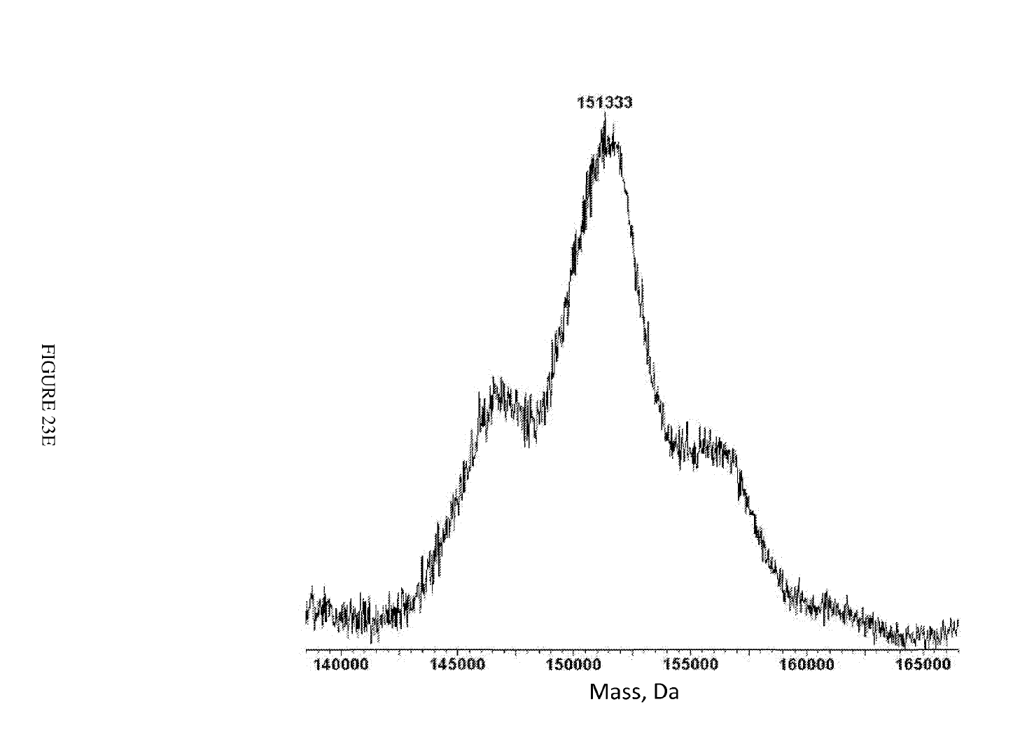

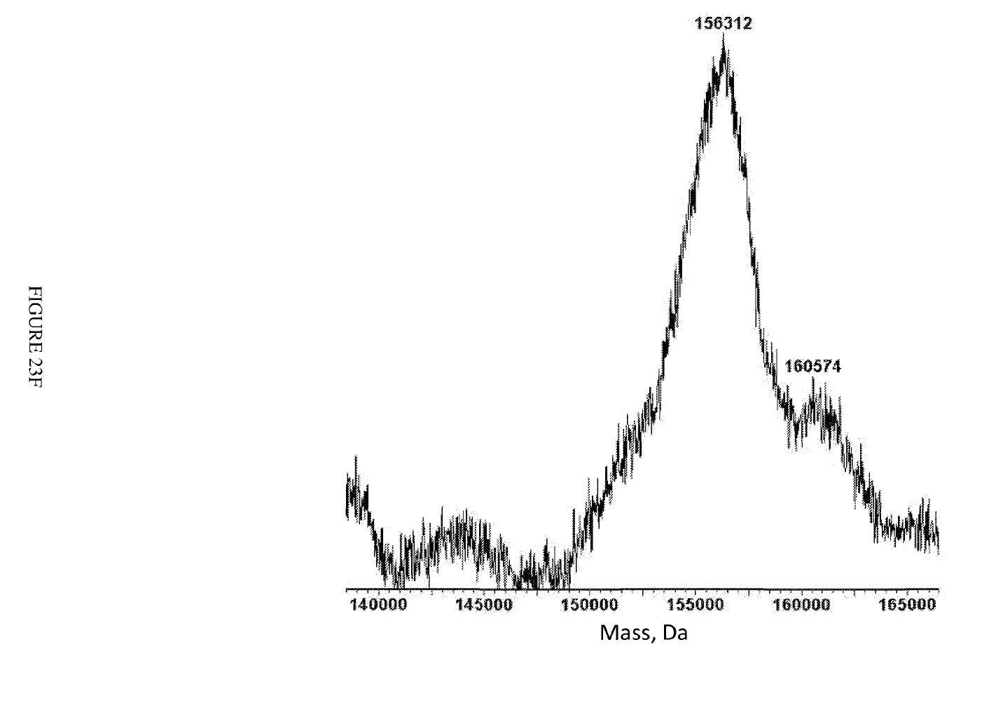

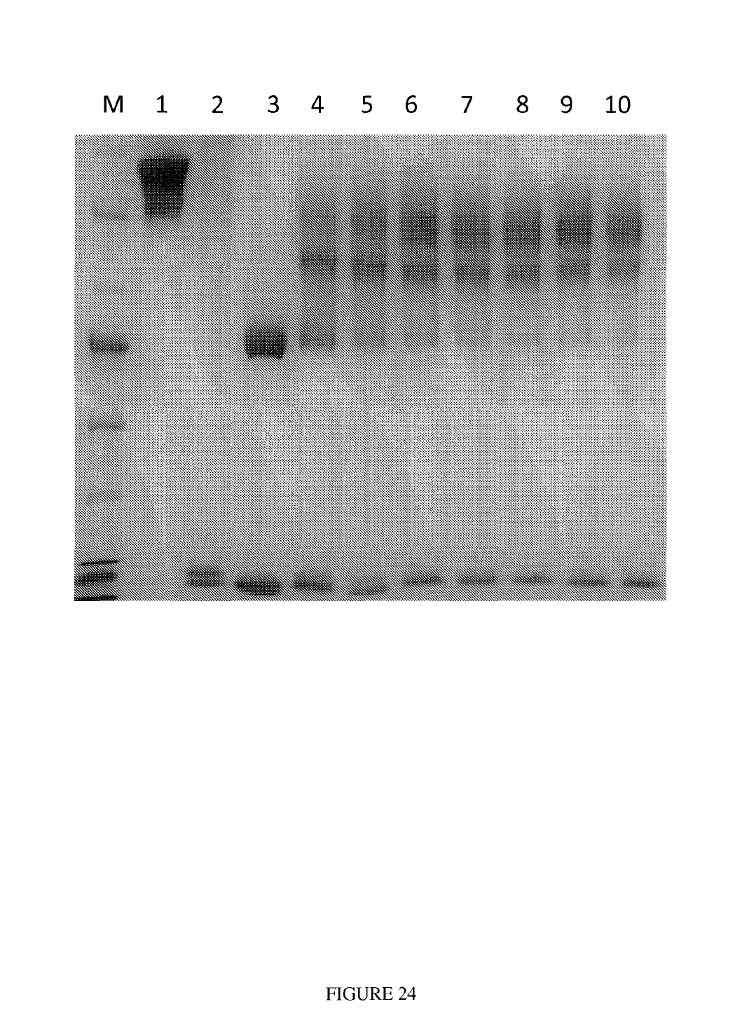

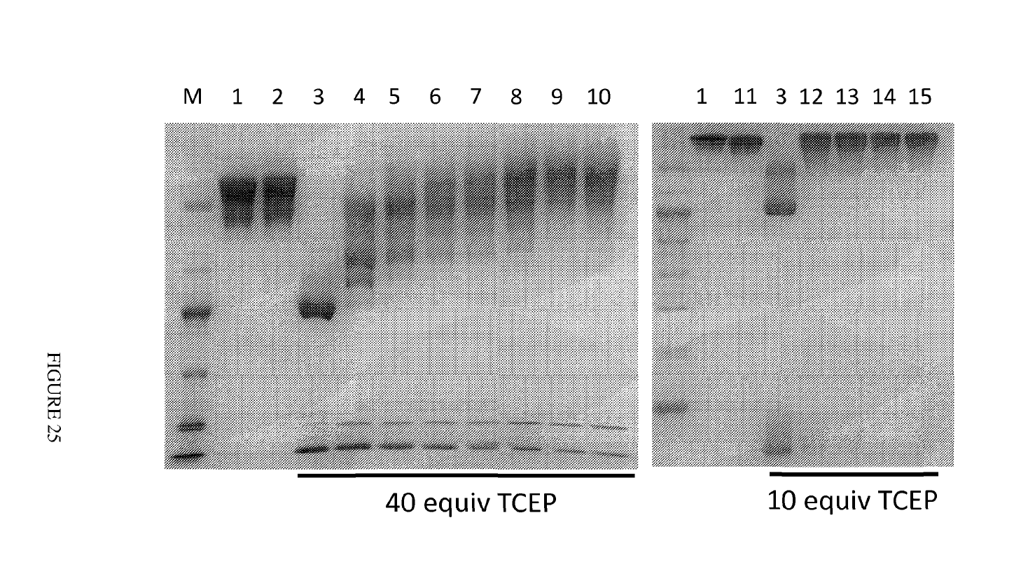

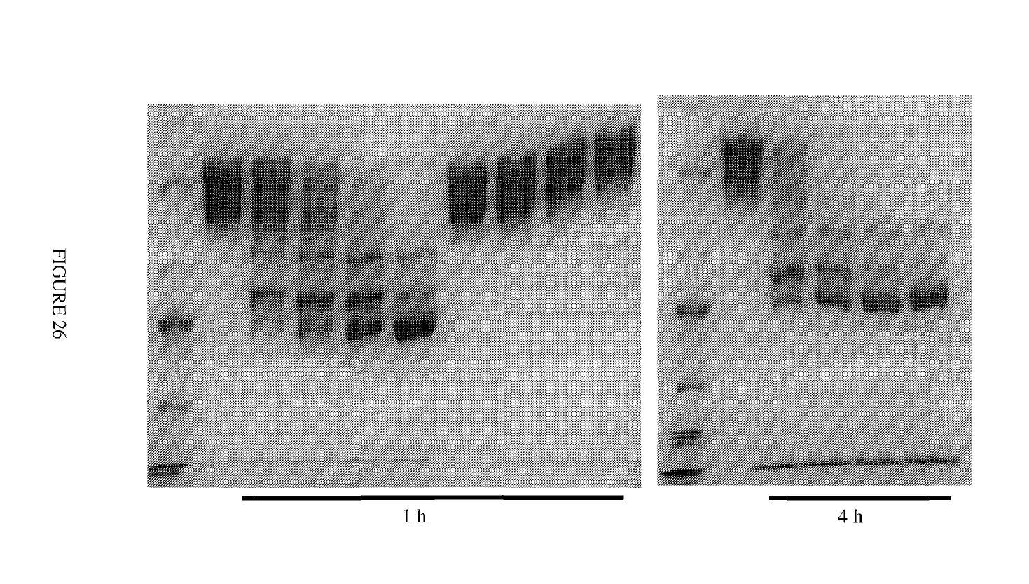

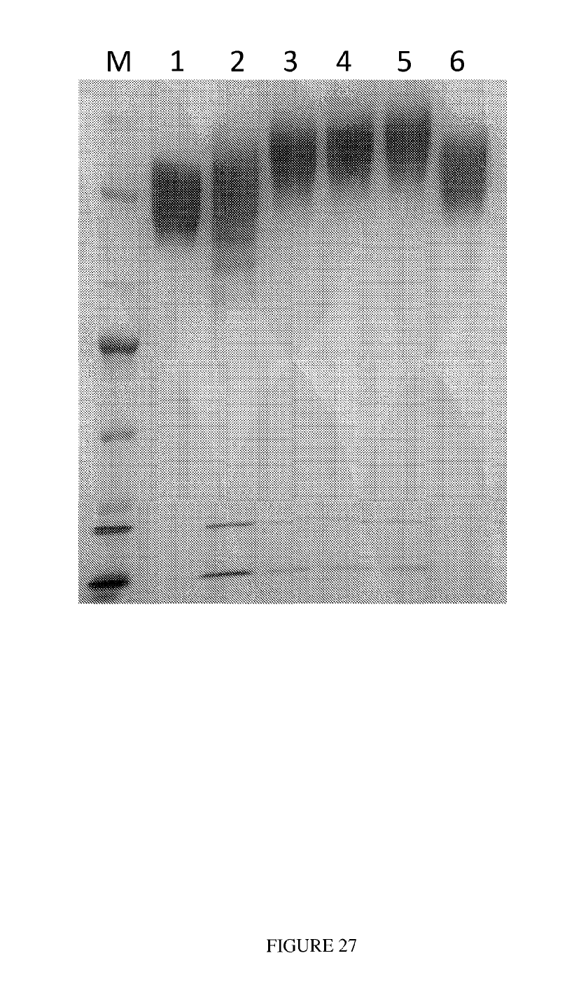

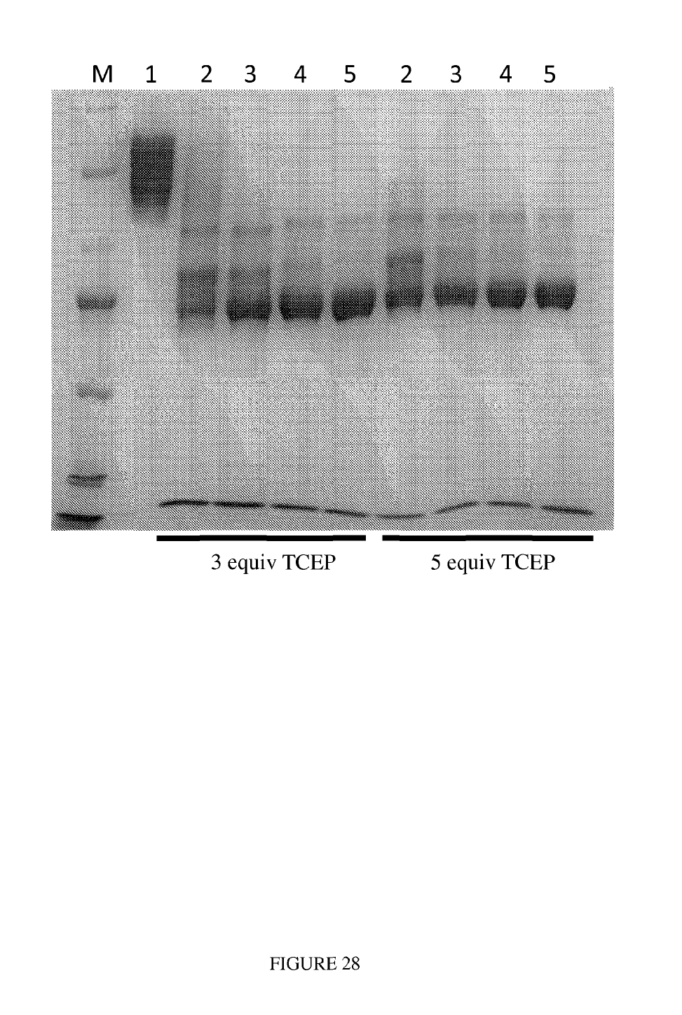

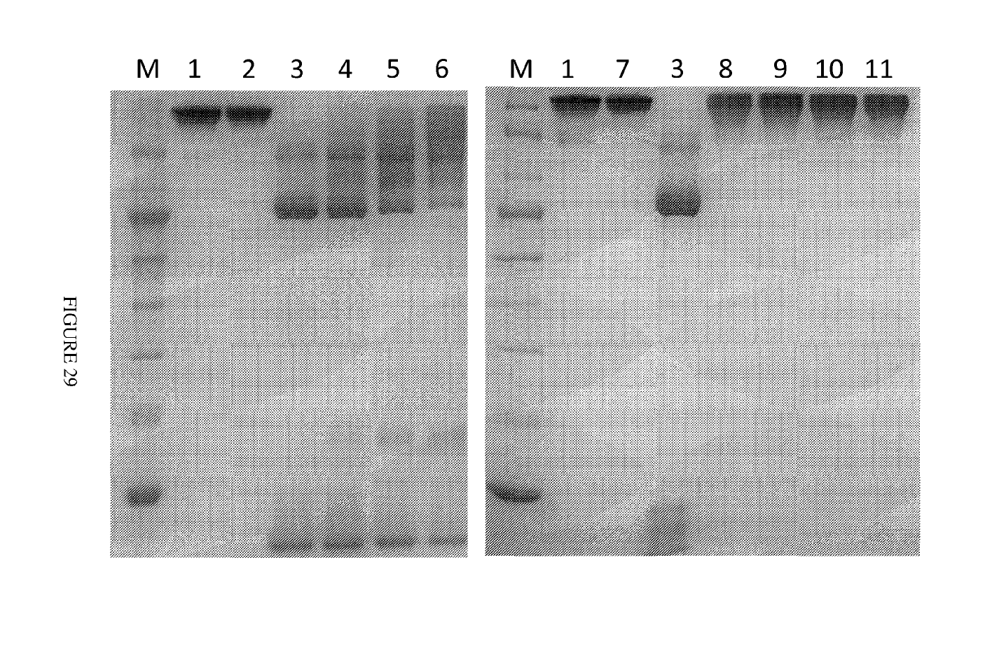

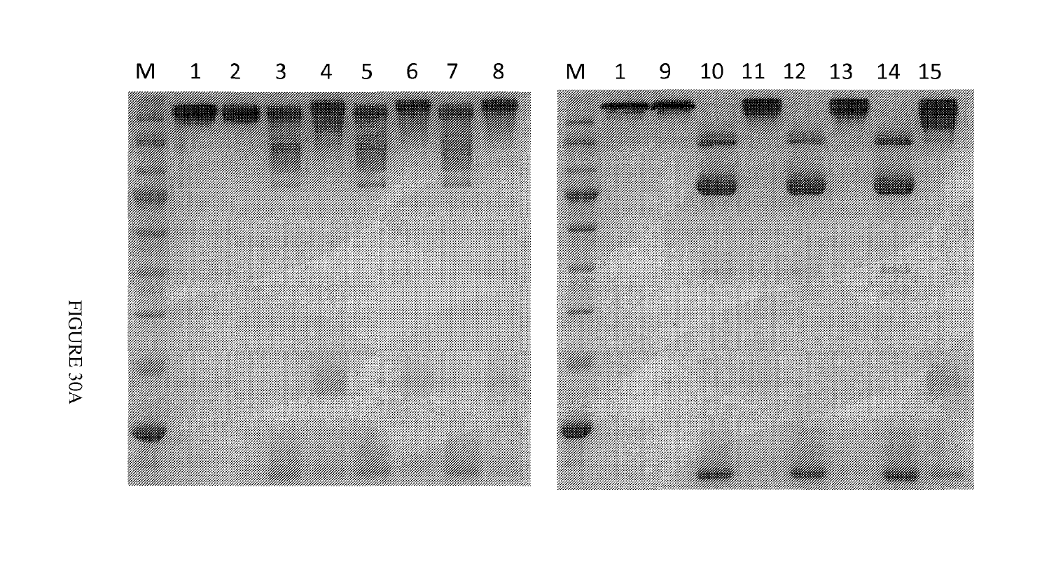

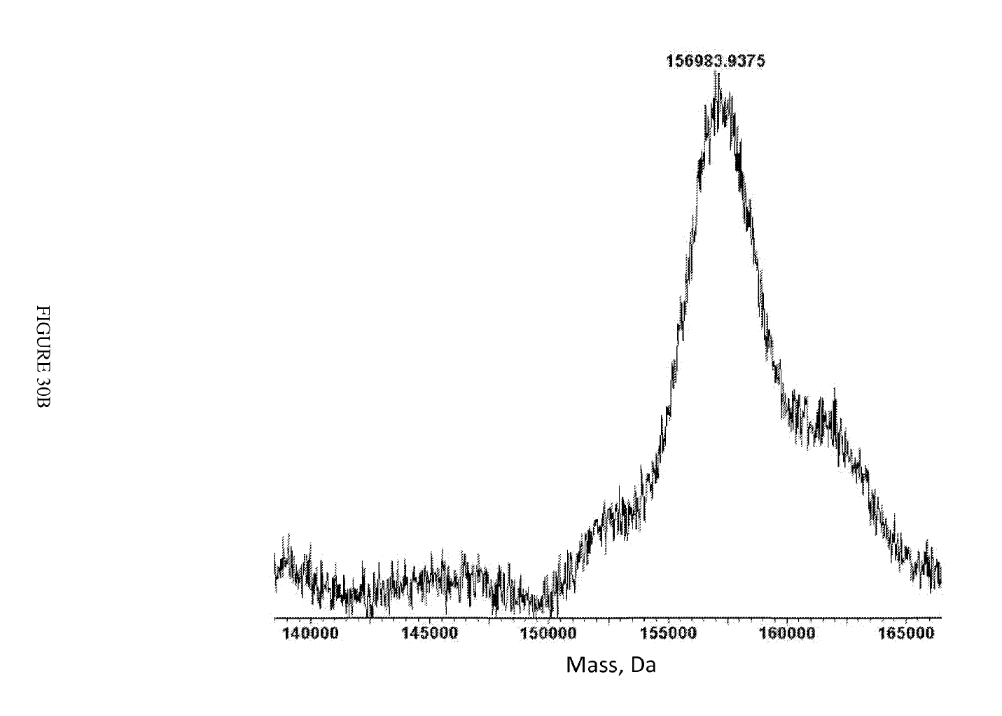

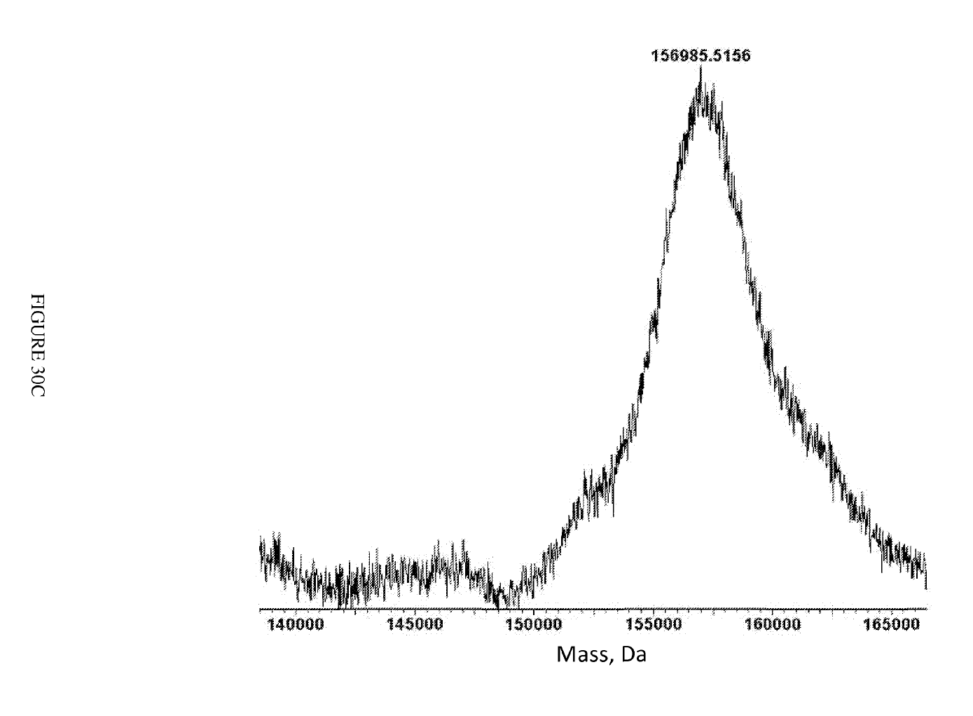

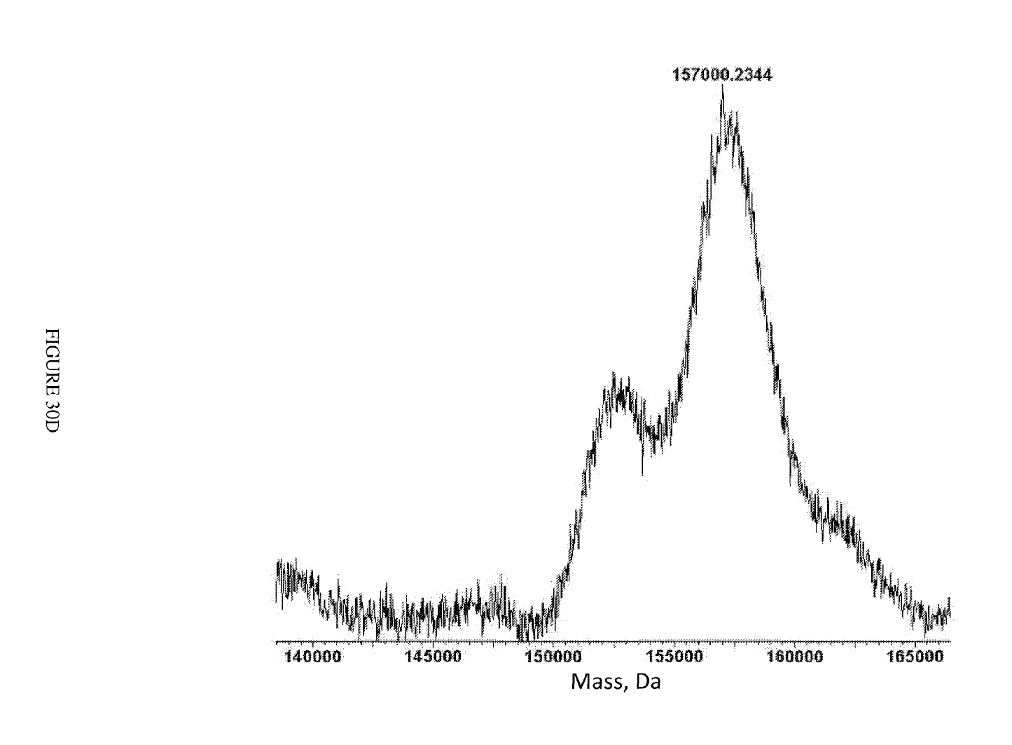

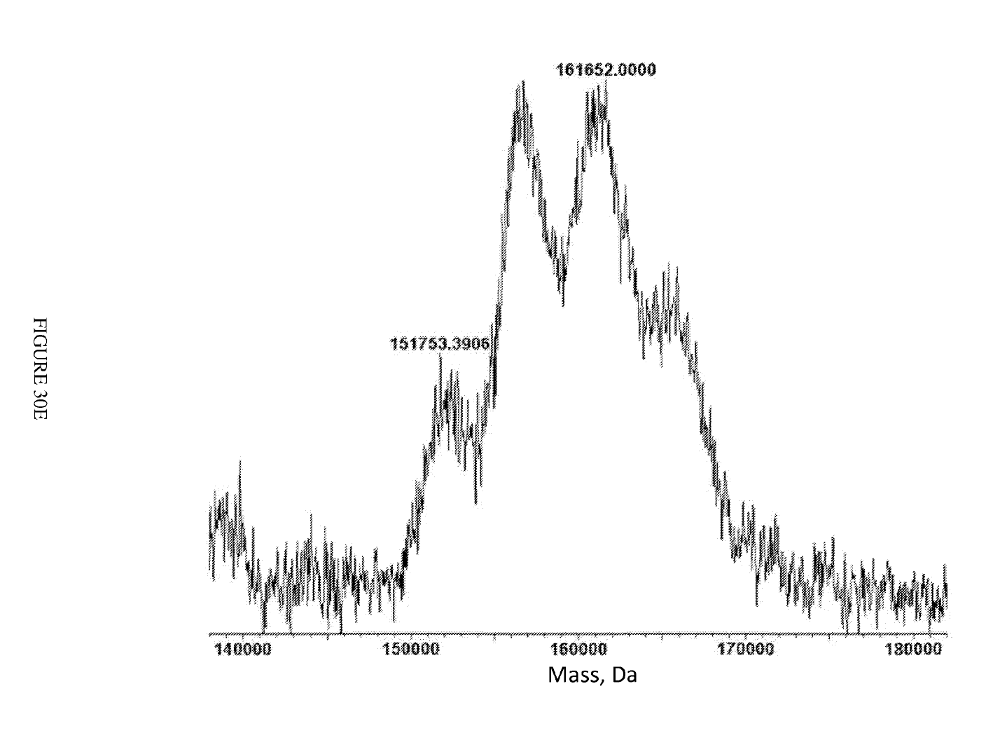

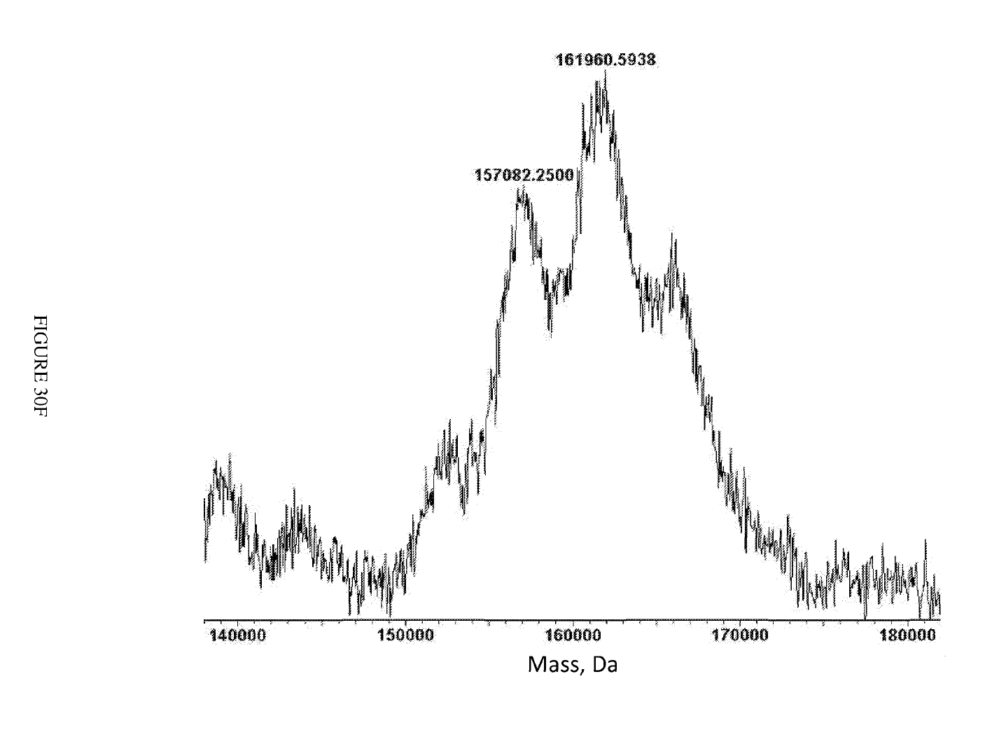

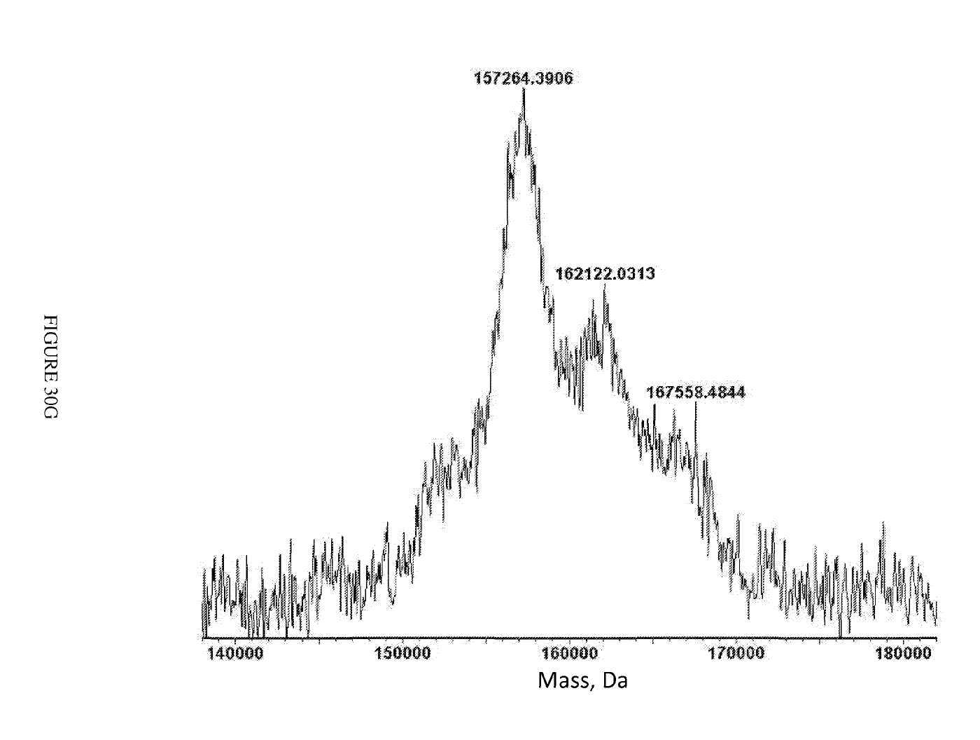

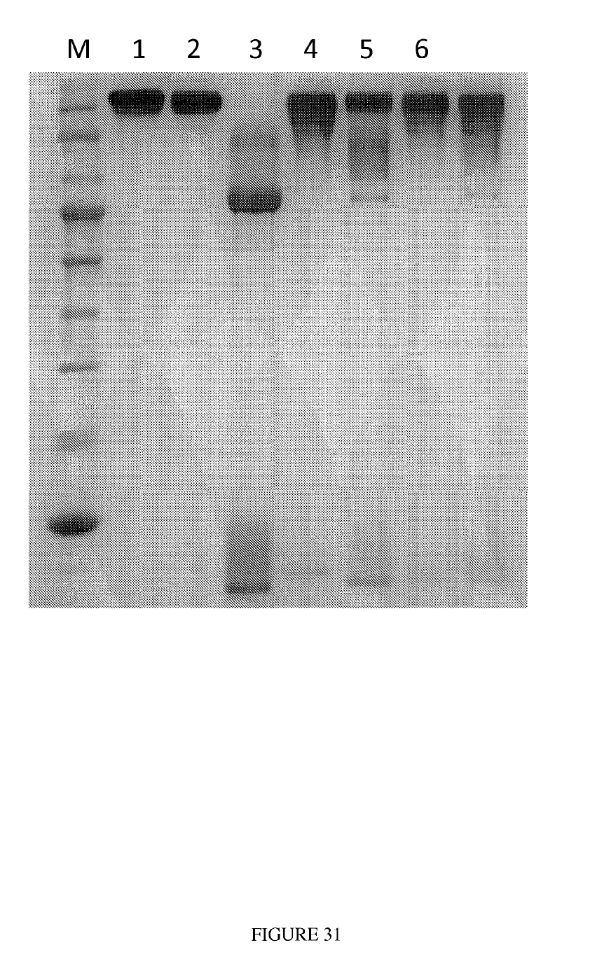

As shown in FIG. 23, reaction with 10 equiv TCEP/20 equiv PEG yielded mainly 0 and 1 modifications (FIG. 23C), reaction with 40 equiv TCEP/80 equiv PEG yielded mainly 0, 1 and 2 modifications (FIG. 23D), reaction with 10 equiv Se/20 equiv PEG yielded mainly 1 modification (FIG. 23E) and reaction with 40 equiv Se/80 equiv PEG yielded mainly 2 modifications (FIG. 23F). Thus, the chemically modified antibody product could be controlled by selecting appropriate reaction conditions.

### 3.6 Sequential Bridging of Rituximab

Rituximab was treated with 40 equiv of TCEP for 1 h at ambient temperature. Then various amounts of dithiophenolmaleimide were added for 30 min at ambient temperature and samples analysed by SDS-PAGE.

Rituximab (prepared without DMF) was treated with 40 equiv of TCEP for 1 h at ambient temperature. Then various amounts of N-PEG5000-dithiophenolmaleimide were added for 30 min at ambient temperature and samples analysed by SDS-PAGE. The experiment was repeated with 10 equiv of TCEP.

Presence of DMF during the reduction step and prior to addition of the maleimide was shown to be sub-optimal.

### 3.7 Alternative Reduction of Rituximab

The antibody (no DMF) was treated with various amounts of either DTT or 2-mercaptoethanol (bME) for 1 h at ambient temperature. All samples were analysed by SDS-PAGE. The experiment was repeated with the same amounts of DTT for 4 h.

### 3.8 Alternative Reduction Sequential PEGylation of Rituximab

Rituximab (no DMF) was reduced with 20 equiv of DTT for 1 h at ambient temperature followed by addition of various amounts of N-PEG5000-dibromomaleimide. The samples were analysed by SDS-PAGE. Successful bridging or rituximab was estimated by inspection of bands expected for full antibody, heavy chain and light chain.

### 3.9 Mixed Reduction of Rituximab

The antibody (no DMF) was treated with 3 or 5 equiv of TCEP for 1 h at ambient temperature. Then various amounts of DTT were added for 3 h at ambient temperature and all reactions analysed by SDS-PAGE.

3.10 Mixed reduction Sequential PEGylation of Rituximab

The antibody (no DMF) was treated with 5 equiv of TCEP for 1 h at ambient temperature. Then 10 equiv of DTT were added for 3 h at ambient temperature followed by various amounts of N-PEG5000-dibromomaleimide. The reaction was analysed by SDS-PAGE. Successful bridging or rituximab was estimated by inspection of bands expected for full antibody, heavy chain and light chain.

3.11 In situ v Sequential Conditions for PEGylation of Rituximab

The optimised established conditions for PEGylation of Rituximab were used side by side for comparison. The antibody was modified in situ using combinations of 40+10, 30+60 and 20+40 equiv of benzeneselenol+N-PEG5000-dithiophenolmaleimide for 1 h each or sequentially with 5 equiv TCEP (1 h)+10 equiv DTT (3 h)+20 equiv N-PEG5000-dibromomaleimide, 20 equiv DTT (4 h)+25 equiv N-PEG5000-dibromomaleimide or 10 equiv TCEP (1 h)+20 equiv N-PEG5000-dithiophenolmaleimide for 30 min each at ambient temperature. All samples were purified with protein A magnetic beads and analysed by SDS-PAGE and MALDI-TOF.

As shown in FIG. 30, reaction with 40 equiv Se+10 equiv PEG yielded mainly 2 modifications (FIG. 30B), reaction with 30 equiv Se+60 equiv PEG yielded mainly 2 modifications (FIG. 30C), reaction with 20 equiv Se+40 equiv PEG yielded mainly 1 and 2 modifications (FIG. 30D), reaction with 5 equiv TCEP/10 equiv DTT/20 equiv PEG yielded a mixture of 1, 2, 3 and 4 modifications (FIG. 30E), reaction with 20 equiv DTT/25 equiv PEG yielded mainly 2, 3 and 4 modifications (FIG. 30F) and reaction with 10 equiv TCEP/20 equiv PEG yielded mainly 2 and 3 modifications (FIG. 30G). Thus, the chemically modified antibody product could be controlled by selecting appropriate reaction conditions.

### 3.12 In Situ Fluorescent Labelling of Rituximab

Maleimide bridged Rituximab was prepared using the in situ method (30 equiv benzeneselenol+60 equiv dithiophenolmaleimide, 1 h) and fluorescent Rituximab was generated by the sequential method (20 equiv DTT 1 h, then 25 equiv N-fluorescein-dibromomaleimide in a volume of DMF to reach a final concentration of 20% v/v in the antibody solution, 30 min) Both samples were purified with protein A magnetic beads and analysed by SDS-PAGE. The fluorescence of Rituximab-fluorescein was recorded at a wavelength of 518 nm (excitation 488 nm) and a concentration of 50 ng/ml. A comparison to N-fluorescein-maleimide labelled somatostatin gave 2.02 molecules of fluorescein per molecule of antibody.

3.13 Papain digest of Rituximab

Rituximab was digested using components of the Pierce Fab Preparation Kit (ThermoScientific) but a thiol-free protocol was established: Immobilised papain was activated with 10 mM DTT (in digest buffer: 50 mM phosphate, 1 mM EDTA, pH 6.8) under argon atmosphere and constant shacking (1,100 rpm) for 1 h at 25° C. in the dark. The resin was washed 4× with digest buffer (without DTT) and 0.5 ml of the antibody solution, which had been transferred into digest buffer via ultrafiltration (5 kDa MWCO), was added. The mixture was incubated for 18 h at 37° C. while shacking (1,100 rpm) in the dark. The resin was separated from the digest using a filter column, washed 3× with PBS (pH 7.4) and the digest combined with the washes. The buffer was exchanged completely for PBS using ultrafiltration columns (5 kDa MWCO), the volume adjusted to 2 ml and the sample applied to a NAb protein A column and incubated at ambient temperature under constant mixing for 1 h. The Fab fraction was eluted according to manufacturers' protocol, the column washed 3× with PBS and the Fc fraction eluted 4× with 0.2 M glycine-HCl (pH 2.5), which was neutralised with 1 M Tris (pH 8.5) solution. The Fab fraction was combined with the washes and both Fab and Fc solutions were buffer-exchanged into PBS using ultrafiltration columns (10 kDa MWCO, Sartorius).

All digests were analysed by SDS-PAGE. The concentration of Fab fragment was determined by UV/Vis using a molecular extinction coefficient of ε₂₈₀=82,905 M⁻¹ cm⁻¹.

### 3.14 Site-Selectivity of Both In Situ and Sequential Rituximab PEGylation

PEGylated Rituximab was prepared either in situ (40 equiv benzeneselenol+10 equiv N-PEG5000-dithiomaleimide, 1 h) or sequential with 20 equiv DTT or 10 equiv TCEP and N-PEG5000-dibromo- and dithiophenolmaleimide. The material was purified on a NAb protein A column (ThermoScientific) and digested with immobilised papain as described. All samples were analysed by SDS-PAGE and MALDI-TOF before and after the digest. Selectivity of the PEGylation is protocol dependent. In situ protocol (benzeneselenol) gives selectivity for FAB disulfides over Fc disulfides.

### 3.15 Stepwise PEGylation of Rituximab (Removal or Excess Reducing Agent Prior to Addition of Maleimide)

The antibody (no DMF) was reduced with 60 equiv TCEP for 1 h at ambient temperature. The reducing agent was removed by purification on a PD G-25 desalting column and 5, 8 or 10 equiv of N-PEG5000-dithiomaleimide were added quickly to the solution for 1 h. Samples were concentrated and analysed by SDS-PAGE and MALDI-TOF. Fast addition gave rise to a mixture of modified full antibody and modified heavy/heavy/light (HHL) species.

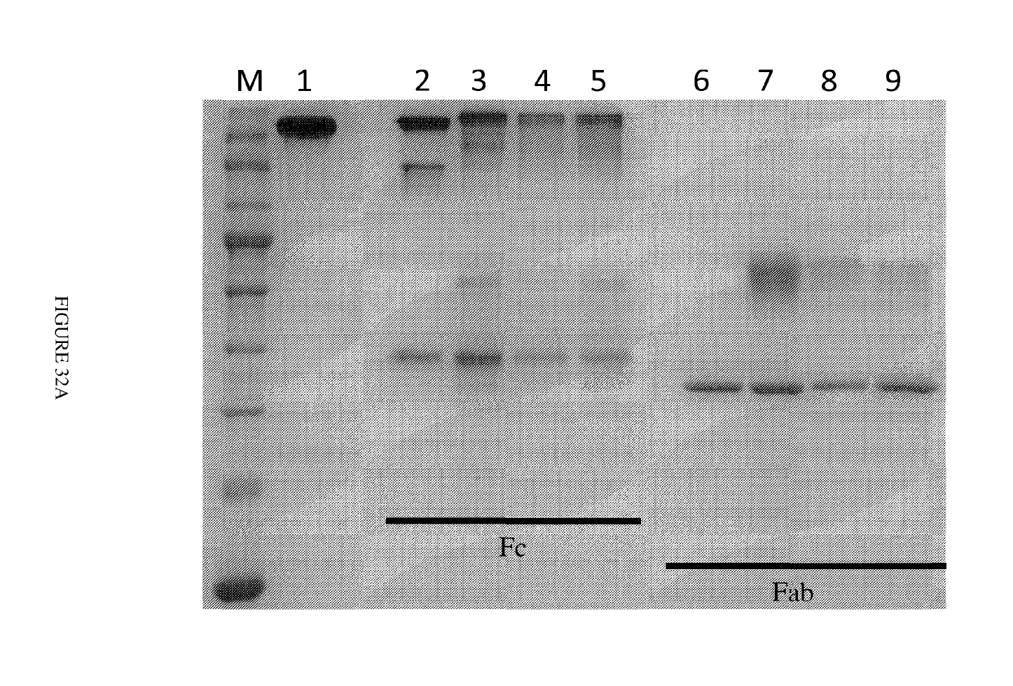

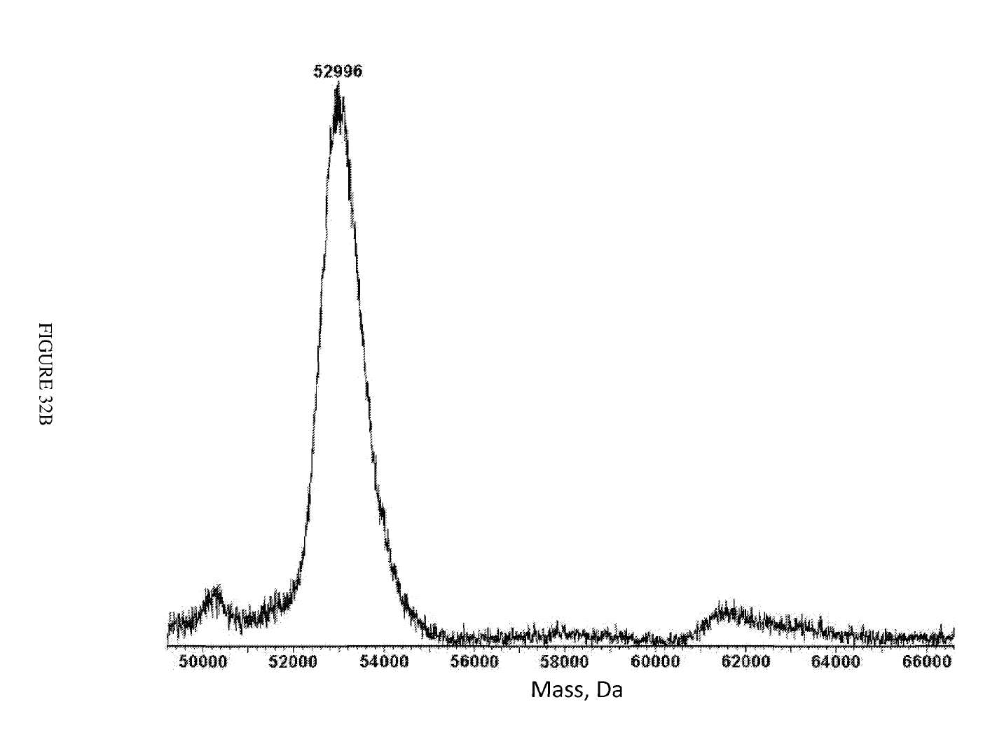

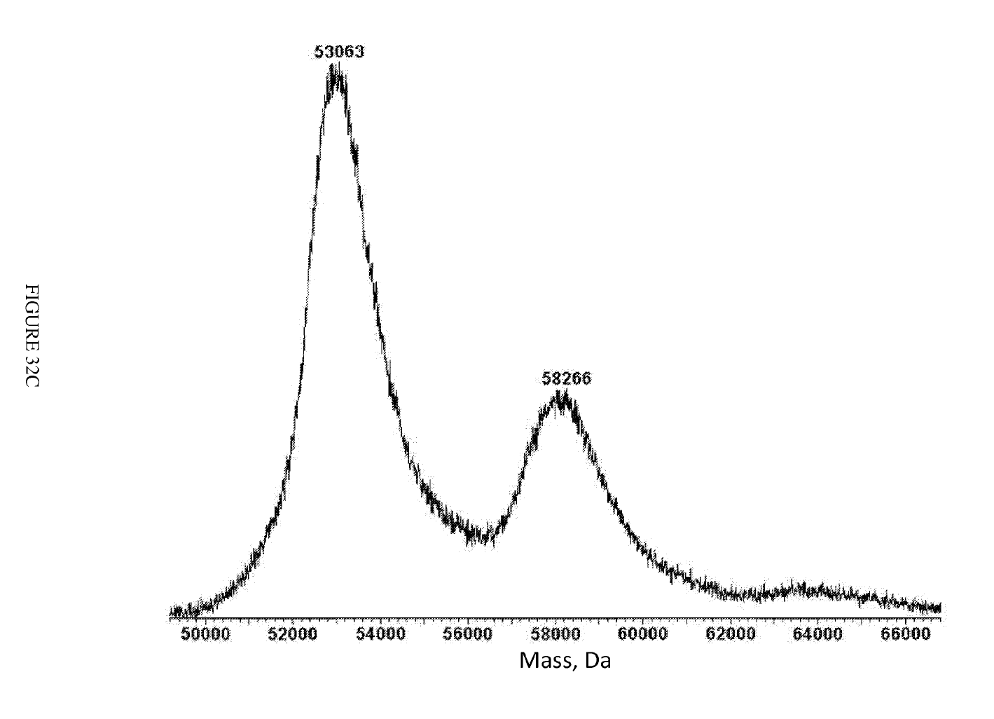

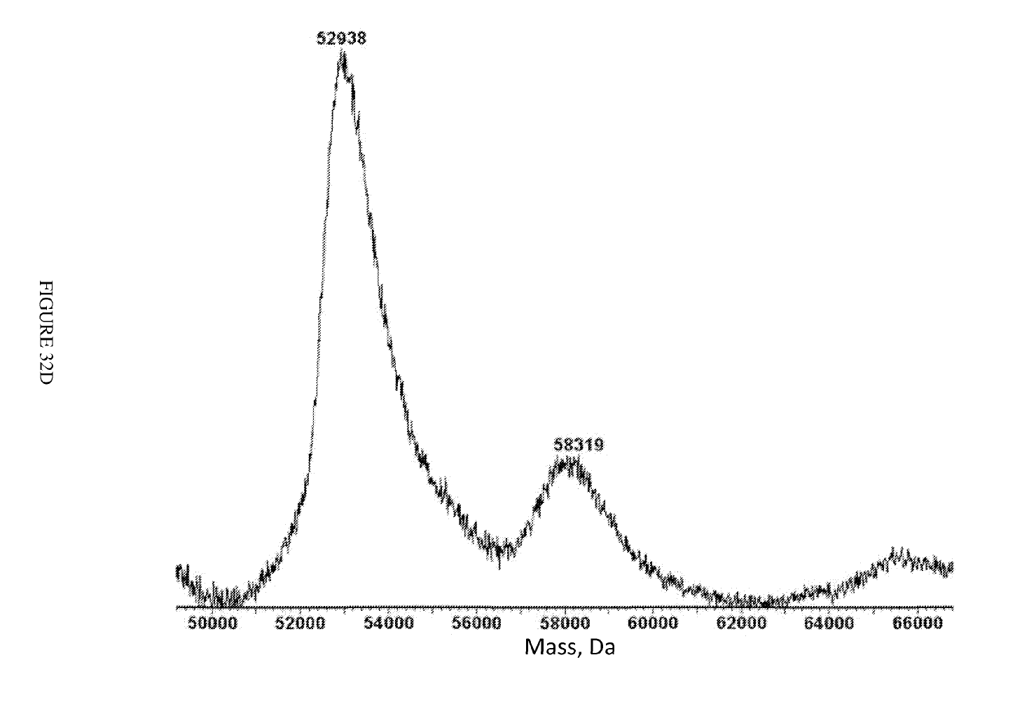

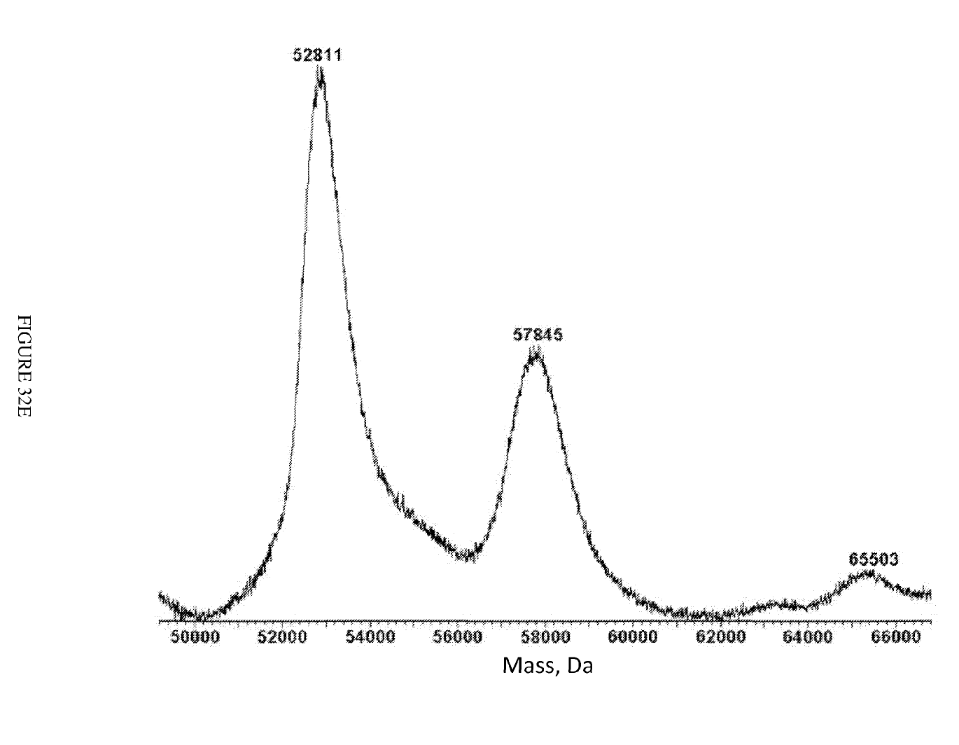

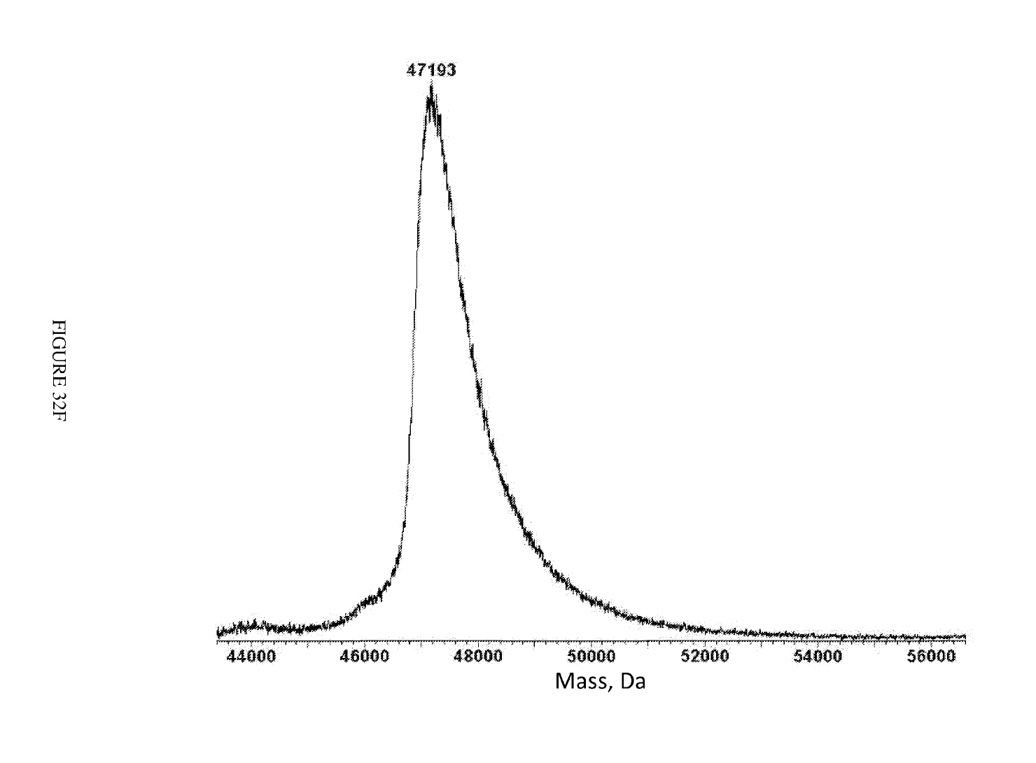

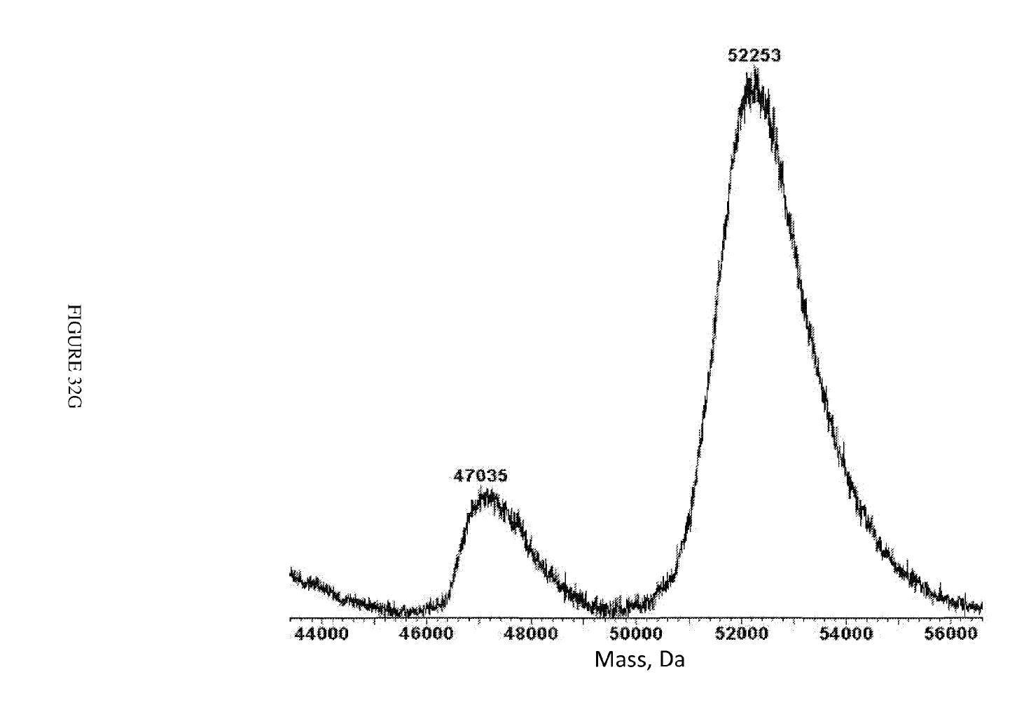

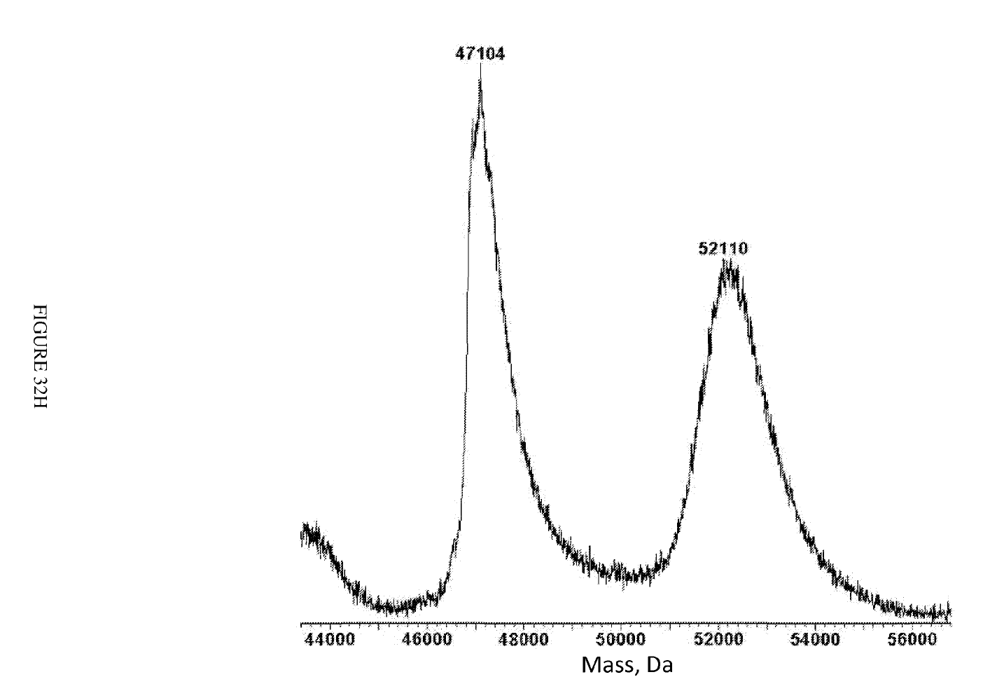

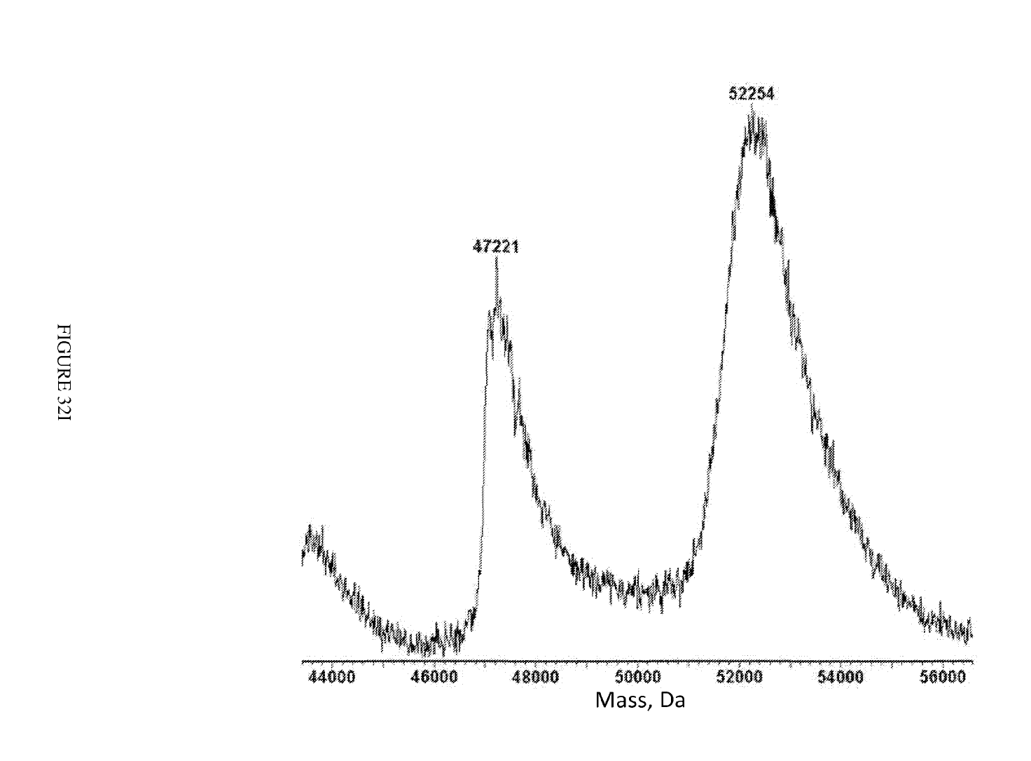

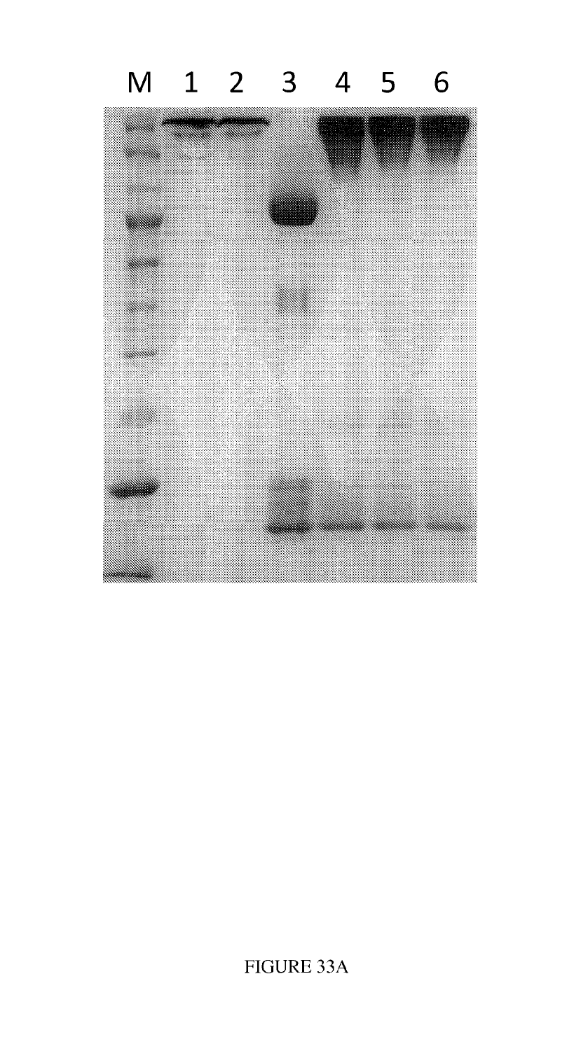

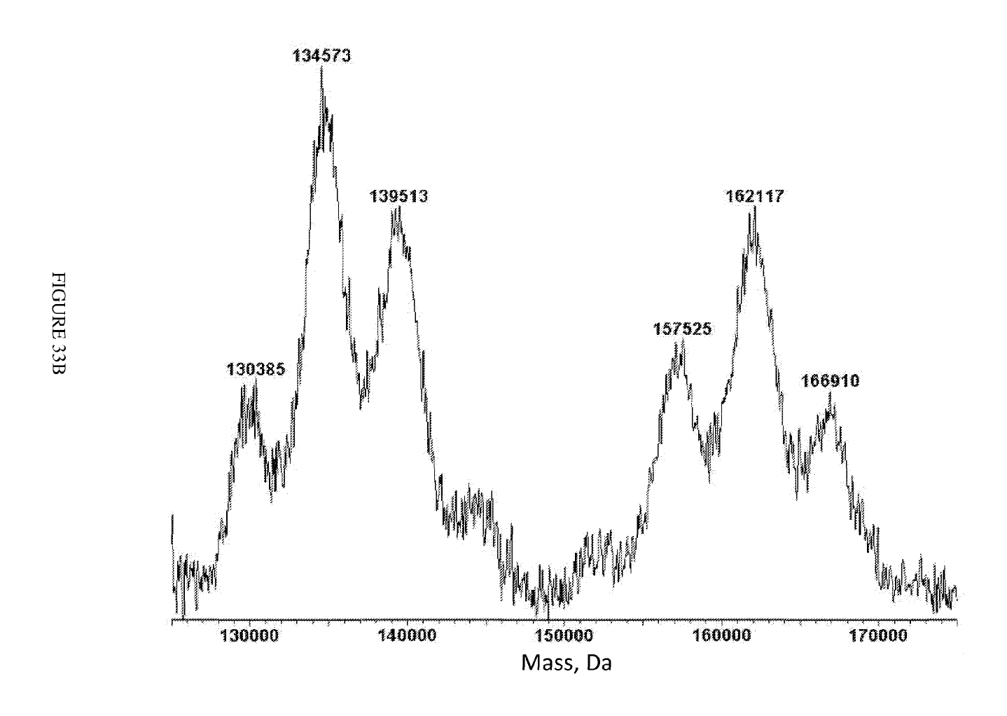

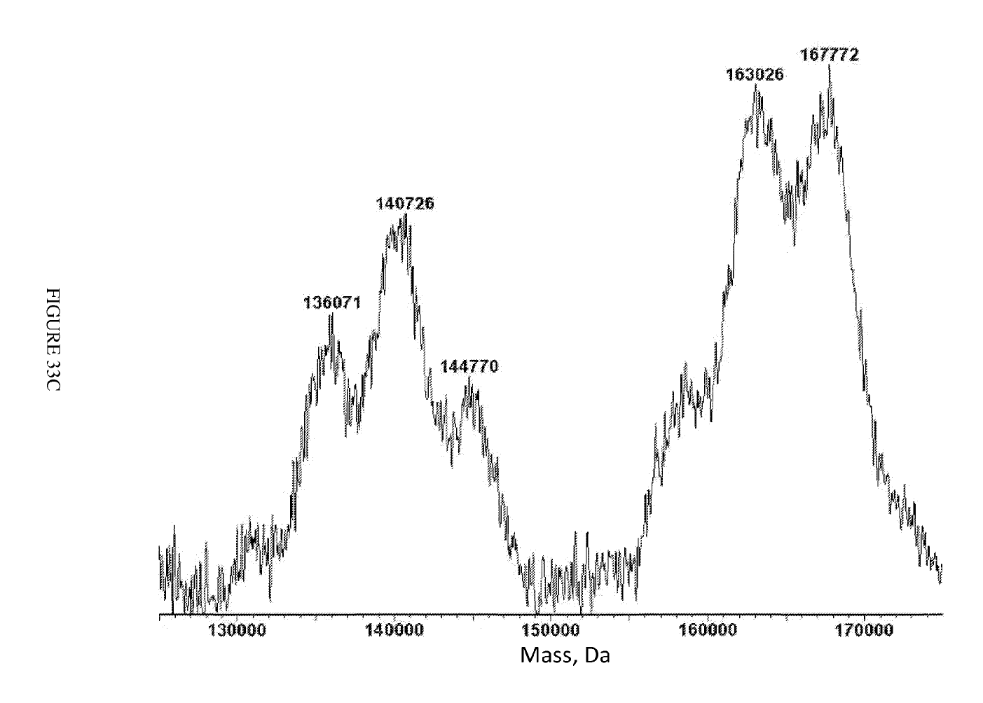

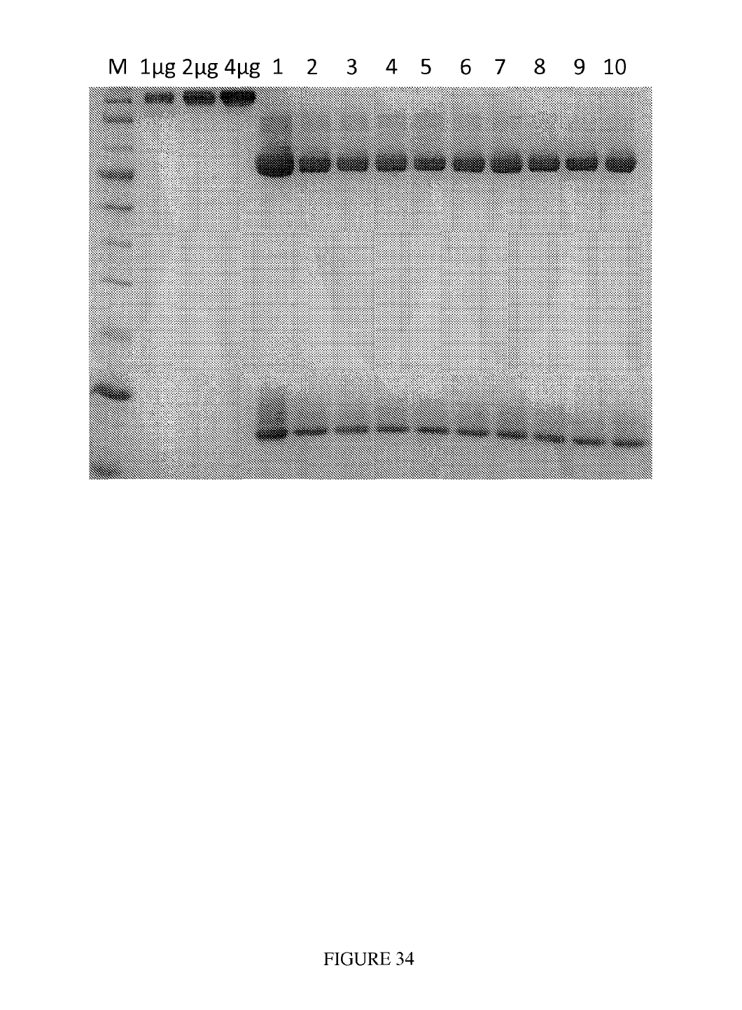

As shown in FIG. 33, reaction with 5 equiv. N-PEG5000-dithiomaleimide yielded a mixture of 2, 3 and 4 modifications (FIG. 33B), while reaction with 10 equiv. N-PEG5000-dithiomaleimide yielded a mainly 3 and 4 modifications (FIG. 33C). Thus, the chemically modified antibody product could be controlled by selecting appropriate reaction conditions.

### 3.16 Re-Oxidation Study

Rituximab (no DMF) was reduced with 60 equiv of TCEP for 1 h at ambient temperature. The sample was run through a PD G-25 desalting column to remove the reducing agent and exchange the buffer for 50 mM phosphate, 1 mM EDTA, pH 6.8. Argon was immediately bubbled through the solution and the reaction sealed and incubated in the dark at ambient temperature for 40 h. Aliquots were withdrawn under a stream of argon at various times and reacted with 40 equiv maleimide (in DMF to a final concentration of 20% v/v) for 30 mM Samples were analysed alongside a standard (1, 2 and 4 μg of the unmodified antibody) via SDS-PAGE and disulfide bond reformation quantified by densiometric analysis of the gel. The reduced disulfides were stable for extended periods of time.

### 3.17 Further Stepwise Modification of Rituximab (Removal of Excess Reducing Agent Prior to Addition of Maleimide)

Reduced antibody was prepared as established in the re-oxidation study and incubated under argon for 24 h in the dark at ambient temperature. To aliquots of the reduced and re-formed antibody were added 4, 8, 12 or 16 equiv of either N-PEG5000-dithiophenolmaleimide (in PBS) or dithiophenolmaleimide (in DMF, final 20% v/v) for 30 mM at ambient temperature. Samples were analysed by SDS-PAGE and MALDI-TOF. Allowing time for the reduced antibody to ‘re-assemble’, post-desalting and prior to maleimide addition, gives superior conversions to quadruple-labelled antibody with less HHL impurities.

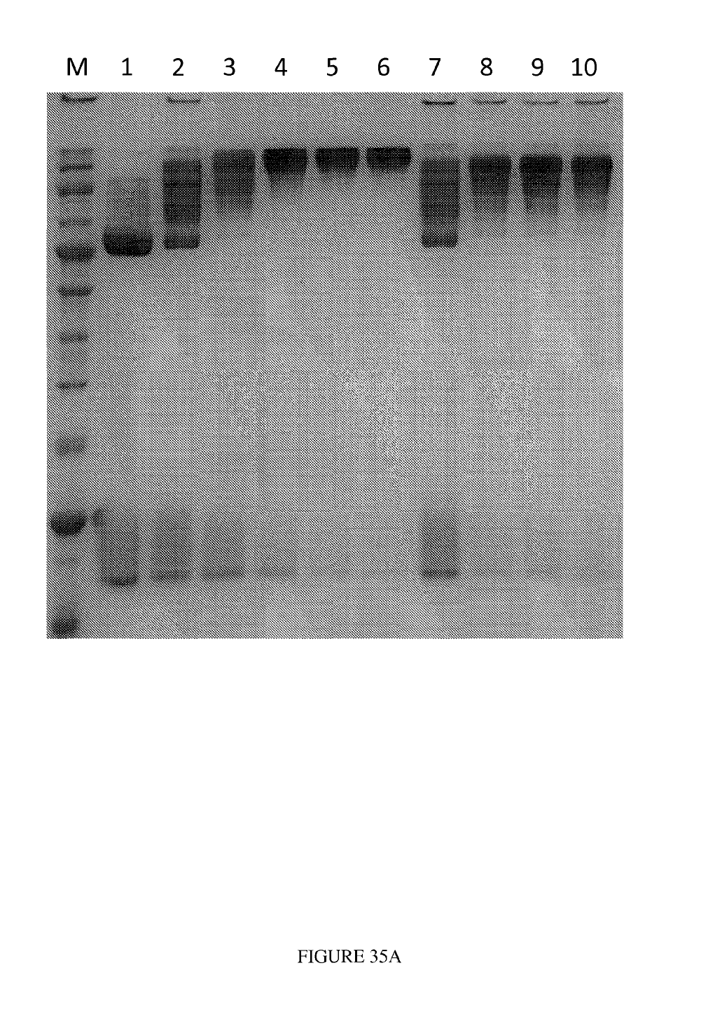

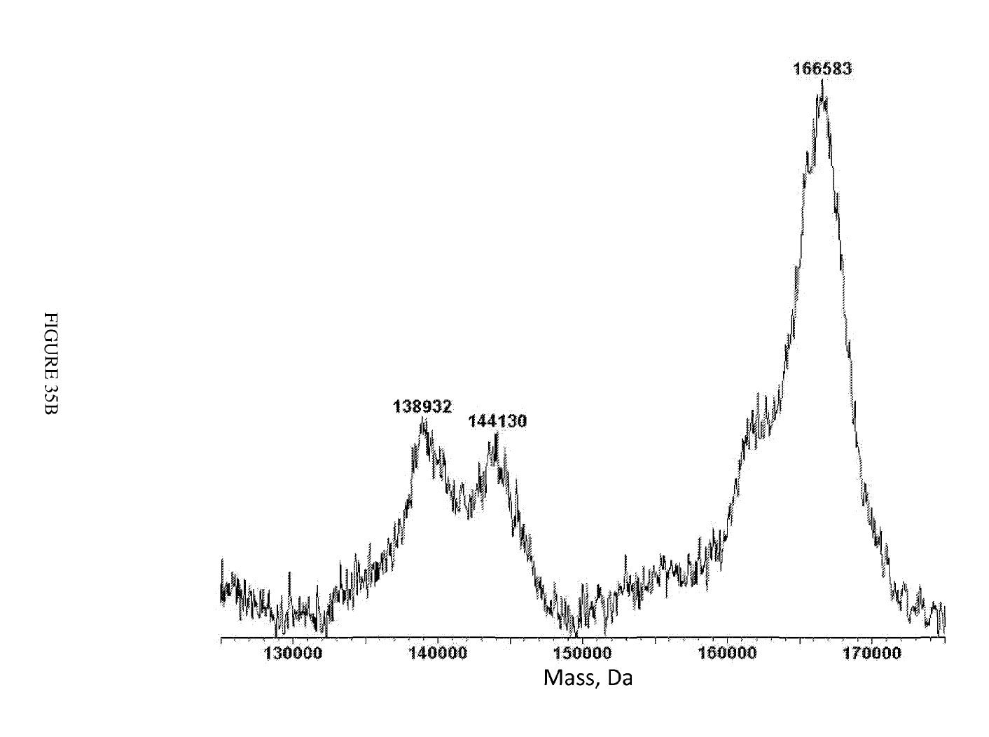

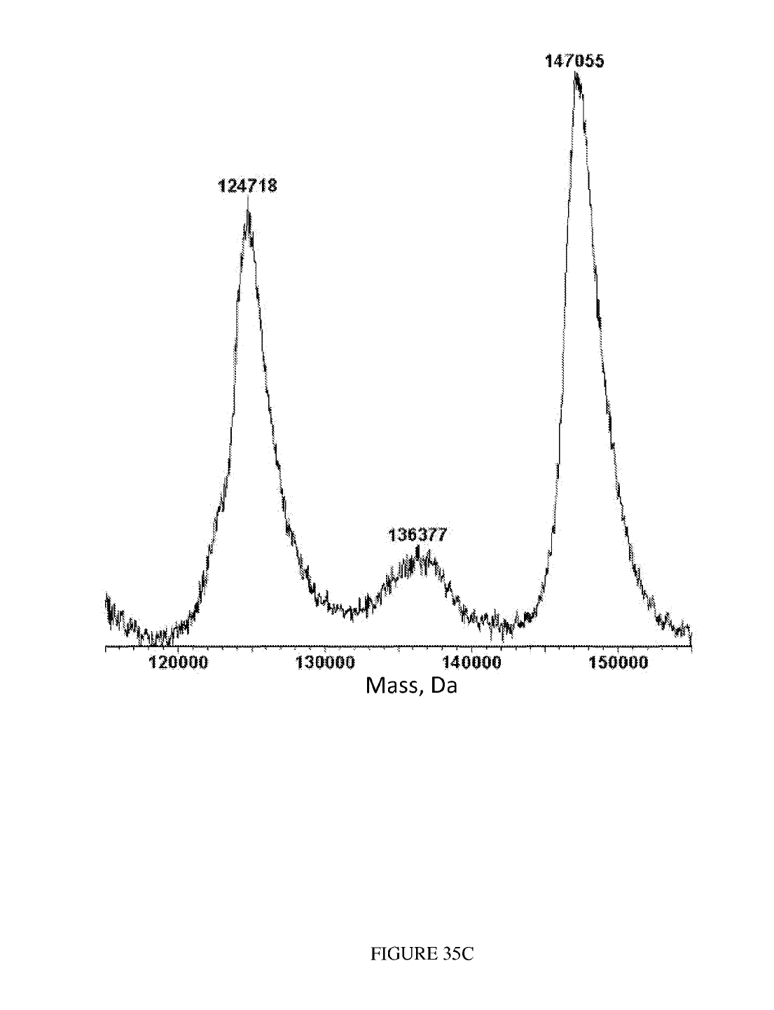

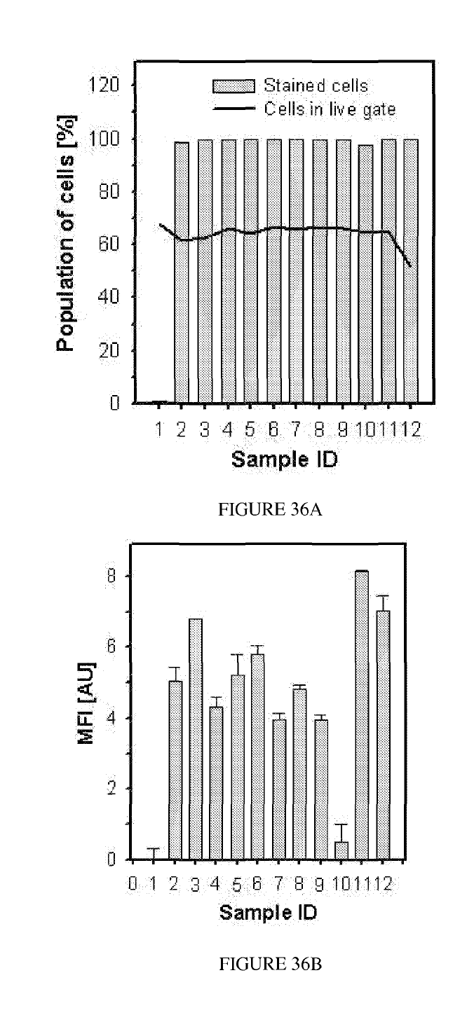

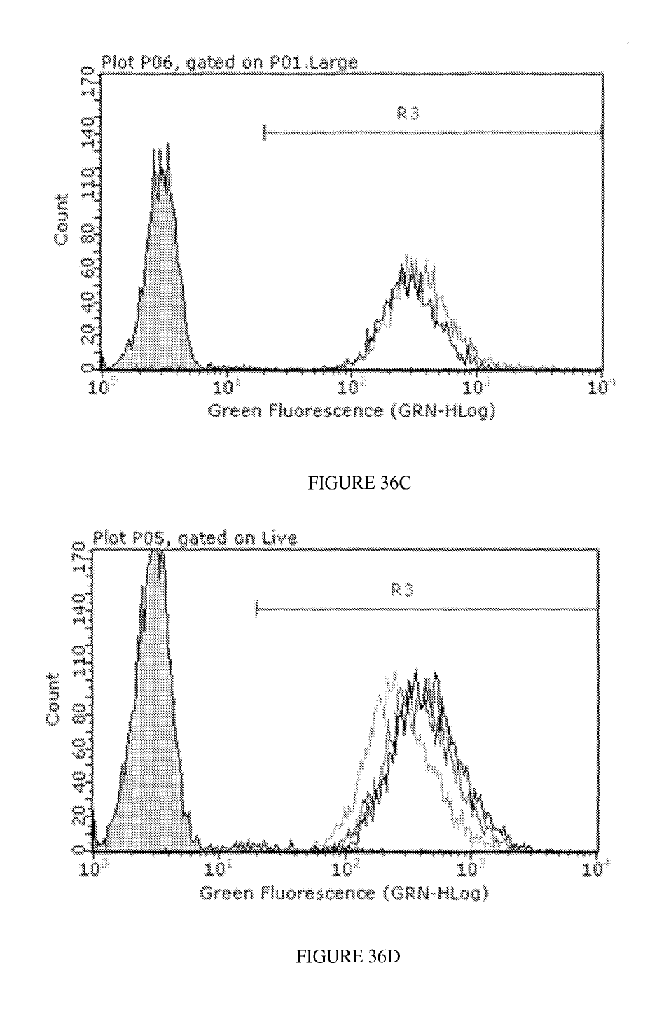

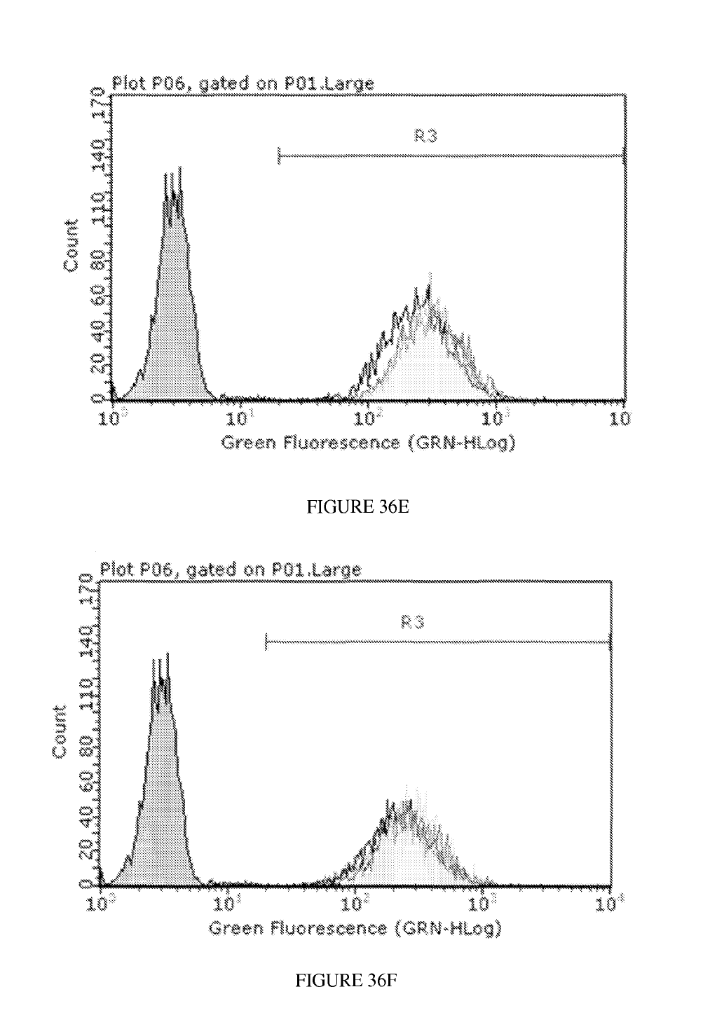

As shown in FIG. 35, reaction with 16 equiv. N-PEG5000-dithiomaleimide yielded mostly 4 modifications.

### 3.18 Functionalised Rituximabs Retain Activity

PEGylated Rituximab was synthesised as outlined under “Optimised PEGylation of Rituximab” and functionalised antibody was synthesised as described under “Functionalisation of Rituximab”. Processed antibody was prepared by subjecting Rituximab to the established in situ bridging conditions without addition of benzeneselenol. All antibody samples were purified with protein A magnetic beads, concentrated and the concentration determined (0.22 mg/ml to 0.39 mg/ml).

Log phase cultures of Raji cells (B cell line) were grown (in RPMI 1640+GlutarMAX, 25 mM HEPES, at 37° C. in humid atmosphere, 5% CO₂), harvested and transferred into buffer (PBS, 4% FCS, 0.02% sodium azide) by centrifugation and plated at 50,000 cells per well in 96 well plates. Cells were treated with 50 μl of 10, 5 or 1 μg/ml primary antibody (the Rituximab samples) in buffer for 1 h at 4° C. As controls Raji cells were also treated with unmodified/unprocessed Rituximab (positive control), an isotype control (mouse chimeric IgG1 κ, 1 μg/ml, negative control), the secondary antibody only (goat FITC conjugated anti-human IgG F(ab)₂, Jackson ImmunoResearch, negative control, 50 μl buffer during primary antibody incubation), and buffer only (in both steps, live gate control). The plate was washed and the secondary antibody was added (1 μl solution in 50 μl buffer per well). Fluorescently labelled Rituximab was added in this step to cells which had previously been treated with buffer only. The samples were incubated for 1 h at 4° C. in the dark, washed and fixed in 2% formaldehyde (in PBS) for 10 mM at ambient temperature. The cells were washed again, resuspended in 200 μl buffer and the plate loaded into the flow cytometer (Guava easyCyte 8HT, Millipore).

Data were acquired (5,000 events) and analysed using the installed software (guaraSoft, InCyte 2.2.2). Settings were adjusted using the unstained cells, positive and negative controls and samples, which had been prepared in duplicates read accordingly. Fluorescent staining was analysed after gating for live cells (forward scatter vs. side scatter). Small shifts in the fluorescent cell population over the antibody dilutions confirmed that saturation had not been reached.

### 3.19 Thermal Stability of Rituximab Analogues

In addition to the PEGylated analogues three different rituximab analogues were synthesised in preparation of a thermal stability test: Maleimide bridged rituximab was prepared by reduction of the antibody with 20 equiv DTT for 4 h at ambient temperature and addition of 25 equiv dibromomaleimide (in DMF to a final concentration of 20%) for 30 mM In analogy bridged and hydrolysed antibody was synthesised by addition of N-phenyl-dibromomaleimide instead of dibromomaleimide and incubation of the material at 37° C. for 16 h. Partial alkylated rituximab was prepared as described in the literature (Sun et al. 2005). In brief the antibody was transferred into a 25 mM NaCl, 25 mM sodium borate, 1 mM EDTA, pH 8.0 buffer, treated with 2.75 equiv TCEP for 2 h at 37° C., cooled to 4° C. and reacted with 4.4 equiv of maleimide for 30 min. All rituximab analogues were purified after the reaction on PD G-25 desalting columns (into PBS) and the concentration was determined by NanoDrop.

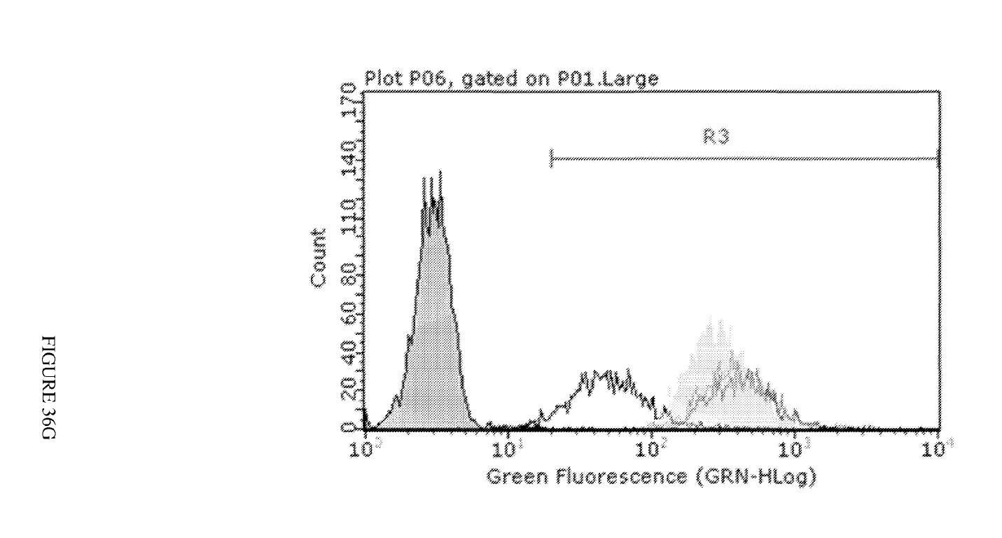

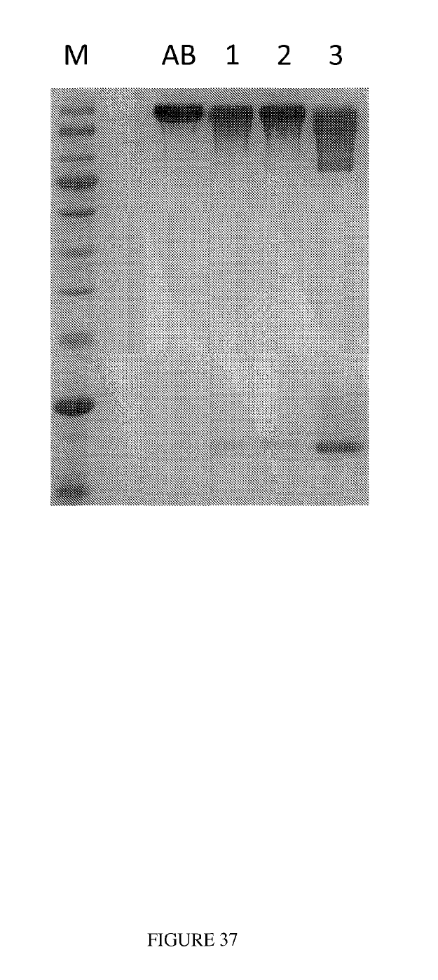

The thermal stability of all rituximab analogues prepared for the flow cytometry activity test, with the exception of the fluorescent antibody, was analysed alongside the specially synthesised samples (see FIG. 37) in a thermal shift assay (see FIG. 38). Unmodified and processed rituximab served as controls. The concentration of the antibody analogues was adjusted to 600 μM or 150 μM and mixed with a pre-diluted (1:100 in PBS) hydrophobic fluorescent dye (Sypro Orange, Sigma-Aldrich) in a 1:10 ratio of dye:antibody solution. 40 μl were transferred into a 96-well plate, which was briefly centrifuged (1,000 rpm) and sealed. The thermal shift assay was performed in a Mx 3005P qPCR machine (Stratagene) by heating the samples from 25° C. to 95° C. at a speed of 1° C. per min. The increase in fluorescence was recorded (excitation wavelength 472 nm, emission wavelength 570 nm) with the installed MxPro Software, the data exported and fitted to a sigmoid curve shape from which a simple melting temperature Tm was calculated. Thermal stability of rituximab was maintained following disulfide bridging.

### 3.20 PEGylation of Rituximab Fragments

The purified Fab and Fc fragments of rituximab were subjected at 37.3 μM and 18.7 μM respectively to the optimised in situ and sequential PEGylation procedures as outlined under “Optimised PEGylation of Rituximab”. Fragment PEGylation was visualised alongside reduction controls by SDS-PAGE, as shown in FIG. 39.

### 3.21 PEGylation of a Mix of Fc and Fab Fragments of Rituximab

The purified Fab and Fc fragments of rituximab were mixed in a 2:1 ratio to a final concentration of the “full antibody” of 18.7 μM. The mixture was PEGylated either in situ with 2, 5 or 10 equiv of N-PEG5000-dithiophenolmaleimide and 30 or 60 equiv benzeneselenol or via the TCEP-based sequential protocol with 2, 4, 6, 8, 10 or 15 equiv TCEP followed by 20 equiv of the PEGylation reagent after 1 h. All samples were analysed alongside reduction controls and single fragment reactions by SDS-PAGE. Results (see FIGS. 40 and 41) show that TCEP enables selective maleimide bridging of heavy-heavy chain disulfides whereas benzeneselenol enables selective maleimide bridging of heavy-light chain disulfides.

### 4. Modification of an IgG1 Full Length Antibody: Trastuzumab

### 4.1 Material and Preparation

Trastuzumab is a chimeric IgG1 full length antibody directed against HER2. The antibody was obtained in its clinical formulation (lyophilised). The powder was dissolved in 10 ml sterile water and the buffer exchanged completely for digest buffer (50 mM phosphate, 1 mM EDTA, pH 6.8) via ultrafiltration (MWCO 50 kDa, Sartorius). The concentration after the exchange was determined by NanoDrop and adjusted to 3.38 mg/ml (22.9 μM) and the protein solution was stored in flash frozen aliquots at −20° C. Prior to experimentation DMF was added to a final concentration of 10% (v/v) if not stated otherwise.

4.2 Reduction study with Trastuzumab

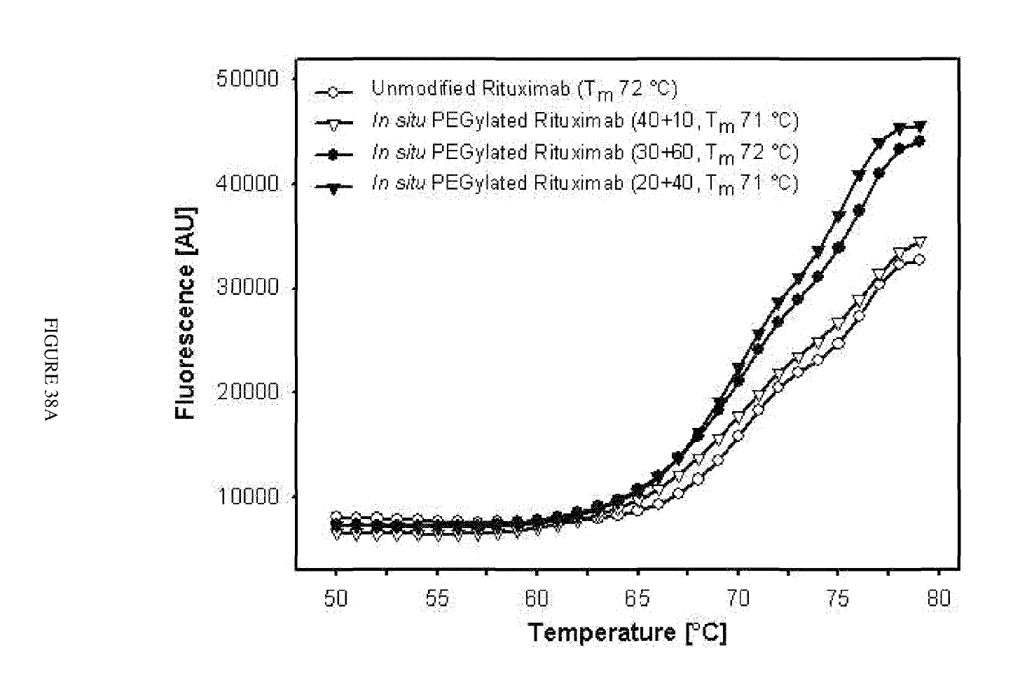

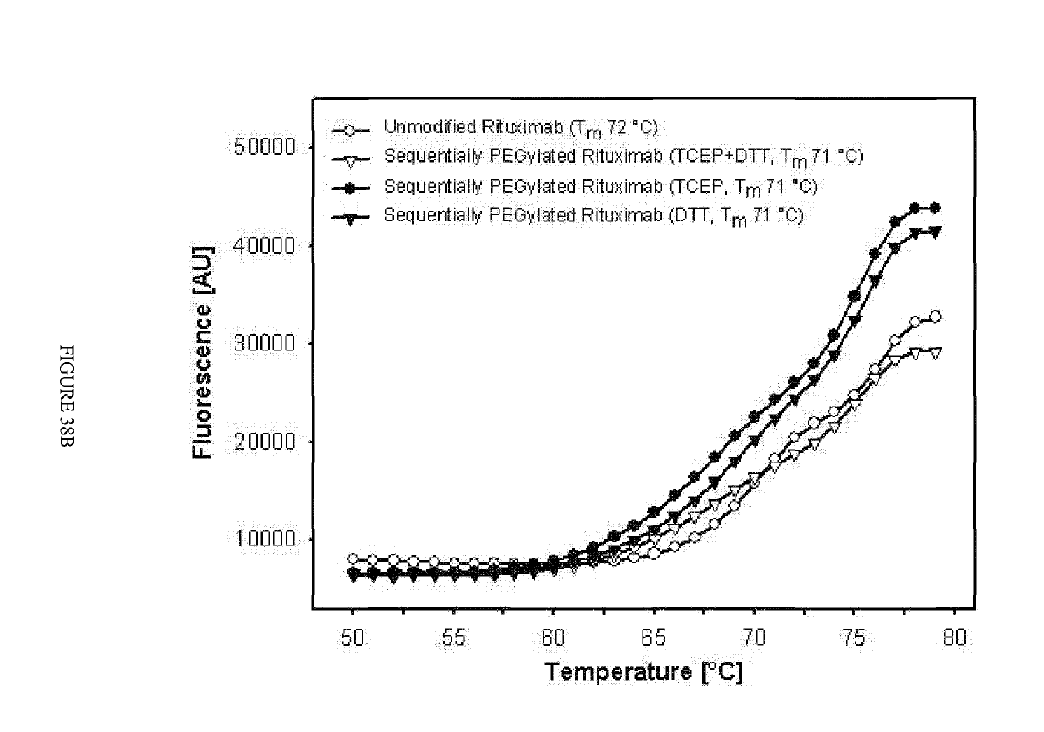

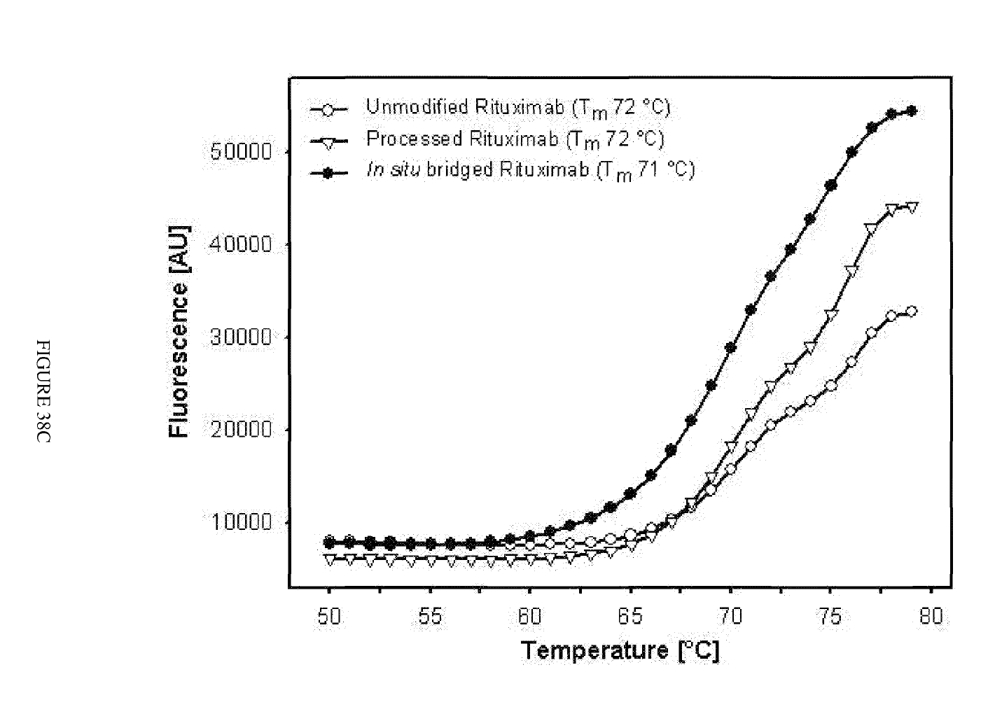

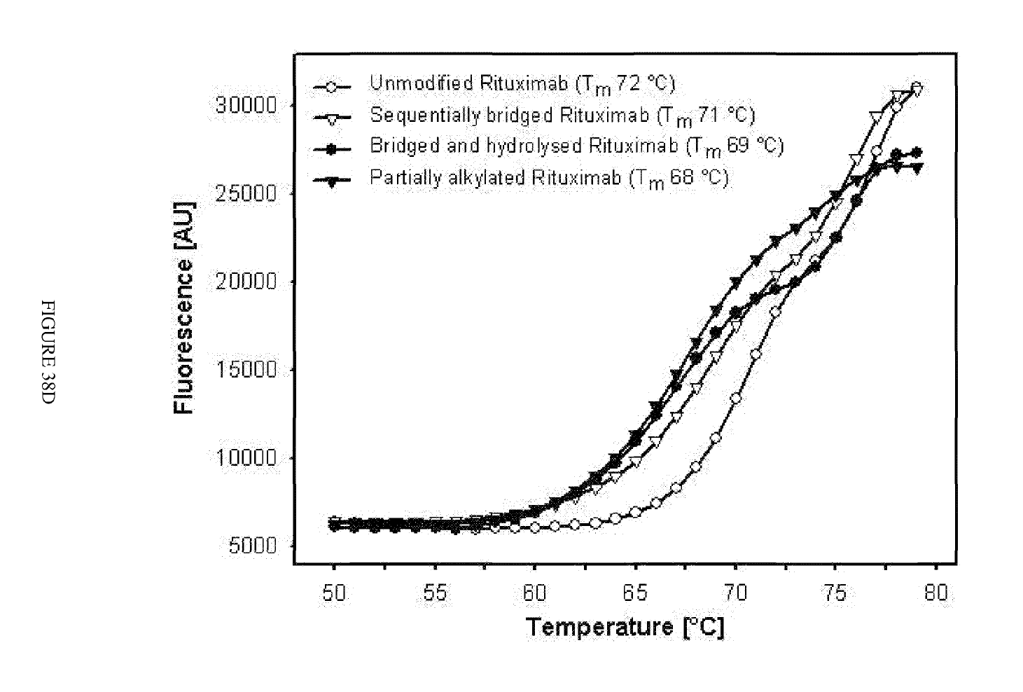

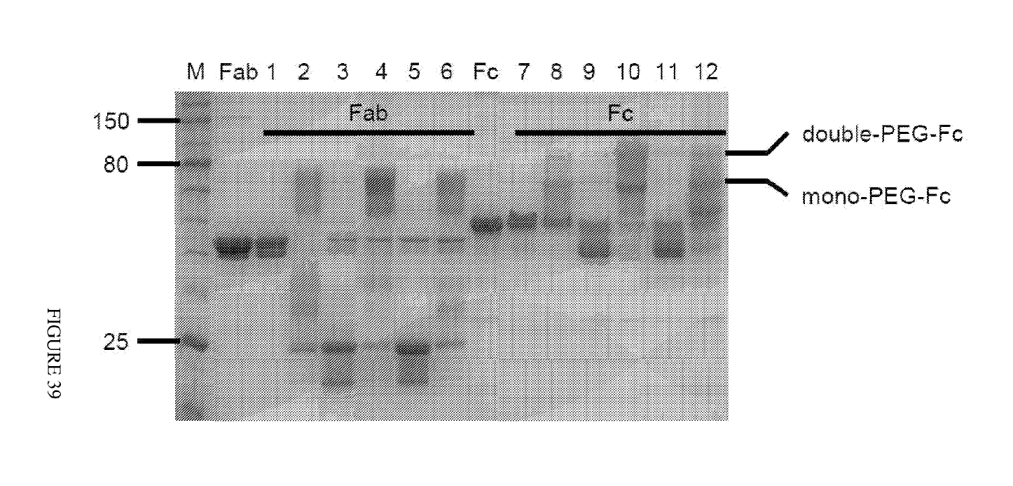

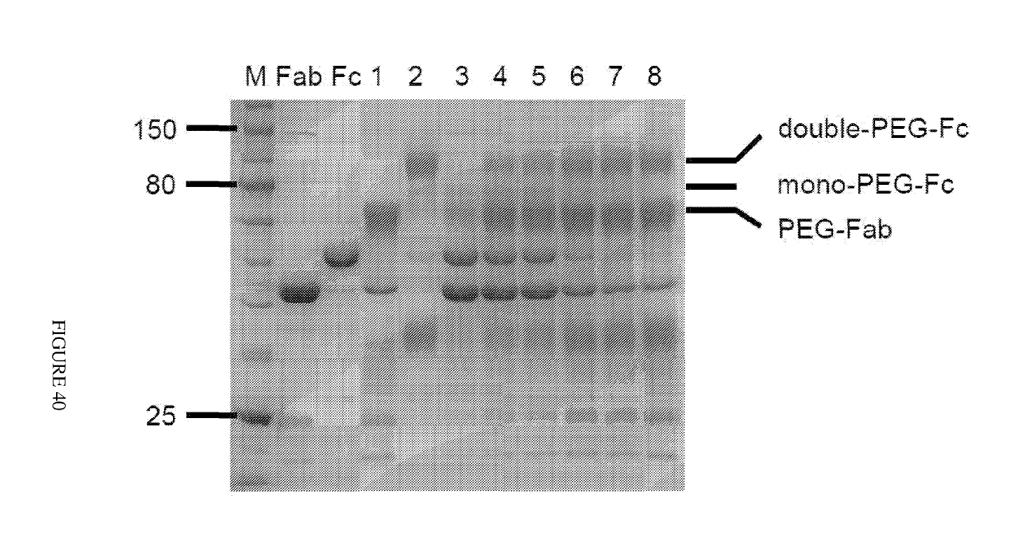

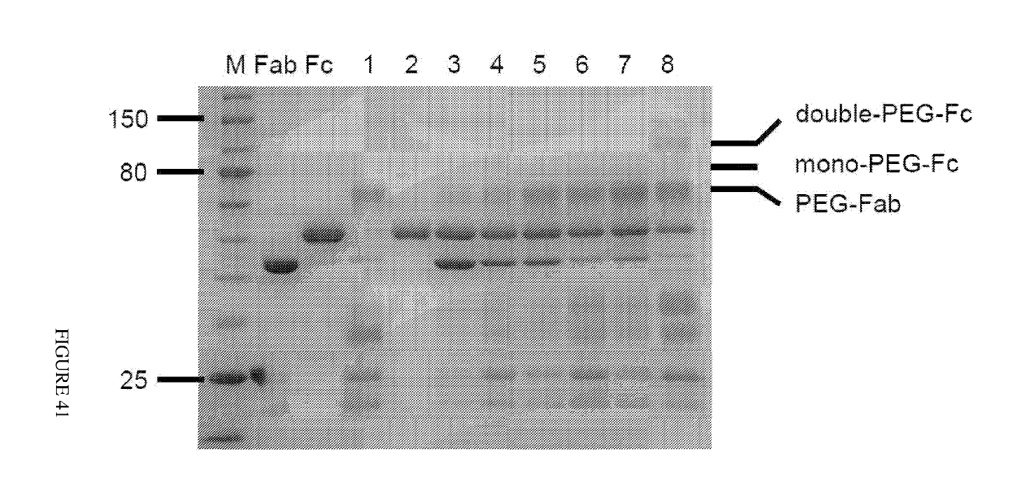

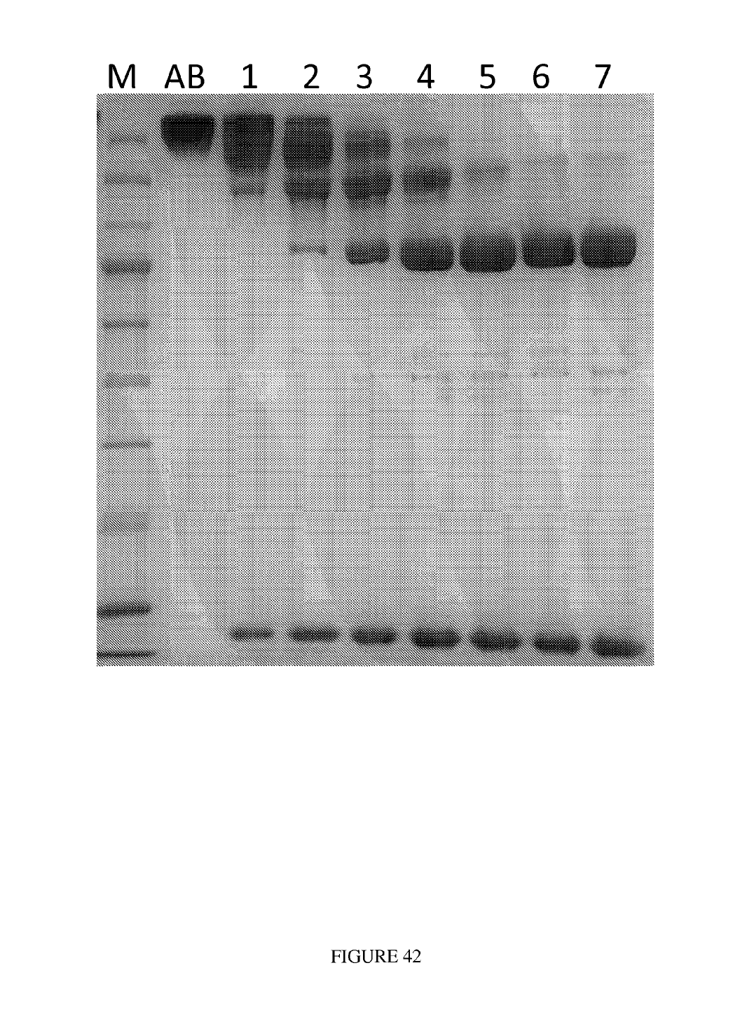

In order to lower the amounts of reducing agent in sequential prepared samples, a reduction study was carried out with Trastuzumab at an increased pH. Trastuzumab was transferred into a borate buffer (25 mM sodium borate, 25 mM NaCl, 1 mM EDTA, pH 8.0) by ultrafiltration (MWCO 10 kDa), the concentration determined with a NanoDrop device and the antibody treated with varying amounts of TCEP for 2 h at 37° C. under mild agitation. The reaction was stopped by addition of 20 equiv of maleimide (in DMF) and analysed by SDS-PAGE (see FIG. 42).

### 4.3 Synthesis of Bridging Reagents

### 4.3.1 General Remarks

All reactions were carried out at atmospheric pressure with stirring at room temperature unless otherwise stated. Reagents and solvents were purchased from commercial sources and used as supplied or purified by conventional methods. Glassware was previously flame dried for reactions that were conducted under argon. Reactions were monitored by TLC analysis carried out on silica gel SIL G/UV254 coated onto aluminium plates purchased from VWR. Visualization was carried out under a UV lamp operating at 254 nm wavelength and by staining with a solution of potassium permanganate (3 g) and potassium carbonate (20 g) in 5% aqueous sodium hydroxide (5 mL) and water (200 mL), followed by heating. Flash column chromatography was carried out with silica gel 60 (0.04-0.063 mm, 230-400 mesh) purchased from Merck. Nuclear magnetic resonance spectra were recorded in CDC3 (unless another solvent is stated) on Brucker NMR spectrometers operating at ambient room temperature probe. 1H spectra were recorded at 400, 500 or 600 MHz and 13C spectra were recorded at 125 or 150 MHz, using residual solvents as internal reference. Were necessary, DEPT135, COSY, HMQC, HMBC and NOESY spectra have been used to ascertain structure. Data is presented as follows for 1H: chemical shift in ppm (multiplicity, J coupling constant in Hz, n° of H, assignment on structure); and on 13C: chemical shift in ppm (assignment on structure). Multiplicity is reported as follows: s (singlet), d (doublet), t (triplet), q (quartet), quint. (quintet), sext. (sextet), oct. (octet), m (multiplet), br (broad), dd (doublet of doublet), dt (doublet of triplets), ABq (AB quartet). Infrared spectra were recorded on a Perkin Elmer Spectrum 100 FTIR spectrometer operating in ATR mode. Melting points were measured on a Gallenkamp apparatus and are uncorrected. Experimental procedures for all isolated compounds are presented. All yields quoted are isolated yields, unless otherwise stated, and when multiple products are obtained, data are presented in terms of order isolated. General methods for reactions are reported.

### 4.3.2 2,3-dibromo-maleimide-N-hexanoic acid 1 DBL-1

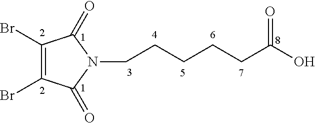 [SMILES: O=C(O)CCCCCN1C(=O)C(Br)=C(Br)C1=O]

In a 10 mL round-bottom flask, 2,3-dibromo maleic anhydride (256 mg, 1 mmol) and 6-aminocaproic acid (131 mg, 1 mmol, 1 eq.) were added. Next, AcOH (2 mL) was added and the mixture was heated at 120° C. with stirring for 3 hours. Then, the mixture was allowed to cool to room temperature. AcOH was removed by concentrating under vacuo at 80° C. and traces of AcOH were removed by adding toluene (10 mL) and concentrating once more to yield a yellow white solid which was purified by flash chromatography on silica with petroleum ether:EtOAc (1:1 v/v) to afford 1 as a white solid (311 mg, 0.84 mmol, 84%). Data for 1: mp=123-124° C. IR (pellet) νₘₐₓ 2936, 2868, 1721, 1695, 1589, 1396, 1046, 946, 842, 733. ¹H NMR (500 MHz, MeOD-d4) 1.34 (quint., J=7.5 Hz, 2H, C5), 1.63 (overlapped quint., J=7.5 Hz, 4H, C4 and C6), 2.29 (t, J=7.5 Hz, 2H, C7), 3.58 (t, J=7.5 Hz, 2H, C3); ¹³C NMR (125 MHz, MeOD-d4) 25.5 (C5), 27.2 (C4), 29.0 (C6), 34.6 (C7), 40.3 (C3), 130.3 (C2), 165.5 (C1), 177.4 (C8). ESI-MS [M]⁺ 365.9, [M+2]⁺ 367.9, [M+4]⁺ 369.9 with a 1:2:1 intensity ratio respectively. HRMS (ESI) [M]⁺ found 365.8986, C₁₀H₁₀NO₄Br₂ requires 365.8977.

### 4.3.3 2,3-dithiophenol-maleimide-N-hexanoic acid 2

### DTL-1

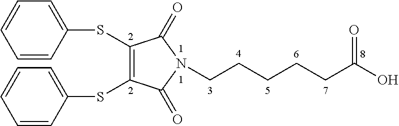 [SMILES: O=C(O)CCCCCN1C(=O)C(Sc2ccccc2)=C(Sc2ccccc2)C1=O]

In a 25 mL round-bottom flask under argon, 2,3-dibromo-maleimide-N-hexanoic acid 1 (369 mg, 1 mmol) was dissolved in MeOH (4 mL). Then, added NaOAc (172 mg, 2.1 mmol, 2.1 eq.). Next, a solution of thiophenol (225 μL, 2.2 mmol, 2.2 eq.) in MeOH (2 mL) under argon was added to the reaction mixture dropwise over 5 minutes, giving an orange solution. The mixture was stirred at room temperature for 20 minutes. Then, quenched with 20 mM HCl (10 mL, 0.2 mmol, 0.2 eq.) and extracted with EtOAc (2×20 mL). The combined organic layer was dried (MgSO₄), filtered and concentrated under vacuo to yield a yellow solid which was purified by flash chromatography on silica with petroleum ether:EtOAc (2:5 v/v) to afford 2 as a yellow solid (371 mg, 0.87 mmol, 87%). Data for 2: IR (pellet) νₘₐₓ 3058, 2940, 2870, 1766, 1697, 1541, 1395, 1176, 1049, 915, 842, 747, 687. ¹H NMR (600 MHz, MeOD-d4) 1.31 (quint., J=7.2 Hz, 2H, C5), 1.57-1.63 (overlapped quint., J=7.2 Hz, 4H, C4 and C6), 2.27 (t, J=7.2 Hz, 2H, C7), 3.51 (t, J=7.2 Hz, 2H, C3), 7.17-7.18 (overlapped m, 4H, Ph), 7.24-7.29 (overlapped m, 6H, Ph); ¹³C NMR (150 MHz, MeOD-d4) 25.5 (C5), 27.3 (C4), 29.1 (C6), 34.8 (C7), 39.5 (C3), 129.2 (Ph), 130.1 (Ph), 130.7 (Ph), 132.4 (Ph), 137.0 (C2), 168.4 (C1), 177.5 (C8). HRMS (ESI) [M]⁺ found 427.09131, C₂₂H₂₄NO₄S₂ requires 427.09065.

### 4.3.4 2,3-dithiophenol-maleimide-N—(N-doxorubicinhexanamide) 3

### DTL-1-DOX

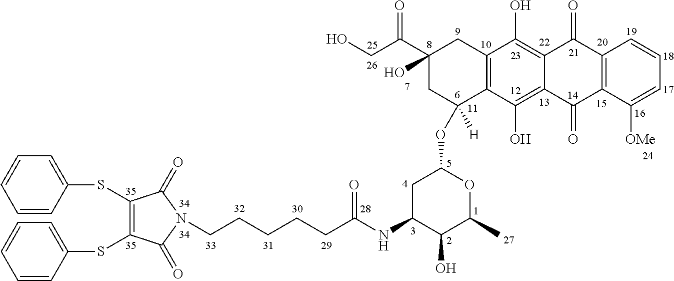 [SMILES: COc1cccc2c1C(=O)c1c(O)c3c(c(O)c1C2=O)C[C@@](O)(C(=O)CO)C[C@@H]3O[C@H]1C[C@H](NC(=O)CCCCCN2C(=O)C(Sc3ccccc3)=C(Sc3ccccc3)C2=O)[C@H](O)[C@H](C)O1]

In a 10 mL round-bottom flask under argon, 2,3-dithiophenol-maleimide-N-hexanoic acid 2 (7.63 mg, 0.0178 mmol, 1.03 eq.), HOBt (0.25 mg, 0.00178 mmol, 0.1 eq.) and HBTU (6.7 mg, 0.0178 mmol, 1.03 eq.) were dissolved in DMF (0.5 mL) to give a yellow solution. Next, a 0.378 M solution of DIPEA in DMF (50 μL, 0.0189 mmol, 1.1 eq.) was added and the mixture was stirred for 3 min. Then, a solution of doxorubicin hydrochloride (10 mg, 0.0172 mmol, 1 eq.) with DIPEA (3.27 μL, 1.1 eq.) in DMF (0.7 mL) was added. The solution turned red upon addition. The solution was stirred at room temperature for 6 hours. Then, concentrated under vacuo, added DCM (20 mL) and washed with aqueous saturated LiCl solution (3×10 mL), 15% K₂CO₃ (10 mL), 15% citric acid solution (10 mL) and water (10 mL). The organic layer was dried (MgSO₄), filtered and concentrated under vacuo to yield a red solid which was purified by flash chromatography on silica with DCM:EtOAc:MeOH (10:10:1 v/v) to afford 3 as a red solid (15.1 mg, 0.016 mmol, 92%) Data for 3: IR (pellet) νₘₐₓ 3469, 2435, 1702, 1617, 1580, 1398, 1207, 1077, 980, 735, 690. ¹H NMR (600 MHz, MeOD-d4+drops of CDCl₃) 1.20 (quint., J=7.2 Hz, 2H, C31), 1.27 (d, J=6.6 Hz, 3H, C27), 1.47-1.59 (overlapped quint., J=7.2 Hz, 4H, C30 and C32), 1.74 (dd, J=13.2, 4.8 Hz, 1H, C4), 1.99 (dt, J=13.2, 3.6 Hz, 1H C4), 2.11 (m, J=4.8 Hz, 1H, C7 overlapped with C29), 2.15 (t, J=7.2 Hz, 2H, C29), 2.33 (d, J=14.4, 1H, C7), 2.85 (d, J=18.6, 1H, C9), 3.01 (d, J=18.6, 1H, C9), 3.38 (t, J=7.2, 2H, C33), 3.61 (s, 1H, C2), 3.95 (s, 3H, C24), 4.14 (dq, J=13.2, 2.4, 1H, C3), 4.25 (q, J=6.6, 1H, C1), 4.74 (ABq, J=19.8, ν_{AB}=17.5, 2H, C26), 5.07 (dt, J=2.4, 1.8, 1H, C6), 5.41 (d, J=3.6, 1H, C5), 7.06-7.07 (overlapped m, 4H, Ph), 7.16-7.23 (overlapped m, 6H, Ph), 7.43 (d, J=8.4, 1H, C17), 7.72 (t, J=8.4, 1H, C18), 7.78 (d, J=7.8, 1H, C19); ¹³C NMR (150 MHz, MeOD-d4+ drops of CDCl₃) 17.4 (C27), 26.4 (C31), 27.2 (C32), 29.1 (C30), 30.5 (C4), 34.1 (C9), 36.7 (C29), 37.3 (C7), 39.5 (C33), 47.0 (C3), 57.1 (C24), 65.8 (C26), 68.6 (C1), 69.9 (C2), 71.2 (C6), 77.4 (C8), 102.2 (C5), 112.2 (C22), 112.4 (C13), 120.2 (C17), 120.5 (C19), 121.5 (C15), 129.2 (Ph), 130.1 (Ph), 130.6 (Ph), 132.5 (Ph), 135.1 (C11), 135.7 (C10), 136.3 (C20), 136.9 (C35), 137.1 (C18), 156.2 (C23), 157.3 (C12), 162.3 (C16), 168.2 (C34), 175.4 (C28), 187.6 (C21), 188.0 (C14), 214.7 (C25). HRMS (ESI) [M+Na]⁺ found 975.2427, C₄₉H₄₈N₂O₁₄S₂Na requires 975.2445.

### 4.3.5 2,3-dibromo-maleimide-N-(p-benzoic acid) 4

### DBL-2

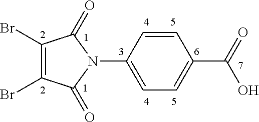 [SMILES: O=C(O)c1ccc(N2C(=O)C(Br)=C(Br)C2=O)cc1]

In a 25 mL round-bottom flask, 2,3-dibromo maleic anhydride (1.024 g, 4 mmol) and p-amino benzoic acid (0.549 g, 4 mmol, 1 eq.) were added. Next, AcOH (12 mL) was added and the mixture was heated at 120° C. with stilling for 40 minutes. The product crashes out from solution in the meantime. Then, the mixture was allowed to cool to room temperature and filtered. The filter cake was washed with cold MeOH (2 mL) and DCM and dried under vacuo to afford 4 as an off-yellow solid (1.181 g, 3.15 mmol, 79%). Data for 4: IR (pellet) νₘₐₓ 2828, 2544, 1778, 1728, 1689, 1591, 1376, 1286, 1100, 826, 723. ¹H NMR (600 MHz, DMSO-d6) 7.51 (d, J=8.4 Hz, 2H, C4), 8.06 (d, J=8.4 Hz, 2H, C5), 13.2 (br, 1H, COOH); ¹³C NMR (150 MHz, DMSO-d6) 126.6 (C4), 129.8 (C3), 130.1 (C5), 130.3 (C6), 135.3 (C2), 163.1 (C1) 166.7 (C7). ESI-MS [M]⁺ 373, [M+2]⁺ 375, [M+4]⁺ 377 with a 1:2:1 intensity ratio respectively. HRMS (ESI) [M]⁺ found 372.85833, C₁₁H₅NO₄Br₂ requires 372.85798.

### 4.3.6 2,3-dithiophenol-maleimide-N-(p-benzoic acid) 5

### DTL-2

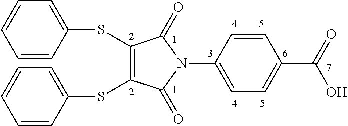 [SMILES: O=C(O)c1ccc(N2C(=O)C(Sc3ccccc3)=C(Sc3ccccc3)C2=O)cc1]

In a 25 mL round-bottom flask, 2,3-dibromo-maleimide-N-(p-benzoic acid) 4 (375 mg, 1 mmol) was dissolved in THF (12 mL). Then, added NaOAc (172 mg, 2.1 mmol, 2.1 eq.). Next, a solution of thiophenol (225 μL, 2.2 mmol, 2.2 eq.) in THF (2 mL) under argon was added to the reaction mixture dropwise over 5 minute. The mixture was stirred at room temperature for 90 minutes, slowly turning yellow overtime. Then, concentrated under vacuo, redissolved in DCM (80 mL) and sonicated for 3 minuets. Then, filtered to remove solids and concentrated the filtrate to give a yellow solid which was purified by flash chromatography on silica with DCM:MeOH (2:5 v/v) to afford 5 as a yellow solid (189 mg, 0.44 mmol, 44%). Data for 5: IR (pellet) νₘₐₓ 3120, 2163, 1708, 1431, 1053, 967, 733. ¹H NMR (500 MHz, DMSO-d6) 7.30 (overlapped m, 10H, Ph), 7.51 (d, J=8.4 Hz, 2H, C4), 8.04 (d, J=8.4 Hz, 2H, C5); ¹³C NMR (125 MHz, DMSO-d6) 126.1 (C4), 128.0 (C3), 128.9 (Ph), 129.0 (Ph), 129.9 (C5), 130.7 (overlapped, Ph, C6), 135.8 (C2), 165.2 (C1) 166.7 (C7). HRMS (ESI) [M-H⁺]⁻ found 432.0360, C₂₃H₁₄NO₄S₂ requires 432.0364.

### 4.3.7 2,3-dithiophenol-maleimide-N—(N-doxorubicin-p-benzamide) 6

### DTL-2-DOX

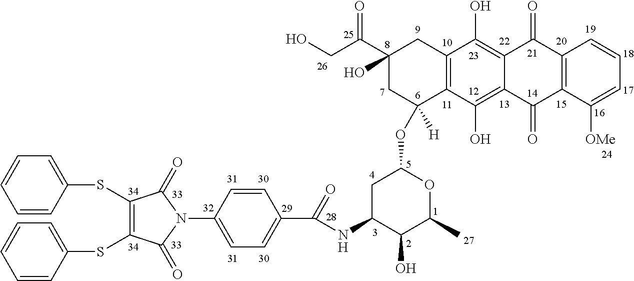 [SMILES: COc1cccc2c1C(=O)c1c(O)c3c(c(O)c1C2=O)C[C@@](O)(C(=O)CO)C[C@@H]3O[C@H]1C[C@H](NC(=O)c2ccc(N3C(=O)C(Sc4ccccc4)=C(Sc4ccccc4)C3=O)cc2)[C@H](O)[C@H](C)O1]

In a 10 mL round-bottom flask under argon, 2,3-dithiophenol-maleimide-N-(p-benzoic acid) 5 (7.46 mg, 0.0172 mmol, 1 eq.), HOBt (0.25 mg, 0.00178 mmol, 0.1 eq.) and HBTU (6.7 mg, 0.0178 mmol, 1.03 eq.) were dissolved in DMF (0.5 mL) to give a yellow solution. Next, a 0.378 M solution of DIPEA in DMF (50 μL, 0.0189 mmol, 1.1 eq.) was added and the mixture was stirred for 3 min. Then, a solution of doxorubicin hydrochloride (10 mg, 0.0172 mmol, 1 eq.) with DIPEA (3.27 μL, 1.1 eq.) in DMF (0.7 mL) was added. The solution turned red upon addition. The solution was stirred at room temperature for 6 hours. Then, added DCM (10 mL) and washed with 0.68 M AcOH:AcONa buffer pH 5 (10 mL) and aqueous saturated LiC1 solution (3×10 mL). The organic layer was dried (MgSO₄), filtered and concentrated under vacuo to yield a red solid which was purified by flash chromatography on silica with DCM:EtOAc:MeOH (20:20:1 v/v) to afford 6 as a red solid (14.9 mg, 0.0155 mmol, 90%) Data for 6: IR (pellet) νₘₐₓ 3516, 3407, 2926, 1714, 1615, 1578, 1374, 1284, 1207, 984, 732. ¹H NMR (600 MHz, DMSO-d6) 1.16 (d, J=6.6 Hz, 3H, C27), 1.54 (dd, J=13.2, 4.2 Hz, 1H, C4), 2.08 (dt, J=13.2, 3.6 Hz, 1H C4), 2.12-2.25 (ABq, J=12.6, ν_{AB}=61, 2H, C7), 3.00 (q, J=18.6, 2H, C9), 3.56 (br, 1H, C2), 3.97 (s, 3H, C24), 4.20 (m, 1H, C3 overlapped with C1), 4.25 (q, J=6.6, 1H, C1 overlapped with C3), 4.59 (d, J=5.4 Hz, 2H, C26), 4.88 (d, J=5.4 Hz, 1H, C2-OH overlapped with C26-OH), 4.90 (t, J=6.0 Hz, 1H, C26-OH overlapped with C2-OH), 4.97 (t, J=4.2 Hz, 1H, C6), 5.28 (d, J=2.4 Hz, 1H, C5), 5.52 (s, 1H, C8-OH), 7.21-7.35 (overlapped m, 10H, Ph), 7.43 (d, J=8.4, 2H, C17 overlapped with NH), 7.65 (t, J=4.8, 1H, C18), 7.90-7.91 (overlapped d, J=7.2 Hz, 4H, C30 and C31), 7.78 (d, J=7.8, 1H, C19), 13.29 (s, 1H, C12-OH), 14.05 (s, 1H, C23-OH); ¹³C NMR (150 MHz, DMSO-d6) 17.1 (C27), 29.5 (C4), 32.1 (C9), 36.8 (C7), 46.2 (C3), 56.6 (C24), 63.7 (C26), 66.7 (C1), 67.9 (C2), 70.1 (C6), 75.0 (C8), 100.5 (C5), 110.7 (C22), 110.9 (C13), 119.0 (C17), 119.8 (C19), 120.1 (C15), 126.1 (C31), 127.9 (C30 overlapped with Ph) 128.0 (Ph overlapped with C30), 128.9 (Ph), 129.0 (Ph overlapped with C29), 133.9 (C29 overlapped with C32), 130.6 (Ph), 134.2 (C11), 134.7 (C10), 135.6 (C35 overlapped with C20), 136.3 (C18), 154.5 (C23), 156.2 (C12), 160.8 (C16), 165.2 (C28), 165.4 (C34), 186.5 (C21), 186.6 (C14), 213.9 (C25). HRMS (ESI) [M+Na]⁺ found 981.1976, C₅₀H₄₂N₂O₁₄S₂Na requires 981.1975.

### 4.3.8 Fmoc-valine-citruline 7

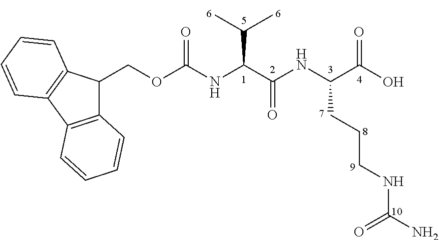 [SMILES: CC(C)[C@H](NC(=O)OCC1c2ccccc2-c2ccccc21)C(=O)N[C@@H](CCCNC(N)=O)C(=O)O]

In a 100 mL round-bottom flask under argon, Fmoc-valine (2.5 g, 7.37 mmol) and N-hydroxy-succinimide (0.86 g, 7.37 mmol, 1 eq.) were dissolved in THF (10 mL). Then, cooled down to 0° C. and added dicyclohexylcarbodiimide (DCC, 1.54 g, 7.37 mmol, 1 eq.). Stirred for 5 minutes and then removed the ice bath, allowing to stir at room temperature for 5 hours. Then, filtered and the filter cake was further washed with THF (30 mL). The combined filtrates were concentrated and dried under vacuo to yield Fmoc-valine-OSu as a white solid. Next, dissolved citrulline (1.36 g, 7.74 mmol, 1.05 eq.) in water (10 mL) to which NaHCO₃ (0.65 g, 7.74 mmol, 1.05 eq.) was added. Then, Fmoc-valine-OSu was suspended in dimethoxyethane (DME, 20 mL) and THF (10 mL) and added over the solution of citrulline over 5 minutes. A precipitate slowly formed over time. The suspension was stirred at room temperature for 16 hours. Next, added 15% citric acid solution (35 mL) and extracted with 10:1 EtOAc:ⁱPrOH (2×50 mL). The combined organic layer was washed with water (2×75 mL), then dried (MgSO₄), filtered, concentrated and dried under vacuo to yield a dirty-white solid. Next, added Et₂O (40 mL), sonicated for 10 minutes, filtered and washed collected solid with Et₂O. Dried under vacuo to yield 7 as a white solid (1.53 g, 3.1 mmol, 42%). Data for 7: IR (pellet) νₘₐₓ 3290, 2960, 1689, 1643, 1535, 1448, 1233, 1031, 738. ¹H NMR (600 MHz, DMSO-d6) 0.85-0.89 (overlapped d, J=6.6 Hz, 6H, C6), 1.38 (m, 2H, C8), 1.48-1.71 (m, 2H, C7), 1.98 (oct., J=6.6 Hz, 1H, C5), 2.95 (q, J=6.6 Hz, 2H, C9), 3.92 (ABq, J=7.2, ν_{AB}=5.4, 1H, C1), 4.14 (m, 1H, Fmoc), 4.21 (m, 2H, Fmoc), 4.28 (m, 1H, C3), 5.40 (br, 2H, ClONH2), 5.95, (t, J=5.4 Hz, 1H, C9NH), 7.32 (m, 2H, Fmoc), 7.42 (m, 2H, Fmoc), 7.32 (m, 3H, Fmoc overlapped with C1NH), 7.75 (t, J=7.8 Hz, 2H, Fmoc), 7.89 (d, J=7.8 Hz, 2H, Fmoc), 8.20 (d, J=7.2 Hz, 1H, C2NH), 12.55 (br, 1H, COOH); ¹³C NMR (150 MHz, DMSO-d6) 18.3 (C6), 19.2 (C6), 26.7 (C7), 28.4 (C8), 30.6 (C5), 38.8 (C9), 46.7 (Fmoc), 51.9 (C3), 59.8 (C1), 64.9 (Fmoc), 65.7 (Fmoc), 125.4 (Fmoc), 127.1 (Fmoc), 127.7 (Fmoc), 140.7 (Fmoc), 143.8 (Fmoc), 143.9 (Fmoc), 156.1 (Fmoc), 158.8 (C10), 171.4, (C4), 173.5 (C2). HRMS (ESI) [M-H⁺] found 495.2261, C₂₆H₃₁N₄O₆ requires 495.2244.

### 4.3.9 Fmoc-valine-citruline-PABOH 8

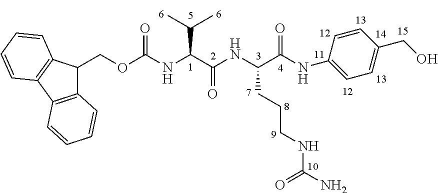 [SMILES: CC(C)[C@H](NC(=O)OCC1c2ccccc2-c2ccccc21)C(=O)N[C@@H](CCCNC(N)=O)C(=O)Nc1ccc(CO)cc1]

In a 100 mL round-bottom flask, Fmoc-valine-citruline 7 (0.994 g, 2 mmol) and p-aminobenzoic alcohol (PABOH, 0.493 g, 4 mmol, 2 eq.) were dissolved in 2:1 DCM:MeOH (36 mL). Next, added 2-ethoxy-1-ethoxycarbonyl-1,2-dihydroquinoline (EEDQ, 0.989 g, 4 mmol, 2 eq.) and left stirring for 16 hours. Then, concentrated under vacuo (40° C.), suspended over Et₂O (75 mL) and sonicated for 5 minutes, filtered and washed collected solid with Et₂O. Dried under vacuo to yield 8 as a white solid (0.958 g, 1.59 mmol, 80%). Data for 8: IR (pellet) νₘₐₓ 3275, 2961, 1687, 1640, 1532, 1249, 1032, 739, 521. ¹H NMR (600 MHz, DMSO-d6) 0.84-0.88 (overlapped d, J=6.6 Hz, 6H, C6), 1.33-1.45 (2m, 2H, C8), 1.56-1.71 (2m, 2H, C7), 1.98 (oct., J=6.6 Hz, 1H, C5), 2.90-3.03 (2m, J=6.6 Hz, 2H, C9), 3.92 (ABq, J=7.5, ν_{AB}=4.9, 1H, C1), 4.22 (m, 2H, Fmoc), 4.30 (m, 1H, Fmoc), 4.42 (d, J=4.0 Hz, 3H, C15 overlapped with C1), 5.09 (t, J=5.5 Hz, 1H, C15OH), 5.40 (br, 2H, C10NH2), 5.95, (t, J=5.5 Hz, 1H, C9NH), 7.22 (d, J=8.5 Hz, 2H, C13), 7.31 (t, J=7.0 Hz, 2H, Fmoc), 7.40 (m, 3H, Fmoc overlapped with C1NH), 7.53 (d, J=8.0 Hz, 2H, C12), 7.73 (t, J=7.5 Hz, 2H, Fmoc), 7.88 (d, J=7.5 Hz, 2H, Fmoc), 8.10 (d, J=7.5 Hz, 1H, C2NH), 9.97 (br, 1H, C11NH); ¹³C NMR (150 MHz, DMSO-d6) 18.3 (C6), 19.2 (C6), 26.8 (C7), 29.5 (C8), 30.4 (C5), 38.8 (C9), 46.7 (Fmoc), 53.0 (C3), 60.1 (C1), 62.6 (C15), 65.7 (Fmoc), 118.8 (C12), 120.1 (Fmoc), 125.4 (Fmoc), 126.9 (C13), 127.6 (Fmoc), 137.4 (C11), 137.5 (C14), 140.7 (Fmoc), 143.8 (Fmoc), 143.9 (Fmoc), 156.1 (Fmoc), 158.8 (C10), 170.4, (C4), 171.2 (C2). HRMS (ESI) [M+Na]⁺ found 624.2788, C₃₃H₃₉N₅O₆Na requires 624.2798.

### 4.3.10 Valine-citruline-PABOH 9

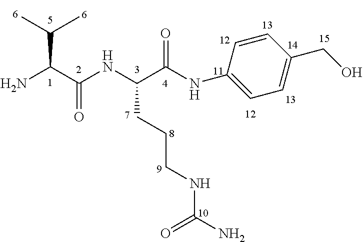 [SMILES: CC(C)[C@H](N)C(=O)N[C@@H](CCCNC(N)=O)C(=O)Nc1ccc(CO)cc1]

In a 50 mL round-bottom flask, Fmoc-valine-citruline-PABOH 8 (1.178 g, 1.59 mmol) was dissolved in DMF (16 mL). Next, diethylamine (3.12 mL, 30 mmol, 19 eq.) was added and left stirring for 16 hours in the dark. Then, concentrated under vacuo (40° C.), suspended over DCM (75 mL), sonicated for 5 minutes and filtered to collect a gum-like solid material that was washed in the filter with DCM. Note: more than one cycle of sonication may be required. Dissolved collected material in MeOH to remove from filter and concentrated under vacuo to yield 9 as a light-brown smuged white solid (0.477 g, 1.25 mmol, 79%). Data for 9: IR (pellet) νₘₐₓ 3282, 2960, 2871, 1644, 1603, 1538, 1513, 1413, 1310, 1008, 823. ¹H NMR (600 MHz, DMSO-d6) 0.78-0.88 (2d, J=6.6 Hz, 6H, C6), 1.32-1.43 (2m, 2H, C8), 1.55-1.70 (2m, 2H, C7), 1.93 (oct., J=6.6 Hz, 1H, C5), 2.92-3.01 (2m, J=6.6 Hz, 2H, C9), 3.92 (m, J=4.8, 1H, C1), 4.42 (d, J=4.8 Hz, 2H, C15), 4.47 (q, J=7.2 Hz, 1H, C3), 5.11 (t, J=5.5 Hz, 1H, C15OH), 5.42 (br, 2H, ClONH2), 5.98, (t, J=5.5 Hz, 1H, C9NH), 7.23 (d, J=8.4 Hz, 2H, C13), 7.54 (d, J=8.4 Hz, 2H, C12), 8.15 (d, J=7.8 Hz, 1H, C2NH), 10.05 (br, 1H, C11NH); ¹³C NMR (150 MHz, DMSO-d6) 16.9 (C6), 19.6 (C6), 26.7 (C7), 30.2 (C8), 31.3 (C5), 38.6 (C9), 52.5 (C3), 59.6 (C1), 62.6 (C15), 118.9 (C12), 126.9 (C13), 137.4 (C11), 137.5 (C14), 158.8 (C10), 170.5, (C4), 174.3 (C2). HRMS (ESI) [M+Na]⁺ found 402.2106, C₁₈H₂₉N₅O₄Na requires 402.2117.

### 4.3.11 DTL-1-Valine-citruline-PABOH 10

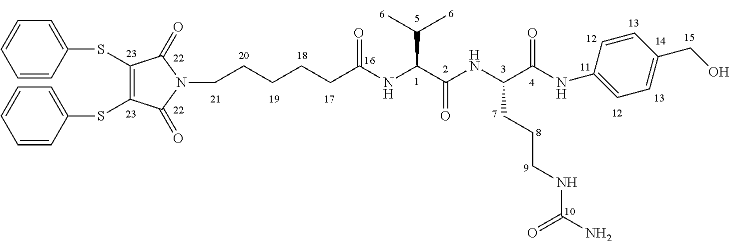 [SMILES: CC(C)[C@H](NC(=O)CCCCCN1C(=O)C(Sc2ccccc2)=C(Sc2ccccc2)C1=O)C(=O)N[C@@H](CCCNC(N)=O)C(=O)Nc1ccc(CO)cc1]

In a 5 mL round-bottom flask, under argon, DTL-1 (85.7 mg, 0.2 mmol), HOBt (2.6 mg, 0.02 mmol, 0.1 eq.) and HBTU (75 mg, 0.2 mmol, 1 eq.) were dissolved in DMF (0.5 mL) to give a yellow solution. Next, DIPEA (37.7 μL, 0.22 mmol, 1.1 eq.) was added and the mixture was stirred for 3 min. Then, added valine-citrulline-PABOH (76.1 mg, 0.2 mmol, 1 eq.) and stirred at room temperature in the dark for 5 hours. Then, concentrated under vacuo, redissolved in 8:1 DCM:MeOH (90 mL) and filtered. Concentrated once more under vacuo to yield a yellow solid which was purified by flash chromatography on silica with DCM:MeOH (9:1 v/v) to afford 10 as a yellow solid (126.8 mg, 0.16 mmol, 80%) Data for 10: IR (pellet) νₘₐₓ 3274, 2933, 2867, 1701, 1633, 1529, 1395, 1213, 1044, 1023, 736, 686. ¹H NMR (600 MHz, DMSO-d6) 0.82-0.86 (2d, J=6.6 Hz, 6H, C6), 1.20 (quint., J=7.2 Hz, 2H, C19), 1.33-1.44 (2m, 2H, C8), 1.49 (overlapped m., 4H, C18 and C20), 1.55-1.70 (2m, 2H, C7), 1.95 (oct., J=6.6 Hz, 1H, C5), 2.09-2.21 (2m, J=7.2 Hz, 2H, C17), 2.92-3.01 (2m, J=6.6 Hz, 2H, C9), 3.38 (t, J=7.2 Hz, 2H, C21), 4.12 (ABq, J=7.2, ν_{AB}=4.3, 1H, C1), 4.19 (ABq, J=8.4, ν_{AB}=10.8, 1H, C3), 4.42 (d, J=5.4 Hz, 2H, C15), 5.11 (t, J=5.4 Hz, 1H, C15OH), 5.42 (br, 2H, C10NH2), 5.98, (t, J=5.4 Hz, 1H, C9NH), 7.21-7.30 (overlapped m, 12H, Ph and C13), 7.54 (d, J=8.4 Hz, 2H, C12), 7.83 (d, J=8.4 Hz, 1H, C1NH), 8.08 (d, J=7.2 Hz, 1H, C2NH), 9.91 (br, 1H, C11NH); ¹³C NMR (150 MHz, DMSO-d6) 18.2 (C6), 19.3 (C6), 24.9 (C20), 25.3 (C19), 25.8 (C18), 26.9 (C8), 27.6 (C17), 29.4 (C7), 30.4 (C5), 34.9 (C21), 38.4 (C9), 53.1 (C3), 57.6 (C1), 62.6 (C15), 118.9 (C12), 126.9 (C13), 127.9 (Ph), 129.1 (Ph), 129.2 (Ph), 130.7 (Ph), 135.4 (C23), 137.4 (C11), 137.5 (C14), 158.9 (C10), 166.5 (C22), 170.4, (C4), 172.3 (C16), 172.8 (C2). HRMS (ESI) [M+Na]⁺ found 811.2917, C₄₀H₄₈N₆O₇S₂Na requires 811.2924.

### 4.3.12 DTL-1-Valine-citruline-PABC-DOX 11

### DTL-3-DOX 11

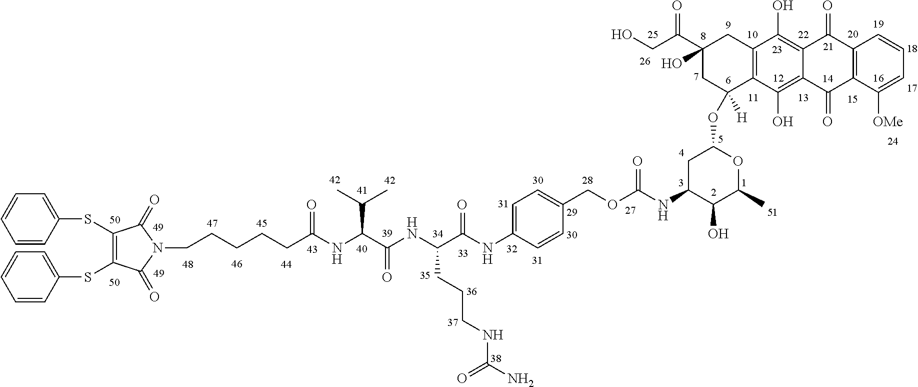 [SMILES: COc1cccc2c1C(=O)c1c(O)c3c(c(O)c1C2=O)C[C@@](O)(C(=O)CO)C[C@@H]3O[C@H]1C[C@H](NC(=O)OCc2ccc(NC(=O)[C@H](CCCNC(N)=O)NC(=O)[C@@H](NC(=O)CCCCCN3C(=O)C(Sc4ccccc4)=C(Sc4ccccc4)C3=O)C(C)C)cc2)[C@H](O)[C@H](C)O1]

In a 10 mL round-bottom flask, under argon, DTL-1-valine-citrulline-PABOH 10 (64.1 mg, 0.08 mmol) was dissolved in pyridine (1.2 mL) to give a yellow solution. The solution was cooled to 0° C. and p-nitrophenyl-chloroformate (48.5 mg, 0.25 mmol, 3 eq.) in DCM (0.8 mL) was added. Stirred at 0 C for 10 minutes and then allowed to warm to room temperature and stirred for an additional 2 hours. The, added EtOAc (20 mL) and washed with 15% citric acid (3×25 mL). The organic layer was dried (MgSO₄), concentrated under vacuo and purified by column chromatography on silica gel 60 with a gradient of DCM:MeOH from 20:1 to 15:1 (v/v). The obtained intermediate DTL-1-valine-citrulline-PABC product (23.99 mg, 0.025 mmol, 30%) was immediately used in the next step by being dissolved under argon in DMF (1.4 mL) to which doxorubicin hydrochloride (16 mg, 0.027 mmol, 1.08 eq.) was added, followed by addition of DIPEA (4.8 μL, 0.0276 mmol, 1.1 eq.). The red mixture was stirred for 16 hours. Then, concentrated under vacuo (40° C.) to give a red solid which was purified by column chromatography on silica gel 60 in DCM:MeOH (10:1 v/v) to afford 11 as a red solid (33 mg, 0.24 mmol, 97%). Data for 11: IR (pellet) νₘₐₓ 3324, 2935, 2411, 1704, 1620, 1579, 1519, 1440, 1400, 1284, 1208, 1017, 984, 736, 686. ¹H NMR (600 MHz, DMSO-d6) 0.80-0.84 (2d, J=6.6 Hz, 6H, C42), 1.11 (d, J=6.6 Hz, 2H, C51), 1.20 (quint., J=7.2 Hz, 2H, C46), 1.32-1.42 (2m, 2H, C36), 1.47 (overlapped m., 4H, C45 and C47), 1.55-1.68 (2m, 2H, C35), 1.83 (dt, J=13.2, 3.6 Hz, 1H, C4), 1.94 (oct., J=6.6 Hz, 1H, C41), 2.08-2.12 (m, 2H, C7), 2.09-2.21 (m, J=7.8 Hz, 2H, C44), 2.92-3.01 (2m, J=6.6 Hz, 2H, C37), 2.98 (d, J=18 Hz, 1H, C9), 3.37 (m, 2H, C48 under water peak), 3.43 (m, 1H, C2), 3.71 (m, J=4.8 Hz, 1H, C3), 3.99 (s, 3H, C24), 4.14 (q, J=6.6 Hz, 1H, C1), 4.18 (t, J=7.8 Hz, 1H, C40), 4.34 (q, J=7.2 Hz, 1H, C34), 4.57 (d, J=6.0 Hz, 2H, C26), 4.72 (d, J=5.4 Hz, 1H, C2OH), 4.88 (m, 3H, C28 overlapped with C260H), 4.94 (t, J=4.2 Hz, 1H, C6), 5.22 (d, J=2.4 Hz, 1H, C5), 5.42 (br, 2H, C38NH2), 5.49 (s, 1H, C8OH), 5.97, (t, J=5.4 Hz, 1H, C37NH), 6.86 (d, J=8.4 Hz, 1H, C3NH), 7.21-7.29 (overlapped m, 12H, Ph overlapped with C30), 7.54 (d, J=8.4 Hz, 2H, C31), 7.67 (dd, J=6.0, 3.0 Hz, 1H, C17), 7.82 (d, J=8.4 Hz, 1H, C40NH), 7.92 (m, 2H, overlapped C18 and C19), 8.09 (d, J=7.2 Hz, 1H, C39NH), 9.97 (br, 1H, C33NH), 13.30 (br, 1H, C12OH), 14.05 (br, 1H, C230H); ¹³C NMR (150 MHz, DMSO-d6) 17.1 (C51), 18.2 (C42), 19.3 (C42), 24.9 (C47), 25.8 (C46), 26.8 (C36), 27.6 (C45), 29.3 (C35), 29.8 (C4), 30.4 (C41), 32.1 (C9), 34.9 (C44 close to C7), 36.7 (C48), 38.2 (C37), 47.1 (C3), 53.1 (C34), 56.6 (C24), 57.5 (C40), 63.7 (C26), 64.9 (C28), 66.4 (C1), 67.9 (C2), 69.9 (C6), 74.9 (C8), 100.3 (C5), 110.7 (C22), 110.9 (C13), 118.9 (C31), 119.1 (C17), 119.8 (C19), 120.1 (C15), 128.0 (Ph), 128.6 (C30), 129.0 (Ph), 129.2 (Ph), 130.7 (Ph), 131.8 (C32), 134.2 (C11), 134.7 (C10), 135.4 (C50), 135.6 (C20), 136.3 (C18), 154.5 (C23), 155.3 (C12), 156.1 (C29), 158.9 (C38), 160.8 (C16), 166.5 (C49), 170.6, (C33), 171.3 (C39 close to C43), 172.3 (C27), 168.6 (C21), 168.7 (C14), 213.9 (C25). HRMS (ESI) [M+Na]⁺ found 1380.4388, C₆₈H₇₅N₇O₁₉S₂Na requires 1380.4457.

### 4.3.13 N-propargyl-3,4-dithiophenolmaleimide (N-alkyne dithiophenolmaleimide)

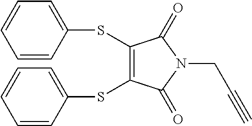 [SMILES: C#CCN1C(=O)C(Sc2ccccc2)=C(Sc2ccccc2)C1=O]

Propargylamine (0.009 mL, 0.135 mmol) was added to a stirred solution of N-methoxycarbonyl-3,4-dithiophenolmaleimide (50 mg, 0.135 mmol) in dichloromethane (6 mL). After 2 h, silica was added and the resulting mixture stirred overnight. Then it was filtered, concentrated and the crude residue was purified by column chromatography to yield the title compound as a yellow oil (46.5 mg, 0.132 mmol, 98%). d_{H} (CDCl₃, 600 MHz) 7.30 (2H, t, J=7.2 Hz, ArH), 7.26 (4H, t, J=7.2 Hz, ArH), 7.22 (4H, d, J=7.2 Hz, ArH), 4.26 (2H, d, J=2.3 Hz, CH₂), 2.21 (1H, t, J=2.3 Hz, CH); d_{C} (CDCl₃, 150 MHz) 165.6 (s), 136.0 (s), 131.9 (d), 129.1 (d), 128.8 (s), 128.7 (d), 76.9 (s), 71.9 (d), 27.7 (t); HRMS: Mass calculated for C₁₉H₁₃O₂NS₂: 351.03822, observed: 351.03865.

### 4.3.14 14-Azido-N-((2S,3S,4S,6R)-3-hydroxy-2-methyl-6-(((1S,3S)-3,5,12-trihydroxy-3-(2-hydroxyacetyl)-10-methoxy-6,11-dioxo-1,2,3,4,6,11-hexahydrotetracen-1-yl)oxy)tetrahydro-2H-pyran-4-yl)-3,6,9,12-tetraoxatetradecan-1-amide (azide-PEG4-DOX)

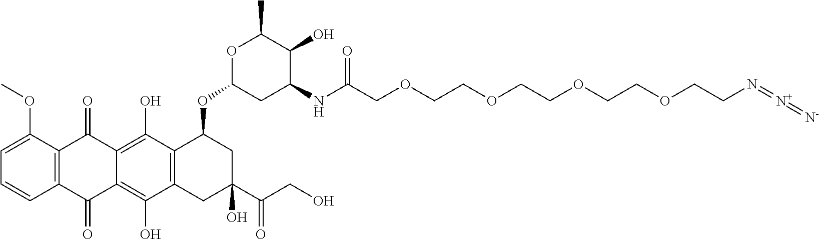 [SMILES: COc1cccc2c1C(=O)c1c(O)c3c(c(O)c1C2=O)C[C@@](O)(C(=O)CO)C[C@@H]3O[C@H]1C[C@H](NC(=O)COCCOCCOCCOCCN=[N+]=[N-])[C@H](O)[C@H](C)O1]

To a solution of 14-azido-3,6,9,12-tetraoxatetradecan-1-oic acid (4.4 mg, 16 μmol) and DIPEA (6.2 μL, 35 μmol) in DMF (1 mL) was added HBTU (6.7 mg, 18 μmol) and the reaction mixture stirred at 21° C. for 5 min After this time, was added doxorubicin (9.3 mg, 16 μmol) and the reaction mixture stirred at 21° C. for 3 h. Then the reaction mixture was diluted with H₂O (10 mL) and DCM (10 mL), extracted with DCM (3×15 mL), the combined organic layers washed with sat. aq. LiCl (2×10 mL) and acetate buffer pH 5, dried (MgSO₄) and concentrated in vacuo. The crude residue was purified by flash column chromatography (5% MeOH/EtOAc) to afford 14-Azido-N-((2S,3S,4S,6R)-3-hydroxy-2-methyl-6-(((1S,3S)-3,5,12-trihydroxy-3-(2-hydroxyacetyl)-10-methoxy-6,11-dioxo-1,2,3,4,6,11-hexahydrotetracen-1-yl)oxy)tetrahydro-2H-pyran-4-yl)-3,6,9,12-tetraoxatetradecan-1-amide (9 mg, 11 μmol, 70%) as a red solid. ¹H NMR (600 MHz, MeOD) d 13.84 (1H, s), 13.05 (1H, s), 7.79 (1H, d, J=7.5 Hz), 7.75 (1H, apt. t, J=7.9 Hz), 7.48 (1H, d, J=8.3 Hz), 5.38-5.42 (1H, m), 5.03-5.07 (1H, m), 4.74 (2H, d, J=5.3 Hz), 4.29 (1H, q, J=6.4 Hz), 4.18-4.23 (1H, m), 3.99 (3H, s), 3.58-3.70 (17H, m), 3.35 (2H, t, J=5.3 Hz), 3.01 (1H, d, J=18.4 Hz), 2.84 (1H, d, J=18.4 Hz), 2.35 (1H, d, J=14.3 Hz), 2.11-2.17 (1H, m), 2.00 (1H, m), 1.77 (1H, dd, J=13.2, 4.5 Hz), 1.26 (3H, d, J=6.8 Hz); ¹³C NMR (150 MHz, MeOD) d 214.8 (C), 187.9 (C), 187.6 (C), 172.0 (C), 162.4 (C), 157.3 (C), 156.1 (C), 137.2 (CH), 136.2 (C), 135.7 (C), 135.1 (C), 121.4 (C), 120.5 (CH), 120.2 (CH), 112.3 (C), 112.1 (C), 102.1 (CH), 77.3 (C), 71.9 (CH₂), 71.6 (CH₂), 71.5 (CH₂), 71.5 (CH₂), 71.3 (CH₂), 71.2 (CH₂), 71.1 (CH), 71.0 (CH₂), 69.8 (CH), 68.6 (CH), 65.7 (CH₂), 57.1 (CH₃), 51.7 (CH₂), 46.7 (CH), 37.3 (CH₂), 34.0 (CH₂), 30.7 (CH₂), 17.3 (CH₃)

4.4 In Situ Bridging and Functionalization with Doxorubicin

Trastuzumab was transferred into a borate buffer (25 mM sodium borate, 25 mM NaCl, 1 mM EDTA, pH 8.0) by ultrafiltration (MWCO 10 kDa) and treated with following in situ protocols. A) 20 equiv N-alkyne-dithiophenolmaleimide+10 equiv benzeneselenol for 1 h at ambient temperature in 15% DMF. B) 20 equiv N-alkyne-dithiophenolmaleimide+10 equiv benzeneselenol for 30 min at ambient temperature in 15% DMF, then 10 equiv benzeneselenol for another 30 mM C) 10 equiv N-alkyne-dithiophenolmaleimide+7 equiv TCEP for 2 h at 37° C. in 15% DMF. D) 15 equiv N-alkyne-dithiophenolmaleimide+10 equiv TCEP for 2 h at 37° C. in 15% DMF. The reaction was stopped in all samples with 20 equiv of maleimide (in DMF) and purified into PBS (pH 7.4) by ultrafiltration (MWCO 10 kDa). After determination of the concentration by UV/Vis (ε₂₈₀=210,000 cm⁻¹ M⁻¹) and dilution of the antibody to 30 μM all samples were treated with 30 equiv azide-PEG₄-DOX in the presence of 150 μM CuSO₄, 750 μM THPTA, 5 mM aminoguanidine hydrochloride and 5 mM sodium ascorbate. The reactions were incubated at 22° C. for 18 h with the exception of A) which was reacted for only 90 mM All samples were purified by size exclusion chromatography (on a HiLoad Sephadex 75 16/60 column, GE Healthcare, equilibrated in PBS) and the drug-to-antibody ratio (DAR) calculated by UV/Vis via the following equation

$$DAR = \frac{\frac{{OD}_{495}}{8030 {M}^{- 1} {cm}^{- 1}}}{\frac{( {OD}_{280} - {OD}_{495} \times 0.724 )}{210000 {M}^{- 1} {cm}^{- 1}}} .$$

| | Yield | Yield click | Overall | |
|---|---|---|---|---|
| Sample | bridging* | reaction* | yield* | DAR |
| A | 84% | 82% | 69% | 1.1 |
| B | 86% | 72% | 62% | 2.0 |
| C | 82% | 69% | 57% | 3.1 |
| D | 86% | 60% | 52% | 4.0 |

*Purification yields, not conversion

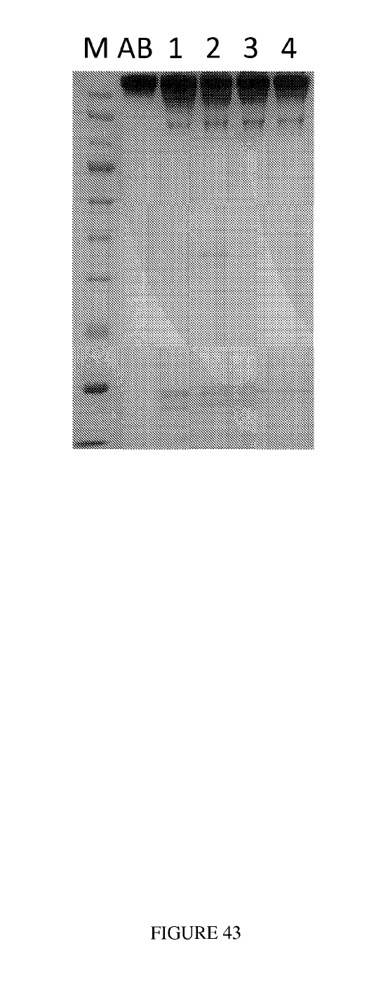

Results are shown in FIG. 43.

### 4.5 ADC Analysis by Capillary Gel Electrophoresis

Capillary gel electrophoresis was used to quantify the fragmentation induced by disulfide bond-based functionalisation. Antibody samples with a DAR of 0, 1, 2, 3 and 4 (of doxorubicin) were prepared as outlined under “In situ Bridging and Functionalisation with Doxorubicin”. In addition a reduction series of Herceptin was prepared by treating the antibody (in 25 mM sodium borate, 25 mM NaCl, 1 mM EDTA, pH 8.0) with 0, 1, 2, 3, 4, 5, 6 or 7 equiv TCEP for 2 h at 37° C. All samples were alkylated with 20 equiv maleimide (in DMF) after the reaction and transferred into PBS (pH 7.4) by ultrafiltration (MWCO 10 kDa).

CGE analysis was carried out on a PEREGRINE I machine (deltaDOT). Samples were diluted to lmg/ml in SDS-MW sample buffer (Proteome Lab) and heated to 65° C. for 20 min. 50 μl were transferred into sample vials after brief centrifugation and loaded into the machine.

Separations were performed in a 50 μm diameter fused silica capillary at 22° C. Separation length was 20.2 cm, run time 45 mM and antibody fragments detected at a wavelength of 214 nm. The capillary was flushed with 0.1 M HCl, water and run buffer before sample loading at 5 psi/16 kV. Noise was recorded for 3 min from the run buffer. To verify comparison-based fragment identification a protein sizing standard (Beckman Coulter) was used.

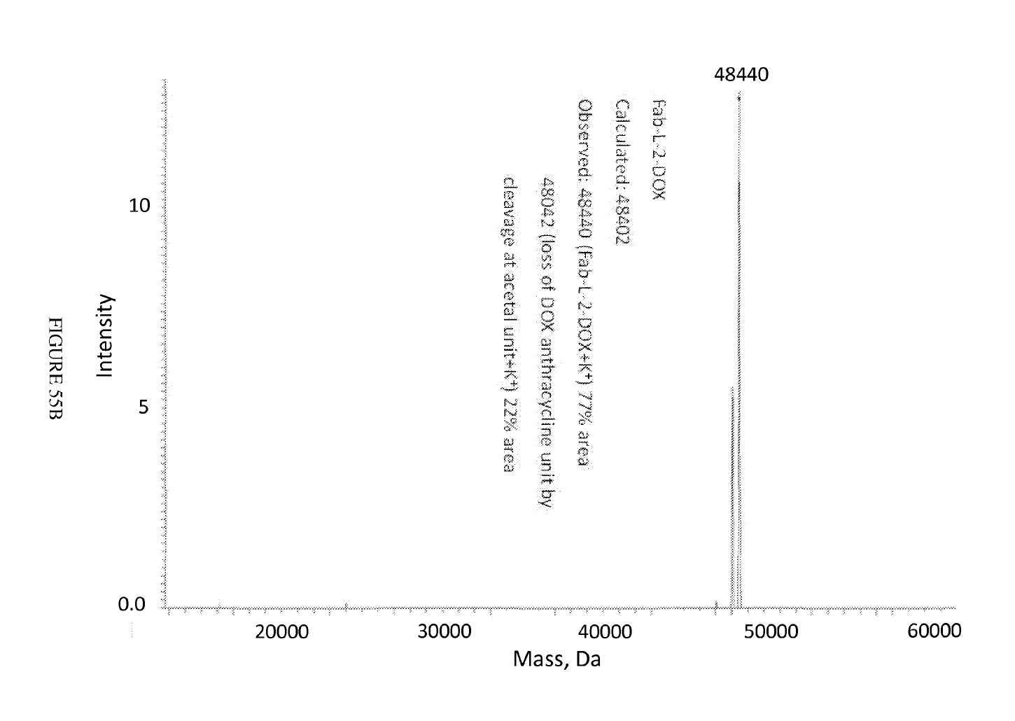

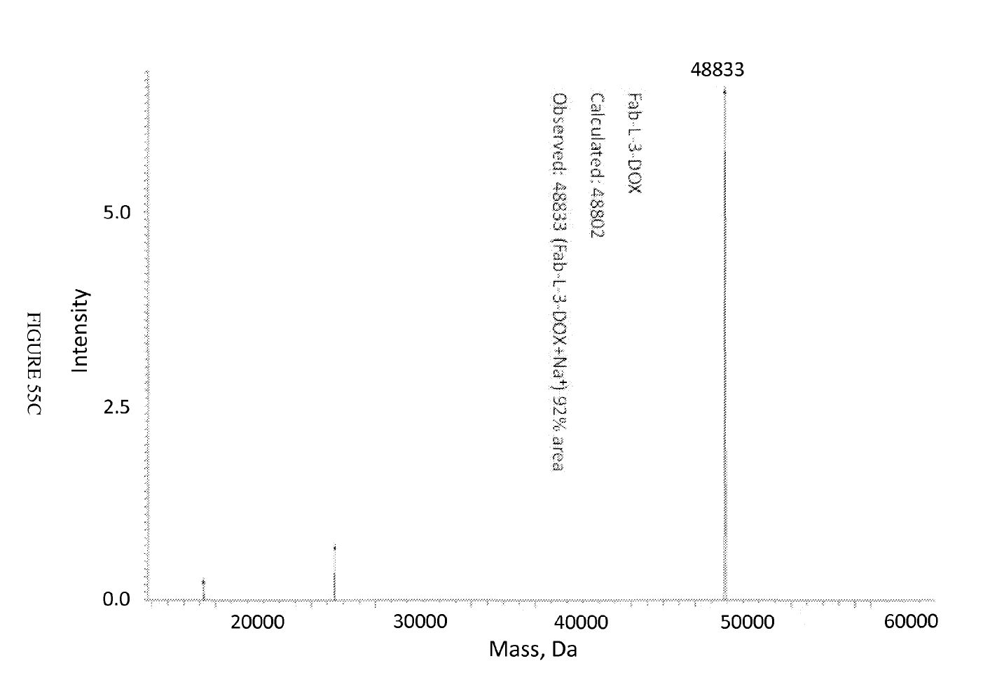

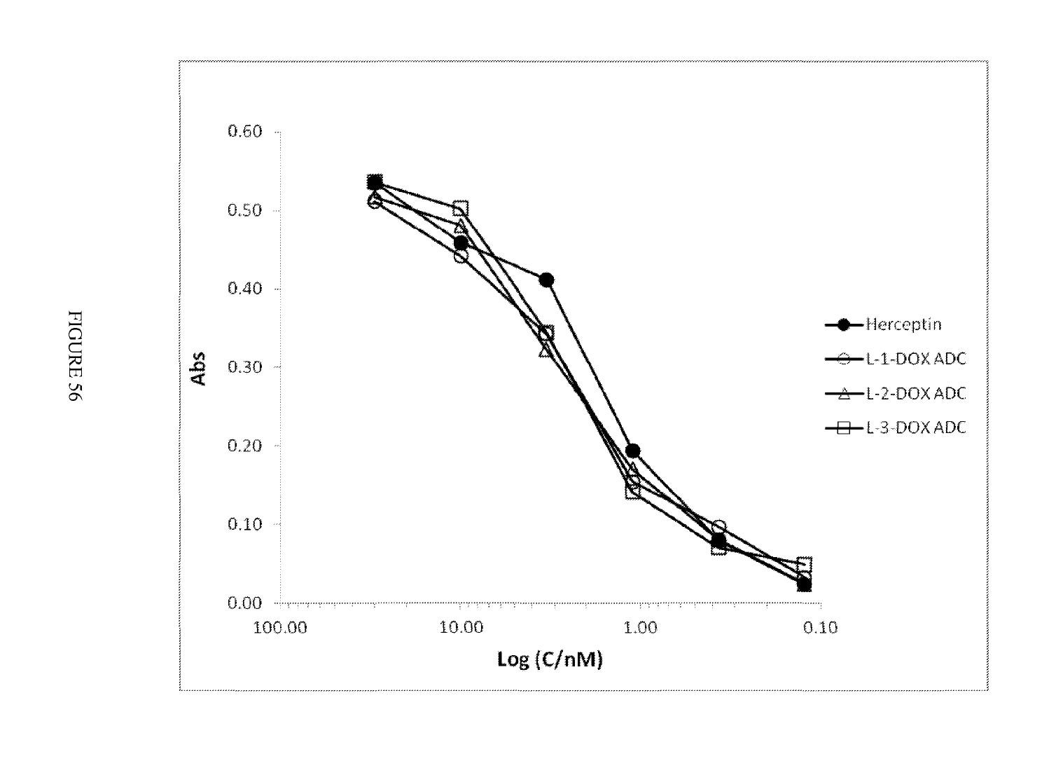

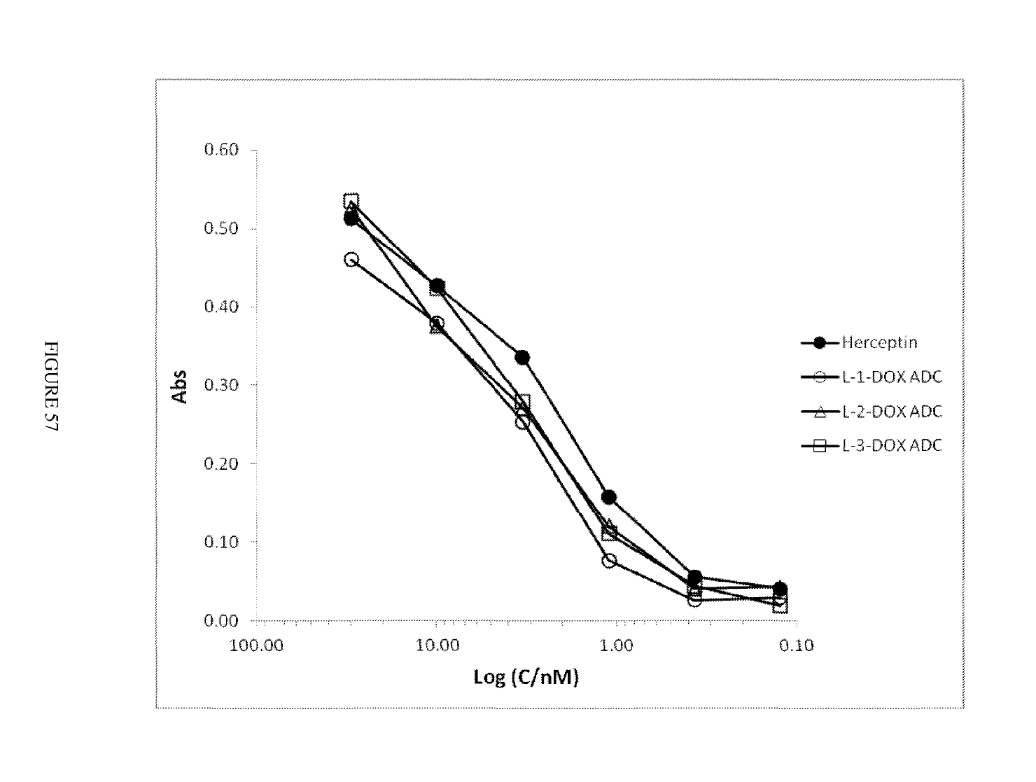

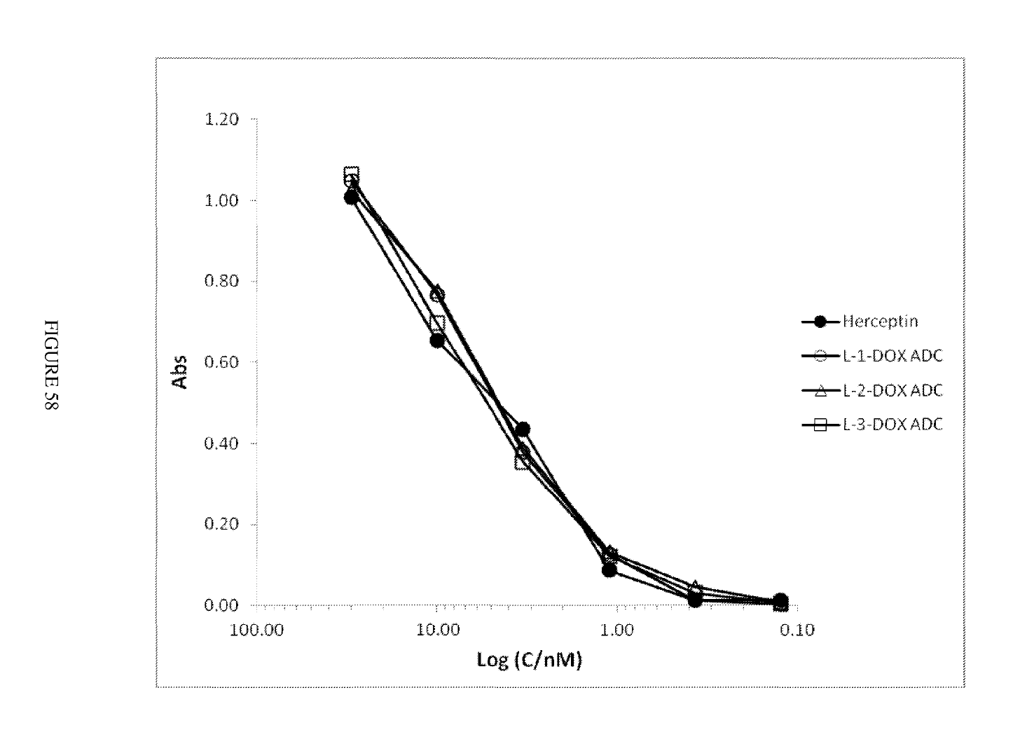

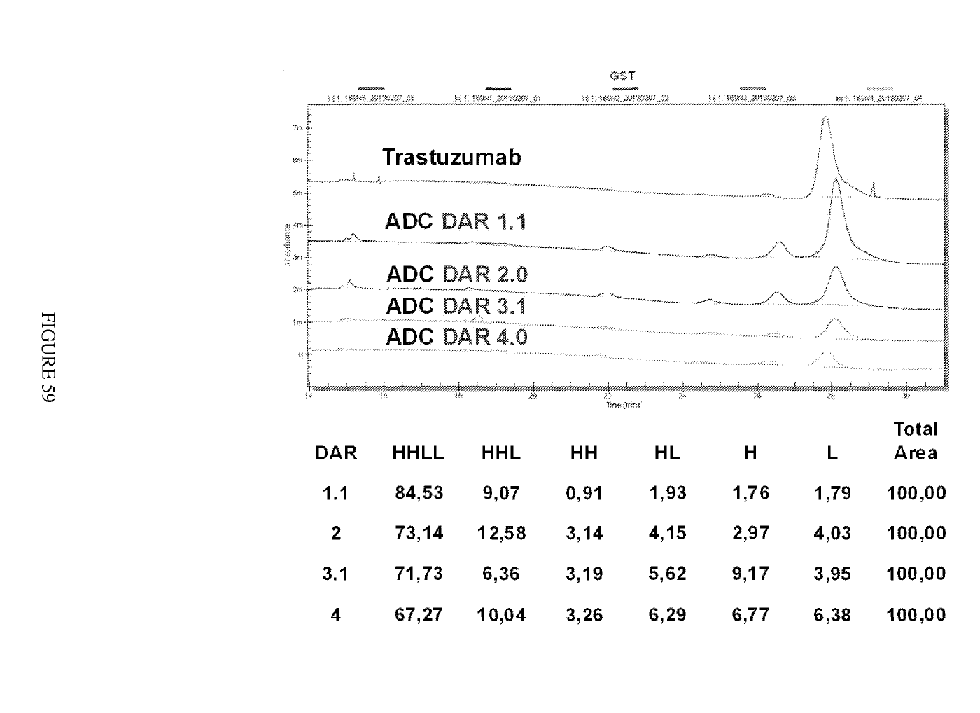

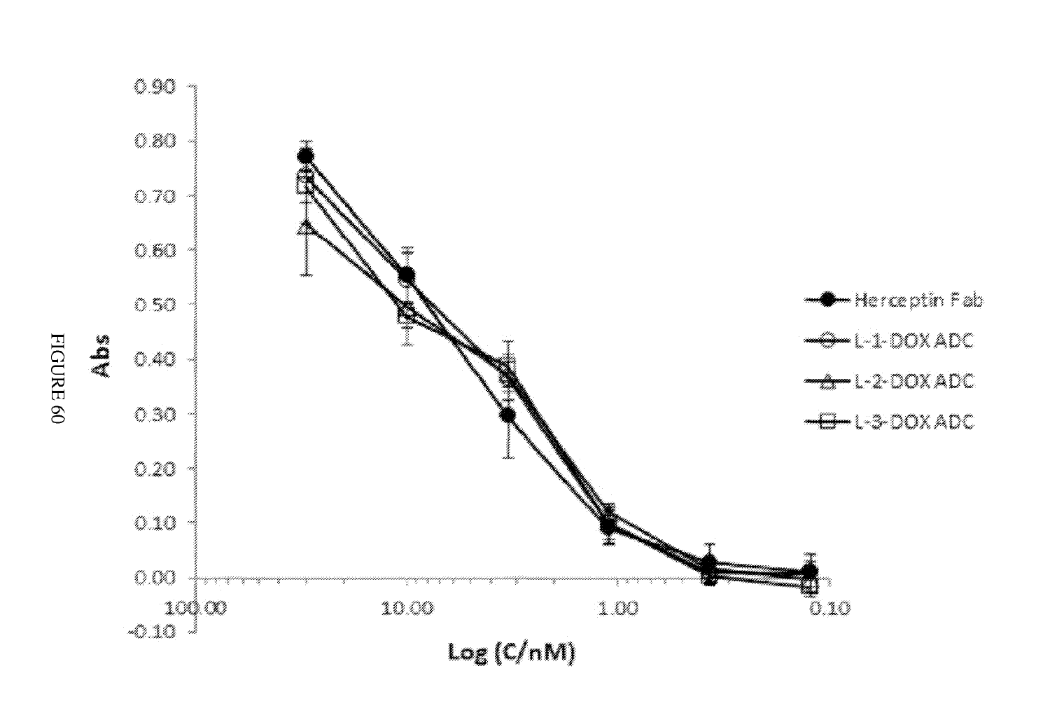

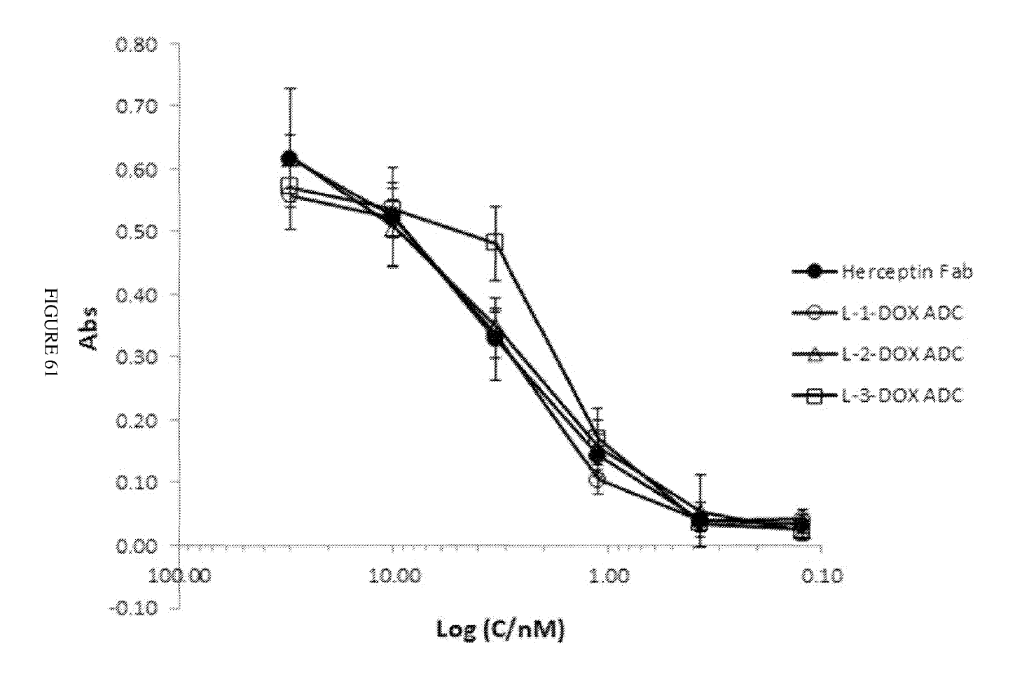

Data analysis was carried out with the EVA software (version 3.1.7, deltaDOT). Run files were loaded and analysed with the GST algorithm at a frequency of 40 and a sensitivity of 1. GST peak search was performed between 13 and 32 min (8,000 to 20,000 scans) based on the peak identification by mass and comparison between unmodified, partially and fully reduced antibody samples. Peaks corresponding to the HHLL, HHL, HH, HL, H and L antibody species were added manually were necessary and peak area boundaries adjusted for all signals. As the peak area (absorbance) varies depending on the size of the antibody fragment a normalisation process was established. A correction factor between the absorbance of the full antibody and the completely disassembled antibody (only H and L fragments) was calculated. This factor was adjusted for the area correction of the remaining fragments (HHL, HH, HL) depending on their disulfide bond status, e.g. only 25% of the correction factor was applied to the peak area of the HHL fragment as 75% of the disulfide bonds were assumed to be intact. The normalisation was established based on the samples of the reduction series and transferred to the samples with varying DARs. In addition the observed fragmentation of the unmodified antibody was also subtracted as a background to calculate the induced fragmentation, which is based only on the functionalisation of the antibody disulfide bonds during ADC synthesis. Analysis showed that all ADCs comprised of >67% fully rebridged antibody (see FIG. 59).

### 4.6 Site-Specificity of Benzeneselenol-Base In Situ Functionalization of Trastuzumab

Trastuzumab-DOX conjugates with a DAR of 2.0 (sample B) and 3.1 (sample C) were prepared as outlined under “In Situ Bridging and Functionalization with Doxorubicin”. These were treated alongside the unmodified antibody with 3, 5 or 7 equiv TCEP for 2 h at 37° C. after a buffer exchange into the pH 8.0 borate buffer by ultrafiltration (MWCO 5 kDa). The resulting fragmentation was visualized by SDS-PAGE, as shown in FIG. 44. Gel shows that heavy-light interactions are stabilised to reducing conditions following benzeneselenol-mediated maleimide bridging. This indicates that benzeneselenol-mediated maleimide bridging of trastuzumab targets heavy-light interchain disulfide bonds.

### 4.7 Digest of a Trastuzumab-DOX Conjugate

A Trastuzumab-DOX conjugate with a DAR of 2.0 (sample B) was prepared as outlined under “In Situ Bridging and Functionalization with Doxorubicin”. The pH of the sample was lowered via a buffer exchange (into 20 mM sodium acetate, pH 3.1) by ultrafiltration (MWCO 10 kDa). Immobilized pepsin (0.15 mL) was washed 4× with the same buffer and Trastuzumab-DOX (0.45 mL, 3.19 mg/mL) was added. The mixture was incubated for 5 h at 37° C. under constant agitation (1,100 rpm). The resin was separated from the digest using a filter column, and washed 3× with digest buffer (50 mM sodium phosphate, 150 mM NaCl, 1 mM EDTA, pH 6.8). The digest was combined with the washes and the volume adjusted to 0.5 mL.

Next immobilised papain (0.5 mL, 0.25 mg/mL) was activated with 10 mM DTT (in digest buffer) under an argon atmosphere and constant agitation (1,100 rpm) for 1 h at 25° C. in the dark. The resin was washed 4× with digest buffer (without DTT) and the 0.5 mL of trastuzumab-DOX-Fab₂ solution was added. The mixture was incubated for 16 h at 37° C. under constant agitation (1,100 rpm) in the dark. The resin was separated from the digest using a filter column, washed 3× with PBS (pH 7.0) and the digest combined with the washes. The buffer was exchanged completely for PBS by ultrafiltration (MWCO 10 kDa) and the volume adjusted to 0.3 mL. In parallel a sample of unmodified Trastuzumab was processed as a control.

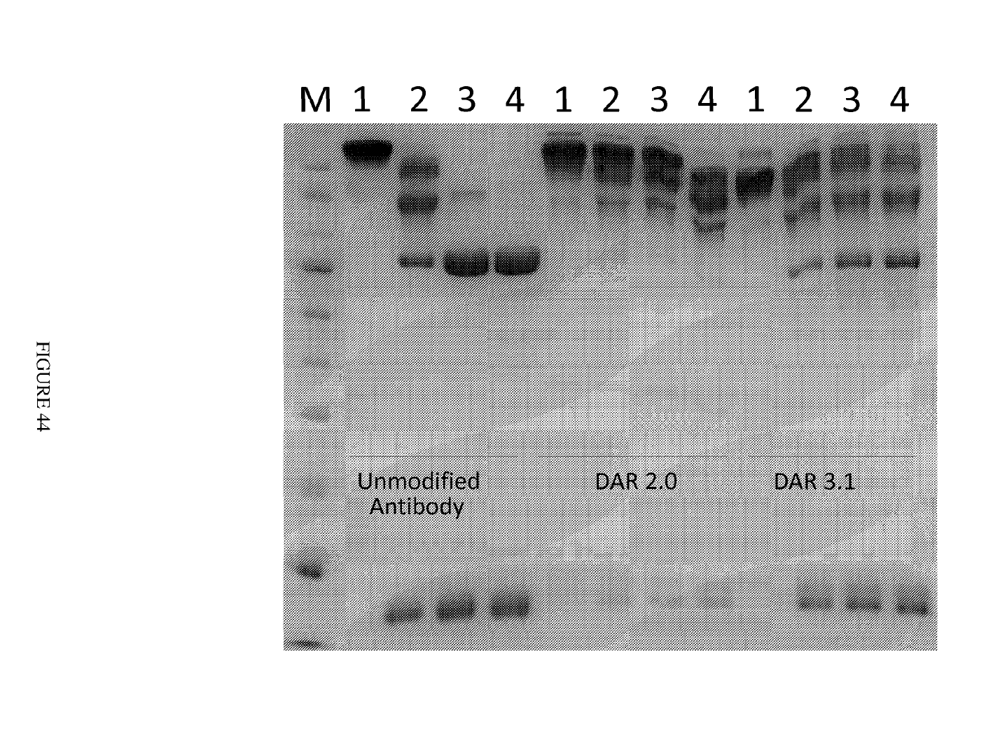

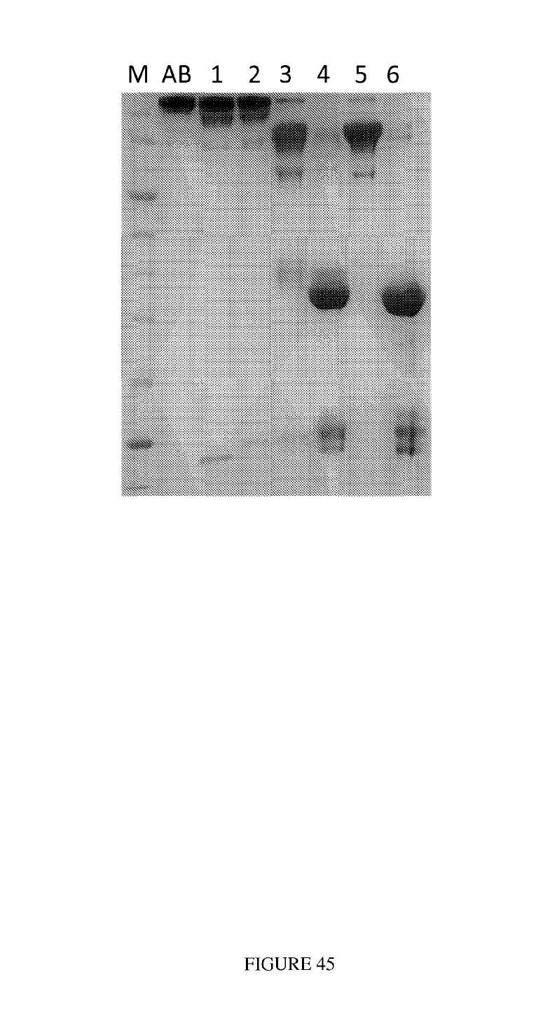

Sample and control were analysed by SDS-PAGE (see FIG. 45). The drug loading of the HER-Fab-DOX was assessed by UV/Vis (ε₂₈₀=68,560 cm⁻¹ M⁻¹). The intact material before the digest had a DAR of 2.06. The isolated Fab-DOX had a DAR of 0.79 suggesting the targeting of approximately 77% of the drug to the Fab-region of the antibody.

4.8 Direct Bridging and Functionalization with Doxorubicin Compounds

Functionalisation of Trastuzumab (average MW 147000) and Trastuzumab Fab (MW 47662 by ES-MS) was carried out through three different protocols employing doxorubicin containing reagents capable of immediate disulfide bridging via cysteine conjugation. Said reagents structure include a 2,3-dithio-maleimide conjugation site available for dual conjugation; a N-functionalised spacer unit between C6 and C25 inclusive also of heteroatoms such as N, 0 and selected structural elements ranging from alkyl, aryl, amide, urea and carbamide arranged in linear or branched faction, tailored to offer hydrolytical stability and/or self-immolative spacer for drug release; Doxorubicin attached to spacer in a stable structure or with a self-immolative spacer for drug release. Exemplification is carried out using bridging reagents DTL-1DOX, DTL-2-DOX and DTL-3-DOX prepared as 9.16 mM or 0.916 mM solutions in DMF for conjugation to Trastuzumab or Trastuzumab Fab respectively. Details of their synthesis including compound characterisation are presented below.

The three protocols are referred to as follows:

Stepwise: where the antibody or antibody fragment have their accessible disulfide bonds reduced, then undergo removal of reducing agent, followed by addition of bridging reagent of choice.

Sequential: where the antibody or antibody fragment have their accessible disulfide bonds reduced, followed by immediate addition of bridging reagent without prior removal of reducing agent.

In situ: where the antibody or antibody fragment have their accessible disulfide bonds reduced while in the presence of both reducing agent and bridging reagent from the onset to afford concomitant reduction and bridging.

### 4.8.1 Stepwise modification of Trastuzumab mAb

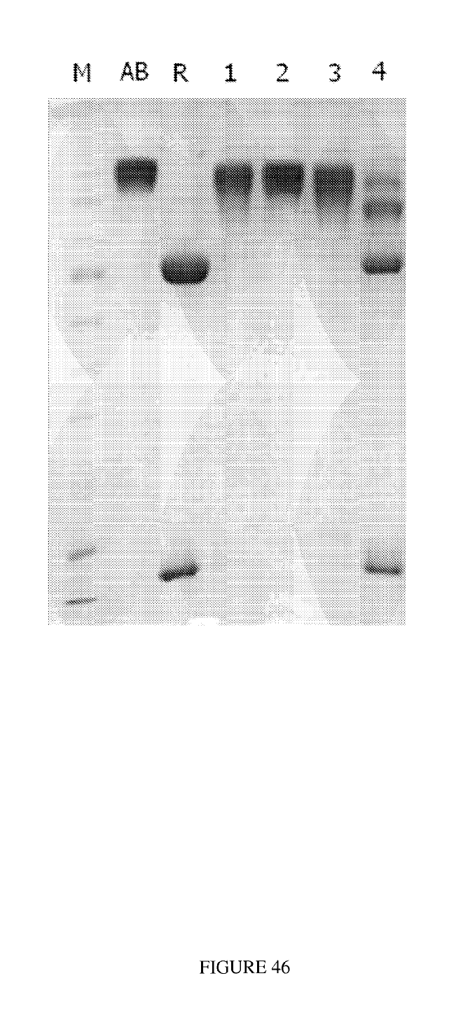

Trastuzumab was transferred into a borate buffer (25 mM sodium borate, 25 mM NaCl, 1 mM EDTA, pH 8.0) by ultrafiltration (MWCO 10 kDa) and the concentration was corrected to 22.9 μM. This solution was treated with TCEP (7 eq.) at 37° C., shaking at 400 rpm for 2 hours. Then, eluted this solution through a PD-G25 buffer swapping column following manufacturer's protocol, equilibrated with the borate buffer described above, as means to separate from excess TCEP. The concentration was assessed by UV/Vis (ε₂₈₀=210,000 cm⁻¹ M⁻¹) and was concentrated back to 22.9 μM. Next, the solution was aliquoted into 200 μL (0.00397 μmol) portions to which were added 2.17 μL of a 9.16 mM solution of A) DTL-1-DOX (5 eq.) diluted into DMF (20 μL), kept at 4° C.; B) DTL-2-DOX (5 eq.) diluted into DMF (20 μL), kept at 37° C. with shaking at 400 rpm; C) DTL-3-DOX (5 eq.) diluted into DMF (20 μL), kept at 37° C. with shaking at 400 rpm. D) No bridging reagent was added, only DMF (22.17 μL), kept at 37° C. with shaking at 400 rpm. The addition of DMF alongside bridging reagents ensured a 10% DMF (v/v) composition for the buffer system. 30 minutes after addition samples (5 μL) were taken from each reaction, quenched with maleimide (20 eq.) and reserved for SDS-PAGE gel analysis. The reaction mixture was immediately buffer swapped into a phosphate buffer (70 mM phosphates, 1 mM EDTA, pH 6.8) by ultrafiltration (MWCO 10 kDa) with at least 6 cycles of concentration by ultrafiltration and dilution. The purified material was analysed by UV/Vis for the purposes of determining yield of recovered antibody and DAR according to the formula described above. Analysis by SDS-PAGE gel was also performed (see FIG. 46).

Yields and DAR for Stepwise Protocol with Trastuzumab mAb

| Reaction | Reagent | Yield* | DAR |
|---|---|---|---|
| A | DTL-1-DOX | 72% | 3.16 |
| B | DTL-2-DOX | 74% | 2.57 |
| C | DTL-3-DOX | 60% | 3.17 |

*Purification yields, not conversion.

### 4.8.2 Sequential Modification of Trastuzumab mAb

### 4.8.2.1 Sequential Modification of Trastuzumab with DTL1-DOX and DTL2-DOX

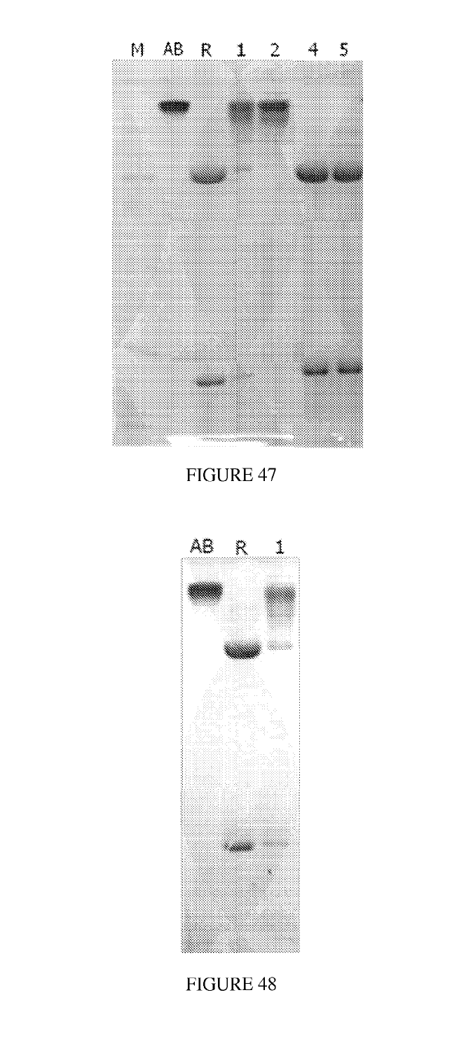

Trastuzumab was transferred into a borate buffer (25 mM sodium borate, 25 mM NaCl, 1 mM EDTA, pH 8.0) by ultrafiltration (MWCO 10 kDa) and the concentration was corrected to 22.9 μM. This solution was treated with TCEP (7 eq.) at 37° C., shaking at 400 rpm for 2 hours. Next, the solution was aliquoted into 200 μL, (0.004576 μmol) portions to which were added 2.50 μL, of a 9.16 mM solution of A) DTL-1-DOX (5 eq.) diluted into DMF (19.7 μL), kept at 4° C.; B) DTL-2-DOX (5 eq.) diluted into DMF (19.7 μL), kept at 37° C. with shaking at 400 rpm; C) No bridging reagent was added, only DMF (22.17 μL), reaction at 4° C.; D) No bridging reagent added, only DMF (22.17 μL), reaction at 37° C. Added DMF alongside bridging reagents ensured a 10% DMF (v/v) composition for the buffer system. 30 minutes after addition samples (5 μL) were taken from each reaction, quenched with maleimide (20 eq.) and reserved for SDS-PAGE gel analysis. The reaction mixture was immediately buffer swapped into a phosphate buffer (70 mM phosphates, 1 mM EDTA, pH 6.8) by ultrafiltration (MWCO 10 kDa) with at least 6 cycles of concentration by ultrafiltration and dilution. The purified material was analysed by UV/Vis for the purposes of determining yield of recovered antibody and DAR according to the formula described above. Analysis by SDS-PAGE gel was also performed (see FIG. 47).

### 4.8.2.2 Sequential Modification of Trastuzumab with DTL3-DOX

An aliquot of reduced Trastuzumab (200 μL, 0.004576 μmol) was prepared as described in section 4.7.2.1. DTL-3-DOX (20 eq.) diluted into DMF (19.7 μL) was added and the mixture kept at 25° C. with shaking at 400 rpm. 30 minutes after addition a sample (5 μL) was taken from the reaction, quenched with maleimide (20 eq.) and reserved for SDS-PAGE gel analysis. The reaction mixture was immediately buffer swapped into a phosphate buffer (70 mM phosphates, 1 mM EDTA, pH 6.8) by ultrafiltration (MWCO 10 kDa) with at least 6 cycles of concentration by ultrafiltration and dilution. The purified material was analysed by UV/Vis for the purposes of determining yield of recovered antibody and DAR according to the formula described above. Analysis by SDS-PAGE gel was also performed (see FIG. 48).

Yields and DAR for Sequential Protocol with Trastuzumab mAb

| Reaction | Reagent | Yield* | DAR |
|---|---|---|---|
| A | DTL-1-DOX | 88% | 3.72 |
| B | DTL-2-DOX | 97% | 3.09 |
| C | DTL-3-DOX | 72% | 3.59 |

*Purification yields, not conversion.

### 4.8.3 In Situ Modification of Trastuzumab mAb

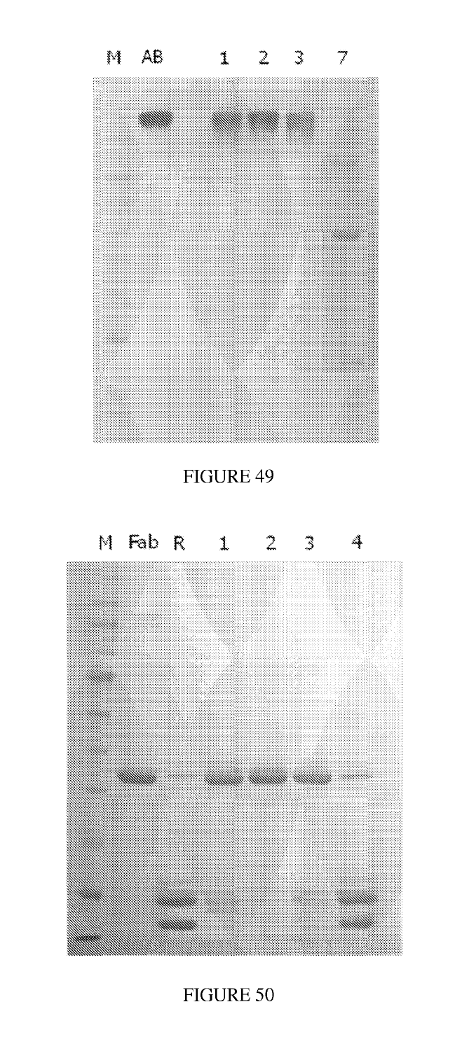

Trastuzumab was transferred into a borate buffer (25 mM sodium borate, 25 mM NaCl, 1 mM EDTA, pH 8.0) by ultrafiltration (MWCO 10 kDa) and the concentration was corrected to 22.9 μM. This solution was treated with TCEP (7 eq.) at 37° C., shaking at 400 rpm for 2 hours in the presence of bridging reagent and DMF to ensure a 10% DMF (v/v) composition of the buffer system A) DTL-1-DOX (10 eq.), kept at 37° C. with shaking at 400 rpm; B) DTL-2-DOX (10 eq.), kept at 37° C. with shaking at 400 rpm; C) DTL-3-DOX (10 eq.), kept at 37° C. with shaking at 400 rpm. D) No bridging reagent was added, only DMF, reaction at 37° C. After 2 hours, samples (5 μL) were taken from each reaction, quenched with maleimide (20 eq.) and reserved for SDS-PAGE gel analysis. The reaction mixture was immediately buffer swapped into a phosphate buffer (70 mM phosphates, 1 mM EDTA, pH 6.8) by ultrafiltration (MWCO 10 kDa) with at least 6 cycles of concentration by ultrafiltration and dilution. The purified material was analysed by UV/Vis for the purposes of determining yield of recovered antibody and DAR according to the formula described above. Analysis by MALDI-TOF was also carried out on selected cases. Analysis by SDS-PAGE gel was also performed (see FIG. 49).

### Yields and DAR for In Situ Protocol for Trastuzumab mAb

| Reaction | Reagent | Yield* | DAR |
|---|---|---|---|
| A | DTL-1-DOX | 79% | 3.69 |
| B | DTL-2-DOX | 98% | 2.39 |
| C | DTL-3-DOX | 89% | 3.58 |

*Purification yields, not conversion.

### 4.8.4 Stepwise Modification of Trastuzumab Fab

Trastuzumab Fab was transferred into a borate buffer (25 mM sodium borate, 25 mM NaCl, 1 mM EDTA, pH 8.0) by ultrafiltration (MWCO 10 kDa) and the concentration was corrected to 22.9 μM. This solution was treated with TCEP (3 eq.) at 37° C., shaking at 400 rpm for 2 hours. Then, eluted this solution through a PD-G25 buffer swapping column following manufacturer's protocol, equilibrated with the borate buffer described above, as means to separate from excess TCEP. The concentration was assessed by UV/Vis (ε₂₈₀=68590 cm⁻¹ M⁻¹) and was concentrated back to 22.9 μM. Next, the solution was aliquoted into 100 μL (0.00229 μmol) portions to which were added 12.5 μL of a 0.916 mM solution of A) DTL-1-DOX (5 eq.), kept at 25° C. with shaking at 400 rpm; B) DTL-2-DOX (5 eq.), kept at 25° C. with shaking at 400 rpm; C) DTL-3-DOX (5 eq.), kept at 25° C. with shaking at 400 rpm. D) No bridging reagent was added, only DMF (12.5 μL), kept at 25° C. with shaking at 400 rpm. E) Addition of bridging reagent in DMF ensured a 10% DMF (v/v) composition for the buffer system. 30 minutes after addition samples (5 μL) were taken from each reaction, quenched with maleimide (20 eq.) and reserved for SDS-PAGE gel analysis. The reaction mixture was immediately diluted with PBS to 400 μL, extracted with EtOAc (2×200 μL) to remove excess bridging reagent. The aqueous layer with Fab ADC was buffer swapped into a phosphate buffer (70 mM phosphates, 1 mM EDTA, pH 6.8) by ultrafiltration (MWCO 10 kDa) with at least 4 cycles of concentration by ultrafiltration and dilution. The purified material was analysed by UV/Vis for the purposes of determining yield of recovered antibody and DAR according to the formula described above, replacing the previous full Trastuzumab ε₂₈₀ with the value for Trastuzumab Fab as indicated above. Analysis by LCMS was also carried out (see FIG. 51). Analysis by SDS-PAGE gel was also performed (see FIG. 50).

Yields and DAR for Stepwise Protocol with Trastuzumab Fab

| Reaction | Reagent | Yield* | DAR |
|---|---|---|---|
| A | DTL-1-DOX | 70% | 1.16 |
| B | DTL-2-DOX | 86% | 0.51 |
| C | DTL-3-DOX | 81% | 0.63 |

*Purification yields, not conversion.

### 4.8.5 Sequential Modification of Trastuzumab Fab

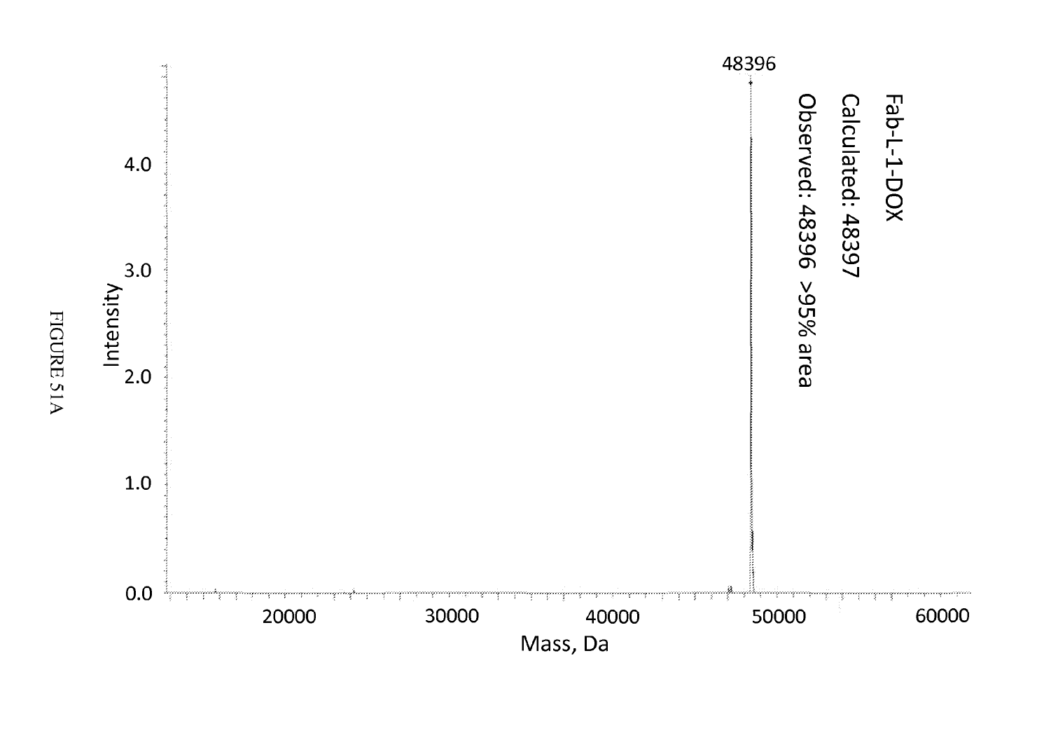

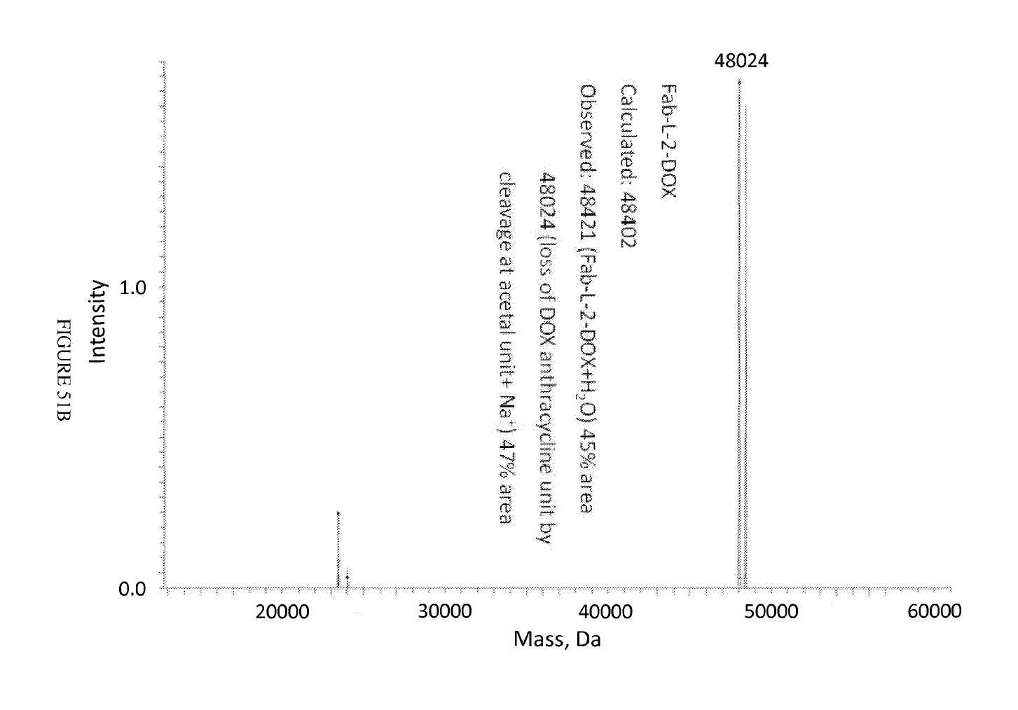

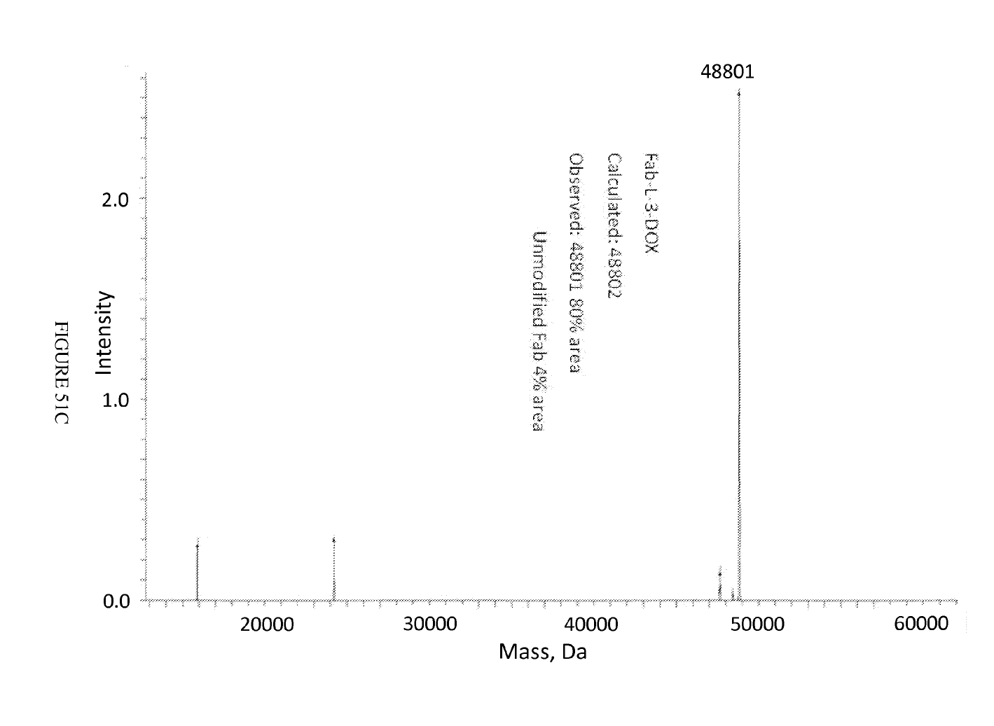

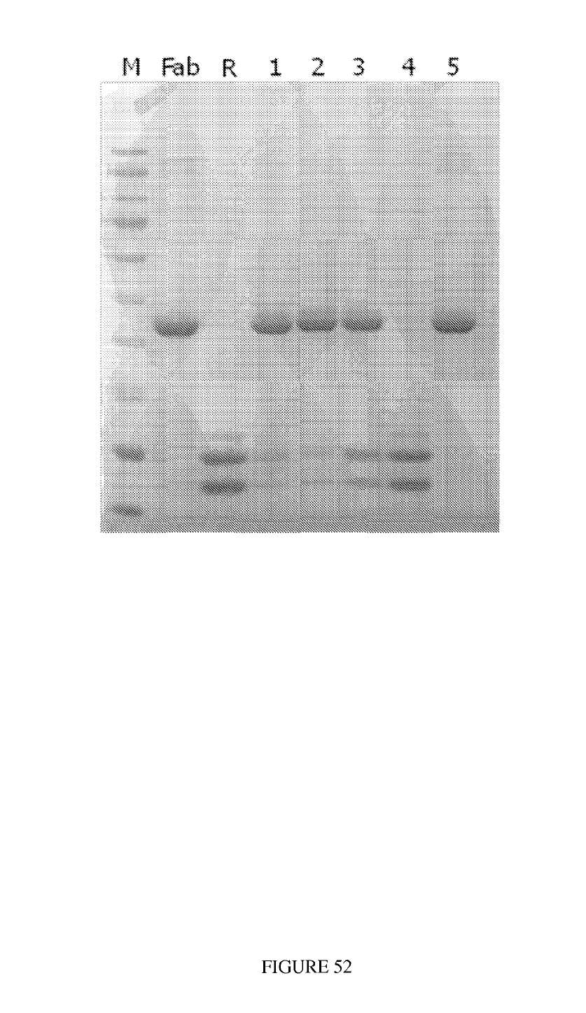

Trastuzumab Fab was transferred into a borate buffer (25 mM sodium borate, 25 mM NaCl, 1 mM EDTA, pH 8.0) by ultrafiltration (MWCO 10 kDa) and the concentration was corrected to 22.9 μM. This solution was treated with TCEP (3 eq.) at 37° C., shaking at 400 rpm for 2 hours. Next, the solution was aliquoted into 100 μL (0.00229 μmol) portions to which were added 12.5 μL of a 0.916 mM solution of A) DTL-1-DOX (5 eq.), kept at 25° C. with shaking at 400 rpm; B) DTL-2-DOX (5 eq.), kept at 25° C. with shaking at 400 rpm; C) DTL-3-DOX (5 eq.), kept at 25° C. with shaking at 400 rpm. D) No bridging reagent was added, only DMF (12.5 μL), kept at 25° C. with shaking at 400 rpm. E) Fab which was incubated in borate buffer at 25° C., shaking at 400 rpm for 2 hours in the absence of TCEP was treated with DTL-1-DOX (5 eq.), 10% (v/v) DMF, 25° C., shaking at 400 rpm. Addition of bridging reagent in DMF ensured a 10% DMF (v/v) composition for the buffer system. 30 minutes after addition samples (5 μL) were taken from each reaction, quenched with maleimide (20 eq.) and reserved for SDS-PAGE gel analysis. The reaction mixture was immediately diluted with PBS to 400 μL, extracted with EtOAc (2×200 μL) to remove excess bridging reagent. The aqueous layer with Fab ADC was buffer swapped into a phosphate buffer (70 mM phosphates, 1 mM EDTA, pH 6.8) by ultrafiltration (MWCO 10 kDa) with at least 4 cycles of concentration by ultrafiltration and dilution. The purified material was analysed by UV/Vis for the purposes of determining yield of recovered antibody and DAR according to the formula described above, replacing the previous full Trastuzumab ε₂₈₀ with the value for Trastuzumab Fab as indicated above. Analysis by LCMS was also carried out (see FIG. 53). Analysis by SDS-PAGE gel was also performed (see FIG. 52).

As can be seen from the control experiments D) and E), bridging reagent is required to reform the Fab (see SDS-PAGE gel) and no addition of bridging reagent takes place unless the Fab is reduced prior to conjugation (see SDS-PAGE gel and DAR table).

Yields and DAR for Sequential Protocol with Trastuzumab Fab

| Reaction | Reagent | Yield* | DAR |
|---|---|---|---|
| A | DTL-1-DOX | 74% | 1.21 |
| B | DTL-2-DOX | 76% | 0.64 |
| C | DTL-3-DOX | 70% | 0.94 |
| E | DTL-1-DOX | 60% | 0 |

*Purification yields, not conversion.

### 4.8.6 In Situ Modification of Trastuzumab Fab

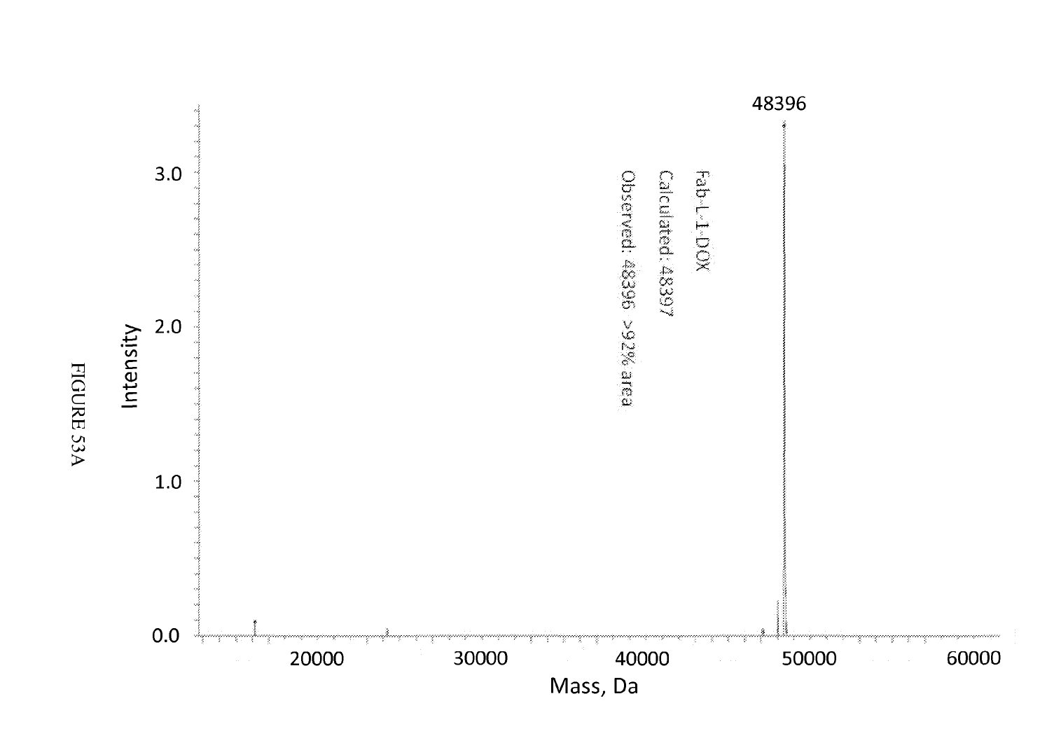

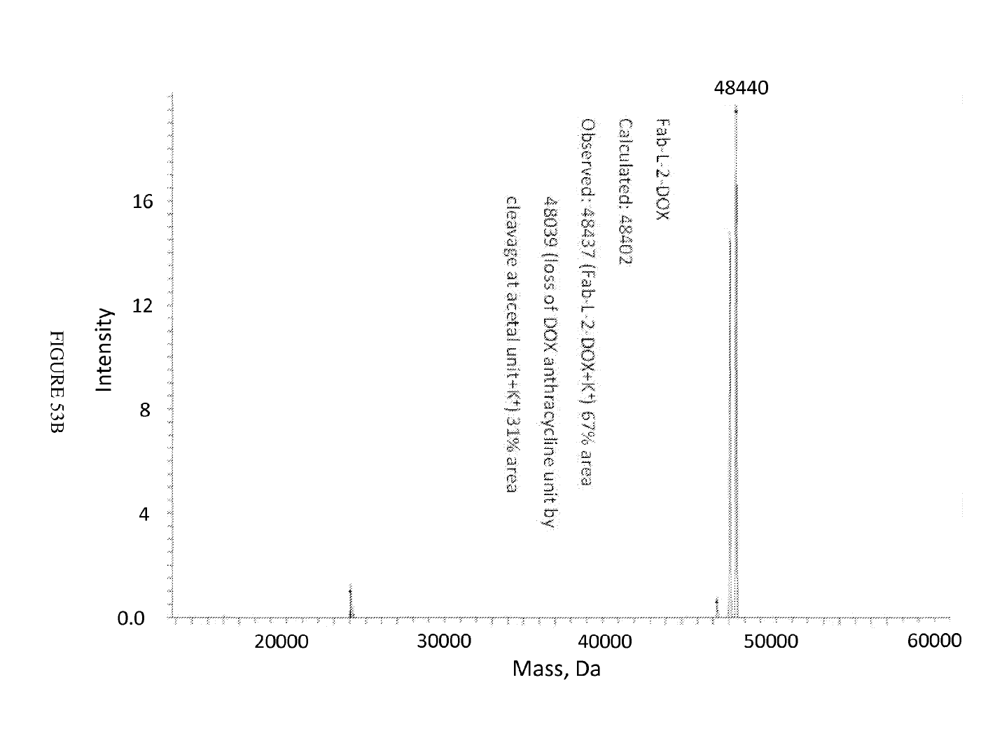

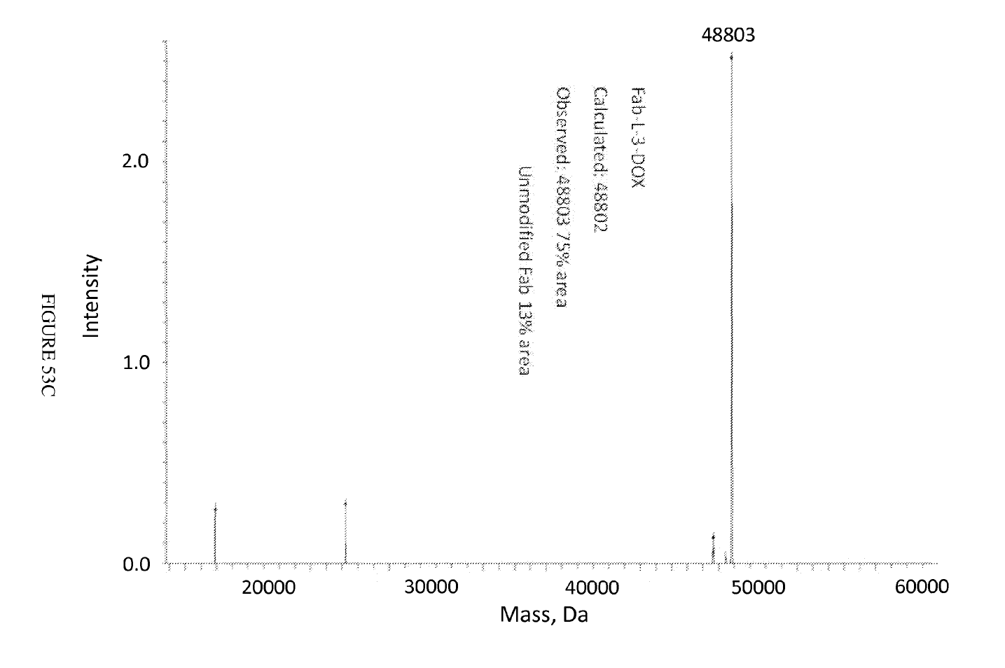

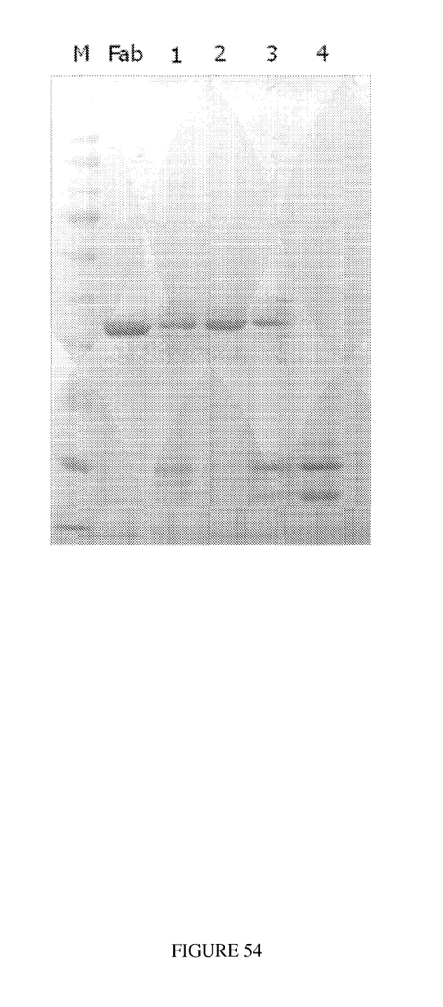

Trastuzumab Fab was transferred into a borate buffer (25 mM sodium borate, 25 mM NaCl, 1 mM EDTA, pH 8.0) by ultrafiltration (MWCO 10 kDa) and the concentration was corrected to 22.9 μM. This solution was treated with TCEP (3 eq.) at 37° C., shaking at 400 rpm for 2 hours in the presence of bridging reagent and DMF to ensure a 10% DMF (v/v) composition of the buffer system A) DTL-1-DOX (5 eq.); B) DTL-2-DOX (5 eq.); C) DTL-3-DOX (5 eq.). D) No bridging reagent was added, only DMF was added. After 2 hours, samples (5 μL) were taken from each reaction, quenched with maleimide (20 eq.) and reserved for SDS-PAGE gel analysis. The reaction mixture was immediately diluted with PBS to 400 μL, extracted with EtOAc (2×200 μL) to remove excess bridging reagent. The aqueous layer with Fab ADC was buffer swapped into a phosphate buffer (70 mM phosphates, 1 mM EDTA, pH 6.8) by ultrafiltration (MWCO 10 kDa) with at least 4 cycles of concentration by ultrafiltration and dilution. The purified material was analysed by UV/Vis for the purposes of determining yield of recovered antibody and DAR according to the formula described above, replacing the previous full Trastuzumab ε₂₈₀ with the value for Trastuzumab Fab as indicated above. Analysis by LCMS was also carried out (FIG. 55). Analysis by SDS-PAGE gel was also performed (FIG. 54).

Yields and DAR for In Situ Protocol with Trastuzumab Fab

| Reaction | Reagent | Yield* | DAR |
|---|---|---|---|
| A | DTL-1-DOX | 75% | 1.43 |
| B | DTL-2-DOX | 88% | 0.74 |
| C | DTL-3-DOX | 78% | 1.12 |

*Purification yields, not conversion.

### 4.9 ELISA Assay for Trastuzumab ADCs

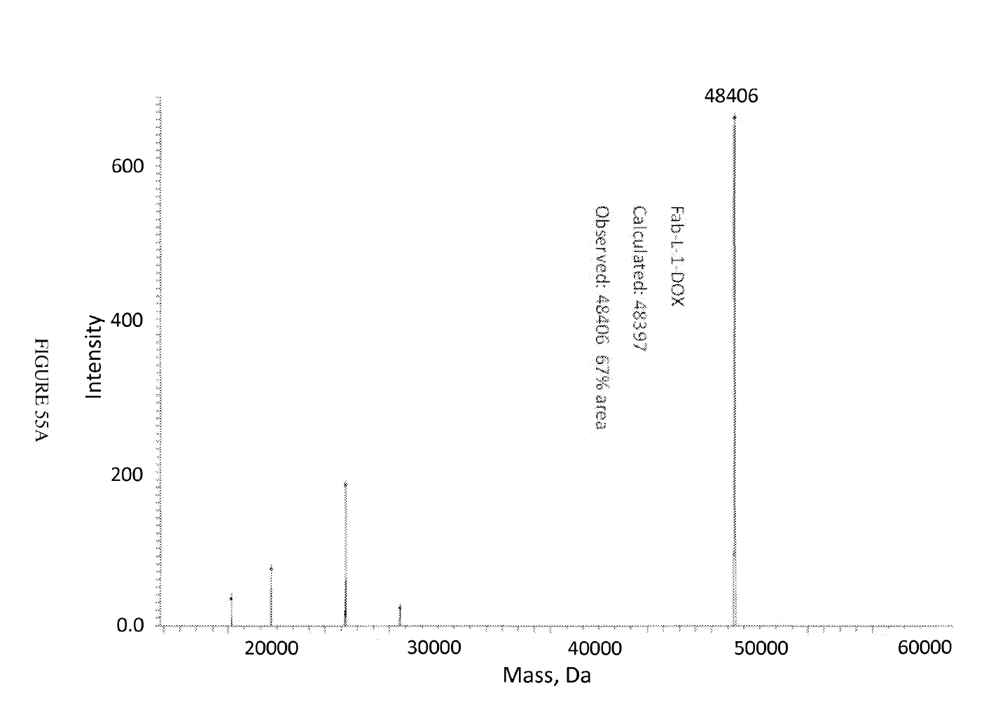

ELISA assays were conducted for the Trastuzumab ADCs and Trastuzumab Fab ADCs with DTL-1-DOX, DTL-2-DOX and DTL-3-DOX conjugated by all three protocols; the results are shown in FIGS. 56 to 58. Typical protocol for ELISA assay: Coated a 96-well plate with Her2 (100 μL of 0.25 ng/mL) including a row for negative PBS controls. Left coating for 2 hours at room temperature then blocked with 200 μL of 1% BSA solution overnight at 4° C. Next day incubated with a dilution series for the test samples (30 μM, 10 μM, 3.33 μM, 1.11 μM, 0.37 μM, 0.12 μM) for 1 hour at room temperature.

Then incubate with detection antibody diluted in PBS (anti-human IgG, Fab-specific-HRP) for 1 hour and finally added 100 μL of o-phenylenediamine hydrochloride 10 mg/20 mL in a phosphate-citrate buffer with sodium perborate. Reaction was stopped by acidifying with 50 μL of 4M HCl. Absorbance was measured at 490 nm Binding of maleimide-bridged trastuzumab ADCs was maintained against the target Her2 antigen.

### 5. Antibody Modification with Pyridazinediones

### 5.1 Pyridazinedione Reagent Synthesis

### 5.1.1 1-Azido-4-methylbenzene

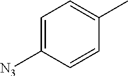

To a solution of p-Toluidine (2.0 g, 18.4 mmol) in 2N HCl (28 mL) at −5° C. was added slowly a solution of sodium nitrite (1.5 g, 22.4 mmol) in H₂O (5 mL) over 5 min making sure that the internal temperature did not rise above 0° C. After completion of addition, the reaction mixture was stirred at −5° C. for 5 min to form a diazonium salt. Then urea (130 mg, 2.2 mmol) was added to neutralise the diazonium salt solution. Following this, the diazonium salt solution was added to a solution of sodium azide (2.4 g, 37.2 mmol) and sodium acetate (4.6 g, 56 mmol) in 30 mL of H₂O at 0° C. over 5 min. The mixture was stirred for 2 h at 0° C. The mixture was extracted into Et₂O (2×60 mL), the combined organic layers dried (MgSO₄) and concentrated in vacuo to afford 1-azido-4-methylbenzene (2.3 g, 17.3 mmol, 94%) as a yellow oil: ¹H NMR (500 MHz, CDCl₃) δ 7.15 (d, J=8.4 Hz, 2H), 6.92 (d, J=8.4 Hz, 2H), 2.33 (s, 3H); ¹³C NMR (125 MHz, CDCl₃) δ 137.2 (CH), 134.7 (CH), 130.4 (CH), 118.9 (CH), 21.0 (CH₃).

### 5.1.2 1-Azido-4-(bromomethyl)benzene

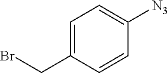 [SMILES: [N-]=[N+]=Nc1ccc(CBr)cc1]

A solution of 1-Azido-4-methylbenzene (0.85 g, 6.4 mmol), N-bromosuccinimide (1.5 g, 8.3 mmol) and azobis(isobutyronitrile) (0.31 g, 1.9 mmol) in dry benzene (20 mL) was heated under reflux under argon in the dark for 8 h. After this time, the mixture was poured into H₂O (20 mL), extracted into Et₂O (2×20 mL), the combined organic layers dried (MgSO₄) and concentrated in vacuo. Purification by flash column chromatography (neat petrol) yielded 1-azido-4-(bromomethyl)benzene (1.1 g, 5.1 mmol, 80%) as a light brown solid: ¹H NMR (300 MHz, CDCl₃) δ 7.38 (d, J=8.3 Hz, 2H), 7.00 (d, J=8.6 Hz, 2H), 4.48 (s, 2H); ¹³C NMR (150 MHz, CDCl₃) δ 140.3 (CH), 134.6 (CH), 130.7 (CH), 119.5 (CH), 33.0 (CH₂); HRMS (ES⁺) calcd for C₇H₆N₃Br [M⁷⁹Br+H]⁺ 211.9740, observed 211.9743.

### 5.1.3 Di-tert-butyl 1-(prop-2-yn-1-yl)hydrazine-1,2-dicarboxylate

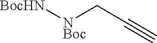 [SMILES: C#CCN(NC(=O)OC(C)(C)C)C(=O)OC(C)(C)C]

To a solution of di-tert-butyl hydrazine-1,2-dicarboxylate (300 mg, 1.29 mmol) in a mixture of toluene (2 mL) and 5% aqueous NaOH (2 mL) was added tetra-n-butylammonium bromide (13 mg, 0.03 mmol) and propargyl bromide (461 mg, 3.87 mmol). The reaction mixture was stirred at 21° C. for 16 h. After this time, H₂O (20 mL) was added and the mixture was extracted with ethyl acetate (3×15 mL). The combined organic layers were washed with brine (15 mL), dried (MgSO₄), and concentrated in vacuo. Purification by flash column chromatography (20% EtOAc/petrol) yielded di-tert-butyl 1-(prop-2-yn-1-yl)hydrazine-1,2-dicarboxylate (435 mg, 1.61 mmol, 85%) as a white solid: m.p. 103-104° C. (lit. m.p. 103.1-103.4° C.)^{Error! Bookmark not defined. 1}H NMR (500 MHz, CDCl₃) δ 6.47 (br s, 0.78H), 6.18 (br s, 0.22H), 4.27 (s, 2H), 2.24 (t, J=2.4 Hz, 1H), 1.48 (s, 18H); ¹³C NMR (150 MHz, CDCl₃) δ 155.0 (C), 82.2 (C), 81.7 (C), 79.0 (C), 77.7 (C), 72.5 (CH), 39.5 (CH₂), 28.5 (CH₃), 28.5 (CH₃).

### 5.1.4 Di-tert-butyl 1-(4-azidobenzyl)-2-(prop-2-yn-1-yl)hydrazine-1,2-dicarboxylate

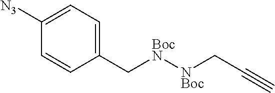 [SMILES: C#CCN(C(=O)OC(C)(C)C)N(Cc1ccc(N=[N+]=[N-])cc1)C(=O)OC(C)(C)C]

To a solution of di-tert-butyl 1-(prop-2-yn-1-yl)hydrazine-1,2-dicarboxylate (200 mg, 0.70 mmol) in DMF (10 mL) was added cesium carbonate (480 mg, 1.50 mmol) and 1-azido-4-(bromomethyl)benzene (230 mg, 1.10 mmol). The reaction mixture was stirred at 21° C. for 16 h. After this time, the reaction mixture was diluted with H₂O (20 mL) and extracted with EtOAc (3×20 mL). The combined organic layers were washed with brine (15 mL), dried (MgSO₄), and concentrated in vacuo. Purification by flash column chromatography (20% Et₂O/petrol) yielded di-tert-butyl 1-(4-azidobenzyl)-2-(prop-2-yn-1-yl)hydrazine-1,2-dicarboxylate (261 mg, 0.65 mmol, 93%) as a viscous dark yellow liquid: ¹H NMR (500 MHz, CDCl₃) δ 7.38 (d, J=8.4 Hz, 2H), 6.97 (d, J=8.4 Hz, 2H), 4.63-3.98 (m, 4H), 2.19 (t, J=2.4 Hz, 1H), 1.47 (s, 11H), 1.30 (s, 9H); ¹³C NMR (150 MHz, CDCl₃) d 154.6 (C), 154.3 (C), 139.5 (C), 133.6 (C), 131.4 (CH), 118.9 (CH), 81.7 (C), 81.6 (C), 78.5 (C), 72.9 (CH), 52.6 (CH₂), 39.3 (CH₂), 28.3 (CH₃), 28.1 (CH₃); HRMS (CI) calcd for C₂₀H₂₇N₅O₄Na [M+Na]⁺ 424.1961, observed 424.1965.

### 5.1.5 1-(4-Azidobenzyl)-4,5-dibromo-2-(prop-2-yn-1-yl)-1,2-dihydropyridazine-3,6-dione

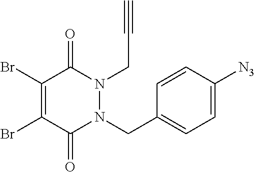 [SMILES: C#CCn1c(=O)c(Br)c(Br)c(=O)n1Cc1ccc(N=[N+]=[N-])cc1]

To a solution of di-tert-butyl 1-(4-azidobenzyl)-2-(prop-2-yn-1-yl)hydrazine-1,2-dicarboxylate (1.8 g, 4.5 mmol) in CH₂Cl₂ (55 mL) was added TFA (18 mL) and the reaction mixture stirred at 21° C. for 30 min After this time, all volatile material was removed in vacuo. The crude residue was added to a solution of 2,3-dibromomaleic anhydride (1.4 g, 5.4 mmol, 1.2 eq) in glacial AcOH (125 mL), and the reaction mixture heated at 130° C. for 16 h. Then the reaction mixture was concentrated in vacuo, and purification by flash column chromatography (15% to 50% Et₂O/petrol) yielded 1-(4-azidobenzyl)-4,5-dibromo-2-(prop-2-yn-1-yl)-1,2-dihydropyridazine-3,6-dione (560 mg, 1.30 mmol, 28%) as a yellow solid: ¹H NMR (500 MHz, CDCl₃) δ 7.25 (d, J=8.5 Hz, 2H), 7.02 (d, J=8.5 Hz, 2H), 5.46 (s, 2H), 4.75 (d, J=2.5 Hz, 2H), 2.45 (t, J=2.4 Hz, 1H); ¹³C NMR (125 MHz, CDCl₃) δ 153.5 (C), 153.0 (C), 140.8 (C), 136.7 (C), 135.8 (C), 130.9 (C), 128.5 (CH), 120.0 (CH), 75.7 (C), 75.2 (CH), 50.3 (CH₂), 37.1 (CH₂).

### 5.1.6 Methyl 3,4-dibromo-2,5-dioxo-2,5-dihydro-1H-pyrrole-1-carboxylate

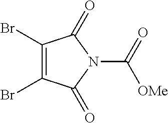 [SMILES: COC(=O)N1C(=O)C(Br)=C(Br)C1=O]

To a solution of dibromomaleimide (1.0 g, 3.9 mmol) and N-methylmorpholine (0.43 mL, 3.9 mmol) in THF (35 mL) was added methylchloroformate (0.31 mL, 3.9 mmol) and the reaction mixture was stirred at 21° C. for 20 min After this time, CH₂Cl₂ (40 mL) was added, and the reaction mixture was washed with H₂O (50 mL), dried (MgSO₄) and concentrated in vacuo to afford methyl 3,4-dibromo-2,5-dioxo-2,5-dihydro-1H-pyrrole-1-carboxylate (1.18 g, 3.80 mmol, 97%) as a pink power: m.p. 115-118° C.; ¹H NMR (500 MHz, CDCl₃) δ 4.00 (s, 3H); ¹³C NMR (125 MHz, CDCl₃) δ 159.3 (C), 147.0 (C), 131.5 (C), 54.9 (CH₃); HRMS (EI) calcd for C₆H₃O₄N⁷⁹Br₂ [M]⁺. 310.8423, observed 310.8427.

### 5.1.7 Tert-butyl 14-azido-3-(tert-butoxycarbonyl)-2-(prop-2-yn-1-yl)-6,9,12-trioxa-2,3-diazatetradecan-1-oate

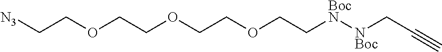 [SMILES: C#CCN(C(=O)OC(C)(C)C)N(CCOCCOCCOCCN=[N+]=[N-])C(=O)OC(C)(C)C]

To a solution of di-tert-butyl 1-(prop-2-yn-1-yl)hydrazine-1,2-dicarboxylate (108 mg, 0.40 mmol) in DMF (3 mL) was added cesium carbonate (156 mg, 0.48 mmol) and 2-(2-(2-(2-azidoethoxyl)ethoxy)ethoxy)ethyl methanesulfonate (130 mg, 0.44 mmol) and the reaction mixture stirred at 21° C. for 16 h. After this time, the reaction mixture was diluted with H₂O (10 mL), extracted with Et₂O (5×10 mL), the combined organic layers washed with sat. aq. LiC1 (2×10 mL), dried (MgSO₄), and concentrated in vacuo. Purification by flash column chromatography (30% EtOAc/petrol) yielded tert-butyl 14-azido-3-(tert-butoxycarbonyl)-2-(prop-2-yn-1-yl)-6,9,12-trioxa-2,3-diazatetradecan-1-oate (177 mg, 0.38 mmol, 94%) as a yellow oil: ¹H NMR (500 MHz, CDCl₃) δ 4.61-3.41 (m, 16H) 3.38 (t, J=5.0 Hz, 2H), 2.27-2.21 (m, 1H) 1.51-1.42 (m, 18H); ¹³C NMR (150 MHz, CDCl₃) d 155.3 (C), 155.3 (C), 154.8 (C), 154.7 (C), 154.5 (C), 154.3 (C), 153.9 (C), 82.2 (C), 82.0 (C), 81.7 (C), 81.7 (C), 81.4 (C), 81.3 (C), 79.3 (C), 79.3 (C), 78.9 (C), 72.5 (CH), 72.3 (CH), 72.1 (CH), 70.8 (CH₂), 70.8 (CH₂), 70.7 (CH₂), 70.5 (CH₂), 70.4 (CH₂), 70.3 (CH₂), 70.1 (CH₂), 68.6 (CH₂), 68.5 (CH₂), 68.4 (CH₂), 50.8 (CH₂), 50.7 (CH₂), 50.7 (CH₂), 49.8 (CH₂), 49.8 (CH₂), 41.3 (CH₂), 41.2 (CH₂), 39.7 (CH₂), 39.6 (CH₂), 28.3 (CH₃), 28.3 (CH₃), 28.3 (CH₃), 28.2 (CH₃), 28.1 (CH₃), 28.0 (CH₃), 28.0 (CH₃), 27.8 (CH₃); HRMS (CI) calcd for [M+Na]⁺ 494.2591, observed 494.2582.

### 5.1.8 1-(2-(2-(2-(2-Azidoethoxy)ethoxy)ethoxy)ethyl)-4,5-dibromo-2-(prop-2-yn-1-yl)-1,2-dihydropyridazine-3,6-dione

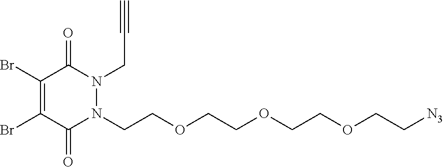 [SMILES: C#CCn1c(=O)c(Br)c(Br)c(=O)n1CCOCCOCCOCCN=[N+]=[N-]]

To a solution of tert-butyl 14-azido-3-(tert-butoxycarbonyl)-2-(prop-2-yn-1-yl)-6,9,12-trioxa-2,3-diazatetradecan-1-oate (100 mg, 0.21 mmol) in CH₂Cl₂ (2 mL) was added TFA (1 mL) and the reaction mixture stirred at 21° C. for 30 min After this time, all volatile material was removed in vacuo. The crude residue was added to a solution of N-methoxycarbonyl-dibromomaleimide (73 mg, 0.23 mmol) and NEt₃ (47 mg, 0.47 mmol) in CH₂Cl₂ (5 mL) and the reaction mixture stirred at 21° C. for 16 h. Then the reaction mixture was concentrated in vacuo, and purification by flash column chromatography (0.2% MeOH/CH₂Cl₂) yielded 1-(2-(2-(2-(2-azidoethoxyl)ethoxy)ethoxy)ethyl)-4,5-dibromo-2-(prop-2-yn-1-yl)-1,2-dihydropyridazine-3,6-dione (25 mg, 0.05 mmol, 23%) as a yellow oil: ¹H NMR (600 MHz, CDCl₃) δ 5.15 (d, J=2.3 Hz, 2H), 4.45 (t, J=4.7 Hz, 2H), 3.77 (t, J=4.7 Hz, 2H), 3.67-3.64 (m, 2H), 3.63-3.54 (m, 8H), 3.39 (t, J=5.1 Hz, 2H), 2.38 (t, J=2.4 Hz, 1H); ¹³C NMR (150 MHz, CDCl₃) δ 152.9 (C), 152.5 (C), 136.4 (C), 135.8 (C), 76.6 (C), 74.5 (CH), 70.8 (CH₂), 70.8 (CH₂), 70.7 (CH₂), 70.6 (CH₂), 70.2 (CH₂), 68.3 (CH₂), 50.7 (CH₂), 48.4 (CH₂), 37.3 (CH₂); HRMS (ES⁺) calcd for C₁₅H₂₀O₅N₅⁷⁹Br₂ [M+1-1]⁺ 507.9831, observed 507.9835.

### 5.1.9 2,2′-((1-(4-Azidobenzyl)-3,6-dioxo-2-(prop-2-yn-1-yl)-1,2,3,6-tetrahydropyridazine-4,5-diyl)bis(sulfanediyl))dibenzoic acid

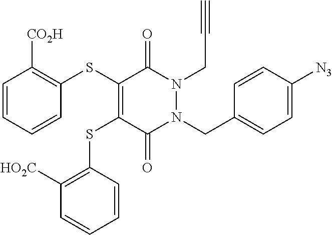 [SMILES: C#CCn1c(=O)c(Sc2ccccc2C(=O)O)c(Sc2ccccc2C(=O)O)c(=O)n1Cc1ccc(N=[N+]=[N-])cc1]

To a solution of 1-(4-azidobenzyl)-4,5-dibromo-2-(prop-2-yn-1-yl)-1,2-dihydropyridazine-3,6-dione (89 mg, 0.20 mmol) in CH₂Cl₂ (5 mL) was added NEt₃ (0.11 mL, 0.80 mmol) and thiosalicylic acid (63 mg, 0.40 mmol) and the mixture was stirred at 21° C. for 30 min. The reaction mixture was then concentrated in vacuo. To the crude residue was added H₂O (10 mL) and the mixture washed with EtOAc (2×10 mL). The aqueous layer acidified to pH 2 by addition 1N aq. HCl, extracted with EtOAc (4×10 mL), the combined organic layers dried (MgSO₄), and concentrated in vacuo to afford 2,2′-((1-(4-azidobenzyl)-3,6-dioxo-2-(prop-2-yn-1-yl)-1,2,3,6-tetrahydropyridazine-4,5-diyl)bis(sulfanediyl))dibenzoic acid (113 mg, 0.19 mmol, 97%) as a yellow solid: ¹H NMR (500 MHz, CDCl₃) δ 8.01-7.93 (m, 2H), 7.47-7.30 (m, 6H), 7.17 (d, J=8.4 Hz, 2H), 6.96 (d, J=8.4 Hz, 2H), 5.33 (s, 2H), 4.64 (s, 2H), 2.41 (t, J=2.4 Hz, 1H); ¹³C NMR (125 MHz, CDCl₃) δ 170.3 (C), 170.2 (C), 156.0 (C), 155.6 (C), 144.2 (C), 143.7 (C), 140.5 (C), 134.6 (C), 134.5 (C), 132.9 (CH), 132.7 (CH), 132.5 (CH), 132.1 (CH), 132.0 (CH), 131.1 (C), 128.6 (CH), 128.1 (CH), 119.8 (CH), 75.9 (C), 74.9 (CH), 49.7 (CH₂), 36.5 (CH₂); HRMS (ES⁻) calcd for C₂₈H₁₈O₆N₅S₂ [M-H]⁻ 584.0699, observed 584.0710.

### 5.1.10 N,N′-(((Oxybis(ethane-2,1-diyl))bis(oxy))bis(ethane-2,1-diyl))bis(1-fluorocyclooct-2-ynecarboxamide)

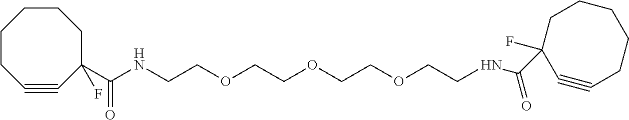 [SMILES: O=C(NCCOCCOCCOCCNC(=O)C1(F)C#CCCCCC1)C1(F)C#CCCCCC1]

To a solution of 1-fluorocyclooct-2-ynecarboxylic acid (230 mg, 1.35 mmol) and DIPEA (0.482 mL, 2.7 mmol) in DMF (10 mL) was added HBTU (616 mg, 1.62 mmol) and the reaction mixture stirred at 21° C. for 5 min After this time, was added 1,11-diamino-3,6,9-trioxaundecane (130 mg, 0.68 mmol) and the reaction mixture stirred at 21° C. for 4 h. Then the reaction mixture was diluted with H₂O (30 mL), extracted with EtOAc (3×15 mL), the combined organic layers were dried (MgSO₄) and concentrated in vacuo. The crude residue was purified by flash column chromatography (50% EtOAc/Et₂O) to afford N,N′-(((oxybis(ethane-2,1-diyl))bis(oxy))bis(ethane-2,1-diyl))bis(1-fluorocyclooct-2-ynecarboxamide) (340 mg, 0.05 mmol, 99%) as a yellow oil. ¹H NMR (500 MHz, CDCl₃) δ ppm 7.21 (br s, 2H), 3.62-3.50 (m, 12H), 3.50-3.35 (m, 4H), 2.35-2.15 (m, 8H), 2.05-1.74 (m, 8H), 1.64-1.55 (m, 2H), 1.42-1.30 (m, 2H); ¹³C NMR (125 MHz, CDCl₃) δ 169.4 (C), 109.6 (C), 93.6 (C), 86.9 (C), 70.1 (CH₂), 70.0 (CH₂), 69.8 (CH₂), 46.6 (CH₂), 46.4 (CH₂), 39.3 (CH₂), 33.8 (CH₂), 28.9 (CH₂), 25.6 (CH₂), 20.5 (CH₂); HRMS (ES⁻) calcd for C₂₆H₃₇O₅N₂F₂ [M-H]⁻ 495.2671, observed 495.2668.

### 5.1.11 1-(4-((4,5-Dibromo-3,6-dioxo-2-(prop-2-yn-1-yl)-2,3-dihydropyridazin-1(6H)-yl)methyl)phenyl)-4-fluoro-N-(1-(1-fluorocyclooct-2-yn-1-yl)-1-oxo-5,8,11-trioxa-2-azatridecan-13-yl)-4,5,6,7,8,9-hexahydro-1H-cycloocta[d][1,2,3]triazole-4-carboxamide

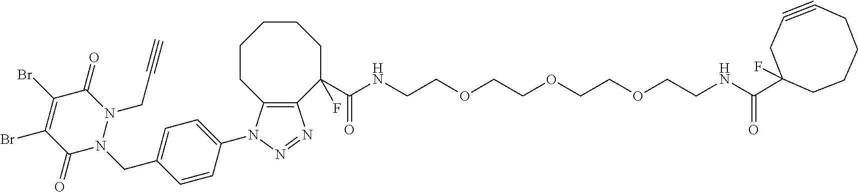 [SMILES: C#CCn1c(=O)c(Br)c(Br)c(=O)n1Cc1ccc(-n2nnc3c2CCCCCC3(F)C(=O)NCCOCCOCCOCCNC(=O)C2(F)CC#CCCCC2)cc1]

To a solution of N,N′-(((oxybis(ethane-2,1-diyl))bis(oxy))bis(ethane-2,1-diyl))bis(1-fluorocyclooct-2-ynecarboxamide) (136 mg, 0.28 mmol) in CH₂Cl₂ (5 mL) was added slowly a solution of 1-(4-azidobenzyl)-4,5-dibromo-2-(prop-2-yn-1-yl)-1,2-dihydropyridazine-3,6-dione (50 mg, 0.11 mmol) in CH₂Cl₂ (3 mL) and the reaction mixture stirred at 21° C. for 16 h. After this time, the reaction mixture was concentrated in vacuo and the crude residue purified by flash column chromatography (1% MeOH/EtOAc) to afford 1-(4-((4,5-dibromo-3,6-dioxo-2-(prop-2-yn-1-yl)-2,3-dihydropyridazin-1(6H)-yl)methyl)phenyl)-4-fluoro-N-(1-(1-fluorocyclooct-2-yn-1-yl)-1-oxo-5,8,11-trioxa-2-azatridecan-13-yl)-4,5,6,7,8,9-hexahydro-1H-cycloocta[d][1,2,3]triazole-4-carboxamide (33 mg, 0.04 mmol, 32%) as an inseparable mixture of diastereo- and regio-isomers as a yellow oil: ¹H NMR (600 MHz, CDCl₃) δ 7.44 (d, J=8.7 Hz, 2H), 7.42 (d, J=8.7 Hz, 2H), 7.32 (br s, 1H), 6.88 (br s, 1H), 5.57 (t, J=17.7 Hz, 2H), 4.77 (s, 2H), 3.71-3.51 (m, 14H), 3.48 (t, J=6.0 Hz, 2H), 3.02-2.92 (m, 1H), 2.92-2.84 (m, 1H), 2.73-2.58 (m, 1H), 2.48 (t, J=2.4 Hz, 1H), 2.44-2.22 (m, 5H), 2.02-1.39 (m, 12H); ¹³C NMR (150 MHz, CDCl₃) δ 171.0, 170.8, 168.7, 168.5, 153.5, 153.0, 143.2, 143.0, 136.7, 136.4, 136.2, 136.1, 135.4, 135.4, 128.2, 127.0, 126.9, 109.4, 109.3, 95.2, 95.2, 94.6, 93.9, 93.9, 93.4, 87.5, 87.3, 75.6, 75.5, 70.7, 70.6, 70.6, 70.5, 70.4, 70.4, 69.7, 69.5, 50.2, 46.6, 46.4, 39.4, 39.3, 37.4, 34.0, 33.3, 33.1, 29.0, 26.5, 25.8, 24.0, 22.3, 22.3, 21.8, 21.2, 20.7, 20.7; HRMS (ES⁺) calcd for C₄₀H₄₈O₇N₇Br₂F₂ [1\4⁷⁹Br⁷⁹Br+H]⁺ 934.1989, observed 934.1950.

### 5.2 General Procedures for the Conjugation of Antibodies Using Pyridazinedione-Based Bridging Reagents

### 5.2.1 General Procedure for the Preparation of the Her-Fab-Pyridazinedione Conjugate (Her-Fab-PD)

To a solution of Her-Fab (50 μL, 30 μM, 1.4 mg/mL, 1 eq) in borate buffer (25 mM sodium borate, 25 mM NaCl, 1 mM EDTA, pH 8.0) was added TCEP (final concentration 90 μM, 3 eq) and the reaction mixture incubated at 37° C. for 90 min After this time, was added a solution of pyridazinedione in DMF (final concentration 3 mM, 10 eq) and the reaction mixture incubated at 37° C. for 1 h. Following this, analysis by LCMS revealed 99% conversion to the conjugate. The excess reagents were then removed by repeated diafiltration into fresh buffer using VivaSpin sample concentrators (GE Healthcare, 10,000 MWCO).

### 5.2.2 General Procedure for Azide-Alkyne Huisgen Cycloaddition (CuAAC)

To a solution of ‘clickable’-Her-Fab-Pyridazinedione (50 μL, 21 μM, 1 mg/mL) in PBS (pH 7.4) containing tris(3-hydroxypropyltriazolylmethyl)amine (THPTA) (500 μM), CuSO₄ (100 μM), aminoguanidine (5 mM) was added a cargo molecule (azide or alkyne) (final concentration 420 μM, 20 eq) and sodium ascorbate (final concentration 5 mM) and the reaction mixture incubated at 25° C. for 1 h. Following this, analysis by LCMS revealed 99% conversion to the conjugate. The excess reagents were then removed by repeated diafiltration into fresh buffer using VivaSpin sample concentrators (GE Healthcare, 10,000 MWCO).

### 5.2.3 General procedure for Strain-Promoted Azide-Alkyne Cycloaddition (SPARC)

To a solution of ‘clickable’-Her-Fab-Pyridazinedione (50 μL, 21 μM, 1 mg/mL) in PBS (pH 7.4) was added a cargo molecule (azide) and the reaction mixture incubated at 25° C. for 4 h. Following this, analysis by LCMS revealed 99% conversion to the conjugate. The excess reagents were then removed by repeated diafiltration into fresh buffer using VivaSpin sample concentrators (GE Healthcare, 10,000 MWCO).

### 5.3 Pyridazinedione Conjugation of Antibody FAB fragments

### 5.3.1 Preparation of her-Fab-AzideAlkyne-Pyridazinedione Conjugate (her-Fab-Azal-PD)

The general procedure for the preparation of the Her-Fab-Pyridazinedione conjugate with 2,2′-((1-(4-azidobenzyl)-3,6-dioxo-2-(prop-2-yn-1-yl)-1,2,3,6-tetrahydropyridazine-4,5-diyl)bis(sulfanediyl))dibenzoic acid as the bridging reagent was followed.

Observed mass: 47925. Expected mass: 47924.

### 5.3.2 Preparation of Her-Fab-PEGAzideAlkyne-Pyridazinedione conjugate (Her-Fab-Pazal-PD)

The general procedure for the preparation of the Her-Fab-Pyridazinedione conjugate with 1-(2-(2-(2-(2-azidoethoxyl)ethoxy)ethoxy)ethyl)-4,5-dibromo-2-(prop-2-yn-1-yl)-1,2-dihydropyridazine-3,6-dione as the bridging reagent was followed.

Observed mass: 47994. Expected mass: 47994.

### 5.3.3 Preparation of her-Fab-AlkyneStrainedAlkyne-Pyridazinedione Conjugate (her-Fab-Astra-PD)

The general procedure for the preparation of the Her-Fab-Pyridazinedione conjugate with 1-(4-((4,5-dibromo-3,6-dioxo-2-(prop-2-yn-1-yl)-2,3-dihydropyridazin-1(6H)-yl)methyl)phenyl)-4-fluoro-N-(1-(1-fluorocyclooct-2-yn-1-yl)-1-oxo-5,8,11-trioxa-2-azatridecan-13-yl)-4,5,6,7,8,9-hexahydro-1H-cycloocta[d][1,2,3]triazole-4-carboxamide as the bridging reagent was followed.

Observed mass: 48418. Expected mass: 48420.

### 5.4 Functionalisation of Fab-Pyridazinedione Conjugates

### 5.4.1 Preparation of Her-Fab-Azal-PD-PEG₄ conjugate

The general procedure for CuAAC with 2-(2-(2-(2-azidoethoxyl)ethoxy)ethoxy)ethanol (PEG₄-N₃) as the cargo molecule and Her-Fab-Azal-PD as the ‘clickable’-Her-Fab-Pyridazinedione was followed.

Observed mass: 48148. Expected mass: 48143.

### 5.4.2 Preparation of her-Fab-Azal-PD-Rhodamine Conjugate

The general procedure for SPAAC with dibenzylcyclooctyne-PEG₄-tetramethylrhodamine (DBCO-PEG₄-TAMRA) as the cargo molecule and Her-Fab-Azal-PD as the ‘clickable’-Her-Fab-Pyridazinedione was followed.

Observed mass: 48864. Expected mass: 48861.

### 5.4.3 Preparation of her-Fab-Azal-PD-Rhodamine-Fluorescein Conjugate

The general procedure for CuAAC with 1424242-(2-azidoethoxyl)ethoxy)ethoxy)ethyl)-3-(3′,6′-dihydroxy-3-oxo-3H-spiro[isobenzofuran-1,9′-xanthen]-5-yl)thiourea (Fluorescein-PEG₄-N₃) as the cargo molecule and Her-Fab-Azal-PD-Rhodamine as the ‘clickable’-Her-Fab-Pyridazinedione was followed.

Observed mass: 49474. Expected mass: 49468.

### 5.4.4 Preparation of Her-Fab-Astra-PD-PEG₄ conjugate

The general procedure for SPAAC with PEG₄-N₃ as the cargo molecule and Her-Fab-Astra-PD as the ‘clickable’-Her-Fab-Pyridazinedione was followed.

Observed mass: 48640. Expected mass: 48639.

### 5.4.5 Preparation of Her-Fab-Astra-PD-PEG₄-PEG₄ conjugate

The general procedure for CuAAC with PEG₄-N₃ as the cargo molecule and Her-Fab-Astra-PD-PEG₄ as the ‘clickable’-Her-Fab-Pyridazinedione was followed.

Observed mass: 48880. Expected mass: 48882.

### 5.4.6 Preparation of her-Fab-Astra-PD-Fluorescein Conjugate

The general procedure for SPAAC with Fluorescein-PEG₄-N₃ as the cargo molecule and Her-Fab-Astra-PD as the ‘clickable’-Her-Fab-Pyridazinedione was followed.

Observed mass: 49032. Expected mass: 49025.

### 5.4.7 Preparation of her-Fab-Astra-PD-Fluorescein-PEG₄ Conjugate

The general procedure for CuAAC with PEG₄-N₃ as the cargo molecule and Her-Fab-Astra-PD-Fluorescein as the ‘clickable’-Her-Fab-Pyridazinedione was followed.

Observed mass: 49252. Expected mass: 49251.

### 5.4.8 Preparation of Her-Fab-Astra-PD-His₆ conjugate

The general procedure for SPAAC with Histidine₆-PEG₄-N₃ as the cargo molecule and Her-Fab-Astra-PD as the ‘clickable’-Her-Fab-Pyridazinedione was followed.

Observed mass: 49518. Expected mass: 49518.

### 5.4.9 Preparation of Her-Fab-Astra-PD-PEG DOX

The general procedure for SPAAC with DOX-PEG₄-N₃ as the cargo molecule and Her-Fab-Astra-PD as the ‘clickable’-Her-Fab-Pyridazinedione was followed.

Observed mass: 49253. Expected mass: 49257.

### 5.5 Pyridazinedione Modification of a Full Antibody

### 5.5.1 Stepwise modification of Trastuzumab mAb

Trastuzumab was transferred into a borate buffer (25 mM sodium borate, 25 mM NaCl, 1 mM EDTA, pH 8.0) by ultrafiltration (MWCO 10 kDa) and the concentration was corrected to 20.6 μM. This solution was treated with TCEP (10 eq.) at 37° C., shaking at 400 rpm for 2 hours. Then, eluted this solution through a PD-G25 buffer swapping column following manufacturer's protocol, equilibrated with the borate buffer described above, as means to separate from excess TCEP. The concentration was assessed by UV/Vis (ε₂₈₀=215,000 cm⁻¹ M⁻¹) and was concentrated back to 20.6 μM. Next, the solution was aliquoted into 40 μL (0.826 μmol) portions to which were added 4 μL of a 10.3 mM solution of A) 4,5-dibromo-1,2-diethyl-1,2-dihydropyridazine-3,6-dione (DiBr-Diet) (50 eq.) diluted into DMF (20 μL), kept at 37° C.; B) 1,2-diethyl-4,5-bis(phenylthio)-1,2-dihydropyridazine-3,6-dione (DiSH-Diet) (50 eq.) diluted into DMF (20 μL), kept at 37° C.; 4 μL of a 1.3 mM solution of C) 4,5-dibromo-1,2-diethyl-1,2-dihydropyridazine-3,6-dione (DiBr-Diet) (6 eq.) diluted into DMF (20 μL), kept at 37° C.; D) 1,2-diethyl-4,5-bis(phenylthio)-1,2-dihydropyridazine-3,6-dione (DiSH-Diet) (5 eq.) diluted into DMF (20 μL), kept at 37° C. The addition of DMF alongside bridging reagents ensured a 10% DMF (v/v) composition for the buffer system. 2 hours after addition samples (5 μL) were taken from each reaction, quenched with maleimide (20 eq.) and reserved for SDS-PAGE gel analysis. The reaction mixture was buffer swapped into a borate buffer (25 mM sodium borate, 25 mM NaCl, 1 mM EDTA, pH 8.0) by ultrafiltration (MWCO 10 kDa) with at least 6 cycles of concentration by ultrafiltration and dilution. The purified material was analysed by UV/Vis for the purposes of determining yield of recovered antibody and pyridazinedione antibody ratio (PAR) according to the formula described below. Dithiopyridazinediones have a strong absorbance at 339 nm. Analysis by SDS-PAGE gel was also performed.

$$PAR = \frac{\frac{{OD}_{339}}{9500 {M}^{- 1} {cm}^{- 1}}}{\frac{( {OD}_{280} - {OD}_{339} \times 0.280 )}{215000 {M}^{- 1} {cm}^{- 1}}} .$$

Yields and PAR for Stepwise Protocol with Trastuzumab mAb

| Reaction | Reagent | DAR |
|---|---|---|
| A | DiBr-Diet | 3.9 |
| B | DiSH-Diet | 4.1 |
| C | DiBr-Diet | 3.8 |
| D | DiSH-Diet | 3.8 |

### 5.5.2 In Situ Modification of Trastuzumab mAb

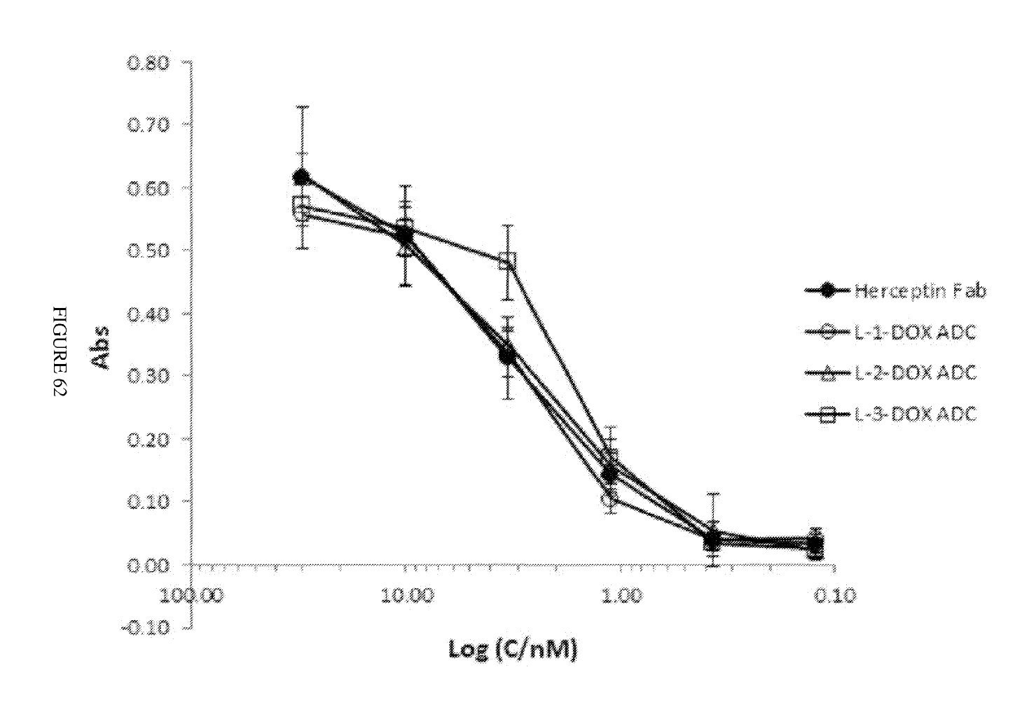

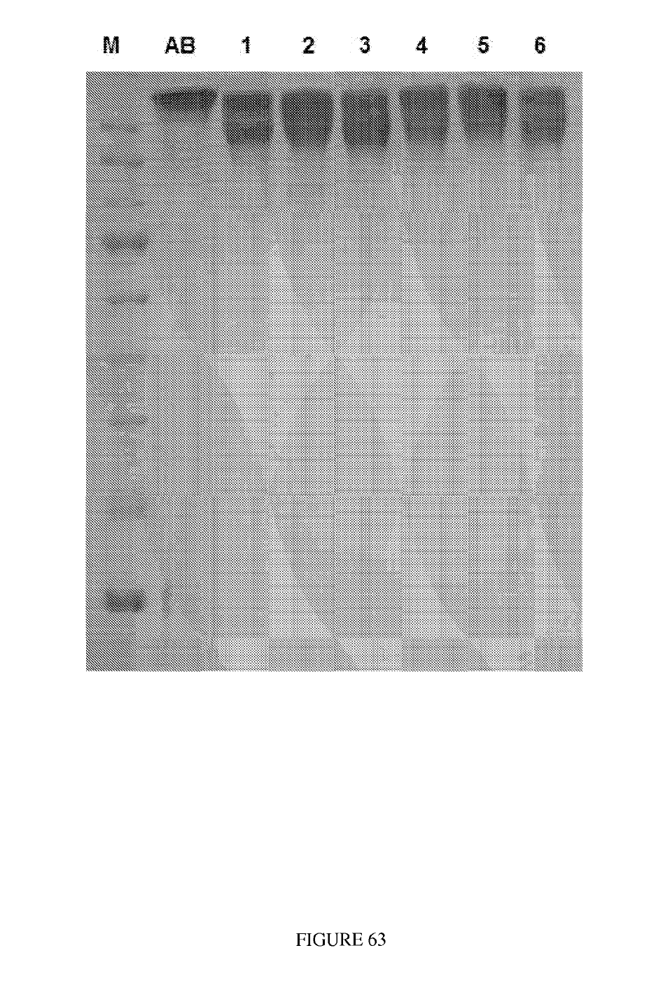

Trastuzumab was transferred into a borate buffer (25 mM sodium borate, 25 mM NaCl, 1 mM EDTA, pH 8.0) by ultrafiltration (MWCO 10 kDa) and the concentration was corrected to 22.9 μM. This solution was treated with TCEP (7 eq.) at 37° C., shaking at 400 rpm for 2 hours in the presence of bridging reagent and DMF to ensure a 10% DMF (v/v) composition of the buffer system A) 1,2-diethyl-4,5-bis(phenylthio)-1,2-dihydropyridazine-3,6-dione (DiSH-Diet) (50 eq.) diluted into DMF (20 μL), kept at 37° C.; B) 1,2-diethyl-4,5-bis(phenylthio)-1,2-dihydropyridazine-3,6-dione (DiSH-Diet) (6 eq.) diluted into DMF (20 μL), kept at 37° C. C) No bridging reagent was added, only DMF, reaction at 37° C. After 2 hours, samples (5 μL) were taken from each reaction, quenched with maleimide (20 eq.) and reserved for SDS-PAGE gel analysis. The reaction mixture was buffer swapped into a borate buffer (25 mM sodium borate, 25 mM NaCl, 1 mM EDTA, pH 8.0) by ultrafiltration (MWCO 10 kDa) with at least 6 cycles of concentration by ultrafiltration and dilution. The purified material was analysed by UV/Vis for the purposes of determining yield of recovered antibody and PAR according to the formula described above. Analysis by SDS-PAGE gel was performed (see FIG. 63).

### Yields and PAR for In Situ Protocol for Trastuzumab mAb

| Reaction | Reagent | DAR |
|---|---|---|
| A | DiSH-Diet | 3.9 |
| B | DiSH-Diet | 3.7 |

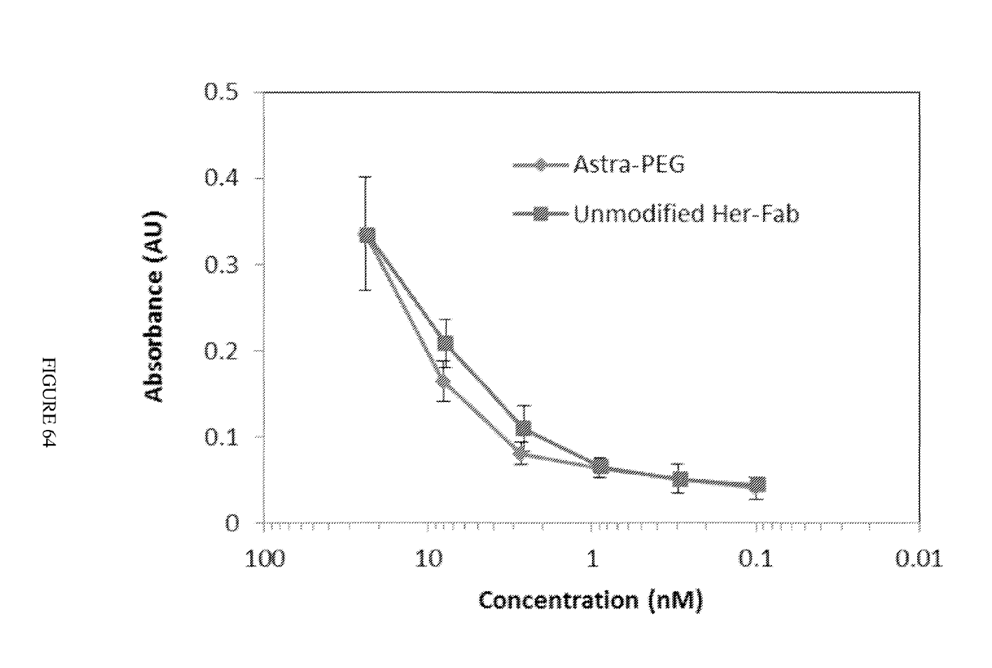

ELISA assays (see FIG. 64) were conducted for Trastuzumab Fab with Her-Fab-Astra-PD-PEG₄ conjugated by sequencial protocols. Typical protocol for ELISA assay: Coated a 96-well plate with Her2 (100 μL of 0.25 ng/mL) including a row for negative PBS controls. Left coating for 2 hours at room temperature then blocked with 200 μL of 1% BSA solution overnight at 4° C. Next day incubated with a dilution series for the test samples (24 μM, 8.1 μM, 2.7 μM, 0.89 μM, 0.30 μM, 0.10 μM) for 1 hour at room temperature. Then incubate with detection antibody diluted in PBS (anti-human IgG, Fab-specific-HRP) for 1 hour and finally added 100 μL of o-phenylenediamine hydrochloride 10 mg/20 mL in a phosphate-citrate buffer with sodium perborate. Reaction was stopped by acidifying with 50 μL of 4M HCl. Absorbance was measured at 490 nm Binding of pyridazinedione-bridged trastuzumab Fab was maintained against the target Her2 antigen.

1. A chemically modified antibody AB that:
(i) is capable of specific binding to an antigen AG;
(ii) comprises four chains, two of which are heavy chains and two of which are light chains; and
(iii) comprises at least one inter-chain bridging moiety of the formula (IA) or at least one inter-chain bridging moiety of the formula (TB) 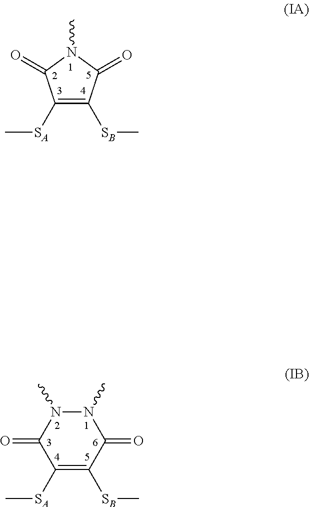
wherein S_{A} and S_{B} are sulfur atoms that are attached to different chains of said chemically modified antibody.

2. A chemically modified antibody according to claim 1, which is an IgG1 antibody.

3. A chemically modified antibody according to claim 2, which has one inter-chain bridging moiety of the formula (IA) or one inter-chain bridging moiety of the formula (TB), and whose chains are otherwise bridged by disulfide bridges —S—S—.

4. A chemically modified antibody according to claim 2, which has two inter-chain bridging moieties of the formula (IA) or two inter-chain bridging moieties of the formula (IB), and whose chains are otherwise bridged by disulfide bridges —S—S—.

5. A chemically modified antibody according to claim 4, wherein each of the two inter-chain bridging moieties of the formula (IA) or each of the two inter-chain bridging moieties of the formula (IB) bridges one of the two heavy chains to one of the two light chains.

6. A chemically modified antibody according to claim 2, which has three inter-chain bridging moieties of the formula (IA) or three inter-chain bridging moieties of the formula (IB), and whose chains are otherwise bridged by disulfide bridges —S—S—.

7. A chemically modified antibody according to claim 2, which has four inter-chain bridging moieties of the formula (IA) or four inter-chain bridging moieties of the formula (IB), and whose chains are not bridged by disulfide bridges —S—S—.

8. A chemically modified antibody according to claim 1, wherein each said at least one inter-chain bridging moiety of the formula (IA) is the same or different and is a moiety of the formula (IA′) and each said at least one inter-chain bridging moiety of the formula (IB) is the same or different and is a moiety of the formula (IB′): 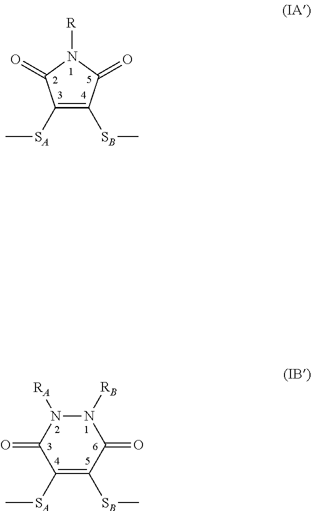
wherein:
R is (i) a hydrogen atom, (ii) a cargo moiety or (iii) a linker moiety, said linker moiety optionally being linked to at least one cargo moiety;
R_{A} and R_{B} are, independently of one another, (i) a chemically inert group, (ii) a cargo moiety or (iii) a linker moiety, said linker moiety optionally being linked to at least one cargo moiety; and
S_{A} and S_{B} are sulfur atoms that are attached to different chains of said chemically modified antibody.

9. A chemically modified antibody according to claim 8, wherein said chemically modified antibody comprises at least one cargo moiety that is a drug moiety.

10. A chemically modified antibody according to claim 9, wherein said drug moiety is a cytotoxic agent.

11. A chemically modified antibody according to claim 10, wherein said antigen is selected from MY9, B4, EpCAM, CD2, CD3, CD4, CD5, CD6, CD11, CD19, CD20, CD22, CD25, CD26, CD30, CD33, CD37, CD38, CD40, CD44, CD56, CD64, CD70, CD74, CD79, CD105, CD138, CD205, CD227, EphA receptors, EphB receptors, EGFR, EGFRvIII, HER2, HER3, BCMA, PSMA, Lewis Y, mesothelin, cripto, alpha(v)beta3, alpha(v)beta5, alpha(v) beta6 integrin, C242, CA125, GPNMB, ED-B, TMEFF2, FAP, TAG-72, GD2, CAIX and 5T4.

12. A chemically modified antibody according to claim 9, wherein said chemically modified antibody comprises at least one said inter-chain bridging moiety of the formula (IB′), in which R_{A} comprises said drug moiety and R_{B} comprises an imaging agent.

13. A chemically modified antibody according to claim 8, wherein said linker moiety (iii) is a moiety of the formula -L(CM)ₘ(Z)ₙ₋ₘ, wherein:
L represents a linking moiety;
each CM is the same or different and represents a cargo moiety;
each Z is the same or different and represents a reactive group attached to L and which is capable of reacting with a cargo moiety such that said cargo moiety becomes linked to L;
n is 1, 2 or 3; and
m is an integer of from zero to n.

14. A chemically modified antibody according to claim 8, wherein said linker moiety (iii) is capable of undergoing chemical fragmentation by enzymatic catalysis, acidic catalysis, basic catalysis, oxidative catalysis or reductive catalysis.

15. A process for selectively producing a chemically modified antibody according to claim 1, which process comprises:
reducing at least one inter-chain disulfide bridge of an antibody in the presence of a reducing agent; and
reacting said antibody with at least one inter-chain bridging reagent of the formula (IIA) or at least one inter-chain bridging reagent of the formula (IIB) 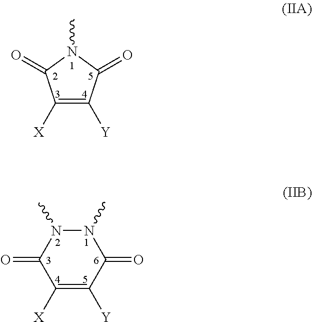
wherein X and Y each independently represent an electrophilic leaving group;
thereby introducing the desired number of inter-chain bridging moieties of the formula (IA) or (IB) at the desired locations of said antibody and producing said chemically modified antibody.

16. A process according to claim 15, wherein said reducing agent is selected from 2-mercaptoethanol, tris(2-carboxyethyl)phosphine, dithiothreitol and benzeneselenol.

17. A chemically modified antibody AB that:
(i) is capable of specific binding to an antigen AG;
(ii) comprises four chains, two of which are heavy chains and two of which are light chains; and
(iii) comprises at least one inter-chain bridging moiety of the formula (III) 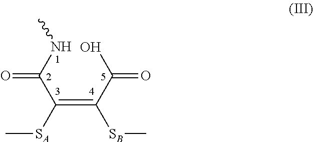
wherein S_{A} and S_{B} are sulfur atoms that are attached to different chains of said chemically modified antibody.

18. A chemically modified antibody fragment AB_{F} that:
(i) is capable of specific binding to an antigen AG;
(ii) comprises at least two chains; and
(iii) comprises at least one inter-chain bridging moiety of the formula (IA_{F}) or at least one inter-chain bridging moiety of the formula (IB_{F}) 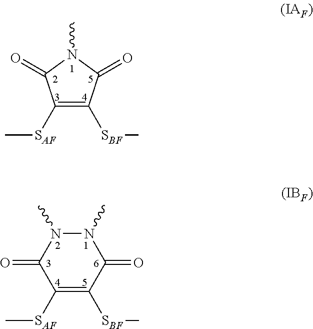
wherein S_{AF} and S_{BF} are sulfur atoms that are attached to different chains of said chemically modified antibody fragment.

19. A chemically modified antibody fragment AB_{F} according to claim 18, which is an scFv antibody fragment in which the heavy chain is bridged to the light chain via said at least one inter-chain bridging moiety of the formula (IA_{F}) or at least one inter-chain bridging moiety of the formula (IB_{F}).

20. A chemically modified antibody fragment AB_{F} according to claim 18, which is a Fab antibody fragment in which the heavy chain is bridged to the light chain via said at least one inter-chain bridging moiety of the formula (IA_{F}) or at least one inter-chain bridging moiety of the formula (IB_{F}).

21. A chemically modified antibody fragment AB_{F} according to claim 18, wherein said chemically modified antibody fragment comprises at least one said inter-chain bridging moiety of the formula (IB_{F}) that is linked to a drug or an imaging agent via the nitrogen atom at the 1-position and to a half-life-extending agent via the nitrogen atom at the 2-position.

22. A process for producing a chemically modified antibody fragment according to claim 18, which process comprises:
reducing at least one inter-chain disulfide bridge of an antibody fragment in the presence of a reducing agent; and
reacting said antibody fragment with at least one inter-chain bridging reagent comprising a moiety of the formula (IIA) or at least one inter-chain bridging reagent comprising a moiety of the formula (IIB) 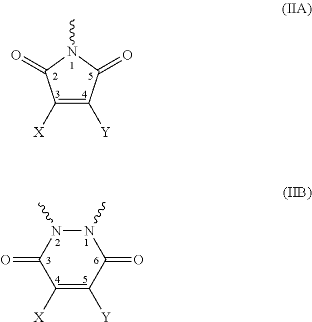
wherein X and Y each independently represent an electrophilic leaving group;
thereby introducing the desired number of inter-chain bridging moieties of the formula (IA_{F}) or (IB_{F}) at the desired locations of said antibody fragment and producing said chemically modified antibody fragment.

23. A chemically modified antibody fragment AB_{F} that:
(i) is capable of specific binding to an antigen AG;
(ii) comprises at least two chains; and
(iii) comprises at least one inter-chain bridging moiety of the formula (III_{F}) 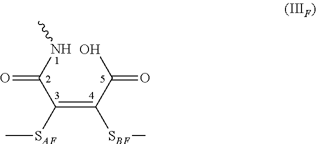
wherein S_{AF} and S_{BF} are sulfur atoms that are attached to different chains of said chemically modified antibody fragment.

24. A composition comprising one or more chemically modified antibodies as defined in claim 1 and which are each capable of binding to the antigen AG, wherein a specific chemically modified antibody of said one or more chemically modified antibodies is:
(i) present in a greater amount by weight than any other of the said one or more chemically modified antibodies; and
(ii) present in an amount of at least 30% by weight of the total amount of said one or more chemically modified antibodies.

25. A composition according to claim 24, wherein said specific chemically modified antibody of said one or more chemically modified antibodies is present in an amount of at least 50% by weight of the total amount of said one or more chemically modified antibodies.

26. (canceled)

27. (canceled)
